(19) United States
(12) Patent Application Publication
(10) Pub. No.: US 2024/0254090 A1
(43) Pub. Date: Aug. 1, 2024

(54) COMPOUNDS WITH ARYLAMINE STRUCTURES

(71) Applicant: Merck Patent GmbH, Darmstadt (DE)

(72) Inventors: Amir PARHAM, Frankfurt am Main (DE); Dominik JOOSTEN, Frankfurt Am Main (DE); Aurélie LUDEMANN, Frankfurt am Main (DE); Tobias GROSSMANN, Darmstadt (DE); Jonas KROEBER, Frankfurt am Main (DE)

(21) Appl. No.: 18/414,554

(22) Filed: Jan. 17, 2024

Related U.S. Application Data
(62) Division of application No. 16/492,634, filed on Sep. 10, 2019, now abandoned.

(30) Foreign Application Priority Data
Mar. 13, 2017 (EP) 17160526.4

(51) Int. Cl. C07D 239/94; C07D 239/84; C07D 401/04; C07D 403/04; C07D 405/04; C07D 405/12; C07D 405/14; C07D 471/04; C07D 491/04; H10K 50/16; H10K 85/60

(52) U.S. Cl. CPC C07D 239/94; C07D 239/84; C07D 401/04; C07D 403/04; C07D 405/04; C07D 405/12; C07D 405/14; C07D 471/04; C07D 491/04; H10K 85/633; H10K 85/636; H10K 85/6572; H10K 50/16

(57) ABSTRACT

The present invention describes arylamine derivatives which are substituted by diazanaphthalene groups, in particular for use in electronic devices. The Invention furthermore relates to a process for the preparation of the compounds according to the Invention and to electronic devices containing same.

The present invention describes arylamine derivatives which are substituted by diazanaphthalene groups, in particular for use in electronic devices. The invention furthermore relates to a process for the preparation of the compounds according to the invention and to electronic devices containing these compounds.

The emitting materials employed in organic electroluminescent devices (OLEDs) are frequently organometallic complexes which exhibit phosphorescence. For quantum-mechanical reasons, an up to four-fold increase in energy and power efficiency is possible using organometallic compounds as phosphorescence emitters. In general, there is still a need for improvement, for example with respect to efficiency, operating voltage and lifetime, in OLEDs, in particular also in OLEDs which exhibit phosphorescence.

The properties of organic electroluminescent devices are not determined only by the emitters employed. In particular, the other materials used, such as host and matrix materials, hole-blocking materials, electron-transport materials, hole-transport materials and electron- and exciton-blocking materials, are also of particular importance here. Improvements in these materials can result in significant improvements in electroluminescent devices.

In accordance with the prior art, the matrix materials employed for phosphorescent compounds and the electron-transport materials are frequently heteroaromatic compounds, such as, for example, quinazoline derivatives. Furthermore, the matrix materials used are also triarylamine derivatives, where compounds which contain both triarylamine structures and also groups derived from quinazoline are also known. However, these compounds do not necessarily contain a substitution by an aryl or heteroaryl group on at least two of the aryl radicals derived from the triarylamine structure which are bonded to the nitrogen atom. Furthermore, not all these compounds are substituted by a further aryl or heteroaryl group on the ring of the diazanaphthalene structure to which the diarylamine group is bonded. Furthermore, some of the compounds form carbazole groups with an aryl radical which is bonded to the nitrogen atom of the diarylamine group.

In general, there is still a need for improvement in these materials, in particular with respect to the lifetime, but also with respect to the efficiency and the operating voltage of the device, for example for use as matrix materials, hole-transport materials or electron-transport materials.

The object of the present invention is therefore the provision of compounds which are suitable for use in an organic electronic device, in particular in an organic electroluminescent device, and which lead to good device properties on use in this device, and the provision of the corresponding electronic device.

In particular, the object of the present invention is to provide compounds which lead to a long lifetime, good efficiency and a low operating voltage. The properties of, in particular, the matrix materials, the hole-transport materials or the electron-transport materials have a significant influence on the lifetime and efficiency of the organic electroluminescent device.

A further object of the present invention can be regarded as being the provision of compounds which are suitable for use in a phosphorescent or fluorescent OLED, in particular as matrix material. In particular, it is an object of the present invention to provide matrix materials which are suitable for red-, yellow- and green-phosphorescent OLEDs.

Furthermore, the compounds should, in particular when used as matrix materials, as hole-transport materials or as electron-transport materials in organic electroluminescent devices, lead to devices which have excellent colour purity.

Furthermore, the compounds should be as easy to process as possible, in particular exhibit good solubility and film formation. For example, the compounds should exhibit increased oxidation stability and an improved glass-transition temperature.

A further object can be regarded as being the provision of electronic devices having excellent performance as inexpensively as possible and in constant quality

Furthermore, it should be possible for the electronic devices to be employed or adapted for many purposes. In particular, the performance of the electronic devices should be retained over a broad temperature range.

Surprisingly, it has been found that certain compounds, described in greater detail below, achieve these objects and overcome the disadvantage from the prior art. The use of the compounds leads to very good properties of organic electroluminescent devices, in particular with respect to the lifetime, the efficiency and the operating voltage. The present invention therefore relates to electronic devices, in particular organic electroluminescent devices, which contain compounds of this type, and to the corresponding preferred embodiments.

The present invention therefore relates to a compound containing at least one structure of the following formula (I),

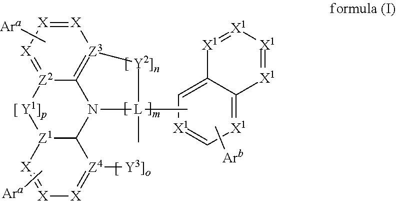

where the following applies to the symbols used:
L is an aromatic or heteroaromatic ring system having 5 to 30 aromatic ring atoms, which may be substituted by one or more non-aromatic radicals R¹;
Z¹, Z², Z³, Z⁴ are, identically or differently, X or C;
Y¹ is BR¹, Si(R¹)₂, NR¹, O, S, S═O and S(═O)₂, where the group NR¹ is preferably not equal to NH, the group BR¹ is preferably not equal to BH and the group Si(R¹)₂ is preferably not equal to Si(H)₂ or SiHR¹;
Y², Y³ are on each occurrence, identically or differently, BR¹, Si(R¹)₂, C(R¹)₂, NR¹, O, S, S═O and S(═O)₂, where the group NR¹ is preferably not equal to NH, the group BR¹ is preferably not equal to BH and the group Si(R¹)₂ is preferably not equal to Si(H)₂ or SiHR¹;
X is on each occurrence, identically or differently, N or CR¹, preferably CR¹, or C if a radical Ar^{a} is bonded to X;
X¹ is on each occurrence, identically or differently, N or CR¹;
m, n, o, p are 0 or 1;
Ar^{a} is on each occurrence, identically or differently, an aromatic or heteroaromatic ring system having 5 to 30 aromatic ring atoms, which may be substituted by one or more radicals R¹, where the radical Ar^{a} does not contain a carbazole group or form a carbazole group with the aryl or heteroaryl group to which Ar^{a} is bonded, including substituents R¹, R² and R³ which may be bonded to the radical Ar^{a};
Ar^{b} is an aromatic or heteroaromatic ring system having 5 to 45 aromatic ring atoms, which may be substituted by one or more radicals R¹;
R¹ is on each occurrence, identically or differently, H, D, F, Cl, Br, I, CN, NO₂, N(Ar¹)₂, N(R²)₂, C(═O)Ar¹, C(═O)R², P(═O)(Ar¹)₂, P(Ar¹)₂, B(Ar¹)₂, Si(Ar¹)₃, Si(R²)₃, a straight-chain alkyl, alkoxy or thioalkoxy group having 1 to 40 C atoms or a branched or cyclic alkyl, alkoxy or thioalkoxy group having 3 to 40 C atoms or an alkenyl group having 2 to 40 C atoms, which may in each case be substituted by one or more radicals R², where one or more non-adjacent CH₂ groups may be replaced by —R²C═CR²—, —C≡C—, Si(R²)₂, C═O, C═S, C═NR², —C(═O)O—, —C(═O)NR²—, NR², P(═O)(R²), —O—, —S—, SO or SO₂ and where one or more H atoms may be replaced by D, F, Cl, Br, I, CN or NO₂, or an aromatic or heteroaromatic ring system having 5 to 40 aromatic ring atoms, which may in each case be substituted by one or more radicals R², or an aryloxy or heteroaryloxy group having 5 to 40 aromatic ring atoms, which may be substituted by one or more radicals R², or an aralkyl or heteroaralkyl group having 5 to 40 aromatic ring atoms, which may be substituted by one or more radicals R², or a combination of these systems; two or more, preferably adjacent substituents R¹ may also form a mono- or polycyclic, aliphatic or aromatic ring system with one another;
Ar¹ is on each occurrence, identically or differently, an aromatic or heteroaromatic ring system having 5 to 30 aromatic ring atoms, which may be substituted by one or more non-aromatic radicals R²; two radicals Ar¹ which are bonded to the same Si atom, N atom, P atom or B atom may also be bridged to one another by a single bond or a bridge selected from B(R²), C(R²)₂, Si(R²)₂, C═O, C═NR², C═C(R²)₂, O, S, S═O, SO₂, N(R²), P(R²) and P(═O)R²;
R² is on each occurrence, identically or differently, H, D, F, Cl, Br, I, CN, B(OR³)₂, NO₂, C(═O)R³, CR³═C(R³)₂, C(═O)OR³, C(═O)N(R³)₂, Si(R³)₃, P(R³)₂, B(R³)₂, N(R³)₂, NO₂, P(═O)(R³)₂, OSO₂R³, OR³, S(═O)R³, S(═O)₂R³, a straight-chain alkyl, alkoxy or thioalkoxy group having 1 to 40 C atoms or a branched or cyclic alkyl, alkoxy or thioalkoxy group having 3 to 40 C atoms, which may in each case be substituted by one or more radicals R³, where one or more non-adjacent CH₂ groups may be replaced by —R³C═CR³—, —C═C—, Si(R³)₂, C═O, C═S, C═NR³, —C(═O)O—, —C(═O)NR³—, NR³, P(═O)(R³), —O—, —S—, SO or SO₂ and where one or more H atoms may be replaced by D, F, Cl, Br, I, CN or NO₂, or an aromatic or heteroaromatic ring system having 5 to 40 aromatic ring atoms, which may in each case be substituted by one or more radicals R³, or an aryloxy or heteroaryloxy group having 5 to 40 aromatic ring atoms, which may be substituted by one or more radicals R³, or a combination of these systems; two or more, preferably adjacent substituents R² may also form a mono- or polycyclic, aliphatic or aromatic ring system with one another;
R³ is selected on each occurrence, identically or differently, from the group consisting of H, D, F, CN, an aliphatic hydrocarbon radical having 1 to 20 C atoms or an aromatic or heteroaromatic ring system having 5 to 30 aromatic ring atoms, in which one or more H atoms may be replaced by D, F, Cl, Br, I or CN and which may be substituted by one or more alkyl groups, each having 1 to 4 carbon atoms; two or more, preferably adjacent substituents R³ may form a mono- or polycyclic, aliphatic ring system with one another;
with the proviso that at least two groups X¹ in formula (I) stand for N, where, in the case where p=1, Z¹, Z² represent C and, in the case where n=1, Z³ represents C, and, in the case where o=1, Z⁴ represents C, and
with the proviso that compounds of the formula (A)

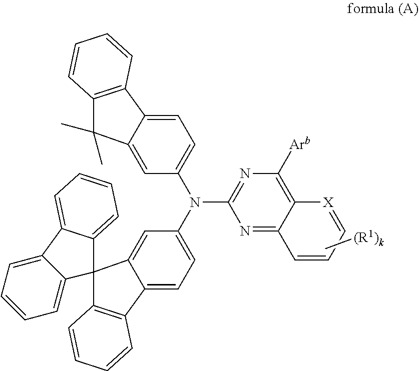

are excluded, where the symbols X, Ar^{b} and R¹ used have the meaning given above and k is 0 or 1.

It can be seen from the above formulation that, if the index n, o, p=0, the corresponding bridge Y¹, Y², Y³ is not present and the corresponding symbol Z¹, Z², Z³, Z⁴ in this case stands for X. By contrast, m=0 means that a single bond is present between the nitrogen atom and the heteroaromatic ring system. Particularly preferably, m=0. This means that the diazanaphthalene group or quinazoline group is bonded directly to the nitrogen atom of the diarylamine group.

Furthermore, it can be seen from the above structure of the formula (I) that the group Ar^{b} and the group L are bonded to the same ring of the diazanaphthyl group, where the two bonding sites on the diazanaphthyl group are not adjacent, but instead are separated by a group X¹. The group L is preferably bonded to a site on the diazanaphthyl group which is adjacent to the N atom of the diazanaphthyl group, so that the symbol X¹ that is adjacent to the bonding site of the group L stands for a nitrogen atom. The group Ar^{b} is preferably bonded to a site on the diazanaphthyl group that is adjacent to the N atom of the diazanaphthyl group, so that the symbol X¹ that is adjacent to the bonding site of the group Ar^{b} stands for a nitrogen atom.

Adjacent carbon atoms in the sense of the present invention are carbon atoms which are linked directly to one another. Furthermore, “adjacent radicals” in the definition of the radicals means that these radicals are bonded to the same carbon atom or to adjacent carbon atoms. These definitions apply correspondingly, inter alia, to the terms “adjacent groups” and “adjacent substituents”.

The formulation that two or more radicals can form a ring with one another is, for the purposes of the present description, intended to be taken to mean, inter alia, that the two radicals are linked to one another by a chemical bond with formal elimination of two hydrogen atoms. This is illustrated by the following scheme:

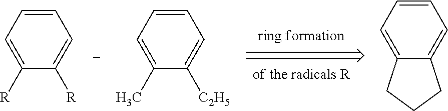

Furthermore, however, the above-mentioned formulation is also intended to be taken to mean that, in the case where one of the two radicals represents hydrogen, the second radical is bonded at the position at which the hydrogen atom was bonded, with formation of a ring. This is intended to be illustrated by the following scheme:

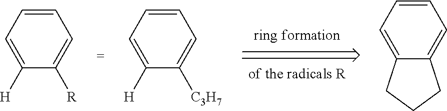

A condensed aryl group, a condensed aromatic ring system or a condensed heteroaromatic ring system in the sense of the present invention is a group in which two or more aromatic groups are condensed onto one another via a common edge, i.e. anellated, so that, for example, two C atoms belong to the at least two aromatic or heteroaromatic rings, as, for example, in naphthalene. By contrast, for example, fluorene is not a condensed aryl group in the sense of the present invention since the two aromatic groups in fluorene do not have a common edge. Corresponding definitions apply to heteroaryl groups and to condensed ring systems, which may also contain heteroatoms, but do not have to do so.

An aryl group in the sense of this invention contains 6 to 40 C atoms; a heteroaryl group in the sense of this invention contains 2 to 40 C atoms and at least one heteroatom, with the proviso that the sum of C atoms and heteroatoms is at least 5. The heteroatoms are preferably selected from N, O and/or S. An aryl group or heteroaryl group here is taken to mean either a simple aromatic ring, i.e. benzene, or a simple heteroaromatic ring, for example pyridine, pyrimidine, thiophene, etc., or a condensed aryl or heteroaryl group, for example naphthalene, anthracene, phenanthrene, quinoline, isoquinoline, etc.

An aromatic ring system in the sense of this invention contains 6 to 40 C atoms in the ring system. A heteroaromatic ring system in the sense of this invention contains 1 to 40 C atoms and at least one heteroatom in the ring system, with the proviso that the sum of C atoms and heteroatoms is at least 5. The heteroatoms are preferably selected from N, O and/or S. An aromatic or heteroaromatic ring system in the sense of this invention is intended to be taken to mean a system which does not necessarily contain only aryl or heteroaryl groups, but instead in which, in addition, a plurality of aryl or heteroaryl groups may be interrupted by a non-aromatic unit (preferably less than 10% of the atoms other than H), such as, for example, a C, N or O atom or a carbonyl group. Thus, for example, systems such as 9,9′-spirobifluorene, 9,9-diarylfluorene, triarylamine, diaryl ether, stilbene, etc., are also intended to be taken to be aromatic ring systems in the sense of this invention, as are systems in which two or more aryl groups are interrupted, for example, by a linear or cyclic alkyl group or by a silyl group. Furthermore, systems in which two or more aryl or heteroaryl groups are bonded directly to one another, such as, for example, biphenyl, terphenyl, quaterphenyl or bipyridine, are likewise intended to be taken to be an aromatic or heteroaromatic ring system.

A cyclic alkyl, alkoxy or thioalkoxy group in the sense of this invention is taken to mean a monocyclic, bicyclic or polycyclic group.

For the purposes of the present invention, a C₁- to C₂₀-alkyl group, in which, in addition, individual H atoms or CH₂ groups may be substituted by the above-mentioned groups, is taken to mean, for example, the radicals methyl, ethyl, n-propyl, i-propyl, cyclopropyl, n-butyl, i-butyl, s-butyl, t-butyl, cyclobutyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2.2.2]octyl, 2-bicyclo[2.2.2]octyl, 2-(2,6-dimethyl)octyl, 3-(3,7-dimethyl)octyl, adamantyl, trifluoromethyl, pentafluoroethyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-ntetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)cyclohex-1-yl, 1-(n-butyl)cyclohex-1-yl, 1-(n-hexyl)cyclohex-1-yl, 1-(n-octyl)cyclohex-1-yl and 1-(n-decyl)cyclohex-1-yl. An alkenyl group is taken to mean, for example, ethenyl, propenyl, butenyl, pentenyl, cyclopentenyl, hexenyl, cyclohexenyl, heptenyl, cycloheptenyl, octenyl, cyclooctenyl or cyclooctadienyl. An alkynyl group is taken to mean, for example, ethynyl, propynyl, butynyl, pentynyl, hexynyl, heptynyl or octynyl. A C₁- to C₄₀-alkoxy group is taken to mean, for example, methoxy, trifluoromethoxy, ethoxy, n-propoxy, i-propoxy, n-butoxy, i-butoxy, s-butoxy, t-butoxy or 2-methylbutoxy.

An aromatic or heteroaromatic ring system having 5-40 aromatic ring atoms, which may also in each case be substituted by the radicals mentioned above and which may be linked to the aromatic or heteroaromatic ring system via any desired positions, is taken to mean, for example, groups derived from benzene, naphthalene, anthracene, benzanthracene, phenanthrene, benzophenanthrene, pyrene, chrysene, perylene, fluoranthene, benzofluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or transindenofluorene, trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

In a preferred embodiment, the compounds according to the invention may contain a structure of the formula (IIa) and/or (IIb),

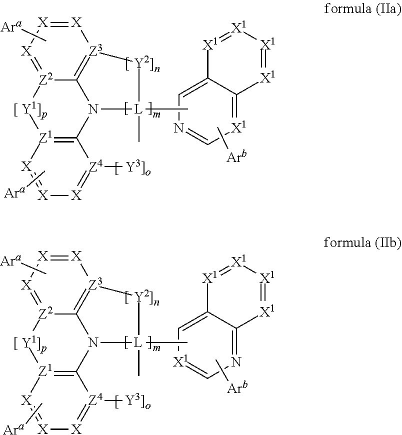

where the symbols m, n, o, p, L, Ar^{a}, Ar^{b}, Y¹, Y², Y, Z¹, Z², Z³, Z⁴, X and X¹ used have the meaning given above, in particular for formula (I), and at least one group X¹ stands for N. Preferably, a maximum of two groups X or X¹ per ring stand for N.

The compounds according to the invention may preferably contain structures of the formula (IIIa) and/or (IIIb),

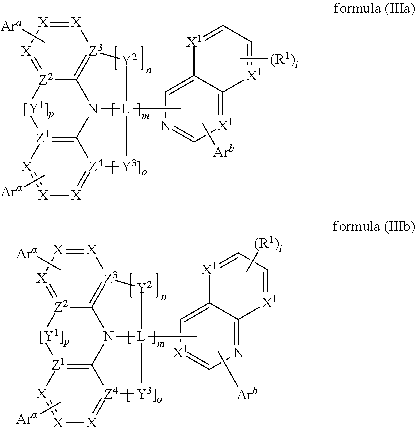

where the symbols m, n, o, p, L, R¹, Ar^{a}, Ar^{b}, Y¹, Y², Y³, Z¹, Z², Z³, Z⁴, X and X¹ used have the meaning given above, in particular for formula (I), at least one group X¹ stands for N and i stands for 0, 1 or 2, preferably for 0 or 1. Preferably, a maximum of two groups X per ring stand for N.

Furthermore, preference is given to compounds containing structures of the formula (IIa), (IIb), (IIIa) or (IIIb) in which at least two, preferably at least three, of the symbols X stand for CR¹, particularly preferably at least three of the symbols X are selected from C—H and C-D.

The compounds according to the invention may preferably contain at least one structure of the formula (IVa) and/or (IVb),

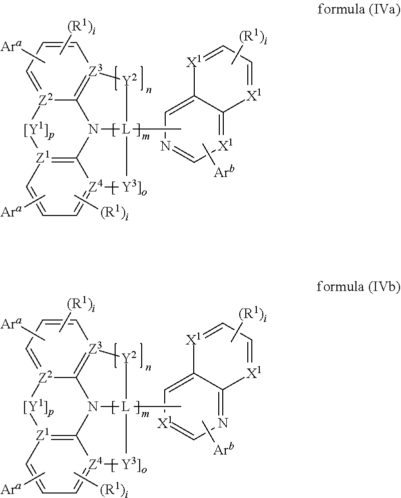

where the symbols m, n, o, p, L, R¹, Ar^{a}, Ar^{b}, Y¹, Y², Y, Z¹, Z², Z³, Z⁴ and X¹ used have the meaning described above, in particular for formula (I), at least one group X¹ stands for N and i stands for 0, 1 or 2, preferably for 0 or 1.

The compounds according to the invention may preferably contain at least one structure of the formula (Va) and/or (Vb),

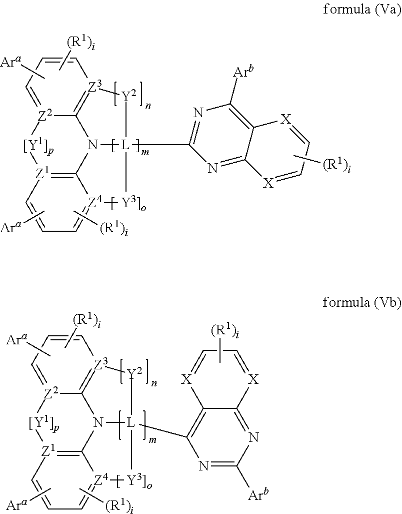

where the symbols m, n, o, p, L, R¹, Ar^{a}, Ar^{b}, Y¹, Y², Y³, Z¹, Z², Z³, Z⁴ and X used have the meaning described above, in particular for formula (I), and i stands for 0, 1 or 2, preferably for 0 or 1.

In addition, preference is given to compounds containing structures of the formulae (I), (IIa), (IIb), (IIIa), (IIIb), (IVa), (IVb), (Va), (Vb) in which the group Y¹ stands for BR¹, Si(R¹)₂, NR¹, O, S, S═O or S(═O)₂, where the group NR¹ is not equal to NH, the group BR¹ is not equal to BH and the group Si(R¹)₂ is not equal to Si(H)₂ or SiHR¹.

Furthermore, it may be provided that, if n=o=0 in the structures of the formulae (I), (IIa), (IIb), (IIIa), (IIIb), (IVa), (IVb), (Va), (Vb), the index p=0, so that the groups Z¹, Z², Z³, Z⁴ stand for X.

In a furthermore preferred embodiment, the compounds according to the invention may contain at least one structure of the formula (VIa) and/or (VIb),

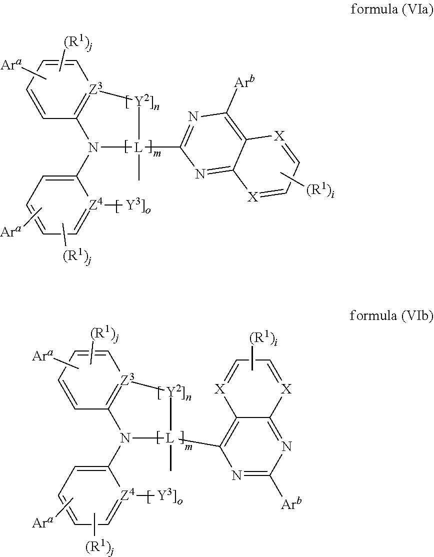

where the symbols m, n, o, L, R¹, Ar^{a}, Ar^{b}, Y¹, Y², Y³, Z³, Z⁴ and X used have the meaning given above, in particular for formula (I), i stands for 0, 1 or 2, preferably for 0 or 1, and j stands for 0, 1, 2 or 3, preferably for 0, 1 or 2, particularly preferably for 0 or 1.

Furthermore, it may be provided that compounds according to the invention contain at least one structure of the formula (VIIa) and/or (VIIb),

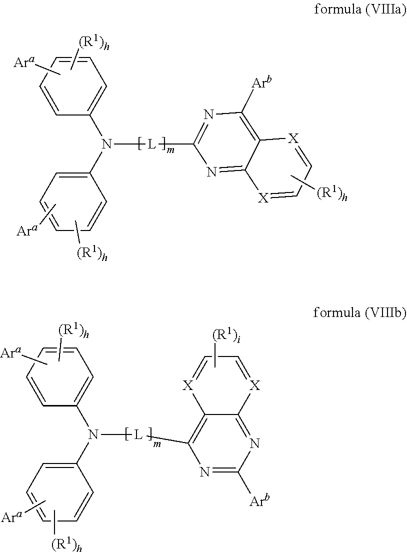

where the symbols m, L, Ar^{a}, Ar^{b}, R¹ and X used have the meaning described above, in particular for formula (I), i stands for 0, 1 or 2, preferably for 0 or 1, and h stands for 0, 1, 2, 3 or 4, preferably for 0, 1 or 2, particularly preferably for 0 or 1.

Furthermore, preference is given to compounds containing at least one structure of the formula (VIIIa) and/or (VIIIb),

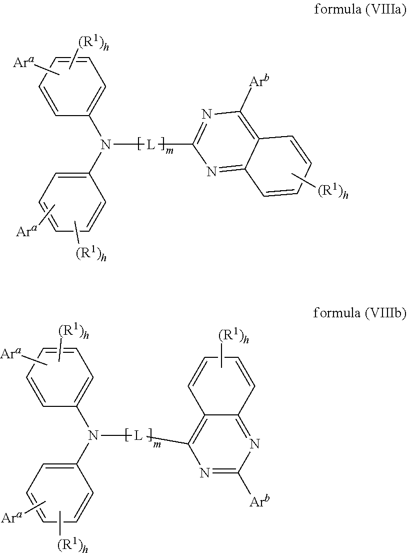

where the symbols m, L, Ar^{a}, Ar^{b} and R¹ used have the meaning described above, in particular for formula (I), and h stands for 0, 1, 2, 3 or 4, preferably for 0, 1 or 2, particularly preferably for 0 or 1.

The radical Ar^{a}, in particular in the formulae (I), (IIa), (IIb), (IIIa), (IIIb), (IVa), (IVb), (Va), (Vb), (VIa), (VIb), (VIIa), (VIIb), (VIIIa), (VIIIb), may contain a spirobifluorene, fluorene, dibenzofuran or dibenzothiophene group or form one of these groups with the aryl or heteroaryl radical to which the radical Ar^{a} is bonded.

In a furthermore preferred embodiment, the compounds according to the invention may contain at least one structure of the formula (IXa) or (IXb),

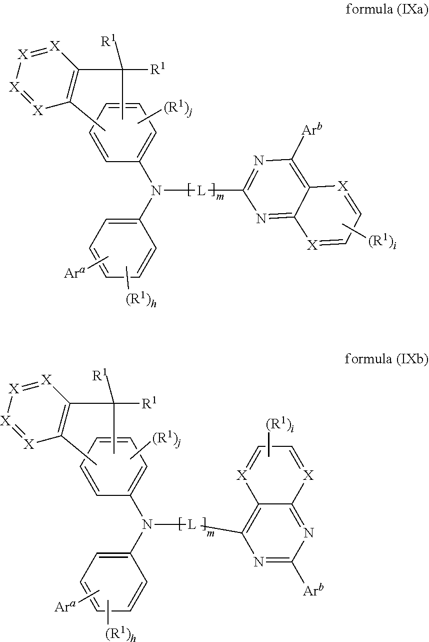

where the symbols m, L, Ar^{a}, Ar^{b}, R¹ and X used have the meaning described above, in particular for formula (I), j stands for 0, 1, 2 or 3, preferably for 0, 1 or 2, particularly preferably for 0 or 1, and h stands for 0, 1, 2, 3 or 4, preferably for 0, 1 or 2, particularly preferably for 0 or 1, and i stands for 0, 1 or 2, preferably for 0 or 1.

Furthermore, the compounds according to the Invention may contain at least one structure of the formula (Xa) or (Xb),

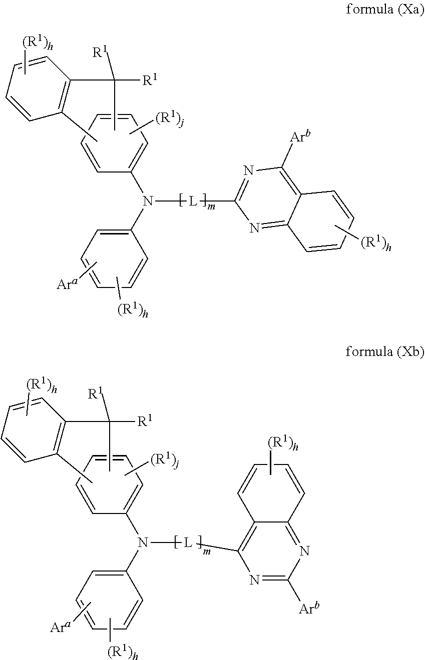

where the symbols m, L, Ar^{a}, Ar^{b} and R¹ used have the meaning described above, in particular for formula (I), j stands for 0, 1, 2 or 3, preferably for 0, 1 or 2, particularly preferably for 0 or 1, and h stands for 0, 1, 2, 3 or 4, preferably for 0, 1 or 2, particularly preferably for 0 or 1.

In a further preferred embodiment, the compounds according to the Invention may contain at least one structure of the formula (XIa), (XIb), (XIc), (XId), (XIe) or (XIf),

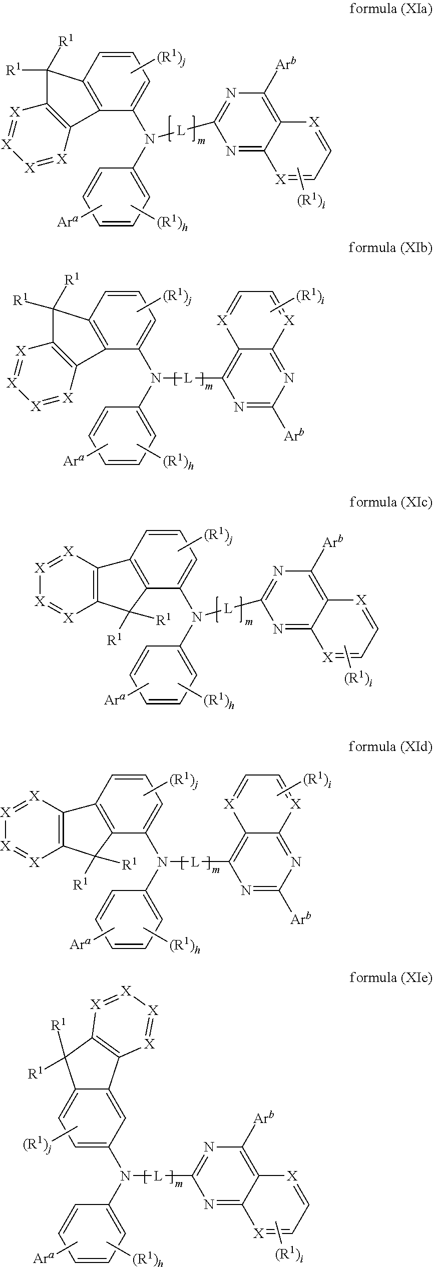

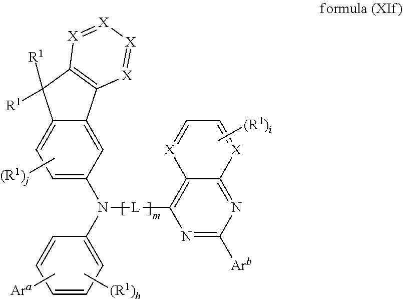

where the symbols m, L, Ar^{a}, Ar^{b}, R¹ and X used have the meaning described above, in particular for formula (I), i stands for 0, 1 or 2, preferably for 0 or 1, j stands for 0, 1, 2 or 3, preferably for 0, 1 or 2, particularly preferably for 0 or 1, and h stands for 0, 1, 2, 3 or 4, preferably for 0, 1 or 2, particularly preferably for 0 or 1.

Furthermore, it may be provided that compounds according to the invention contain at least one structure of the formula (XIIa) or (XIIb),

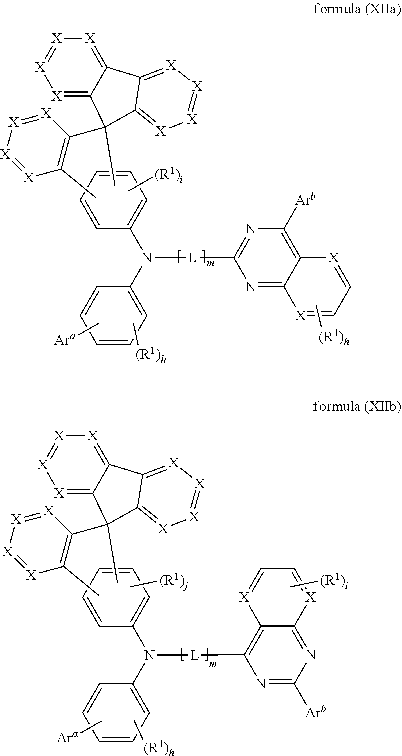

where the symbols m, L, Ar^{a}, Ar^{b}, R¹ and X used have the meaning described above, in particular for formula (I), i stands for 0, 1 or 2, preferably for 0 or 1, j stands for 0, 1, 2 or 3, preferably for 0, 1 or 2, particularly preferably for 0 or 1, and h stands for 0, 1, 2, 3 or 4, preferably for 0, 1 or 2, particularly preferably for 0 or 1.

The group L may form a continuous conjugation with the diazanaphthalene radical to which the group L is bonded in the formulae (I), (IIa), (IIb), (IIIa), (IIIb), (IVa), (IVb), (Va), (Vb), (VIa), (VIb), (VIIila), (VIIb), (VIIIa), (VIIIb), (IXa), (IXb), (Xa), (Xb), (XIa), (XIb), (XIc), (XId), (XIe), (XIf), (XIIa), (XIIb), and with the diarylamine group in these formulae. In a further preferred embodiment of the invention, L stands for an aromatic or heteroaromatic ring system having 5 to 14 aromatic or heteroaromatic ring atoms, preferably an aromatic or heteroaromatic ring system having 6 to 13 carbon atoms, which may be substituted by one or more radicals R¹, but is preferably unsubstituted, where R¹ can have the meaning given above, in particular for formula (I). L particularly preferably stands for an aromatic ring system having 6 to 10 aromatic ring atoms or a heteroaromatic ring system having 6 to 13 heteroaromatic ring atoms, which may in each case be substituted by one or more radicals R¹, but is preferably unsubstituted, where R¹ can have the meaning given above, in particular for formula (I).

The symbol L described, inter alia, in the structures of the formulae (I), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIc), (XId), (XIe), (XIf), (XIIa) and/or (XIIb) furthermore preferably stands, identically or differently on each occurrence, for a bond, which corresponds to m=0, or an aryl or heteroaryl radical having 5 to 24 ring atoms, preferably 6 to 13 ring atoms, particularly preferably 6 to 10 ring atoms, so that an aromatic or heteroaromatic group of an aromatic or heteroaromatic ring system is bonded directly, i.e. via an atom of the aromatic or heteroaromatic group, to the respective atom of the further group.

Furthermore, it may be provided that the group L described, inter alia, in the structures of the formulae (I), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIc), (XId), (XIe), (XIf), (XIIa) and/or (XIIb) contains an aromatic ring system having at most two condensed aromatic and/or heteroaromatic six-membered rings, preferably no condensed aromatic or heteroaromatic ring system. Accordingly, naphthyl structures are preferred to anthracene structures. Furthermore, fluorenyl, spirobifluorenyl, dibenzofuranyl and/or dibenzothienyl structures are preferred to naphthyl structures.

Particular preference is given to structures which have no condensation, such as, for example, phenyl, biphenyl, terphenyl and/or quaterphenyl structures. Especially preference for the group L described in formulae (I), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIc), (XId), (XIe), (XIf), (XIIa) and/or (XIIb) is given to phenyl, biphenyl, dibenzofuranyl and/or dibenzothienyl structures, which may be substituted by one or more radicals R¹, as defined above in formula (I). Examples of suitable aromatic or heteroaromatic ring systems L are selected from the group consisting of ortho-, meta- or para-phenylene, ortho-, meta- or para-biphenylene, terphenylene, in particular branched terphenylene, quaterphenylene, in particular branched quaterphenylene, fluorenylene, spirobifluorenylene, dibenzofuranylene, dibenzothienylene and carbazolylene, which may in each case be substituted by one or more radicals R², but are preferably unsubstituted.

Furthermore, it may be provided that the group L described, inter alia, in the structures of the formulae (I), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIc), (XId), (XIe), (XIf), (XIIa) and/or (XIIb) contains at most 1 nitrogen atom, preferably at most 2 heteroatoms, particularly preferably at most one heteroatom and particularly preferably no heteroatom.

Preference is given to compounds containing structures of the formulae (I), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIc), (XId), (XIe), (XIf), (XIIa) and/or (XIIb) in which the group L stands for a group selected from the formulae (L-1) to (L-15),

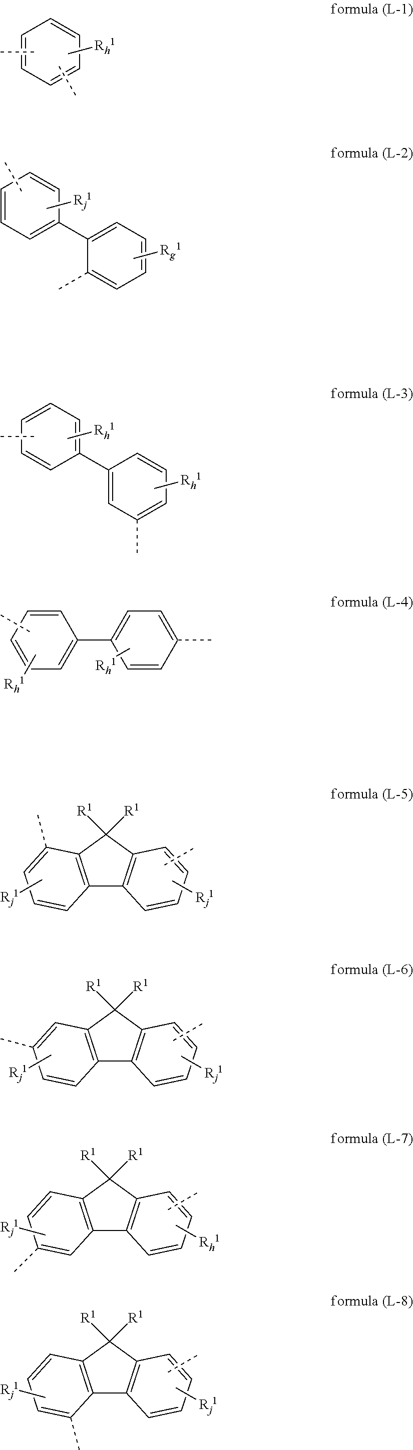

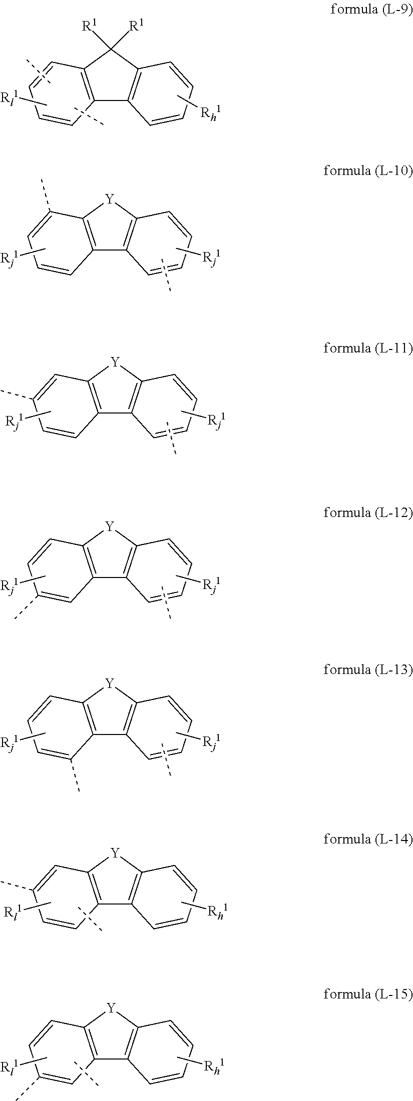

where the dashed bonds in each case mark the bonding positions, the index I is 0, 1 or 2, the index j is on each occurrence, independently, 0, 1, 2 or 3, the index h is on each occurrence, independently, 0, 1, 2, 3 or 4, the index g is 0, 1, 2, 3, 4 or 5, the symbol Y is O, S or NR², preferably O or S, and the symbol R¹ has the meaning given above, in particular for formula (I).

Preference is given here, in particular, to the formulae (L-1) to (L-4) and (L-10) to (L-15), where Y in the preferred formulae (L-10) to (L-15) stands for O or S. It may preferably be provided that the sum of the indices k, I, g, h and j in the structures of the formula (L-1) to (L-15) is in each case at most 3, preferably at most 2 and particularly preferably at most 1.

In a preferred embodiment, the compounds according to the invention may contain a structure of the formula (XIII),

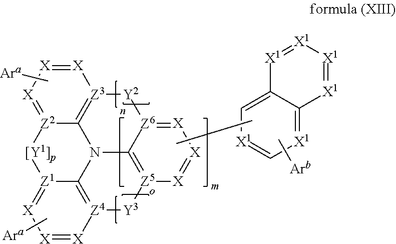

where Z⁵ and Z⁶ stand, identically or differently, for X or C, the symbols m, n, o, p, Ar^{a}, Ar^{b}, Y¹, Y², Y³, Z¹, Z², Z³, Z⁴, X and X¹ used have the meaning given above, in particular for formula (I), and at least two groups X¹ stand for N, where, in the case where n=1, Z³, Z⁶ represent C, and, in the case where o=1, Z⁴, Z⁵ represent C. Preferably, a maximum of two groups X or X¹ per ring stand for N.

The compounds according to the invention may preferably contain at least one structure of the formula (XIVa) and/or (XIVb),

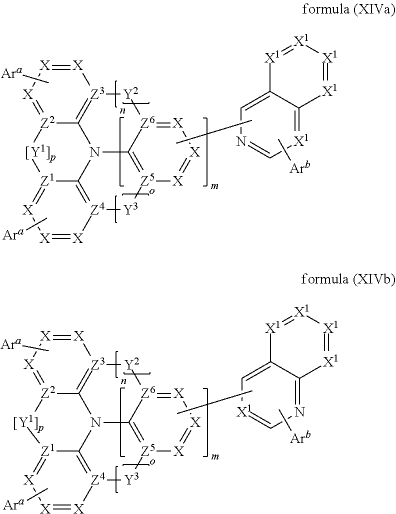

where the symbols m, n, o, p, Ar^{a}, Ar^{b}, Y¹, Y², Y³, Z¹, Z², Z³, Z⁴, Z⁵, Z⁶, X and X¹ used have the meaning given above, in particular for formula (I) or formula (XIII), and at least one group X¹ stands for N. Preferably, a maximum of two groups X or X¹ per ring stand for N.

The compounds according to the invention may preferably contain structures of the formula (XVa) and/or (XVb),

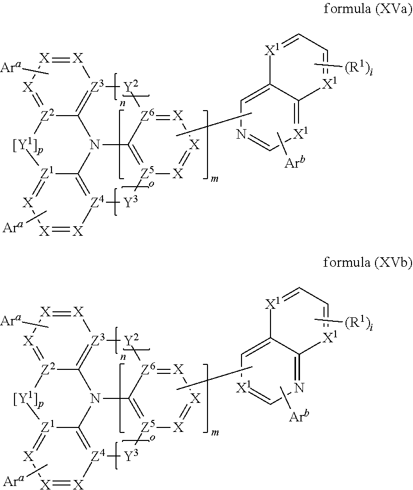

where the symbols m, n, o, p, Ar^{a}, Ar^{b}, Y¹, Y², Y³, Z¹, Z², Z³, Z⁴, Z⁵, Z⁶, X and X¹ used have the meaning given above, in particular for formula (I) or formula (XIII), at least one group X¹ stands for N and i stands for 0, 1 or 2, preferably for 0 or 1. Preferably, a maximum of two groups X per ring stand for N.

Furthermore, preference is given to compounds containing structures of the formula (XIVa), (XIVb), (XVa) or (XVb) in which at least six, preferably at least eight, of the symbols X stand for CR¹, particularly preferably at least six of the symbols X are selected from C—H and C-D.

The compounds according to the invention may preferably contain at least one structure of the formula (XVIa) and/or (XVIb),

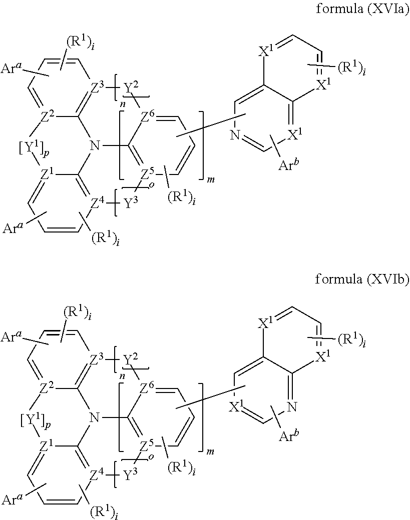

where the symbols m, n, o, p, Ar^{a}, Ar^{b}, Y¹, Y², Y³, Z¹, Z², Z³, Z⁴, Z⁵, Z⁶ and X¹ used have the meaning given above, in particular for formula (I) or formula (XIII), at least one group X¹ stands for N and i stands for 0, 1 or 2, preferably for 0 or 1.

The compounds according to the invention may preferably contain at least one structure of the formula (XVIIa) and/or (XVIIb),

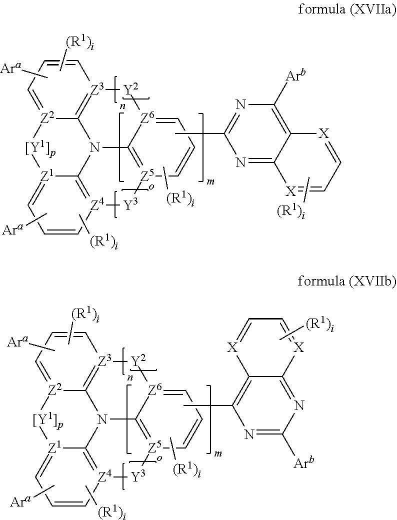

where the symbols m, n, o, p, Ar^{a}, Ar^{b}, Y¹, Y², Y³, Z¹, Z², Z³, Z⁴, Z⁵, Z⁶ and X used have the meaning given above, in particular for formula (I) or formula (XIII), and i stands for 0, 1 or 2, preferably for 0 or 1.

In addition, preference is given to compounds containing structures of the formulae (I), (IIa), (IIb), (IIIa), (IIIb), (IVa), (IVb), (Va), (Vb), (XIII), (XIVa), (XIVb), (XVa), (XVb), (XVIa), (XVIb), (XVIIa), (XVIIb) in which the group Y¹ stands for BR¹, Si(R¹)₂, NR¹, O, S, S═O or S(═O)₂, where the group NR¹ is not equal to NH, the group BR¹ is not equal to BH and the group Si(R¹)₂ is not equal to Si(H)₂ or SiHR¹.

Furthermore, it may be provided that, if n=o=0 in the structures of the formulae (I), (IIa), (IIb), (IIIa), (IIIb), (IVa), (IVb), (Va), (Vb), (XIII), (XIVa), (XIVb), (XVa), (XVb), (XVIa), (XVIb), (XVIIa), (XVIIb), the index p=0, so that the groups Z¹, Z², Z³, Z⁴, Z⁵, Z⁶ stand for X.

In a furthermore preferred embodiment, the compounds according to the invention may contain at least one structure of the formula (XVIIIa) and/or (XVIIIb),

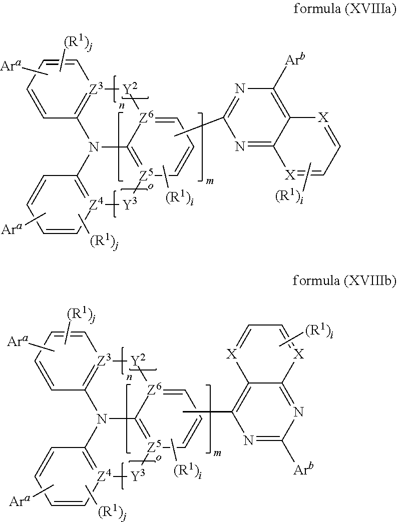

where the symbols m, n, o, Ar^{a}, Ar^{b}, Y¹, Y², Y³, Z³, Z⁴, Z⁵, Z⁶ and X used have the meaning given above, in particular for formula (I) or formula (XIII), i stands for 0, 1 or 2, preferably for 0 or 1, and j stands for 0, 1, 2 or 3, preferably for 0, 1 or 2, particularly preferably for 0 or 1.

Furthermore, it may be provided that the compounds according to the invention contain at least one structure of the formula (XIXa) and/or (XIXb),

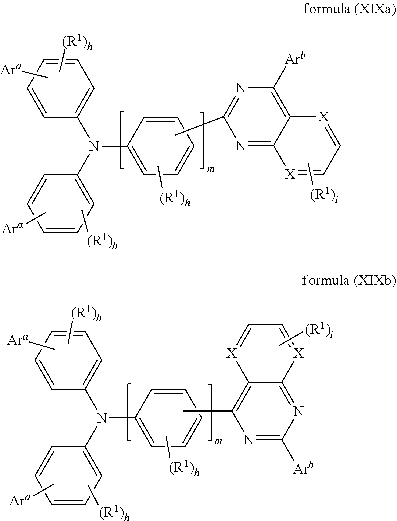

where the symbols m, Ar^{a}, Ar^{b}, R¹ and X used have the meaning described above, in particular for formula (I), i stands for 0, 1 or 2, preferably for 0 or 1, and h stands for 0, 1, 2, 3 or 4, preferably for 0, 1 or 2, particularly preferably for 0 or 1.

Furthermore, preference is given to compounds containing at least one structure of the formula (XXa) and/or (XXb),

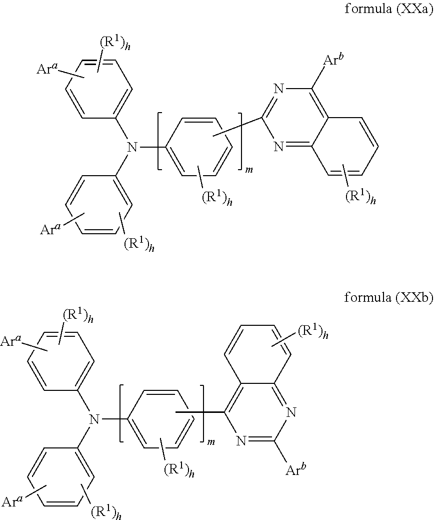

where the symbols m, Ar^{a}, Ar^{b} and R¹ used have the meaning described above, in particular for formula (I), and h stands for 0, 1, 2, 3 or 4, preferably for 0, 1 or 2, particularly preferably for 0 or 1.

The radical Ar^{a}, identically or differently on each occurrence, in particular in the formulae (XIII), (XIVa), (XIVb), (XVa), (XVb), (XVIa), (XVIb), (XVIIa), (XVIIb), (XVIIIa), (XVIIIb), (XIXa), (XIXb), (XXa) and/or (XXb), may preferably contain a spirobifluorene, fluorene, dibenzofuran or dibenzothiophene group or form one of these groups with the aryl or heteroaryl radical to which the radical Ar^{a} is bonded.

In a furthermore preferred embodiment, the compounds according to the invention may comprise at least one structure of the formula (XXIa) or (XXIb),

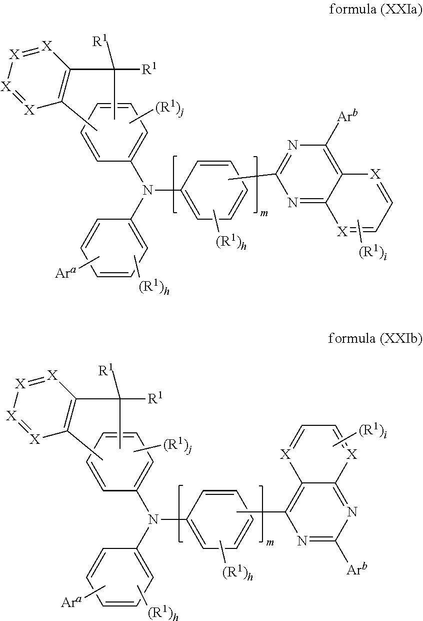

where the symbols m, Ar^{a}, Ar^{b}, R¹ and X used have the meaning described above, in particular for formula (I), j stands for 0, 1, 2 or 3, preferably for 0, 1 or 2, particularly preferably for 0 or 1, h stands for 0, 1, 2, 3 or 4, preferably for 0, 1 or 2, particularly preferably for 0 or 1, and i stands for 0, 1 or 2, preferably for 0 or 1.

Furthermore, the compounds according to the Invention may contain at least one structure of the formula (XXIIa) or (XXIIb),

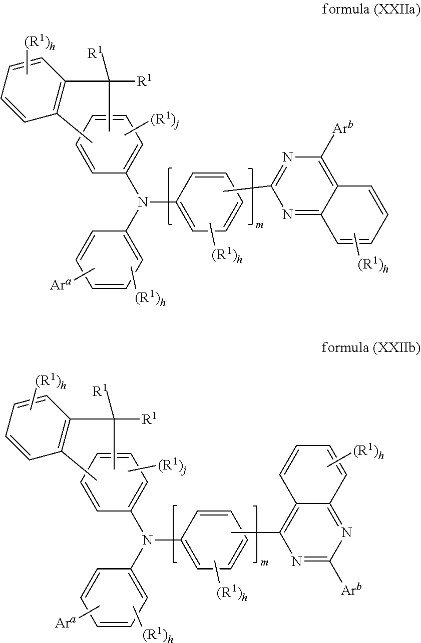

where the symbols m, Ar^{a}, Ar^{b} and R¹ used have the meaning described above, in particular for formula (I), j stands for 0, 1, 2 or 3, preferably for 0, 1 or 2, particularly preferably for 0 or 1, and h stands for 0, 1, 2, 3 or 4, preferably for 0, 1 or 2, particularly preferably for 0 or 1.

In a furthermore preferred embodiment, the compounds according to the invention may contain at least one structure of the formula (XXIIIa), (XXIIIb), (XXIIIc), (XXIIId), (XXIIIe) or (XXIIIf),

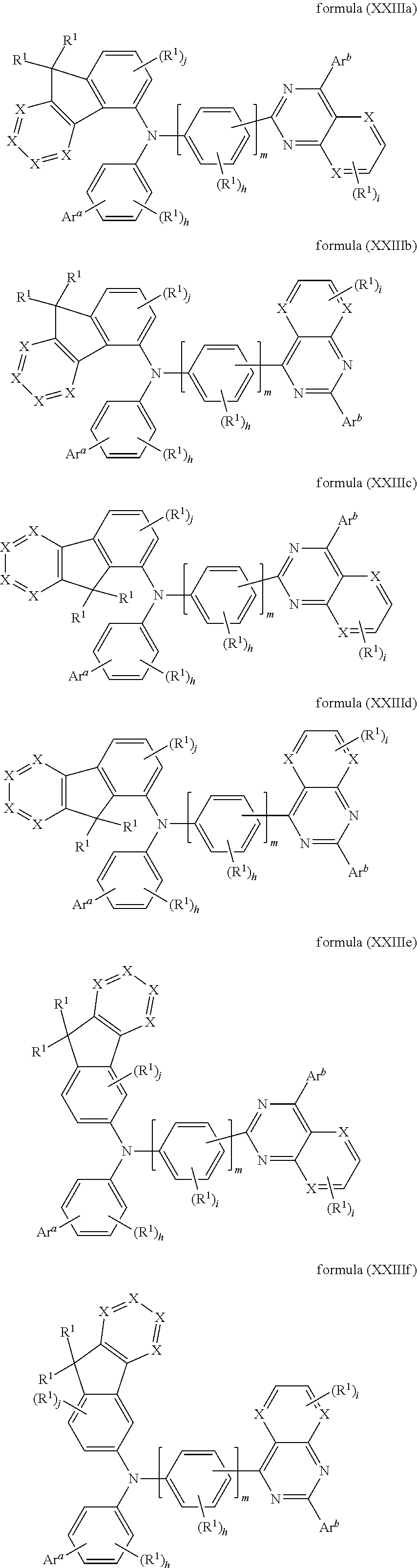

where the symbols m, Ar^{a}, Ar^{b}, R¹ and X used have the meaning described above, in particular for formula (I), i stands for 0, 1 or 2, preferably for 0 or 1, j stands for 0, 1, 2 or 3, preferably for 0, 1 or 2, particularly preferably for 0 or 1, and h stands for 0, 1, 2, 3 or 4, preferably for 0, 1 or 2, particularly preferably for 0 or 1.

Furthermore, it may be provided that compounds according to the invention contain at least one structure of the formula (XXIVa) or (XXIVb),

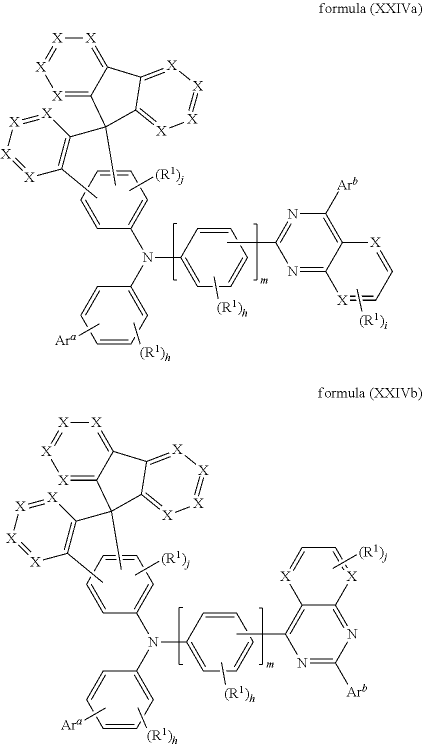

where the symbols m, Ar^{a}, Ar^{b}, R¹ and X used have the meaning described above, in particular for formula (I), i stands for 0, 1 or 2, preferably for 0 or 1, j stands for 0, 1, 2 or 3, preferably for 0, 1 or 2, particularly preferably for 0 or 1, and h stands for 0, 1, 2, 3 or 4, preferably for 0, 1 or 2, particularly preferably for 0 or 1.

Furthermore, preference is given to compounds containing at least one structure of the formula (XXVa), (XXVb), (XXVc), (XXVd), (XXVe), (XXVf), (XXVg) or (XXVh),

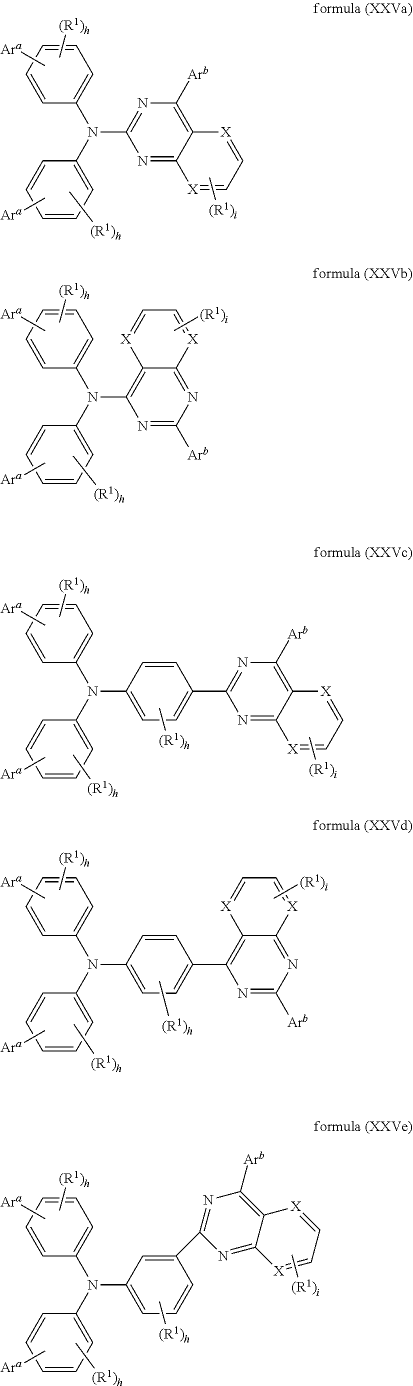

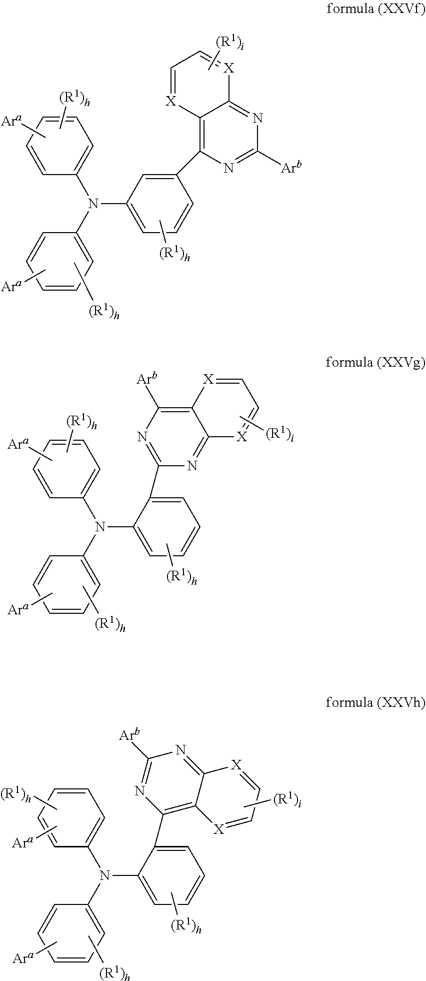

where the symbols Ar^{a}, Ar^{b}, R¹ and X used have the meaning described above, in particular for formula (I), i stands for 0, 1 or 2, preferably for 0 or 1, and h stands for 0, 1, 2, 3 or 4, preferably for 0, 1 or 2, particularly preferably for 0 or 1.

In a preferred embodiment, it may be provided that the index m in formula (I) and the preferred embodiments based thereon is 0, so that the nitrogen atom of the diarylamine group is bonded directly to the diazanaphthyl group. Particular preference is therefore given, in particular, to compounds containing structures of the formulae (XXVa) and/or (XXVb).

In a preferred embodiment of the invention, a maximum of four symbols X¹ per structure of the formulae shown above stand for N. Particularly preferably, a maximum of three symbols X¹ stand for N. Very particularly preferably, precisely two symbols X¹ in the structures of the above-mentioned formulae (I) and (XIII) stand for N and precisely one symbol X¹ in structures of the above-mentioned formulae (IIa), (IIb), (IIIa), (IIIb), (IVa), (IVb), (XIVa), (XIVb), (XVa), (XVb), (XVIa) or (XVIb) stands for N.

In a further preferred embodiment of the invention, a maximum of four symbols X per structure of the above-mentioned formulae stand for N. Particularly preferably, a maximum of two symbols X stand for N. Very particularly preferably, none of the symbols X in structures of the above-mentioned formulae stand for N.

The radical Ar^{a} represents an aromatic or heteroaromatic ring system having 5 to 30 aromatic ring atoms, which may be substituted by one or more radicals R¹. For reasons of completeness, it should be noted that the radical Ar^{a} can form an aromatic or heteroaromatic ring system together with the aryl or heteroaryl group to which the radical Ar^{a} is bonded, where the number of ring atoms of the ring system formed can increase correspondingly.

The radical Ar^{a}, in particular in the formulae (XXVa), (XXVb), (XXVc), (XXVd), (XXVe), (XXVf), (XXVg) or (XXVh), may contain a spirobifluorene, fluorene, dibenzofuran or dibenzothiophene group or form one of these groups with the aryl or heteroaryl radical to which the radical Ar^{a} is bonded.

It may preferably be provided that the radical Ar^{a}, in particular in the formulae (I), (IIa), (IIb), (IIIa), (IIIb), (IVa), (IVb), (Va), (Vb), (VIa), (VIb), (Vila), (VIIb), (VIIIa), (VIIIb), (IXa), (IXb), (Xa), (Xb), (XIa), (XIb), (XIc), (XId), (XIe), (XIf), (XIIa), (XIIb), (XIII), (XIVa), (XIVb), (XVa), (XVb), (XVIa), (XVIb), (XVIIa), (XVIIb), (XVIIIa), (XVIIIb), (XIXa), (XIXb), (XXa), (XXb), (XXIa), (XXIb), (XXIIa), (XXIIb), (XXIIIa), (XXIIIb), (XXIIIc), (XXIIId), (XXIIIe), (XXIIIf), (XXIVa), (XXIVb), (XXVa), (XXVb), (XXVc), (XXVd), (XXVe), (XXVf), (XXVg) or (XXVh), represents an aryl or heteroaryl radical having 5 to 30 aromatic ring atoms, which may be substituted by one or more radicals R¹, where the radical Ar^{a} does not contain a carbazole group or does not form a carbazole group with the aryl or heteroaryl group to which Ar^{a} is bonded, including substituents R¹, R² and R³ which may be bonded to the radical Ar^{a}. The radical Ar^{a} particularly preferably contains at most 3, especially preferably at most 2, radicals R¹ and, in a very particularly preferred embodiment, may be unsubstituted.

The aromatic or heteroaromatic group of the aromatic or heteroaromatic ring system represented by the symbol Ar^{a} is preferably bonded directly, i.e. via an atom of the aromatic or heteroaromatic group, to the respective atom of the further group, where the symbol Ar^{a} particularly preferably represents an aryl or heteroaryl radical.

In a further preferred embodiment of the invention, Ar^{a} stands, identically or differently on each occurrence, for an aromatic or heteroaromatic ring system, preferably an aryl or heteroaryl radical having 5 to 24 aromatic ring atoms, preferably having 6 to 18 aromatic ring atoms, particularly preferably for an aromatic ring system, preferably an aryl radical having 6 to 12 aromatic ring atoms, or a heteroaromatic ring system, preferably a heteroaryl group having 5 to 13 aromatic ring atoms, which may in each case be substituted by one or more radicals R¹, but is preferably unsubstituted, where R¹ can have the meaning given above, in particular in formula (I).

The radical Ar^{a} does not contain a carbazole group or does not form a carbazole group with the aryl or heteroaryl group to which Ar^{a} is bonded, including substituents R¹, R² and R³ which may be bonded to the radical Ar^{a}. It may preferably be provided that substituents R¹ which substitute the aryl or heteroaryl group to which the radical Ar^{a} is bonded and which is bonded to the nitrogen atom of the diarylamine group do not contain a carbazole group or do not form a carbazole group with the aryl or heteroaryl group to which Ar^{a} is bonded, including substituents R² and R³ which may be bonded to the radical R¹.

It may preferably be provided that the radical Ar^{b}, in particular in the formulae (I), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIc), (XId), (XIe), (XIf), (XIIa), (XIIb), (XIII), (XIVa), (XIVb), (XVa), (XVb), (XVIa), (XVIb), (XVIIa), (XVIIb), (XVIIIa), (XVIIIb), (XIXa), (XIXb), (XXa), (XXb), (XXIa), (XXIb), (XXIIa), (XXIIb), (XXIIIa), (XXIIIb), (XXIIIc), (XXIIId), (XXIIIe), (XXIIIf), (XXIVa), (XXIVb), (XXVa), (XXVb), (XXVc), (XXVd), (XXVe), (XXVf), (XXVg) or (XXVh), represents an aryl or heteroaryl radical having 5 to 30 aromatic ring atoms, which may be substituted by one or more radicals R¹. The radical Ar^{b} particularly preferably contains at most 3, especially preferably at most 2, radicals R¹ and, in a very particularly preferred embodiment, may be unsubstituted.

The aromatic or heteroaromatic group of the aromatic or heteroaromatic ring system represented by the symbol Ar^{b} is preferably bonded directly, i.e. via an atom of the aromatic or heteroaromatic group, to the respective atom of the further group, where the symbol Ar^{b} particularly preferably represents an aryl or heteroaryl radical.

In a further preferred embodiment of the invention, Ar^{b} stands, identically or differently on each occurrence, for an aromatic or heteroaromatic ring system, preferably an aryl or heteroaryl radical having 5 to 24 aromatic ring atoms, preferably having 6 to 18 aromatic ring atoms, particularly preferably for an aromatic ring system, preferably an aryl radical having 6 to 12 aromatic ring atoms, or a heteroaromatic ring system, preferably a heteroaryl group having 5 to 13 aromatic ring atoms, which may in each case be substituted by one or more radicals R¹, but is preferably unsubstituted, where R¹ can have the meaning given above, in particular in formula (I).

It may furthermore be provided that, in the structures of the formulae (I), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIc), (XId), (XIe), (XIf), (XIIa), (XIIb), (XIII), (XIVa), (XIVb), (XVa), (XVb), (XVIa), (XVIb), (XVIIa), (XVIIb), (XVIIIa), (XVIIIb), (XIXa), (XIXb), (XXa), (XXb), (XXIa), (XXIb), (XXIIa), (XXIIb), (XXIIIa), (XXIIIb), (XXIIIc), (XXIIId), (XXIIIe), (XXIIIf), (XXIVa), (XXIVb), (XXVa), (XXVb), (XXVc), (XXVd), (XXVe), (XXVf), (XXVg) and/or (XXVh), the group Ar^{b} represents a group of the formula (Ar^{b}-1),

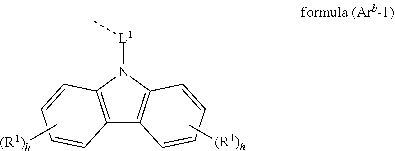

in which L1 is a bond or an aromatic or heteroaromatic ring system having 5 to 30 aromatic ring atoms, which may be substituted by one or more radicals R¹, the symbol R¹ has the meaning given above, in particular for formula (I), h is 0, 1, 2, 3 or 4, preferably 0, 1 or 2, and the dashed line represents the bond.

According to a preferred embodiment, compounds according to the invention can be depicted by structures of the formula (I), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIc), (XId), (XIe), (XIf), (XIIa), (XIIb), (XIII), (XIVa), (XIVb), (XVa), (XVb), (XVIa), (XVIb), (XVIIa), (XVIIb), (XVIIIa), (XVIIIb), (XIXa), (XIXb), (XXa), (XXb), (XXIa), (XXIb), (XXIIa), (XXIIb), (XXIIIa), (XXIIIb), (XXIIIc), (XXIIId), (XXIIIe), (XXIIIf), (XXIVa), (XXIVb), (XXVa), (XXVb), (XXVc), (XXVd), (XXVe), (XXVf), (XXVg) and/or (XXVh). Compounds according to the invention, in particular containing structures of the formula (I), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIc), (XId), (XIe), (XIf), (XIIa), (XIIb), (XIII), (XIVa), (XIVb), (XVa), (XVb), (XVIa), (XVIb), (XVIIa), (XVIIb), (XVIIIa), (XVIIIb), (XIXa), (XIXb), (XXa), (XXb), (XXIa), (XXIb), (XXIIa), (XXIIb), (XXIIIa), (XXIIIb), (XXIIIc), (XXIIId), (XXIIIe), (XXIIIf), (XXIVa), (XXIVb), (XXVa), (XXVb), (XXVc), (XXVd), (XXVe), (XXVf), (XXVg) and/or (XXVh), preferably have a molecular weight of less than or equal to 5000 g/mol, preferably less than or equal to 4000 g/mol, particularly preferably less than or equal to 3000 g/mol, especially preferably less than or equal to 2000 g/mol and very particularly preferably less than or equal to 1200 g/mol.

Preferred compounds according to the invention are furthermore distinguished by the fact that they are sublimable. These compounds generally have a molecular weight of less than about 1200 g/mol.

If X stands for CR¹ or if the aromatic and/or heteroaromatic groups are substituted by substituents R¹, these substituents R¹ are then preferably selected from the group consisting of H, D, F, CN, N(Ar¹)₂, C(═O)Ar¹, P(═O)(Ar¹)₂, a straight-chain alkyl or alkoxy group having 1 to 10 C atoms or a branched or cyclic alkyl or alkoxy group having 3 to 10 C atoms or an alkenyl group having 2 to 10 C atoms, which may in each case be substituted by one or more radicals R², where one or more non-adjacent CH₂ groups may be replaced by O and where one or more H atoms may be replaced by D or F, an aromatic or heteroaromatic ring system having 5 to 24 aromatic ring atoms, which may in each case be substituted by one or more radicals R², but is preferably unsubstituted, or an aralkyl or heteroaralkyl group having 5 to 25 aromatic ring atoms, which may be substituted by one or more radicals R²; two substituents R¹ which are bonded to the same carbon atom or to adjacent carbon atoms may form a monocyclic or polycyclic, aliphatic, aromatic or heteroaromatic ring system, which may be substituted by one or more radicals R¹. Ar¹, identically or differently on each occurrence, preferably represents an aryl or heteroaryl group having 5 to 24, preferably 5 to 12, aromatic ring atoms, which may in each case be substituted by one or more radicals R², but is preferably unsubstituted.

These substituents R¹ are particularly preferably selected from the group consisting of H, D, F, CN, N(Ar¹)₂, a straight-chain alkyl group having 1 to 8 C atoms, preferably having 1, 2, 3 or 4 C atoms, or a branched or cyclic alkyl group having 3 to 8 C atoms, preferably having 3 or 4 C atoms, or an alkenyl group having 2 to 8 C atoms, preferably having 2, 3 or 4 C atoms, which may in each case be substituted by one or more radicals R², but is preferably unsubstituted, or an aromatic or heteroaromatic ring system having 5 to 24 aromatic ring atoms, preferably having 6 to 18 aromatic ring atoms, particularly preferably having 6 to 13 aromatic ring atoms, which may in each case be substituted by one or more non-aromatic radicals R¹, but is preferably unsubstituted; two substituents R¹ which are bonded to the same carbon atom or to adjacent carbon atoms may form a monocyclic or polycyclic, aliphatic ring system, which may be substituted by one or more radicals R², but is preferably unsubstituted, where Ar¹ can have the meaning given above.

The substituents R¹ are very particularly preferably selected from the group consisting of H or an aromatic or heteroaromatic ring system having 6 to 18 aromatic ring atoms, preferably having 6 to 13 aromatic ring atoms, which may in each case be substituted by one or more non-aromatic radicals R², but is preferably unsubstituted. Examples of suitable substituents

R¹ are selected from the group consisting of phenyl, ortho-, meta- or para-biphenyl, terphenyl, in particular branched terphenyl, quaterphenyl, in particular branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl and 1-, 2-, 3- or 4-carbazolyl, which may in each case be substituted by one or more radicals R², but are preferably unsubstituted.

The radicals R¹ preferably do not form a condensed aromatic or heteroaromatic ring system, preferably do not form a condensed ring system, with the ring atoms of the aryl group or heteroaryl group to which the radicals R¹ are bonded. This includes the formation of a condensed ring system with possible substituents R² which may be bonded to the radicals R¹.

Furthermore, it may be provided that the radicals R¹ preferably do not form a condensed aromatic or heteroaromatic ring system, preferably do not form a condensed ring system, with further groups. This includes the formation of a condensed ring system with possible substituents R² which may be bonded to the radicals R¹. These further groups may be spatially adjacent or remote, where these groups contain the ring systems and radicals depicted in formula (I) and their preferred embodiments. In particular, in preferred embodiments, no further bridges occur besides the groups Y¹, Y², Y³.

The aromatic or heteroaromatic group of the aromatic or heteroaromatic ring system represented by the symbol Ar¹ is preferably bonded directly, i.e. via an atom of the aromatic or heteroaromatic group, to the respective atom of the further group, where the symbol Ar¹ particularly preferably represents an aryl or heteroaryl radical.

In a further preferred embodiment of the invention, Ar¹ stands, identically or differently on each occurrence, for an aromatic or heteroaromatic ring system, preferably an aryl or heteroaryl radical having 5 to 24 aromatic ring atoms, preferably having 6 to 18 aromatic ring atoms, particularly preferably for an aromatic ring system, preferably an aryl radical having 6 to 12 aromatic ring atoms, or a heteroaromatic ring system, preferably a heteroaryl group having 5 to 13 aromatic ring atoms, which may in each case be substituted by one or more radicals R², but is preferably unsubstituted, where R² can have the meaning given above, in particular in formula (I).

Examples of suitable groups Ar¹ are selected from the group consisting of phenyl, ortho-, meta- or para-biphenyl, terphenyl, in particular branched terphenyl, quaterphenyl, in particular branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl and 1-, 2-, 3- or 4-carbazolyl, which may in each case be substituted by one or more radicals R², but are preferably unsubstituted.

In a further embodiment, it may be provided that the radicals Ar^{a} or the radical Ar^{b} are in each case substituted by radicals R² instead of by radicals R¹.

Furthermore, it may be provided that, in a structure of the formula (I), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIc), (XId), (XIe), (XIf), (XIIa), (XIIb), (XIII), (XIVa), (XIVb), (XVa), (XVb), (XVIa), (XVIb), (XVIIa), (XVIIb), (XVIIIa), (XVIIIb), (XIXa), (XIXb), (XXa), (XXb), (XXIa), (XXIb), (XXIIa), (XXIIb), (XXIIIa), (XXIIIb), (XXIIIc), (XXIIId), (XXIIIe), (XXIIIf), (XXIVa), (XXIVb), (XXVa), (XXVb), (XXVc), (XXVd), (XXVe), (XXVf), (XXVg) and/or (XXVh), at least one radical R¹, Ar^{a} or Ar^{b} stands for a group selected from the formulae (R¹-1) to (R¹-80),

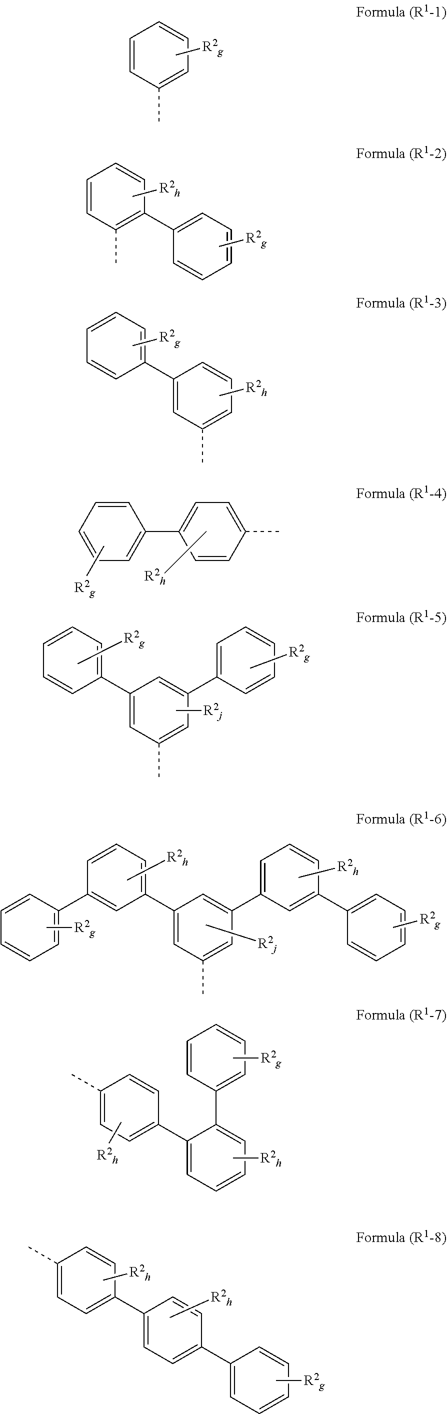

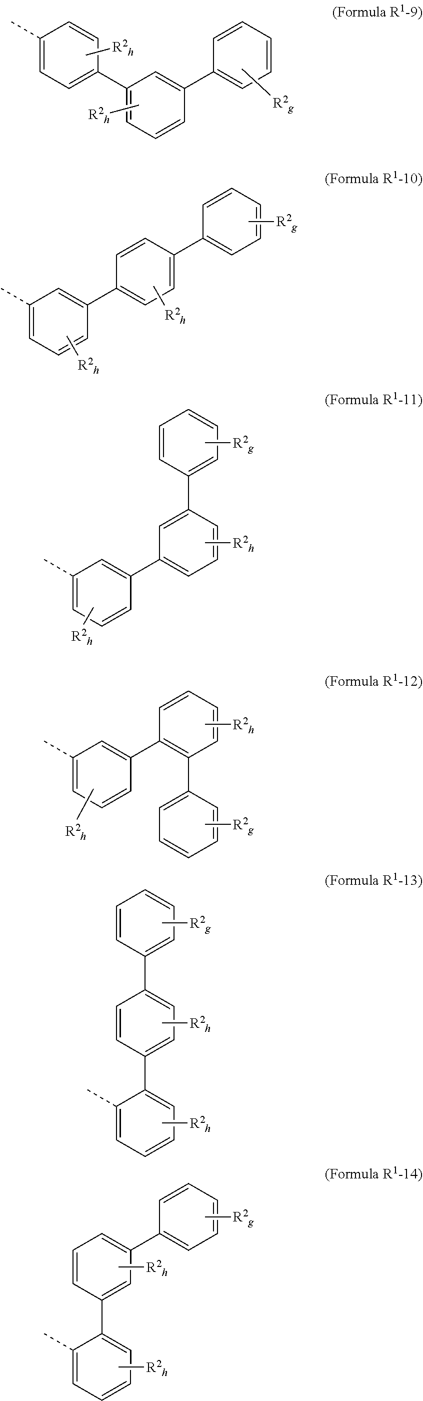

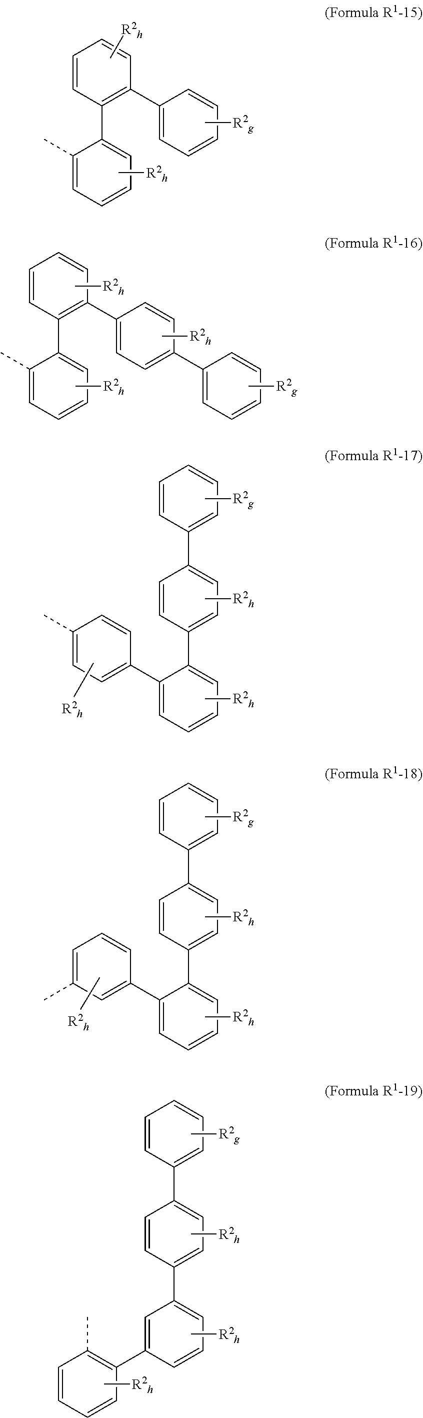

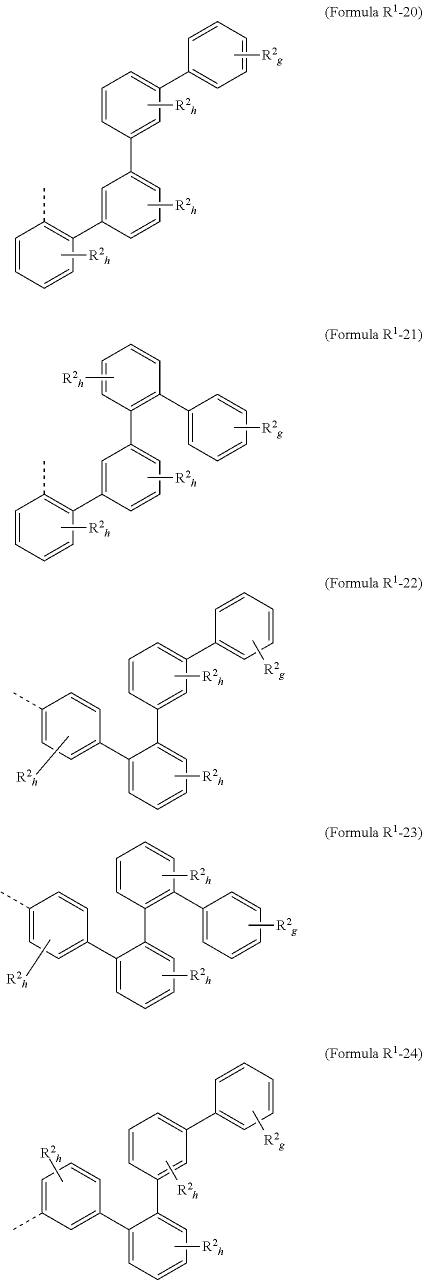

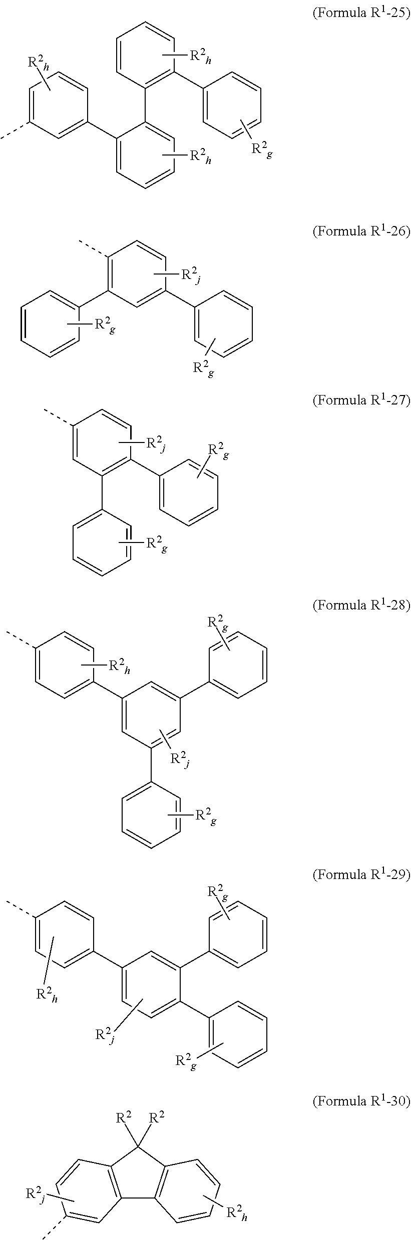

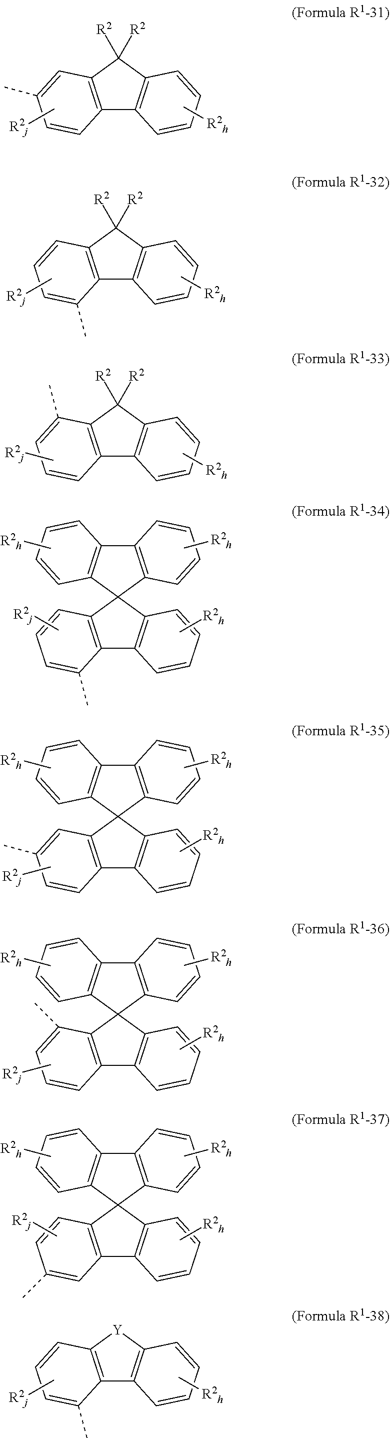

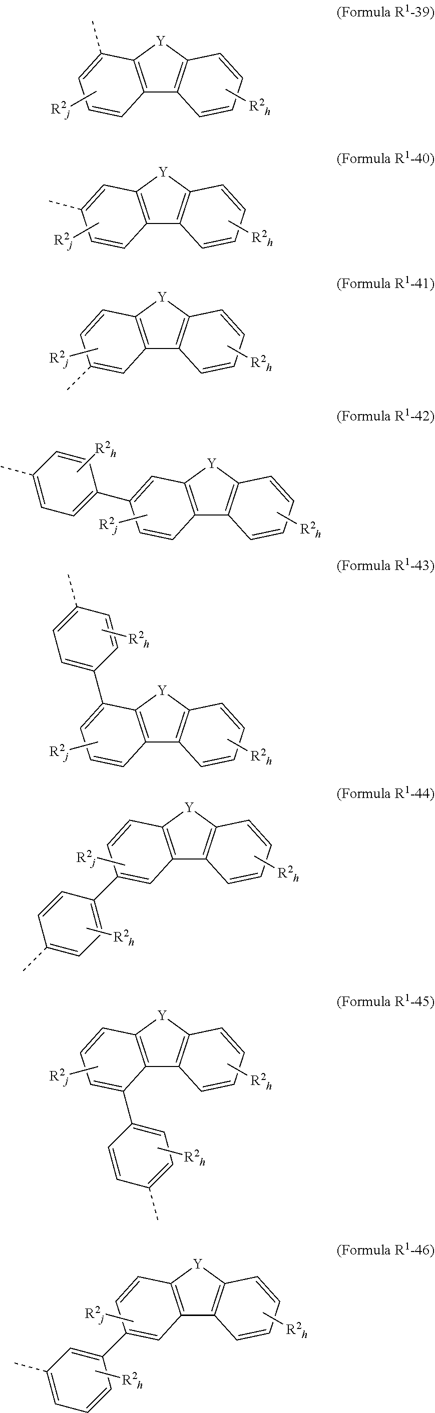

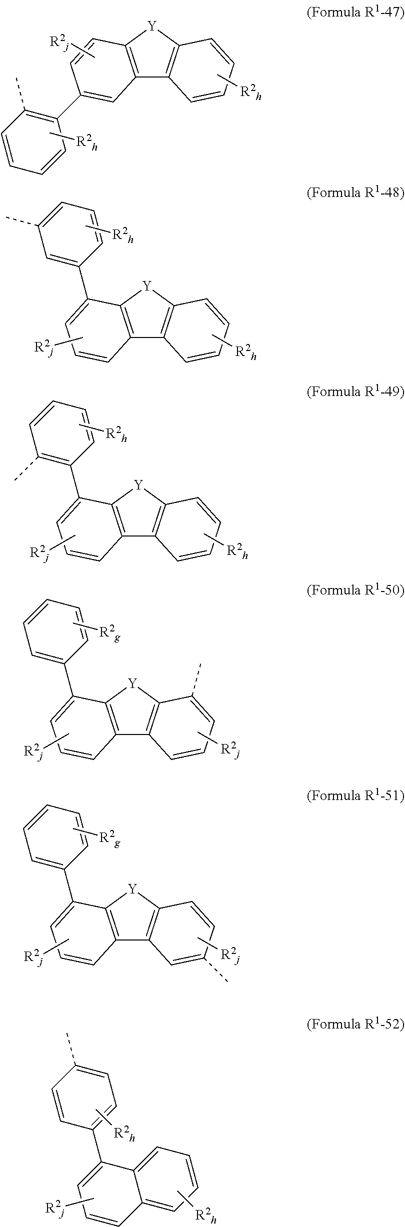

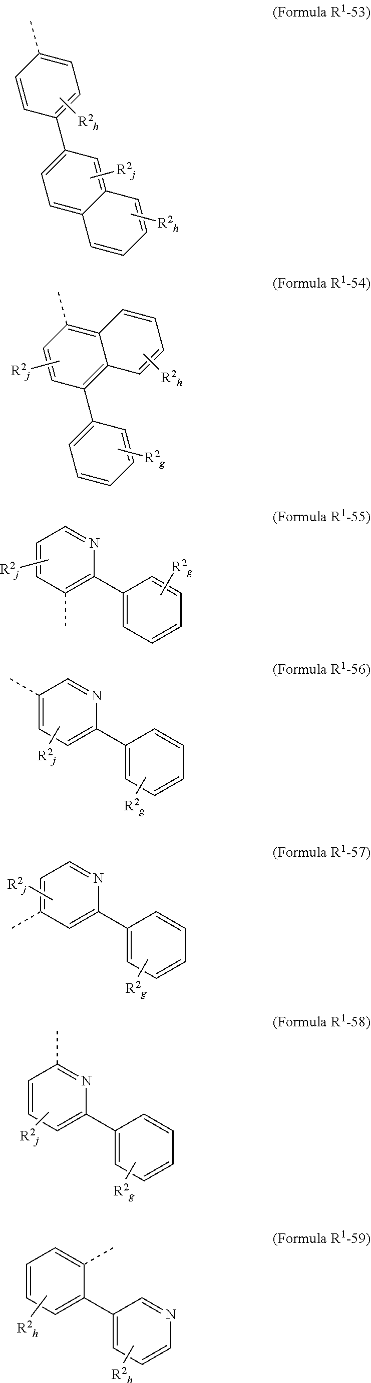

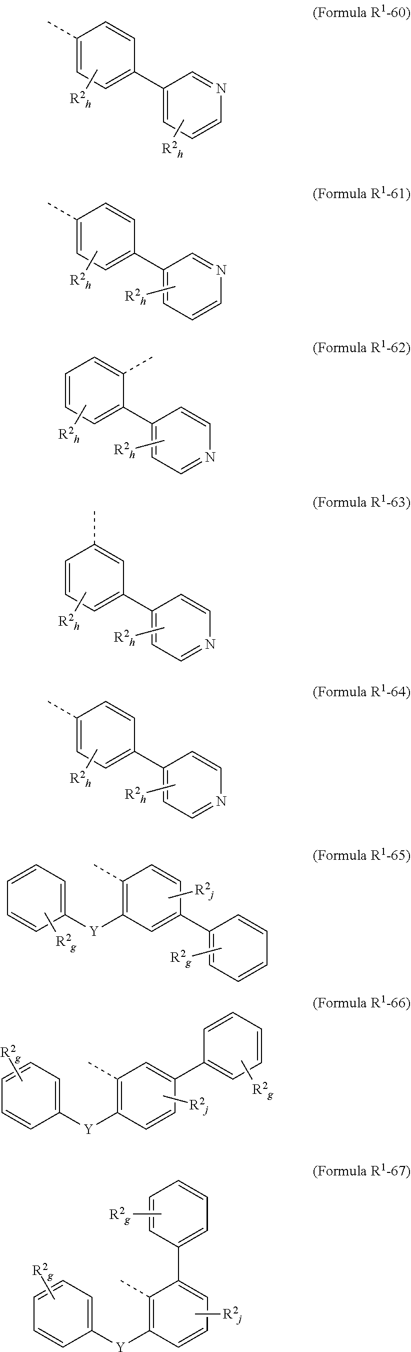

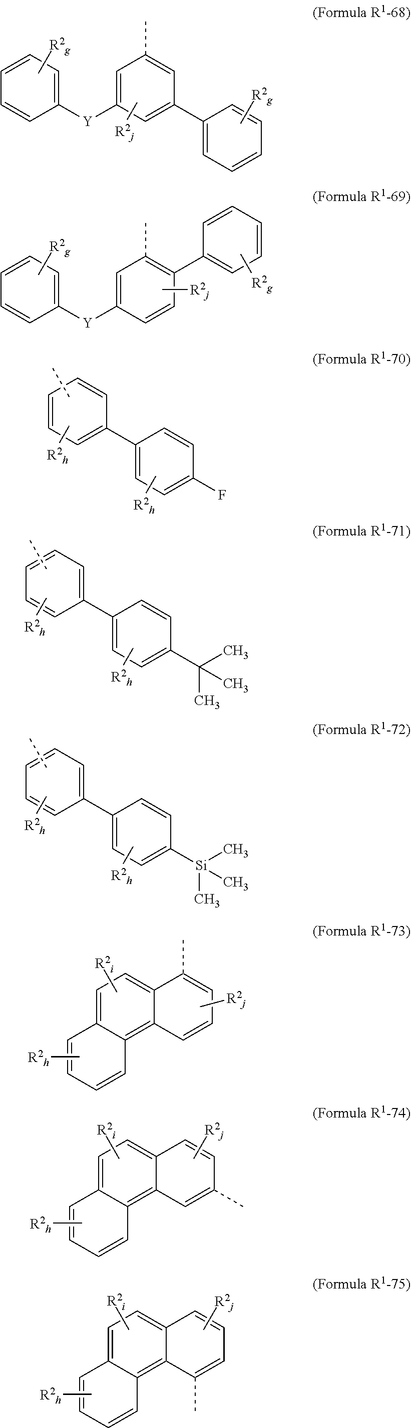

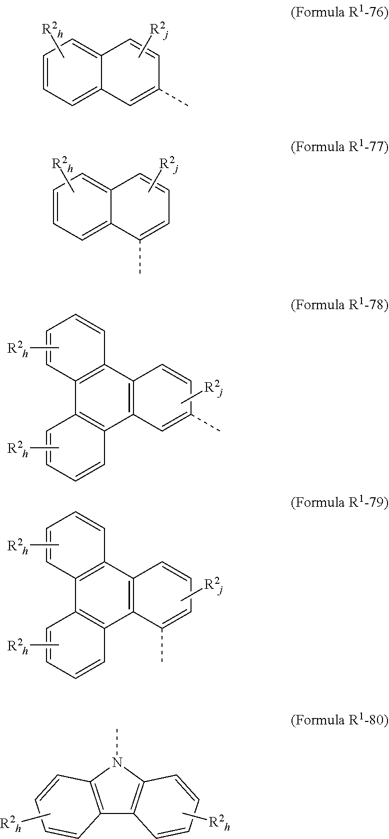

where the following applies to the symbols used:
Y is O, S or NR², preferably O or S;
i is on each occurrence, independently, 0, 1 or 2;
j is on each occurrence, independently, 0, 1, 2 or 3;
h is on each occurrence, independently, 0, 1, 2, 3 or 4;
g is on each occurrence, independently, 0, 1, 2, 3, 4 or 5;
R² can have the meaning given above, in particular given for formula (I), and
the dashed bond marks the bonding position.

The groups of the formulae R¹-1 to R¹-51 are preferred here, where the groups R¹-1, R¹-3, R¹-5, R¹-6, R¹-15, R¹-29, R¹-30, R¹-31, R¹-32, R¹-33, R¹-38, R¹-39, R¹-40, R¹-41, R¹-42, R¹-43, R¹-44 and/or R¹-45 are particularly preferred.

It may preferably be provided that the sum of the indices i, j, h and g in the structures of the formulae (R¹-1) to (R¹-80) is in each case at most 3, preferably at most 2 and particularly preferably at most 1.

The radicals R² in the formulae (R¹-1) to (R¹-80) do not form a condensed aromatic or aromatic ring system, preferably do not form a condensed ring system, with the ring atoms of the aryl group or heteroaryl group to which the radicals R² are bonded. This includes the formation of a condensed ring system with possible substituents R³ which may be bonded to the radicals R².

In the formulae R¹-1 to R¹-80, preferred groups Ar^{a} and Ar^{b} containing radicals R² are depicted. Of course, radicals which essentially conform to the formulae R¹-1 to R¹-80, but contain radicals R¹ instead of R² as substituents, represent preferred groups groups Ar^{a} and Ar^{b}. The preferences given above for these formulae R¹-1 to R¹-80, but which contain radicals R¹ instead of R² as substituents, likewise apply here.

The group L1 can preferably form a continuous conjugation with the diazanaphthalene radical to which the group L1 of the formula (Ar^{b}-1) is bonded and with the carbazole group of the formula (Ar^{b}-1). Further preferences for the group L1 in formula (Ar^{b}-1) have been described above in connection with the group L1 depicted, inter alia, in formulae (I), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIc), (XId), (XIe), (XIf), (XIIa), (XIIb), which also apply to the formula (Ar^{b}-1).

Furthermore, it may be provided that the group L is substituted by radicals R² instead of by radicals R¹.

Preference is given to compounds containing structures of the formulae (I), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIc), (XId), (XIe), (XIf), (XIIa), (XIIb) and (Ar^{b}-1) in which the group L or L1 stands for a bond or for a group selected from the formulae (L1-1) to (L1-108),

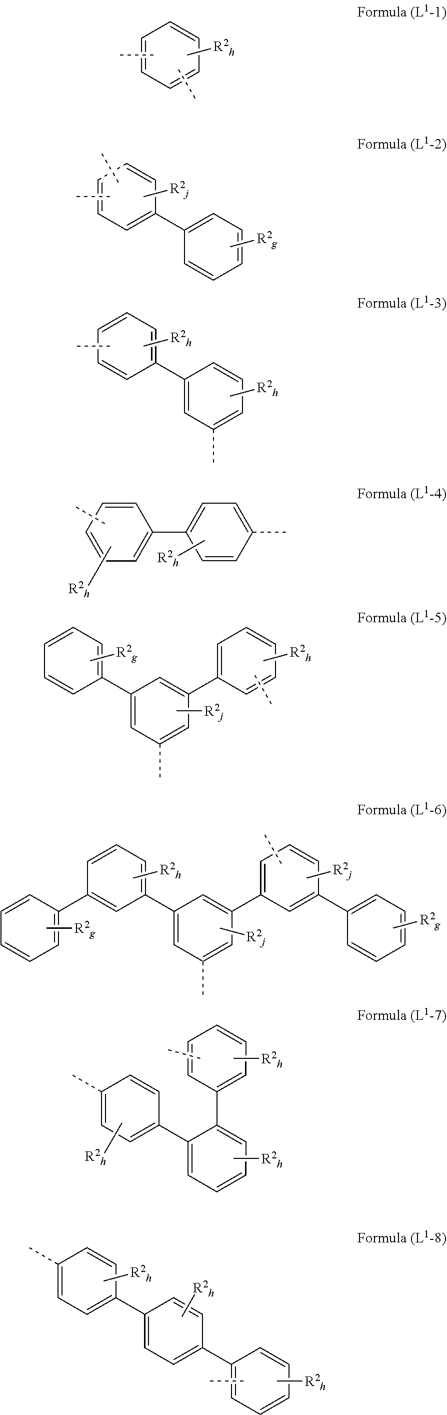

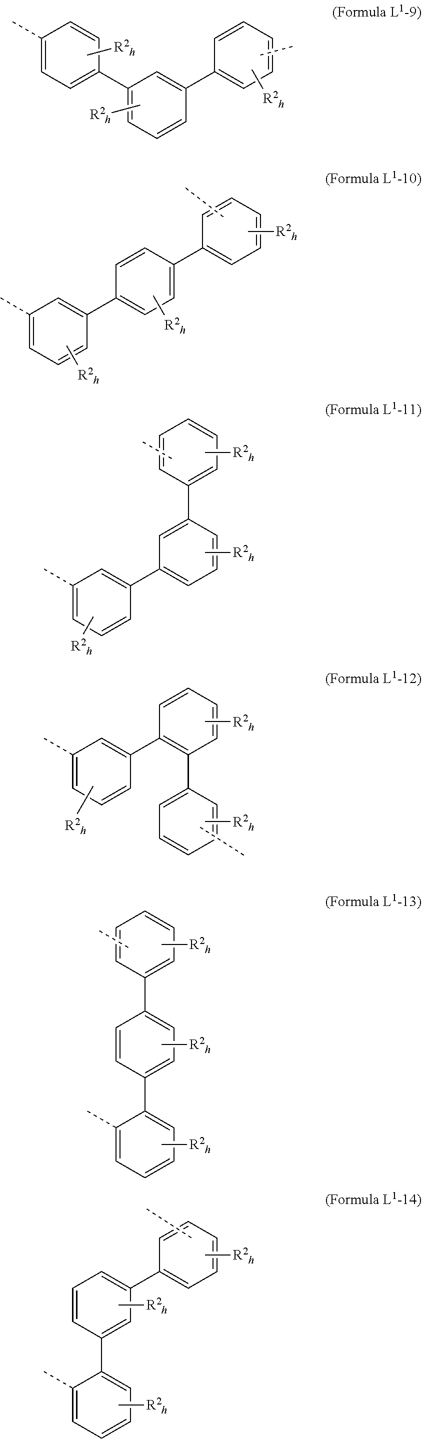

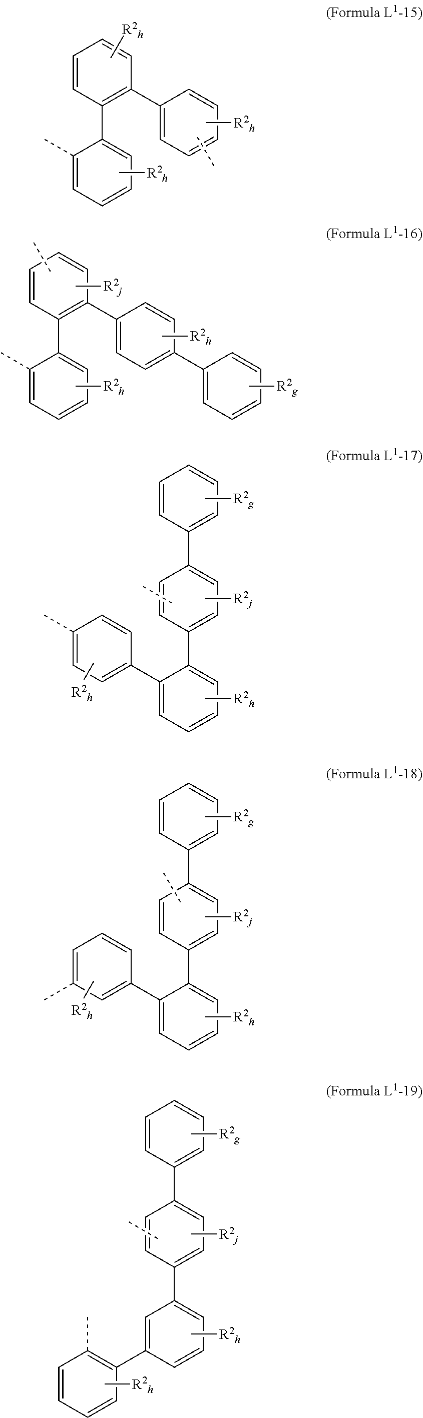

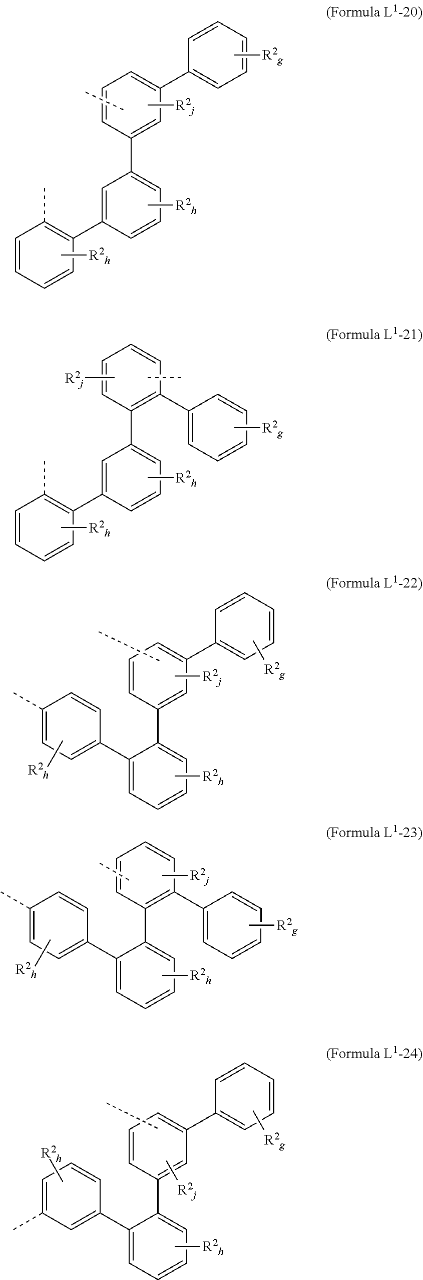

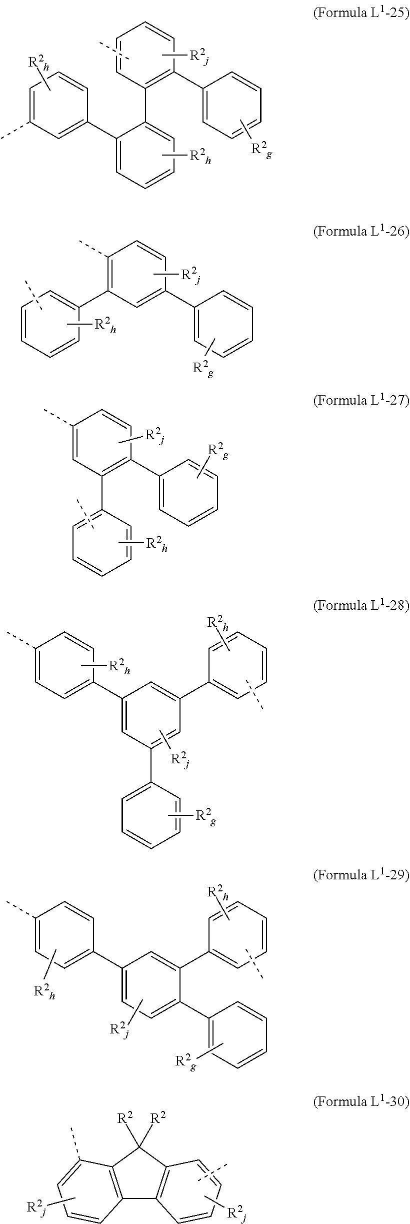

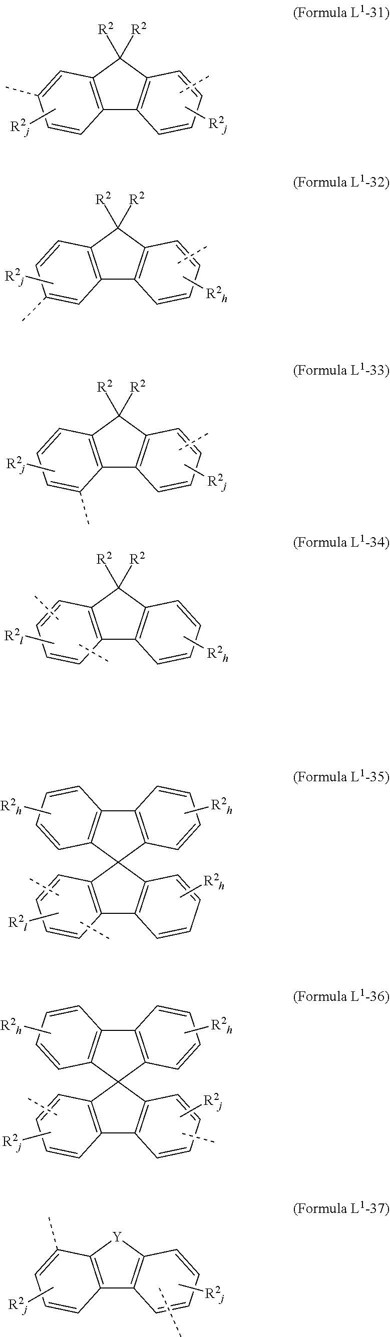

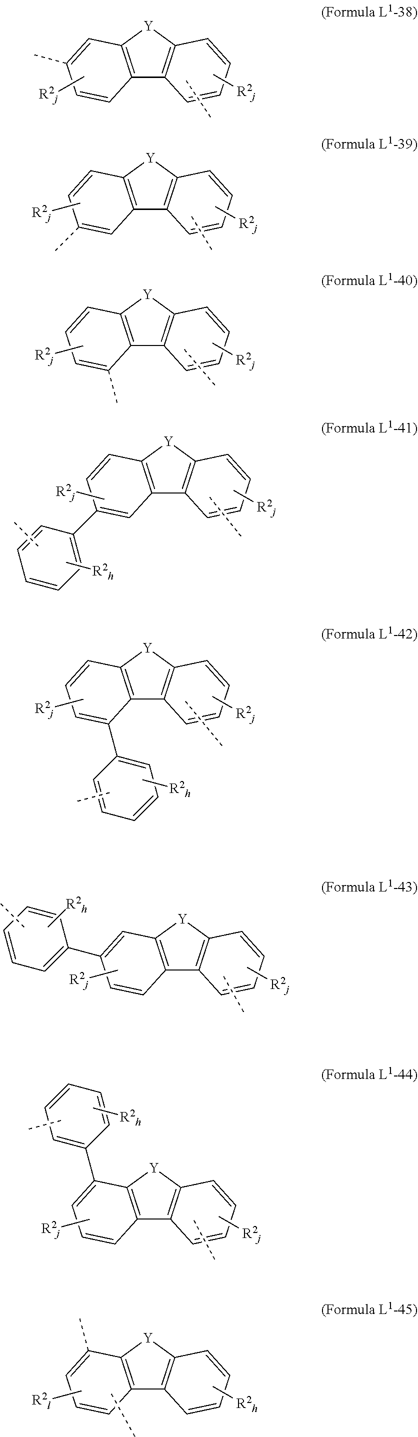

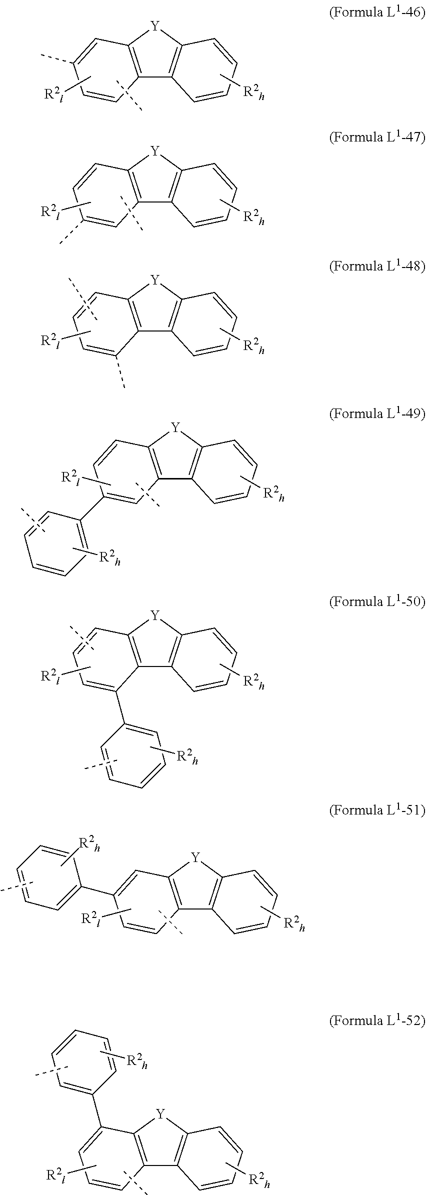

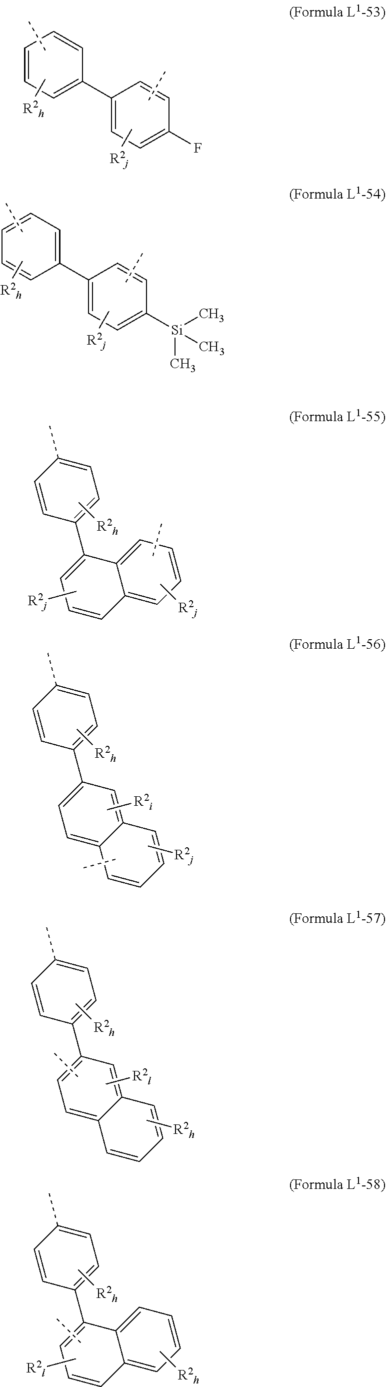

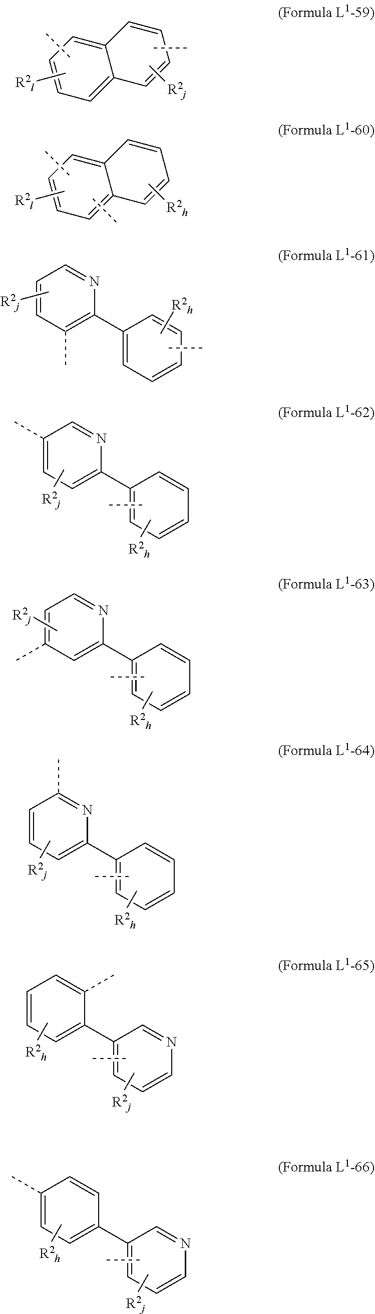

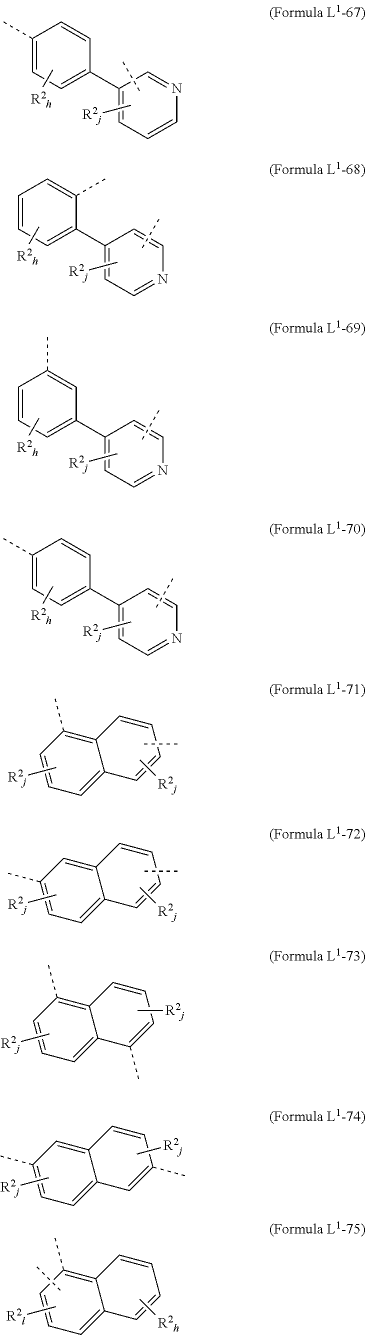

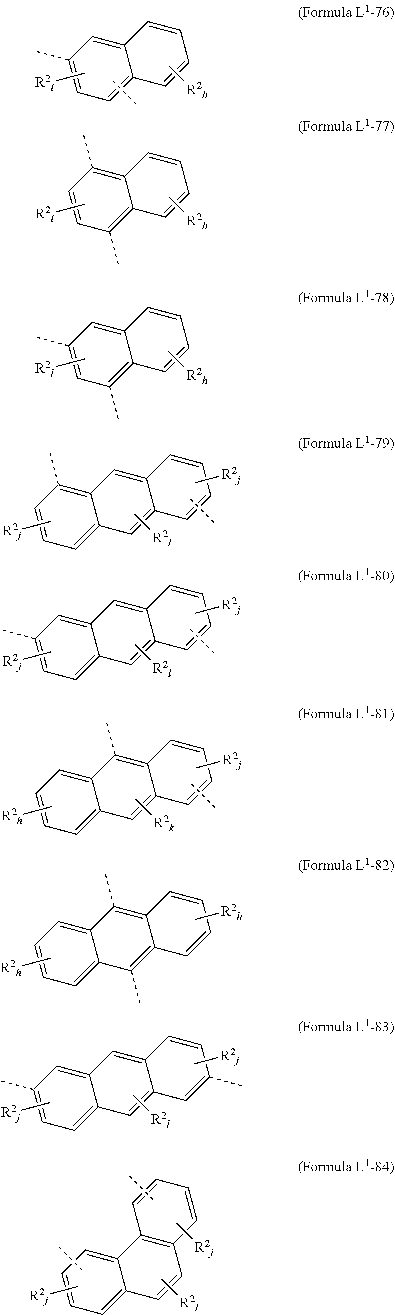

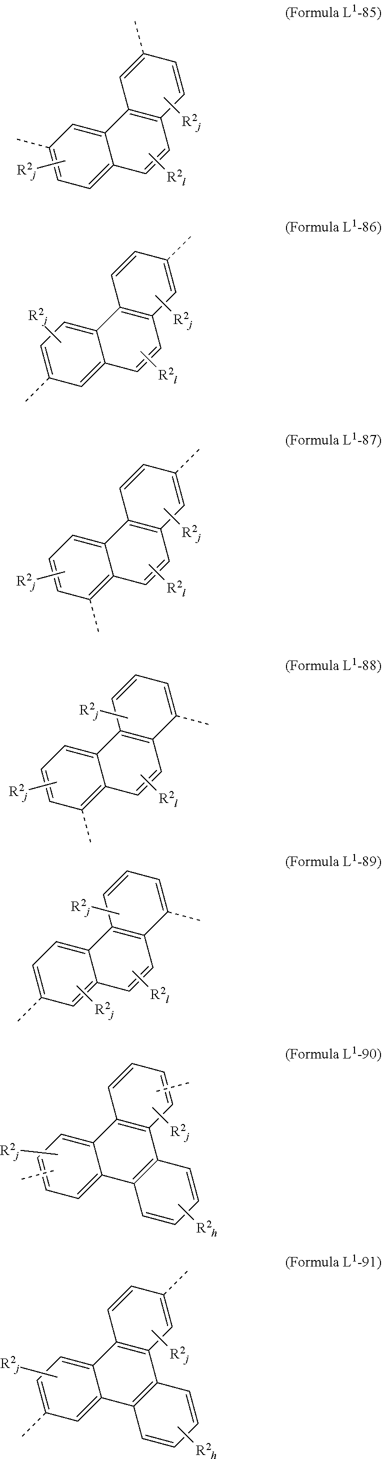

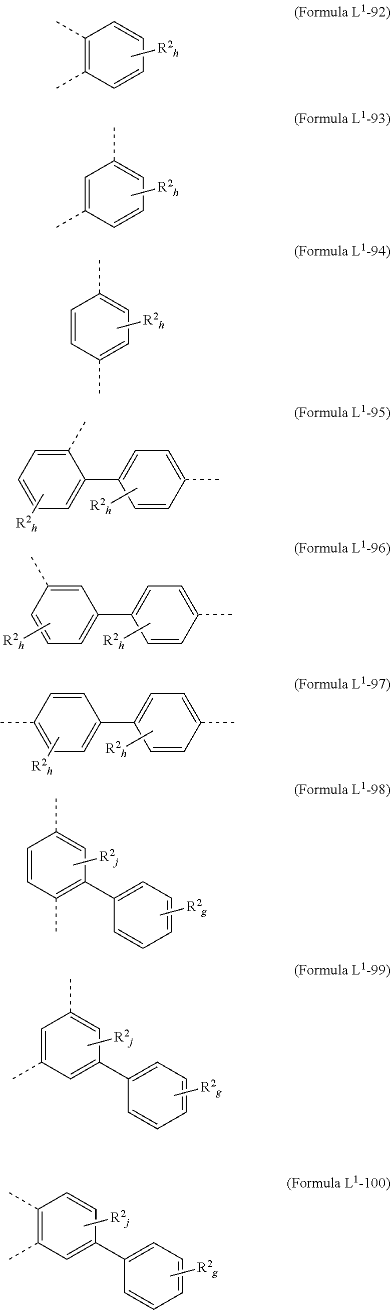

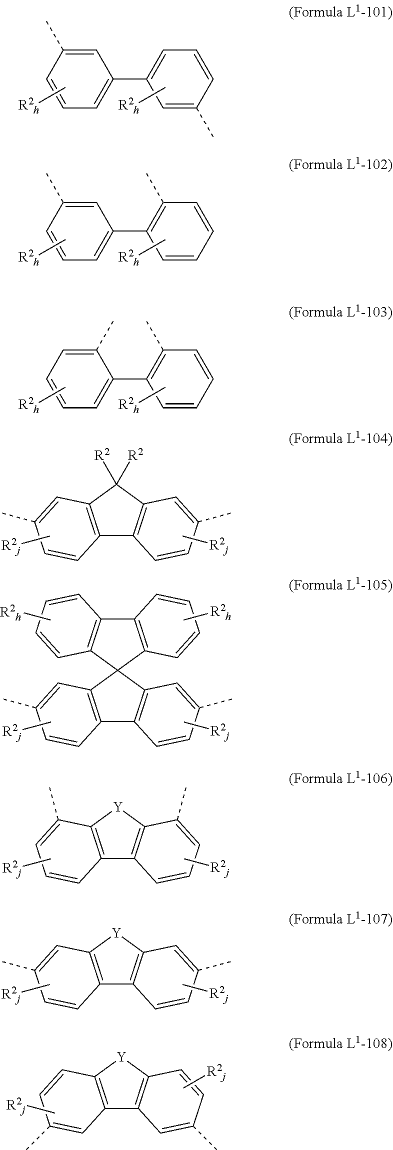

where the dashed bonds in each case mark the bonding positions, the index k is 0 or 1, the index I is 0, 1 or 2, the index j is on each occurrence, independently, 0, 1, 2 or 3; the index h is on each occurrence, independently, 0, 1, 2, 3 or 4, the index g is 0, 1, 2, 3, 4 or 5; the symbol Y is O, S or NR², preferably O or S; and the symbol R² has the meaning given above, in particular for formula (I).

It may preferably be provided that the sum of the indices k, I, g, h and j in the structures of the formula (L1-1) to (L1-108) is in each case at most 3, preferably at most 2 and particularly preferably at most 1.

Preferred compounds according to the invention contain a group L which represents a bond, i.e. in which m=0, or which is selected from one of the formulae (L1-1) to (L1-78) and/or (L1-92) to (L1-108), preferably of the formulae (L1-1) to (L1-54) and/or (L1-92) to (L1-108), especially preferably of the formulae (L1-1) to (L1-29) and/or (L1-92) to (L1-103). The sum of the indices k, I, g, h and j in the structures of the formulae (L1-1) to (L1-78) and/or (L1-92) to (L1-108), preferably of the formulae (L1-1) to (L1-54) and/or (L1-92) to (L1-108), especially preferably of the formulae (L1-1) to (L1-29) and/or (L1-92) to (L1-103), can advantageously in each case be at most 3, preferably at most 2 and particularly preferably at most 1.

The radicals R² in the formulae (L1-1) to (L1-108) preferably do not form a condensed aromatic or heteroaromatic ring system, preferably do not form a condensed ring system, with the ring atoms of the aryl group or heteroaryl group to which the radicals R² are bonded. This includes the formation of a condensed ring system with possible substituents R³ which may be bonded to the radicals R².

In the formulae L1-1 to L1-108, preferred groups L containing radicals R² are depicted. Of course, radicals which essentially conform to the formulae L1-1 to L1-108, but contain radicals R¹ instead of R² as substituents, represent preferred groups L. The preferences given above for these formulae L1-1 to L1-108, but which contain radicals R¹ instead of R² as substituents, likewise apply here.

If the compound according to the invention is substituted by aromatic or heteroaromatic groups R¹ or R², these preferably do not contain any aryl or heteroaryl groups having more than two aromatic six-membered rings condensed directly onto one another. The substituents particularly preferably contain absolutely no aryl or heteroaryl groups having six-membered rings condensed directly onto one another. This preference is due to the low triplet energy of such structures. Condensed aryl groups having more than two aromatic six-membered rings condensed directly onto one another which are nevertheless also suitable in accordance with the invention are phenanthrene and triphenylene, since these also have a high triplet level.

In a further preferred embodiment of the invention, R² is, for example in a structure of the formula (I) and preferred embodiments of this structure or the structures in which reference is made to these formulae, selected on each occurrence, identically or differently, from the group consisting of H, D, an aliphatic hydrocarbon radical having 1 to 10 C atoms, preferably having 1, 2, 3 or 4 C atoms, or an aromatic or heteroaromatic ring system having 5 to 30 aromatic ring atoms, preferably having 5 to 24 aromatic ring atoms, particularly preferably having 5 to 13 aromatic ring atoms, which may be substituted by one or more alkyl groups, each having 1 to 4 carbon atoms, but is preferably unsubstituted.

The radicals R² preferably do not form a condensed aromatic or heteroaromatic ring system, preferably do not form a condensed ring system, with the ring atoms of the aryl group or heteroaryl group to which the radicals R² are bonded. This includes the formation of a condensed ring system with possible substituents R³ which may be bonded to the radicals R².

In a further preferred embodiment of the invention, R³ is, for example in a structure of the formula (I) and preferred embodiments of this structure or the structures in which reference is made to these formulae, selected on each occurrence, identically or differently, from the group consisting of H, D, F, CN, an aliphatic hydrocarbon radical having 1 to 10 C atoms, preferably having 1, 2, 3 or 4 C atoms, or an aromatic or heteroaromatic ring system having 5 to 30 aromatic ring atoms, preferably having 5 to 24 aromatic ring atoms, particularly preferably having 5 to 13 aromatic ring atoms, which may be substituted by one or more alkyl groups, each having 1 to 4 carbon atoms, but is preferably unsubstituted.

### Compounds of the Formula (A)

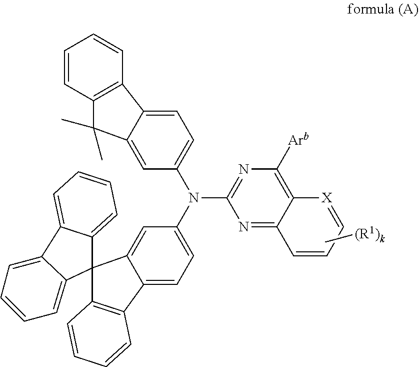

are excluded in accordance with the invention, where the symbols used have the meanings given above, in particular for formula (I).

### Compounds of the Formula (A-1)

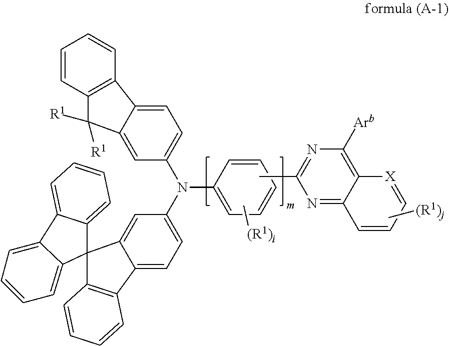

are preferably excluded, where the symbols used have the meanings given above, in particular the formula (I) or formulae (XXIa) and (XXIb).

### Compounds of the Formula (A-2)

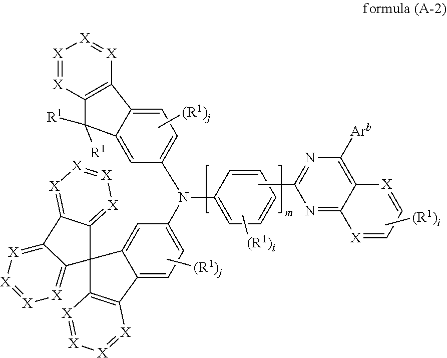

are particularly preferably excluded, where the symbols used have the meanings given above, in particular the formula (I) or formulae (XXIa) and (XXIb).

In a particular embodiment of the present invention, a compound of the formula (I) or a preferred embodiment of this formula contains at most one group Ar^{a} which contains a 4-spirobifluorene, 4-fluorene, 1-dibenzofuran or 1-dibenzothiophene group or forms one of these groups with the aryl or heteroaryl radical to which the radical Ar^{a} is bonded. Particularly preferably, a compound of the formula (I) or a preferred embodiment of this formula contains at most one group Ar^{a} which contains a spirobifluorene, fluorene, dibenzofuran or dibenzothiophene group or forms one of these groups with the aryl or heteroaryl radical to which the radical Ar^{a} is bonded.

Furthermore, it may be preferred for at least one of the radicals Ar^{a} of the formula (I) or a preferred embodiment of this formula to be unbridged, so that precisely one bond is present to the aryl radical which is bonded to the nitrogen atom of the diaryl group.

Examples of suitable compounds according to the invention are the structures of the following formulae 1 to 131 shown below:

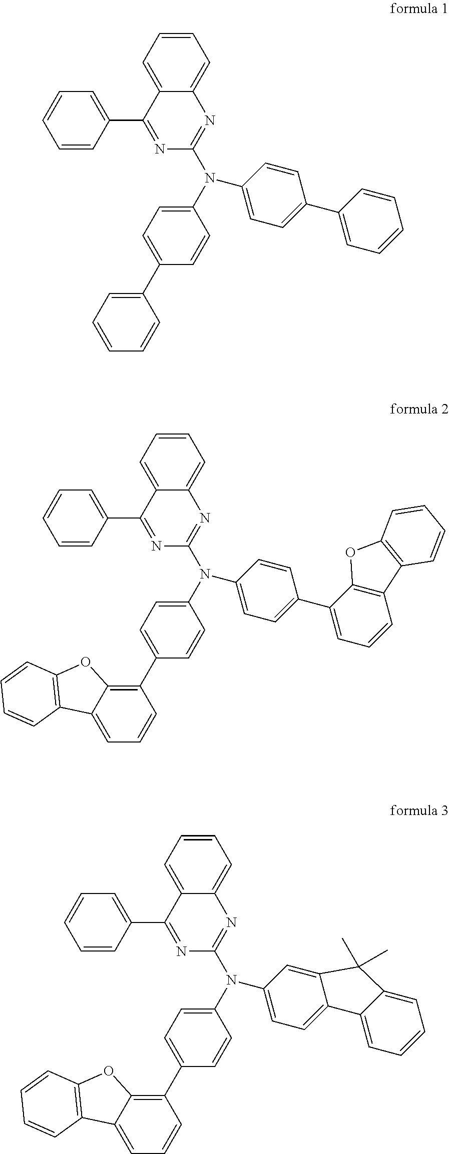 [SMILES: c1ccc(-c2ccc(N(c3ccc(-c4ccccc4)cc3)c3nc(-c4ccccc4)c4ccccc4n3)cc2)cc1 ; c1ccc(-c2nc(N(c3ccc(-c4cccc5c4oc4ccccc45)cc3)c3ccc(-c4cccc5c4oc4ccccc45)cc3)nc3ccccc23)cc1 ; CC1(C)c2ccccc2-c2ccc(N(c3ccc(-c4cccc5c4oc4ccccc45)cc3)c3nc(-c4ccccc4)c4ccccc4n3)cc21]

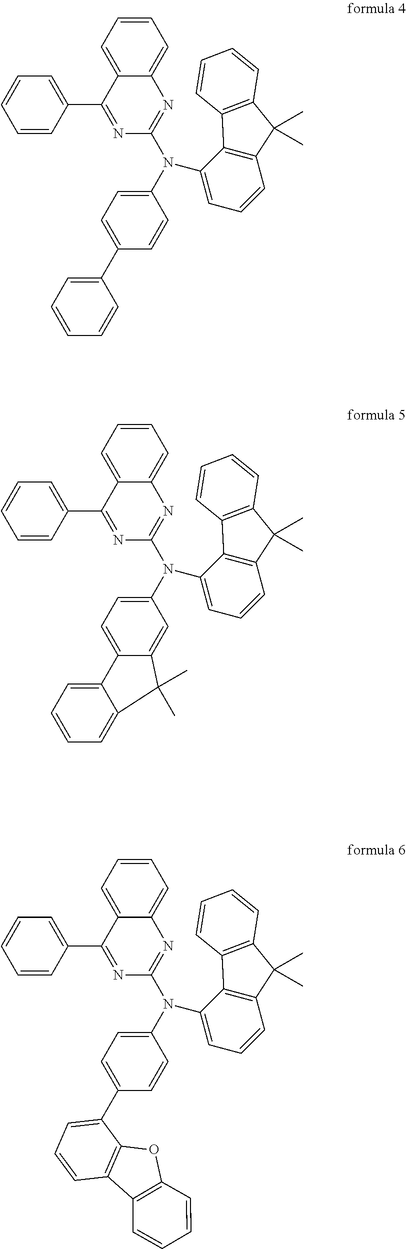 [SMILES: CC1(C)c2ccccc2-c2c(N(c3ccc(-c4ccccc4)cc3)c3nc(-c4ccccc4)c4ccccc4n3)cccc21 ; CC1(C)c2ccccc2-c2ccc(N(c3nc(-c4ccccc4)c4ccccc4n3)c3cccc4c3-c3ccccc3C4(C)C)cc21 ; CC1(C)c2ccccc2-c2c(N(c3ccc(-c4cccc5c4oc4ccccc45)cc3)c3nc(-c4ccccc4)c4ccccc4n3)cccc21]

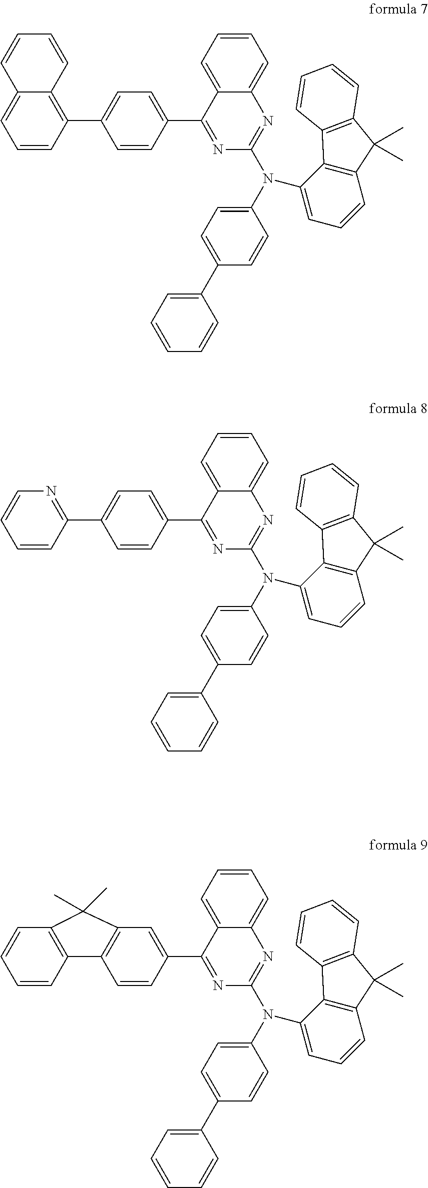 [SMILES: CC1(C)c2ccccc2-c2c(N(c3ccc(-c4ccccc4)cc3)c3nc(-c4ccc(-c5cccc6ccccc56)cc4)c4ccccc4n3)cccc21 ; CC1(C)c2ccccc2-c2c(N(c3ccc(-c4ccccc4)cc3)c3nc(-c4ccc(-c5ccccn5)cc4)c4ccccc4n3)cccc21 ; CC1(C)c2ccccc2-c2ccc(-c3nc(N(c4ccc(-c5ccccc5)cc4)c4cccc5c4-c4ccccc4C5(C)C)nc4ccccc34)cc21]

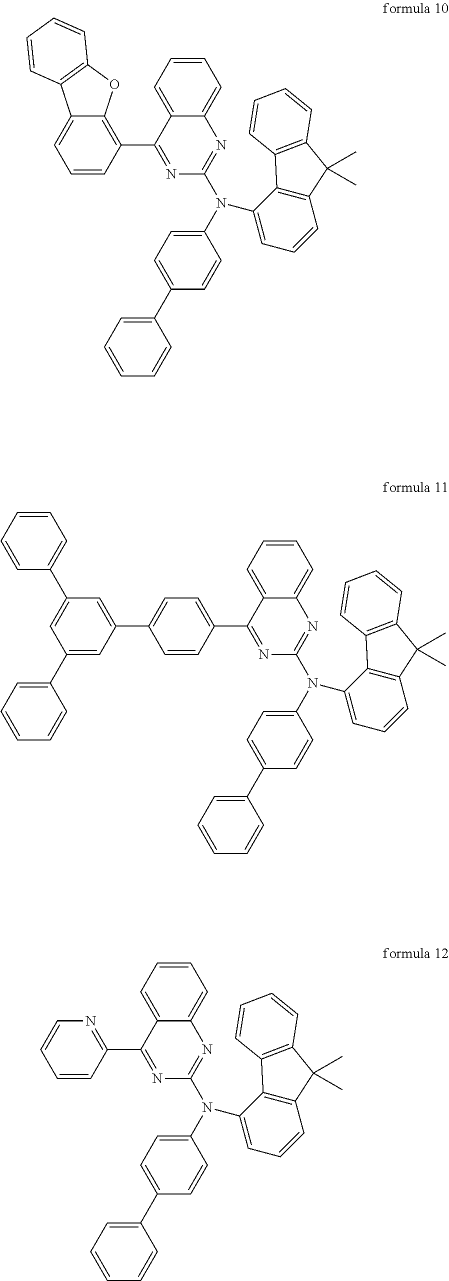 [SMILES: CC1(C)c2ccccc2-c2c(N(c3ccc(-c4ccccc4)cc3)c3nc(-c4cccc5c4oc4ccccc45)c4ccccc4n3)cccc21 ; CC1(C)c2ccccc2-c2c(N(c3ccc(-c4ccccc4)cc3)c3nc(-c4ccc(-c5cc(-c6ccccc6)cc(-c6ccccc6)c5)cc4)c4ccccc4n3)cccc21 ; CC1(C)c2ccccc2-c2c(N(c3ccc(-c4ccccc4)cc3)c3nc(-c4ccccn4)c4ccccc4n3)cccc21]

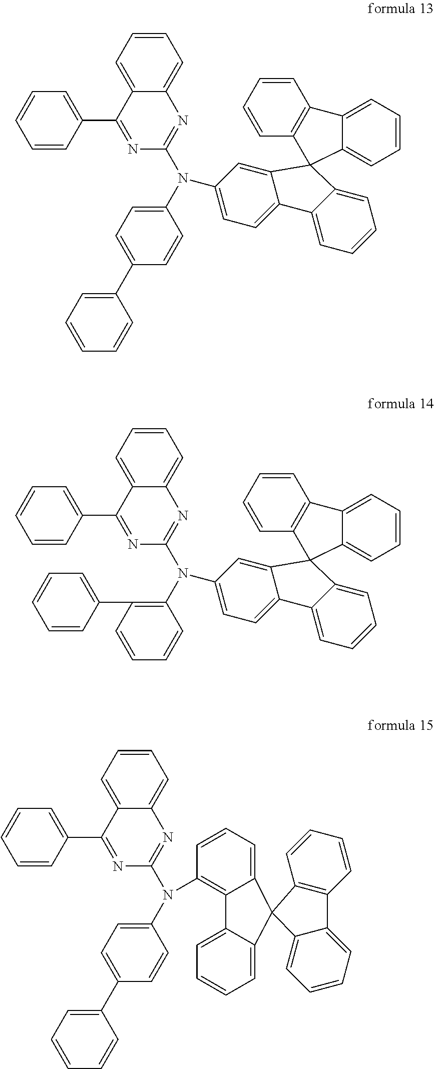 [SMILES: c1ccc(-c2ccc(N(c3ccc4c(c3)C3(c5ccccc5-c5ccccc53)c3ccccc3-4)c3nc(-c4ccccc4)c4ccccc4n3)cc2)cc1 ; c1ccc(-c2ccccc2N(c2ccc3c(c2)C2(c4ccccc4-c4ccccc42)c2ccccc2-3)c2nc(-c3ccccc3)c3ccccc3n2)cc1 ; c1ccc(-c2ccc(N(c3nc(-c4ccccc4)c4ccccc4n3)c3cccc4c3-c3ccccc3C43c4ccccc4-c4ccccc43)cc2)cc1]

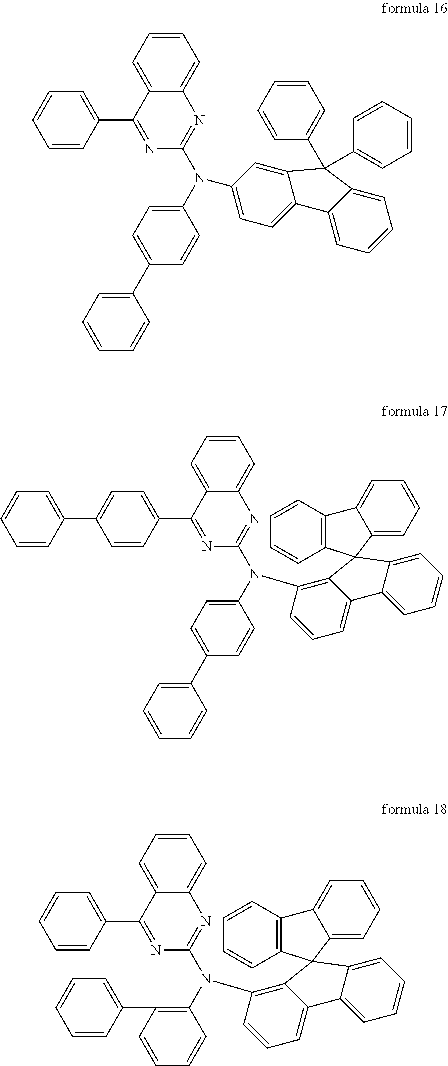 [SMILES: c1ccc(-c2ccc(N(c3ccc4c(c3)C(c3ccccc3)(c3ccccc3)c3ccccc3-4)c3nc(-c4ccccc4)c4ccccc4n3)cc2)cc1 ; c1ccc(-c2ccc(-c3nc(N(c4ccc(-c5ccccc5)cc4)c4cccc5c4C4(c6ccccc6-c6ccccc64)c4ccccc4-5)nc4ccccc34)cc2)cc1 ; c1ccc(-c2ccccc2N(c2nc(-c3ccccc3)c3ccccc3n2)c2cccc3c2C2(c4ccccc4-c4ccccc42)c2ccccc2-3)cc1]

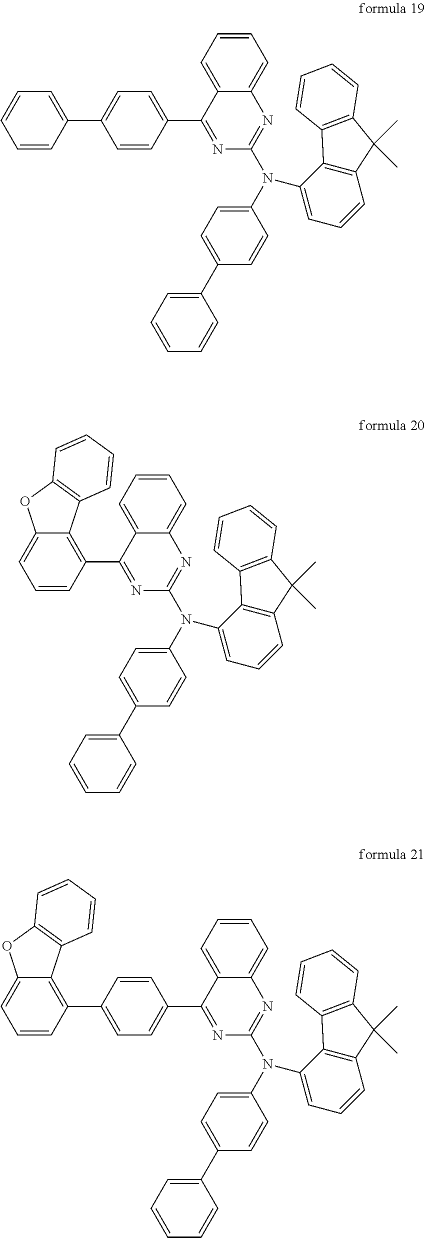 [SMILES: CC1(C)c2ccccc2-c2c(N(c3ccc(-c4ccccc4)cc3)c3nc(-c4ccc(-c5ccccc5)cc4)c4ccccc4n3)cccc21 ; CC1(C)c2ccccc2-c2c(N(c3ccc(-c4ccccc4)cc3)c3nc(-c4cccc5oc6ccccc6c45)c4ccccc4n3)cccc21 ; CC1(C)c2ccccc2-c2c(N(c3ccc(-c4ccccc4)cc3)c3nc(-c4ccc(-c5cccc6oc7ccccc7c56)cc4)c4ccccc4n3)cccc21]

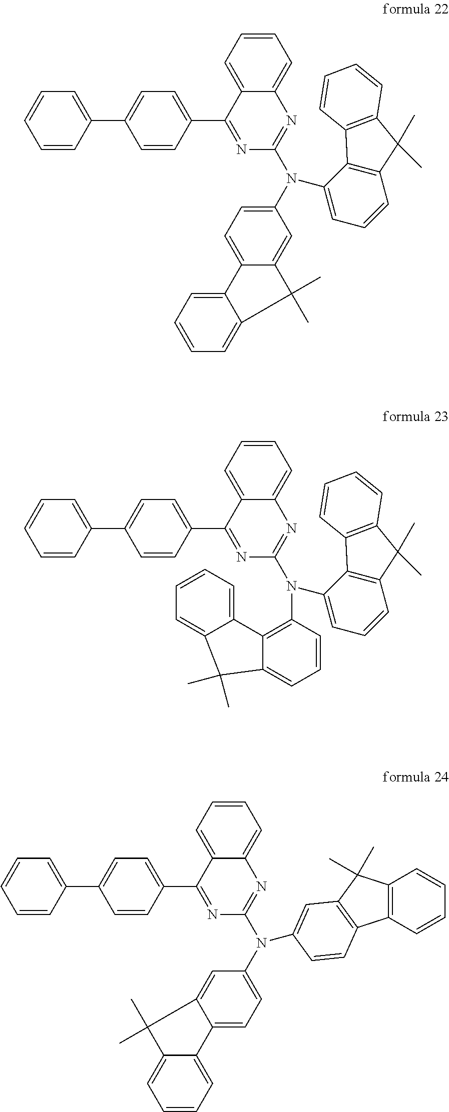 [SMILES: CC1(C)c2ccccc2-c2ccc(N(c3nc(-c4ccc(-c5ccccc5)cc4)c4ccccc4n3)c3cccc4c3-c3ccccc3C4(C)C)cc21 ; CC1(C)c2ccccc2-c2c(N(c3nc(-c4ccc(-c5ccccc5)cc4)c4ccccc4n3)c3cccc4c3-c3ccccc3C4(C)C)cccc21 ; CC1(C)c2ccccc2-c2ccc(N(c3ccc4c(c3)C(C)(C)c3ccccc3-4)c3nc(-c4ccc(-c5ccccc5)cc4)c4ccccc4n3)cc21]

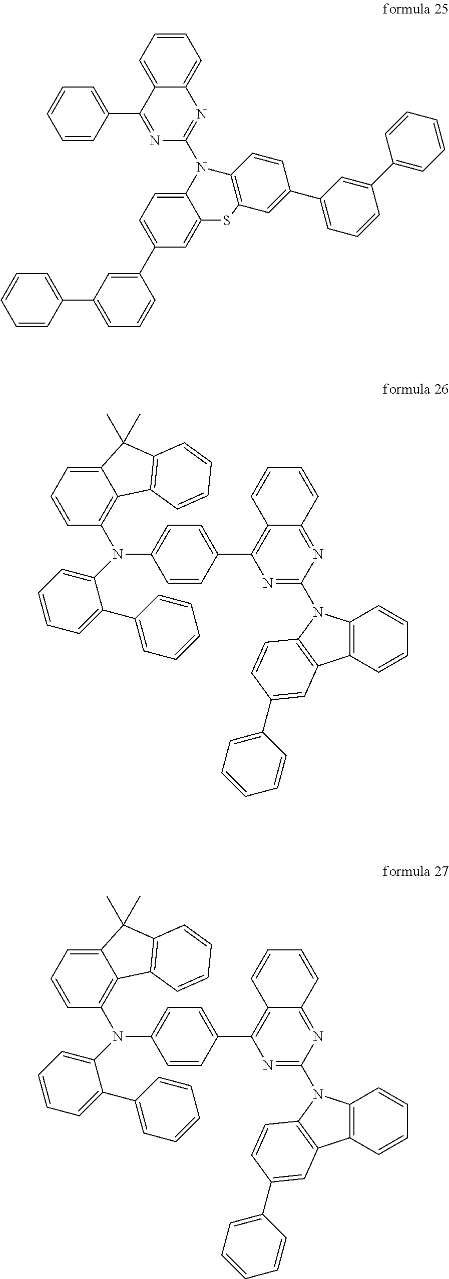 [SMILES: c1ccc(-c2cccc(-c3ccc4c(c3)Sc3cc(-c5cccc(-c6ccccc6)c5)ccc3N4c3nc(-c4ccccc4)c4ccccc4n3)c2)cc1 ; CC1(C)c2ccccc2-c2c(N(c3ccc(-c4nc(-n5c6ccccc6c6cc(-c7ccccc7)ccc65)nc5ccccc45)cc3)c3ccccc3-c3ccccc3)cccc21 ; CC1(C)c2ccccc2-c2c(N(c3ccc(-c4nc(-n5c6ccccc6c6cc(-c7ccccc7)ccc65)nc5ccccc45)cc3)c3ccccc3-c3ccccc3)cccc21]

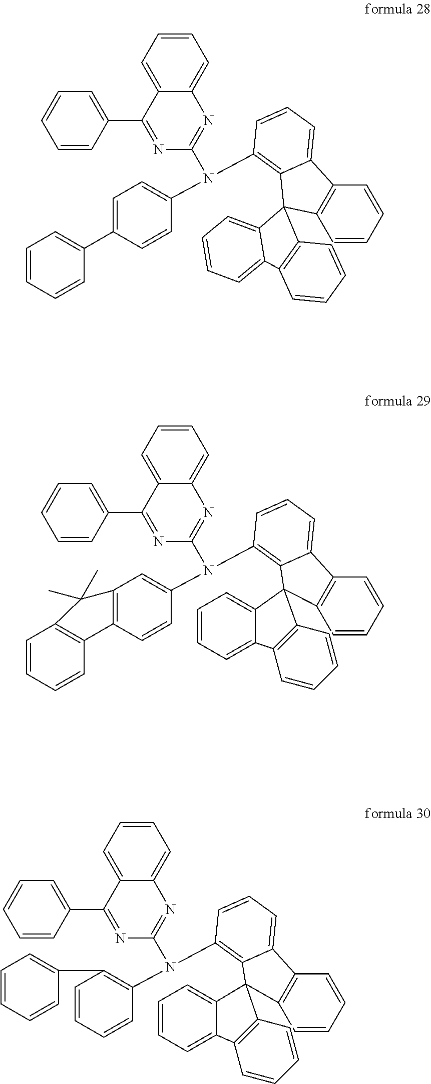 [SMILES: c1ccc(-c2ccc(N(c3nc(-c4ccccc4)c4ccccc4n3)c3cccc4c3C3(c5ccccc5-c5ccccc53)c3ccccc3-4)cc2)cc1 ; CC1(C)c2ccccc2-c2ccc(N(c3nc(-c4ccccc4)c4ccccc4n3)c3cccc4c3C3(c5ccccc5-c5ccccc53)c3ccccc3-4)cc21 ; c1ccc(-c2ccccc2N(c2nc(-c3ccccc3)c3ccccc3n2)c2cccc3c2C2(c4ccccc4-c4ccccc42)c2ccccc2-3)cc1]

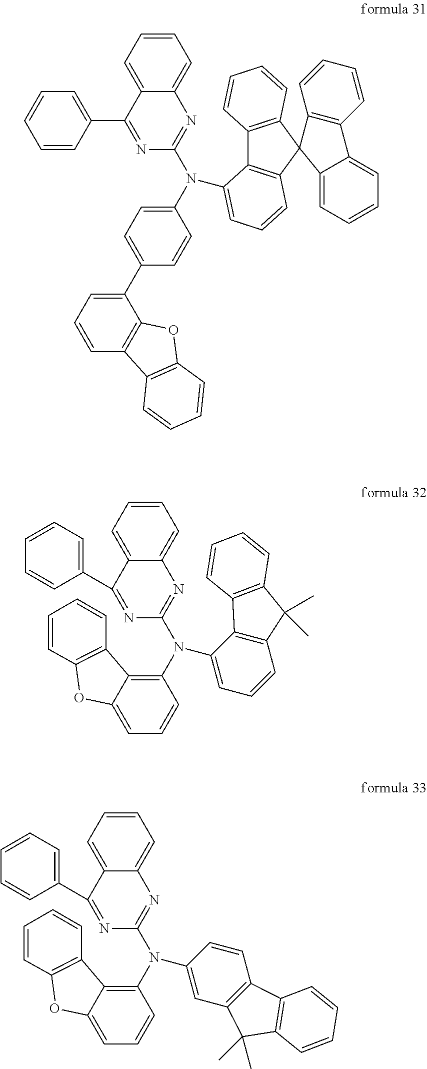 [SMILES: c1ccc(-c2nc(N(c3ccc(-c4cccc5c4oc4ccccc45)cc3)c3cccc4c3-c3ccccc3C43c4ccccc4-c4ccccc43)nc3ccccc23)cc1 ; CC1(C)c2ccccc2-c2c(N(c3nc(-c4ccccc4)c4ccccc4n3)c3cccc4oc5ccccc5c34)cccc21 ; CC1(C)c2ccccc2-c2ccc(N(c3nc(-c4ccccc4)c4ccccc4n3)c3cccc4oc5ccccc5c34)cc21]

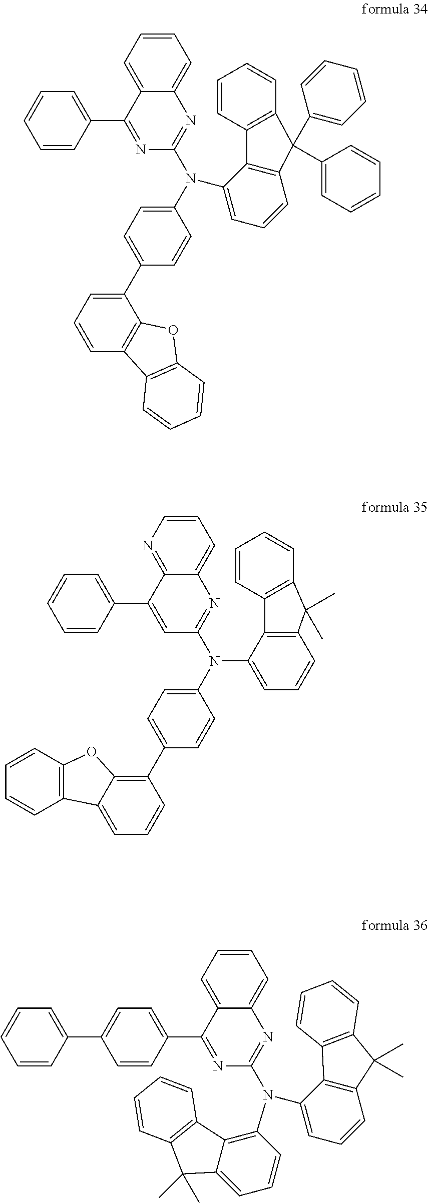 [SMILES: c1ccc(-c2nc(N(c3ccc(-c4cccc5c4oc4ccccc45)cc3)c3cccc4c3-c3ccccc3C4(c3ccccc3)c3ccccc3)nc3ccccc23)cc1 ; CC1(C)c2ccccc2-c2c(N(c3ccc(-c4cccc5c4oc4ccccc45)cc3)c3cc(-c4ccccc4)c4ncccc4n3)cccc21 ; CC1(C)c2ccccc2-c2c(N(c3nc(-c4ccc(-c5ccccc5)cc4)c4ccccc4n3)c3cccc4c3-c3ccccc3C4(C)C)cccc21]

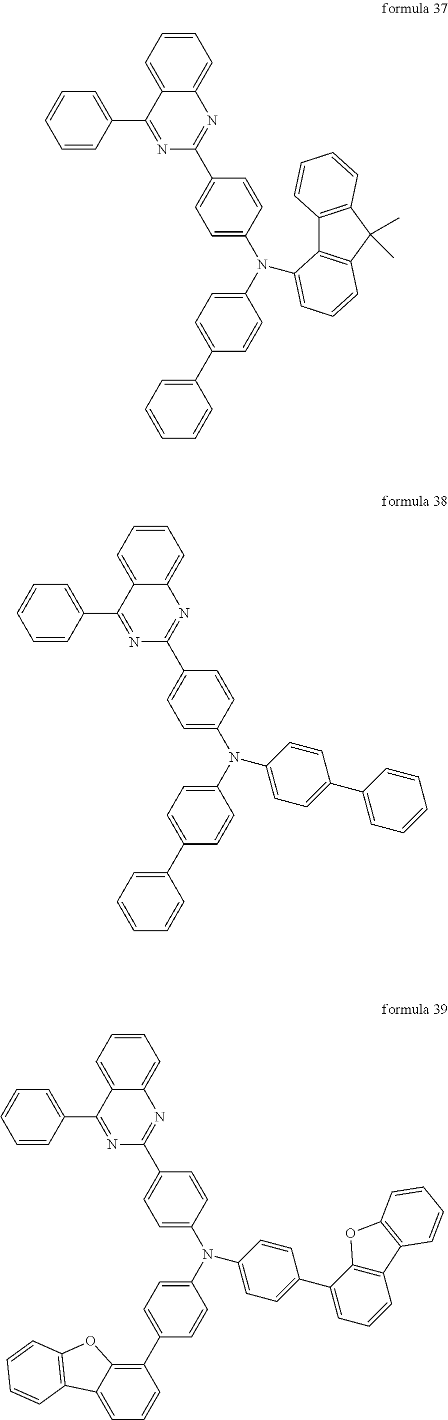 [SMILES: CC1(C)c2ccccc2-c2c(N(c3ccc(-c4ccccc4)cc3)c3ccc(-c4nc(-c5ccccc5)c5ccccc5n4)cc3)cccc21 ; c1ccc(-c2ccc(N(c3ccc(-c4ccccc4)cc3)c3ccc(-c4nc(-c5ccccc5)c5ccccc5n4)cc3)cc2)cc1 ; c1ccc(-c2nc(-c3ccc(N(c4ccc(-c5cccc6c5oc5ccccc56)cc4)c4ccc(-c5cccc6c5oc5ccccc56)cc4)cc3)nc3ccccc23)cc1]

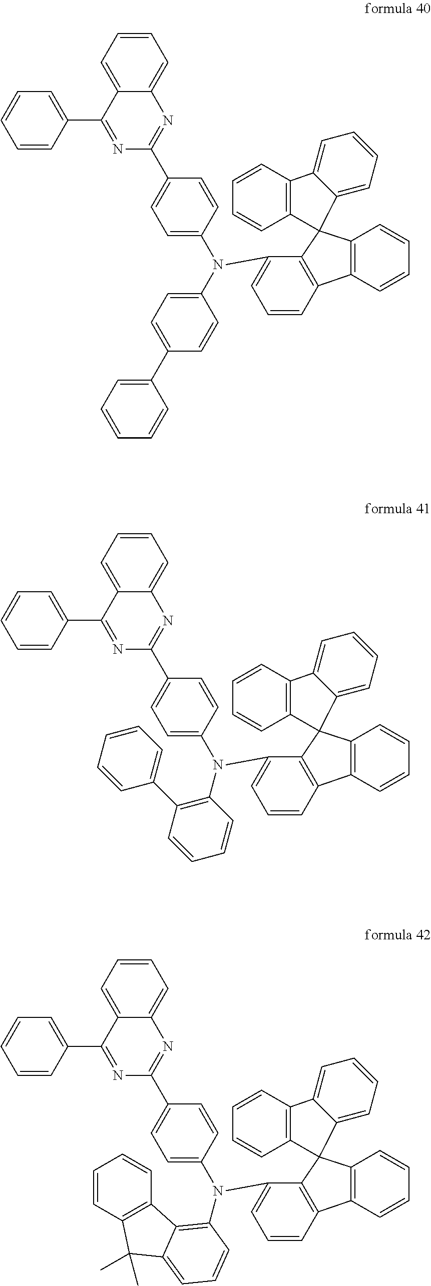 [SMILES: c1ccc(-c2ccc(N(c3ccc(-c4nc(-c5ccccc5)c5ccccc5n4)cc3)c3cccc4c3C3(c5ccccc5-c5ccccc53)c3ccccc3-4)cc2)cc1 ; c1ccc(-c2ccccc2N(c2ccc(-c3nc(-c4ccccc4)c4ccccc4n3)cc2)c2cccc3c2C2(c4ccccc4-c4ccccc42)c2ccccc2-3)cc1 ; CC1(C)c2ccccc2-c2c(N(c3ccc(-c4nc(-c5ccccc5)c5ccccc5n4)cc3)c3cccc4c3C3(c5ccccc5-c5ccccc53)c3ccccc3-4)cccc21]

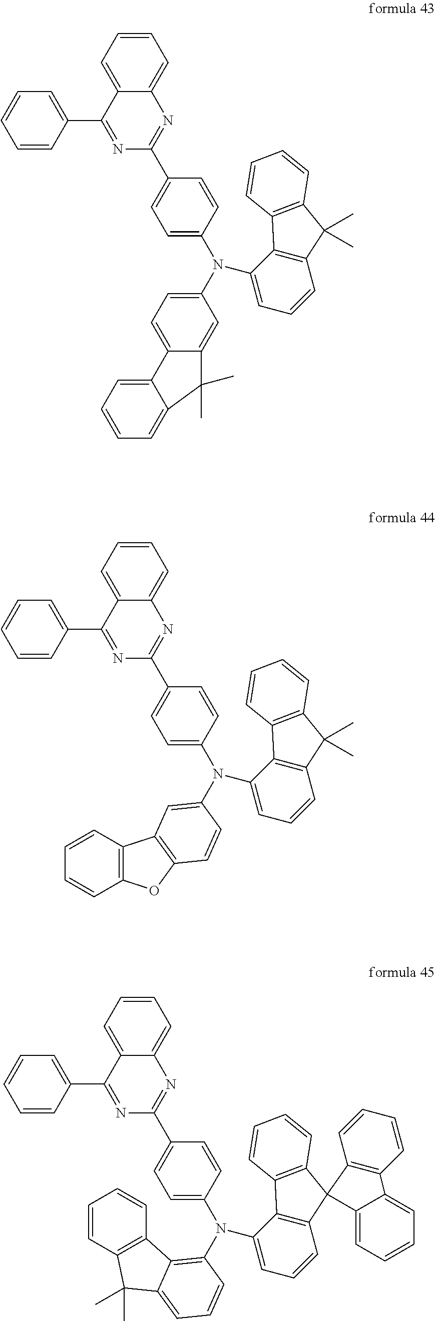 [SMILES: CC1(C)c2ccccc2-c2ccc(N(c3ccc(-c4nc(-c5ccccc5)c5ccccc5n4)cc3)c3cccc4c3-c3ccccc3C4(C)C)cc21 ; CC1(C)c2ccccc2-c2c(N(c3ccc(-c4nc(-c5ccccc5)c5ccccc5n4)cc3)c3ccc4oc5ccccc5c4c3)cccc21 ; CC1(C)c2ccccc2-c2c(N(c3ccc(-c4nc(-c5ccccc5)c5ccccc5n4)cc3)c3cccc4c3-c3ccccc3C43c4ccccc4-c4ccccc43)cccc21]

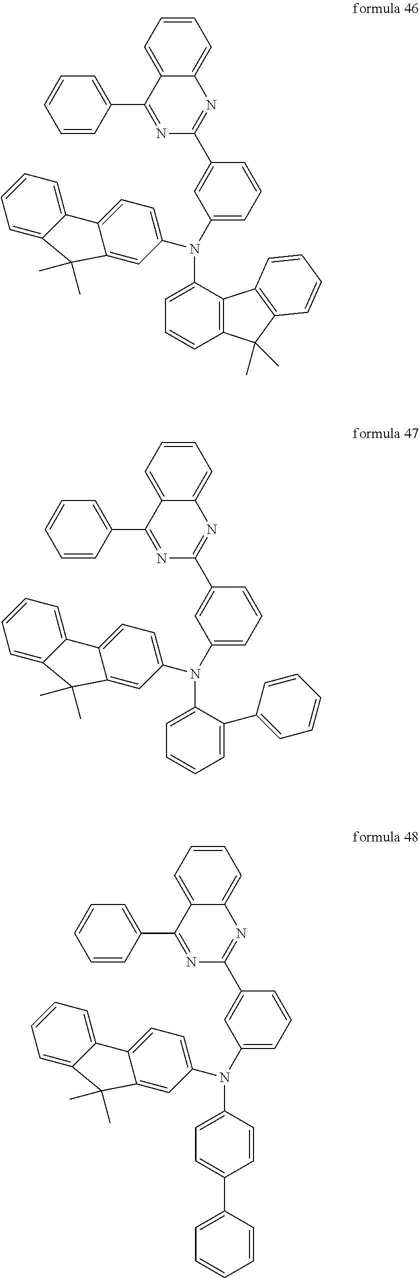 [SMILES: CC1(C)c2ccccc2-c2ccc(N(c3cccc(-c4nc(-c5ccccc5)c5ccccc5n4)c3)c3cccc4c3-c3ccccc3C4(C)C)cc21 ; CC1(C)c2ccccc2-c2ccc(N(c3cccc(-c4nc(-c5ccccc5)c5ccccc5n4)c3)c3ccccc3-c3ccccc3)cc21 ; CC1(C)c2ccccc2-c2ccc(N(c3ccc(-c4ccccc4)cc3)c3cccc(-c4nc(-c5ccccc5)c5ccccc5n4)c3)cc21]

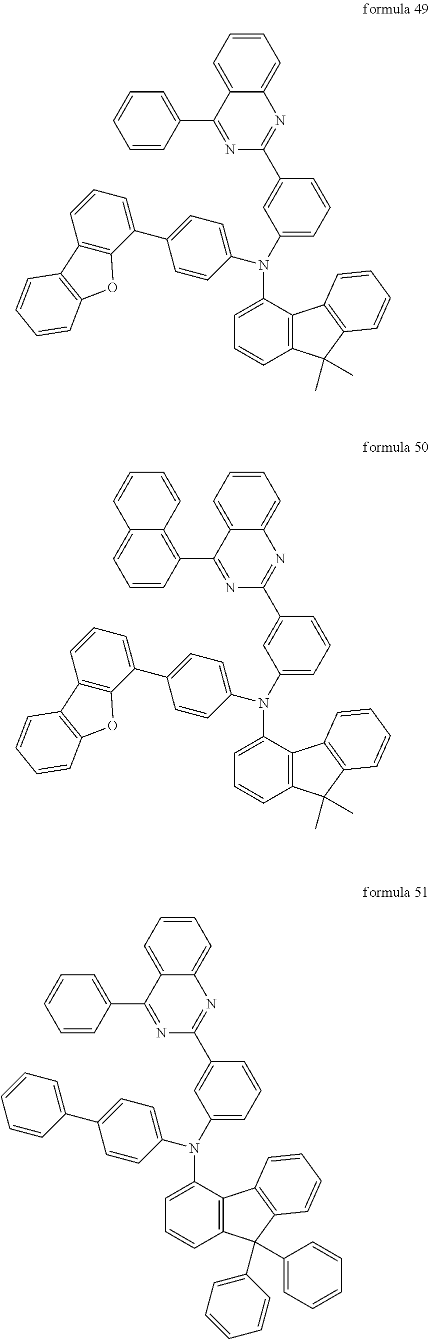 [SMILES: CC1(C)c2ccccc2-c2c(N(c3ccc(-c4cccc5c4oc4ccccc45)cc3)c3cccc(-c4nc(-c5ccccc5)c5ccccc5n4)c3)cccc21 ; CC1(C)c2ccccc2-c2c(N(c3ccc(-c4cccc5c4oc4ccccc45)cc3)c3cccc(-c4nc(-c5cccc6ccccc56)c5ccccc5n4)c3)cccc21 ; c1ccc(-c2ccc(N(c3cccc(-c4nc(-c5ccccc5)c5ccccc5n4)c3)c3cccc4c3-c3ccccc3C4(c3ccccc3)c3ccccc3)cc2)cc1]

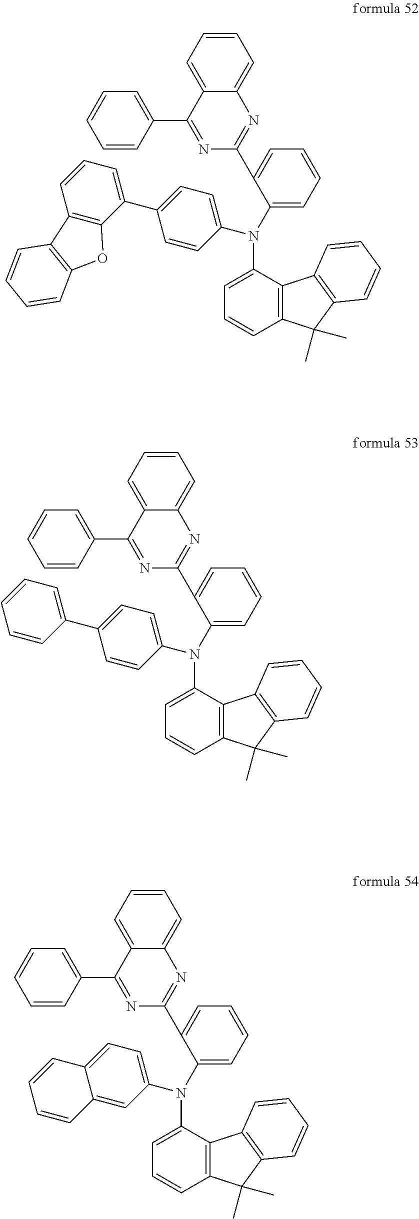 [SMILES: CC1(C)c2ccccc2-c2c(N(c3ccc(-c4cccc5c4oc4ccccc45)cc3)c3ccccc3-c3nc(-c4ccccc4)c4ccccc4n3)cccc21 ; CC1(C)c2ccccc2-c2c(N(c3ccc(-c4ccccc4)cc3)c3ccccc3-c3nc(-c4ccccc4)c4ccccc4n3)cccc21 ; CC1(C)c2ccccc2-c2c(N(c3ccc4ccccc4c3)c3ccccc3-c3nc(-c4ccccc4)c4ccccc4n3)cccc21]

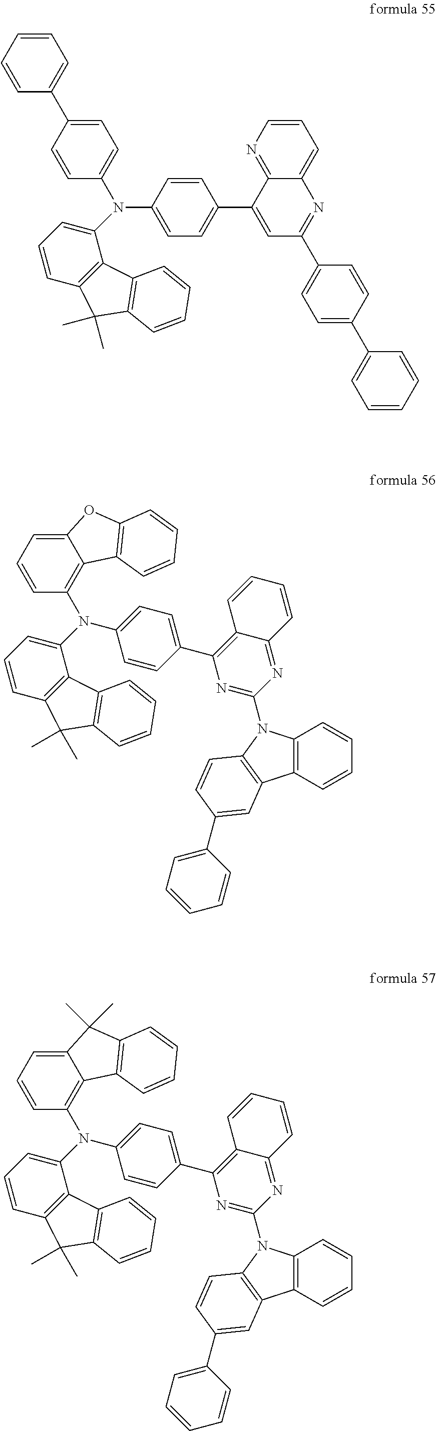 [SMILES: CC1(C)c2ccccc2-c2c(N(c3ccc(-c4ccccc4)cc3)c3ccc(-c4cc(-c5ccc(-c6ccccc6)cc5)nc5cccnc45)cc3)cccc21 ; CC1(C)c2ccccc2-c2c(N(c3ccc(-c4nc(-n5c6ccccc6c6cc(-c7ccccc7)ccc65)nc5ccccc45)cc3)c3cccc4oc5ccccc5c34)cccc21 ; CC1(C)c2ccccc2-c2c(N(c3ccc(-c4nc(-n5c6ccccc6c6cc(-c7ccccc7)ccc65)nc5ccccc45)cc3)c3cccc4c3-c3ccccc3C4(C)C)cccc21]

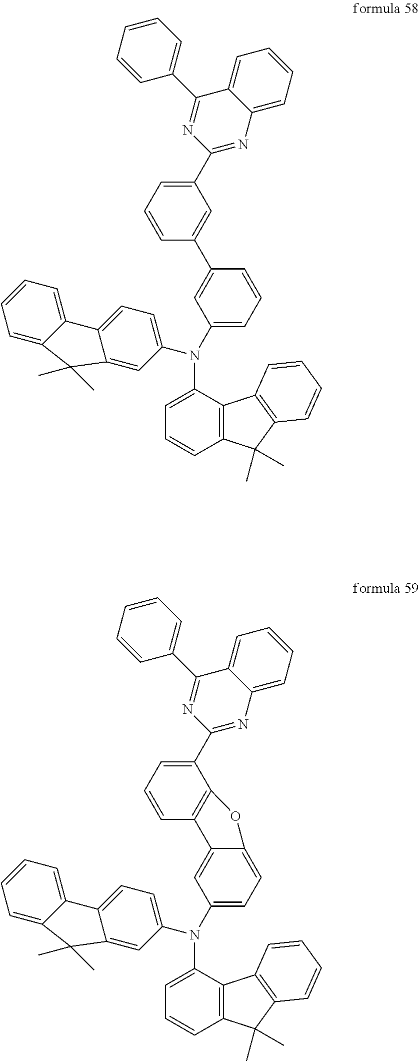 [SMILES: CC1(C)c2ccccc2-c2ccc(N(c3cccc(-c4cccc(-c5nc(-c6ccccc6)c6ccccc6n5)c4)c3)c3cccc4c3-c3ccccc3C4(C)C)cc21 ; CC1(C)c2ccccc2-c2ccc(N(c3ccc4oc5c(-c6nc(-c7ccccc7)c7ccccc7n6)cccc5c4c3)c3cccc4c3-c3ccccc3C4(C)C)cc21]

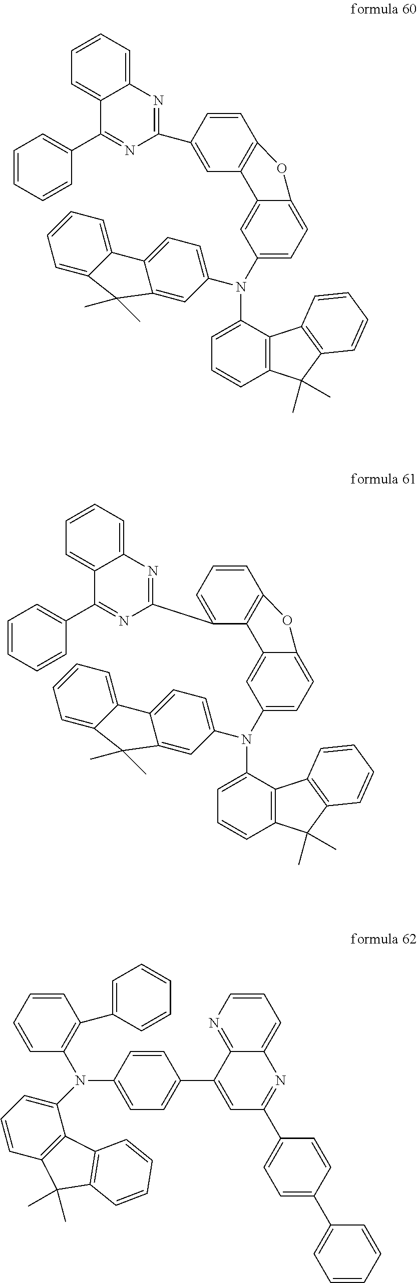 [SMILES: CC1(C)c2ccccc2-c2ccc(N(c3ccc4oc5ccc(-c6nc(-c7ccccc7)c7ccccc7n6)cc5c4c3)c3cccc4c3-c3ccccc3C4(C)C)cc21 ; CC1(C)c2ccccc2-c2ccc(N(c3ccc4oc5cccc(-c6nc(-c7ccccc7)c7ccccc7n6)c5c4c3)c3cccc4c3-c3ccccc3C4(C)C)cc21 ; CC1(C)c2ccccc2-c2c(N(c3ccc(-c4cc(-c5ccc(-c6ccccc6)cc5)nc5cccnc45)cc3)c3ccccc3-c3ccccc3)cccc21]

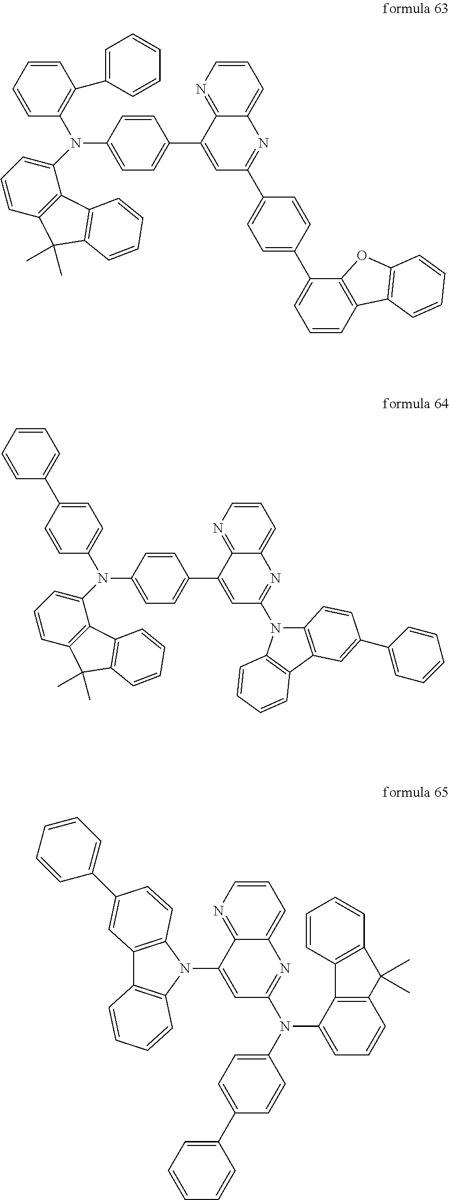 [SMILES: CC1(C)c2ccccc2-c2c(N(c3ccc(-c4cc(-c5ccc(-c6cccc7c6oc6ccccc67)cc5)nc5cccnc45)cc3)c3ccccc3-c3ccccc3)cccc21 ; CC1(C)c2ccccc2-c2c(N(c3ccc(-c4ccccc4)cc3)c3ccc(-c4cc(-n5c6ccccc6c6cc(-c7ccccc7)ccc65)nc5cccnc45)cc3)cccc21 ; CC1(C)c2ccccc2-c2c(N(c3ccc(-c4ccccc4)cc3)c3cc(-n4c5ccccc5c5cc(-c6ccccc6)ccc54)c4ncccc4n3)cccc21]

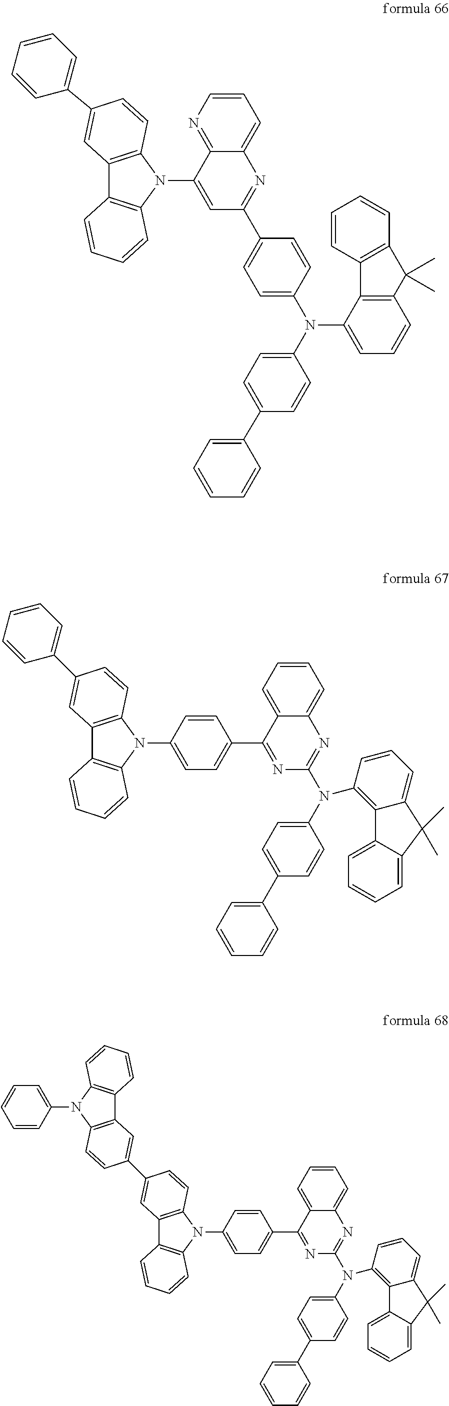 [SMILES: CC1(C)c2ccccc2-c2c(N(c3ccc(-c4ccccc4)cc3)c3ccc(-c4cc(-n5c6ccccc6c6cc(-c7ccccc7)ccc65)c5ncccc5n4)cc3)cccc21 ; CC1(C)c2ccccc2-c2c(N(c3ccc(-c4ccccc4)cc3)c3nc(-c4ccc(-n5c6ccccc6c6cc(-c7ccccc7)ccc65)cc4)c4ccccc4n3)cccc21 ; CC1(C)c2ccccc2-c2c(N(c3ccc(-c4ccccc4)cc3)c3nc(-c4ccc(-n5c6ccccc6c6cc(-c7ccc8c(c7)c7ccccc7n8-c7ccccc7)ccc65)cc4)c4ccccc4n3)cccc21]

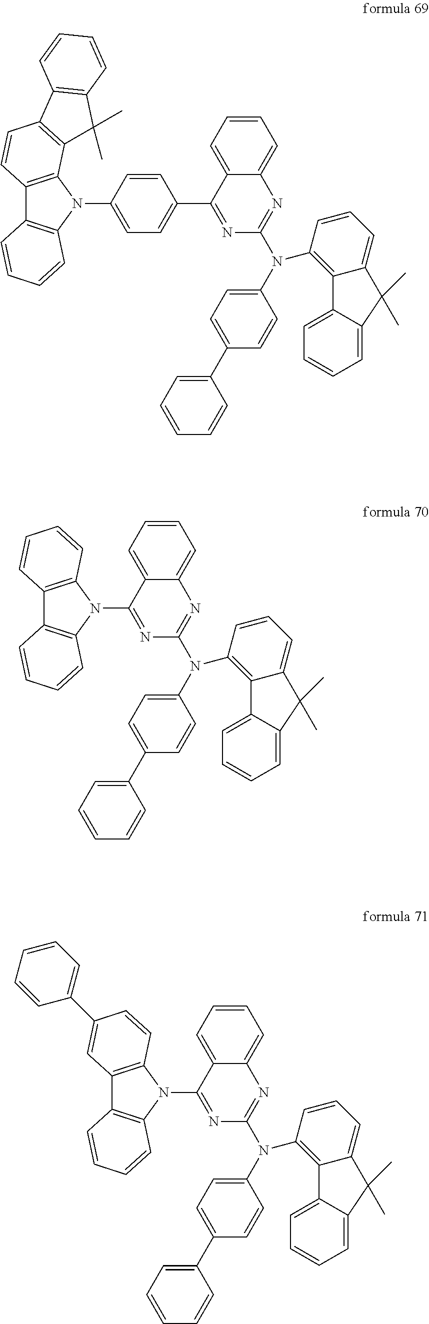 [SMILES: CC1(C)c2ccccc2-c2c(N(c3ccc(-c4ccccc4)cc3)c3nc(-c4ccc(-n5c6ccccc6c6ccc7c(c65)C(C)(C)c5ccccc5-7)cc4)c4ccccc4n3)cccc21 ; CC1(C)c2ccccc2-c2c(N(c3ccc(-c4ccccc4)cc3)c3nc(-n4c5ccccc5c5ccccc54)c4ccccc4n3)cccc21 ; CC1(C)c2ccccc2-c2c(N(c3ccc(-c4ccccc4)cc3)c3nc(-n4c5ccccc5c5cc(-c6ccccc6)ccc54)c4ccccc4n3)cccc21]

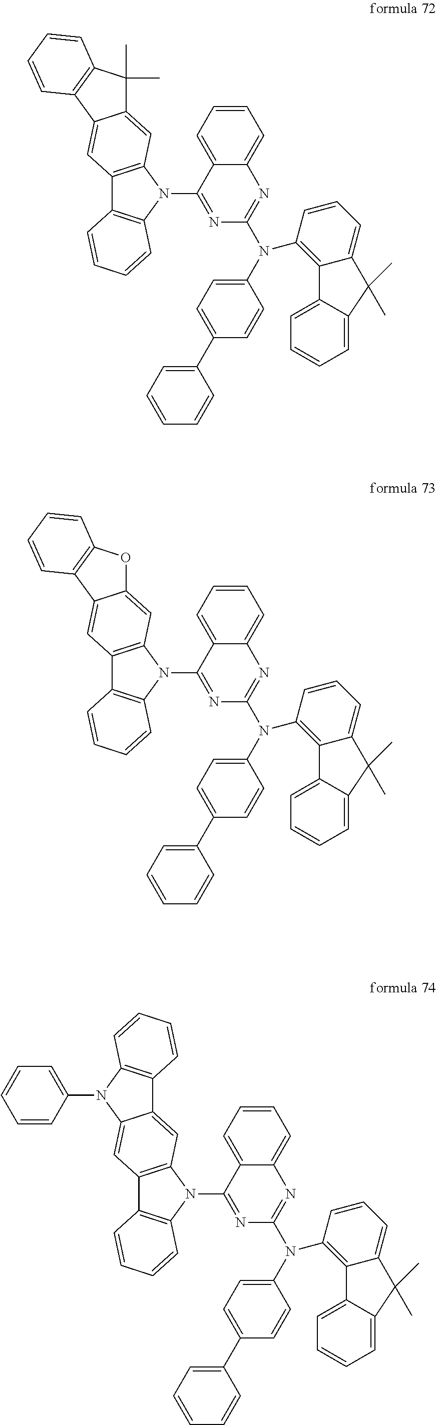 [SMILES: CC1(C)c2ccccc2-c2cc3c4ccccc4n(-c4nc(N(c5ccc(-c6ccccc6)cc5)c5cccc6c5-c5ccccc5C6(C)C)nc5ccccc45)c3cc21 ; CC1(C)c2ccccc2-c2c(N(c3ccc(-c4ccccc4)cc3)c3nc(-n4c5ccccc5c5cc6c(cc54)oc4ccccc46)c4ccccc4n3)cccc21 ; CC1(C)c2ccccc2-c2c(N(c3ccc(-c4ccccc4)cc3)c3nc(-n4c5ccccc5c5cc6c(cc54)c4ccccc4n6-c4ccccc4)c4ccccc4n3)cccc21]

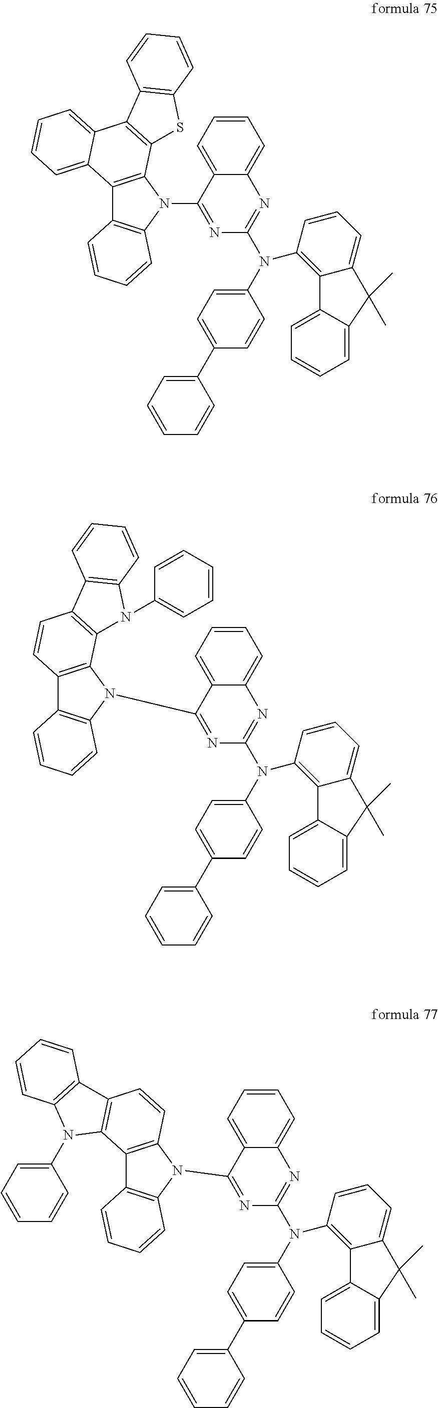 [SMILES: CC1(C)c2ccccc2-c2c(N(c3ccc(-c4ccccc4)cc3)c3nc(-n4c5ccccc5c5c6ccccc6c6c7ccccc7sc6c54)c4ccccc4n3)cccc21 ; CC1(C)c2ccccc2-c2c(N(c3ccc(-c4ccccc4)cc3)c3nc(-n4c5ccccc5c5ccc6c7ccccc7n(-c7ccccc7)c6c54)c4ccccc4n3)cccc21 ; CC1(C)c2ccccc2-c2c(N(c3ccc(-c4ccccc4)cc3)c3nc(-n4c5ccccc5c5c4ccc4c6ccccc6n(-c6ccccc6)c45)c4ccccc4n3)cccc21]

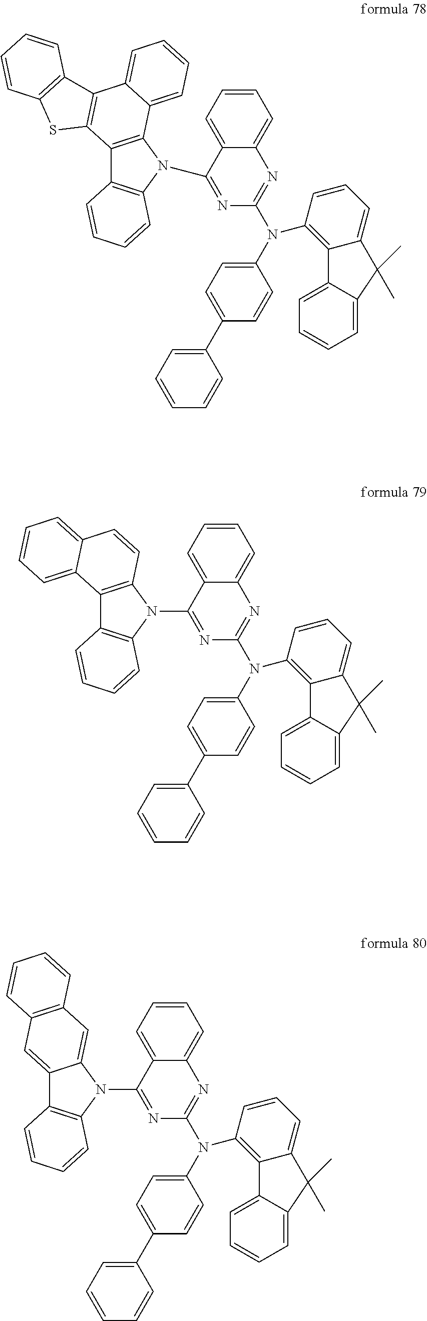 [SMILES: CC1(C)c2ccccc2-c2c(N(c3ccc(-c4ccccc4)cc3)c3nc(-n4c5ccccc5c5c6sc7ccccc7c6c6ccccc6c54)c4ccccc4n3)cccc21 ; CC1(C)c2ccccc2-c2c(N(c3ccc(-c4ccccc4)cc3)c3nc(-n4c5ccccc5c5c6ccccc6ccc54)c4ccccc4n3)cccc21 ; CC1(C)c2ccccc2-c2c(N(c3ccc(-c4ccccc4)cc3)c3nc(-n4c5ccccc5c5cc6ccccc6cc54)c4ccccc4n3)cccc21]

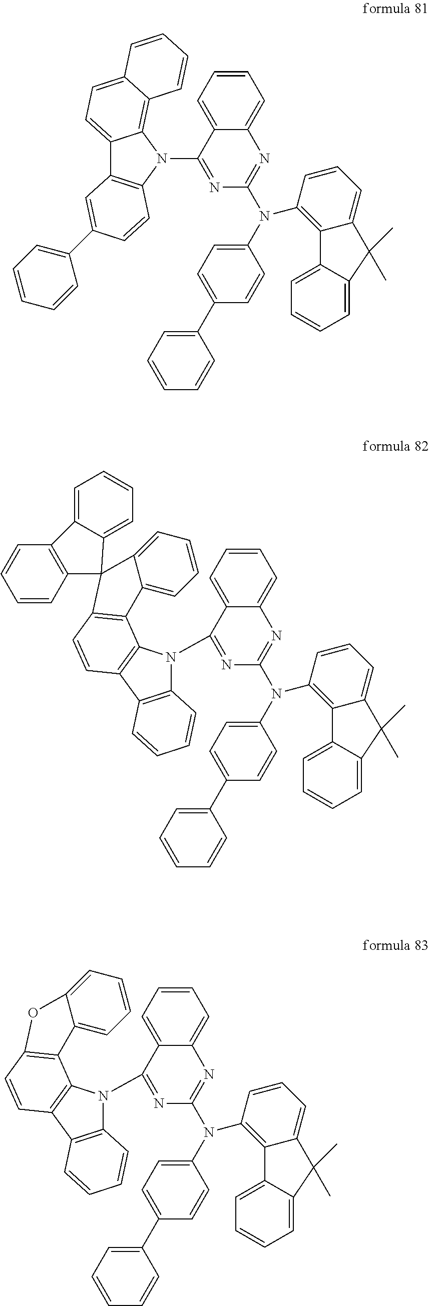 [SMILES: CC1(C)c2ccccc2-c2c(N(c3ccc(-c4ccccc4)cc3)c3nc(-n4c5ccc(-c6ccccc6)cc5c5ccc6ccccc6c54)c4ccccc4n3)cccc21 ; CC1(C)c2ccccc2-c2c(N(c3ccc(-c4ccccc4)cc3)c3nc(-n4c5ccccc5c5ccc6c(c54)-c4ccccc4C64c5ccccc5-c5ccccc54)c4ccccc4n3)cccc21 ; CC1(C)c2ccccc2-c2c(N(c3ccc(-c4ccccc4)cc3)c3nc(-n4c5ccccc5c5ccc6oc7ccccc7c6c54)c4ccccc4n3)cccc21]

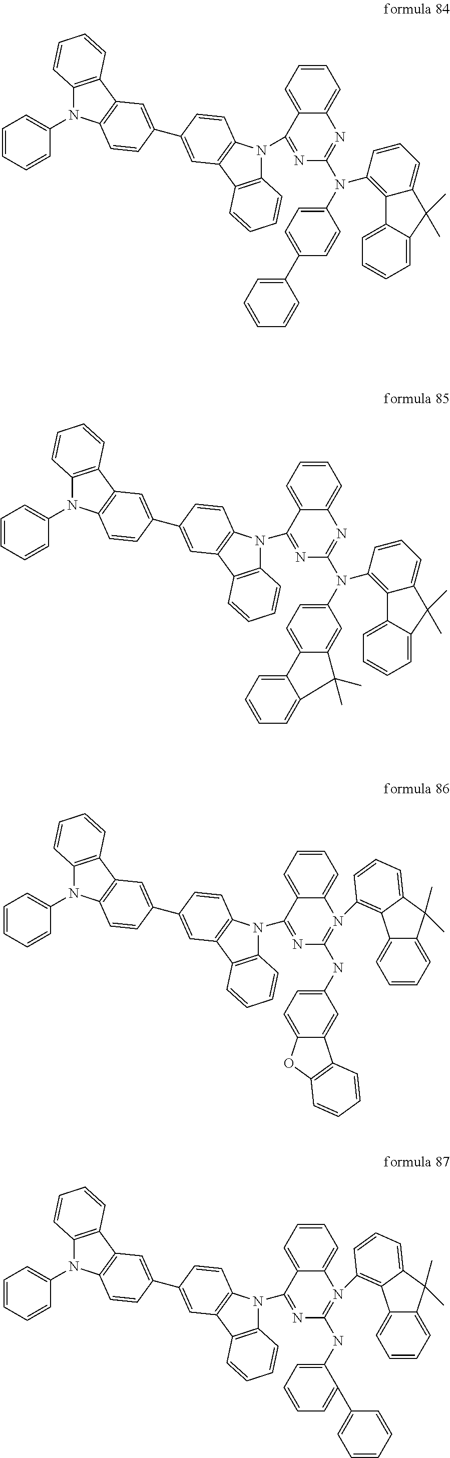

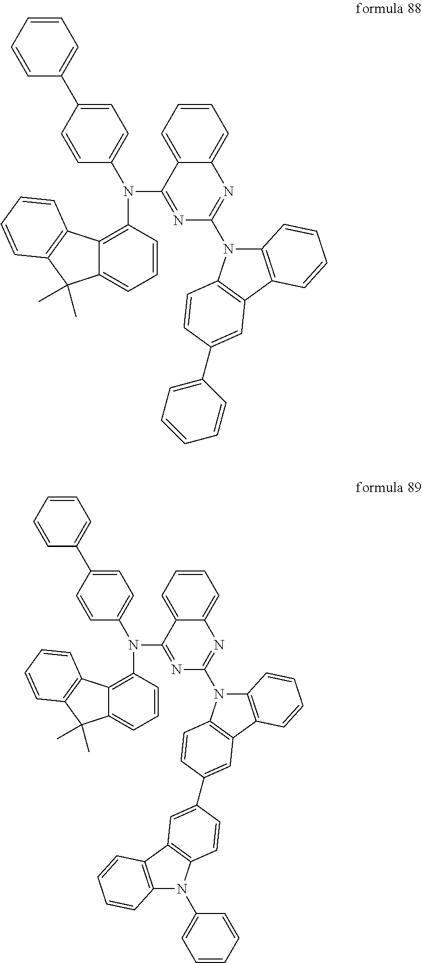 [SMILES: CC1(C)c2ccccc2-c2c(N(c3ccc(-c4ccccc4)cc3)c3nc(-n4c5ccccc5c5cc(-c6ccccc6)ccc54)nc4ccccc34)cccc21 ; CC1(C)c2ccccc2-c2c(N(c3ccc(-c4ccccc4)cc3)c3nc(-n4c5ccccc5c5cc(-c6ccc7c(c6)c6ccccc6n7-c6ccccc6)ccc54)nc4ccccc34)cccc21]

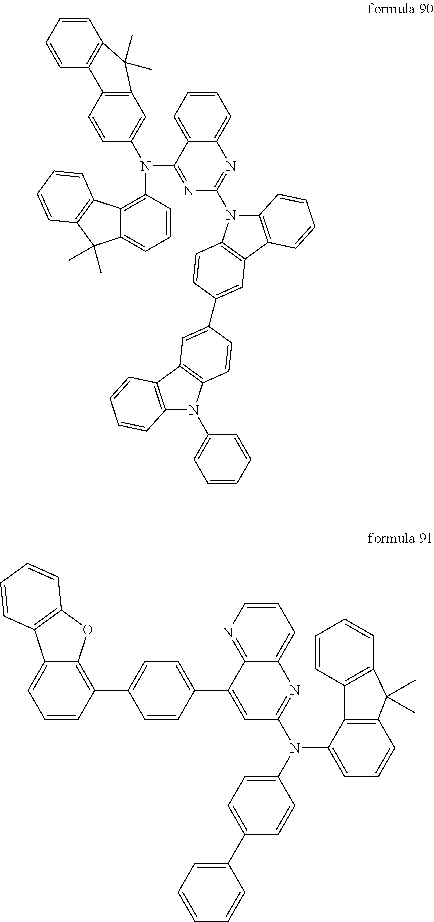 [SMILES: CC1(C)c2ccccc2-c2ccc(N(c3cccc4c3-c3ccccc3C4(C)C)c3nc(-n4c5ccccc5c5cc(-c6ccc7c(c6)c6ccccc6n7-c6ccccc6)ccc54)nc4ccccc34)cc21 ; CC1(C)c2ccccc2-c2c(N(c3ccc(-c4ccccc4)cc3)c3cc(-c4ccc(-c5cccc6c5oc5ccccc56)cc4)c4ncccc4n3)cccc21]

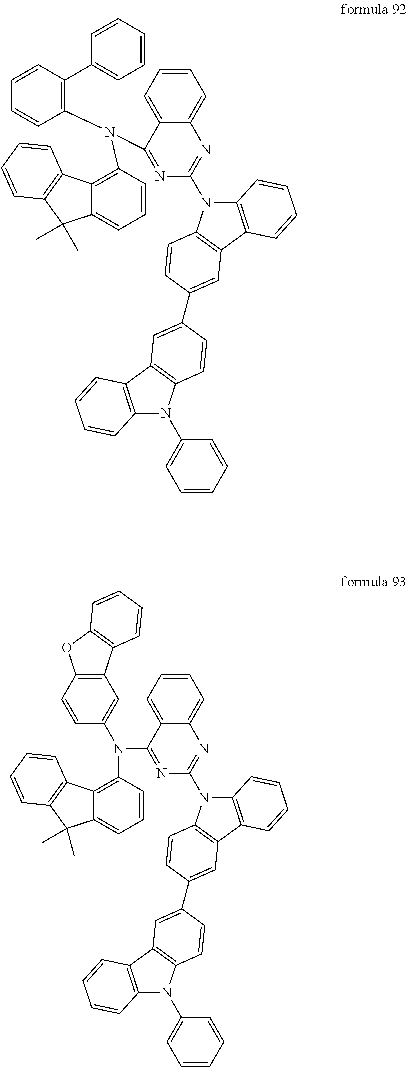 [SMILES: CC1(C)c2ccccc2-c2c(N(c3ccccc3-c3ccccc3)c3nc(-n4c5ccccc5c5cc(-c6ccc7c(c6)c6ccccc6n7-c6ccccc6)ccc54)nc4ccccc34)cccc21 ; CC1(C)c2ccccc2-c2c(N(c3ccc4oc5ccccc5c4c3)c3nc(-n4c5ccccc5c5cc(-c6ccc7c(c6)c6ccccc6n7-c6ccccc6)ccc54)nc4ccccc34)cccc21]

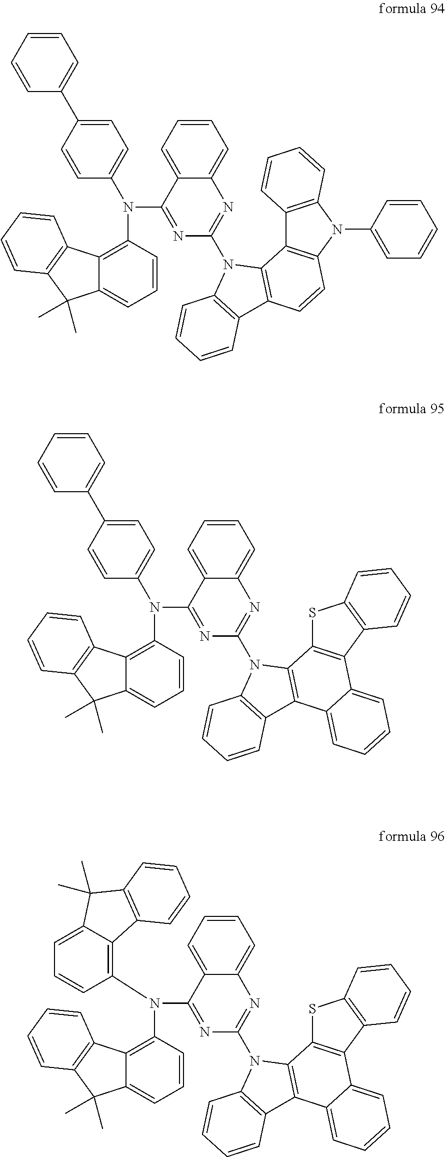 [SMILES: CC1(C)c2ccccc2-c2c(N(c3ccc(-c4ccccc4)cc3)c3nc(-n4c5ccccc5c5ccc6c(c7ccccc7n6-c6ccccc6)c54)nc4ccccc34)cccc21 ; CC1(C)c2ccccc2-c2c(N(c3ccc(-c4ccccc4)cc3)c3nc(-n4c5ccccc5c5c6ccccc6c6c7ccccc7sc6c54)nc4ccccc34)cccc21 ; CC1(C)c2ccccc2-c2c(N(c3cccc4c3-c3ccccc3C4(C)C)c3nc(-n4c5ccccc5c5c6ccccc6c6c7ccccc7sc6c54)nc4ccccc34)cccc21]

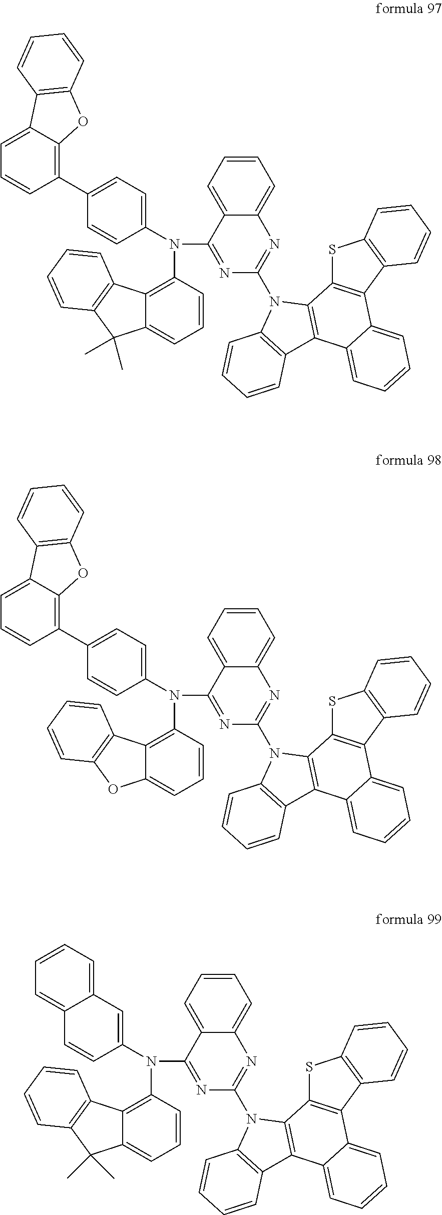 [SMILES: CC1(C)c2ccccc2-c2c(N(c3ccc(-c4cccc5c4oc4ccccc45)cc3)c3nc(-n4c5ccccc5c5c6ccccc6c6c7ccccc7sc6c54)nc4ccccc34)cccc21 ; c1ccc2c(N(c3ccc(-c4cccc5c4oc4ccccc45)cc3)c3cccc4oc5ccccc5c34)nc(-n3c4ccccc4c4c5ccccc5c5c6ccccc6sc5c43)nc2c1 ; CC1(C)c2ccccc2-c2c(N(c3ccc4ccccc4c3)c3nc(-n4c5ccccc5c5c6ccccc6c6c7ccccc7sc6c54)nc4ccccc34)cccc21]

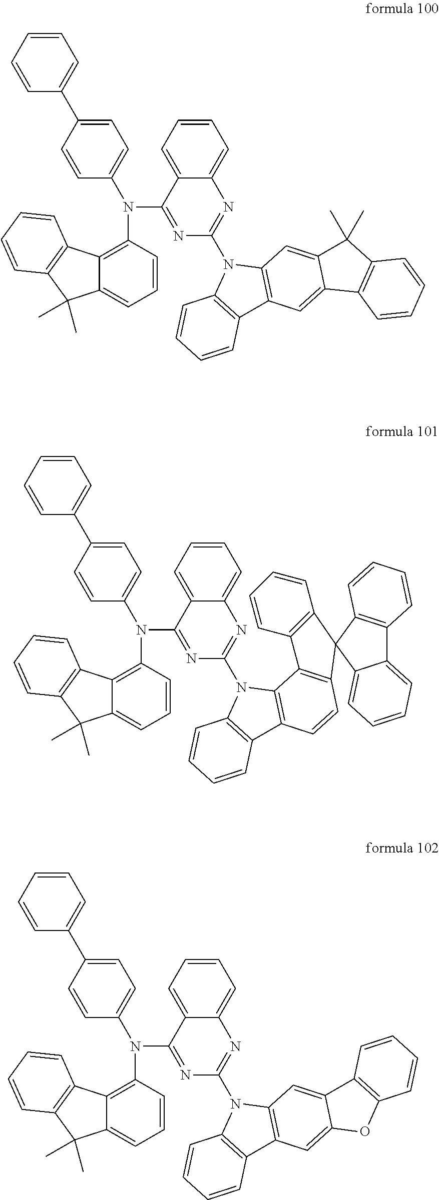 [SMILES: CC1(C)c2ccccc2-c2cc3c4ccccc4n(-c4nc(N(c5ccc(-c6ccccc6)cc5)c5cccc6c5-c5ccccc5C6(C)C)c5ccccc5n4)c3cc21 ; CC1(C)c2ccccc2-c2c(N(c3ccc(-c4ccccc4)cc3)c3nc(-n4c5ccccc5c5ccc6c(c54)-c4ccccc4C64c5ccccc5-c5ccccc54)nc4ccccc34)cccc21 ; CC1(C)c2ccccc2-c2c(N(c3ccc(-c4ccccc4)cc3)c3nc(-n4c5ccccc5c5cc6oc7ccccc7c6cc54)nc4ccccc34)cccc21]

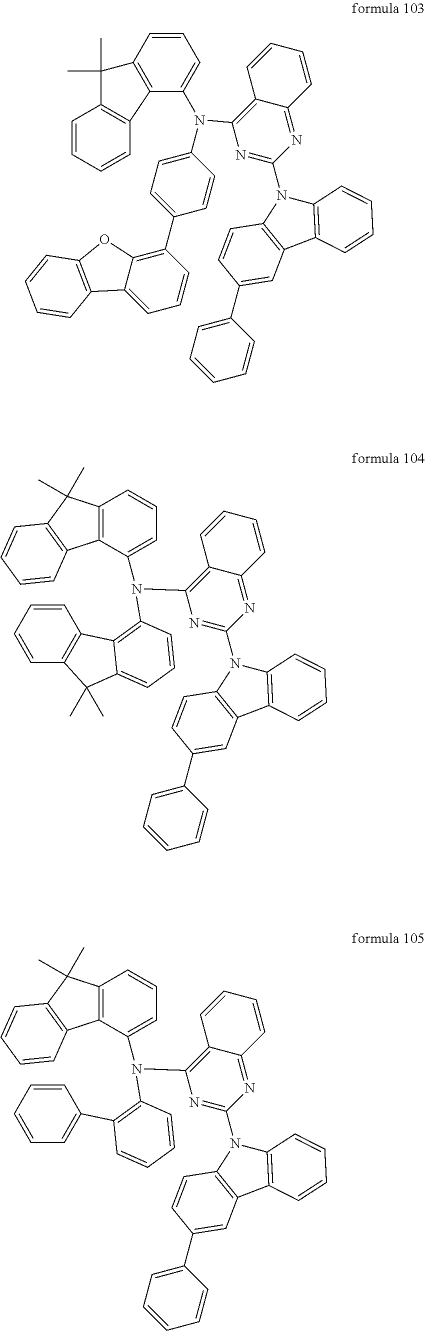 [SMILES: CC1(C)c2ccccc2-c2c(N(c3ccc(-c4cccc5c4oc4ccccc45)cc3)c3nc(-n4c5ccccc5c5cc(-c6ccccc6)ccc54)nc4ccccc34)cccc21 ; CC1(C)c2ccccc2-c2c(N(c3cccc4c3-c3ccccc3C4(C)C)c3nc(-n4c5ccccc5c5cc(-c6ccccc6)ccc54)nc4ccccc34)cccc21 ; CC1(C)c2ccccc2-c2c(N(c3ccccc3-c3ccccc3)c3nc(-n4c5ccccc5c5cc(-c6ccccc6)ccc54)nc4ccccc34)cccc21]

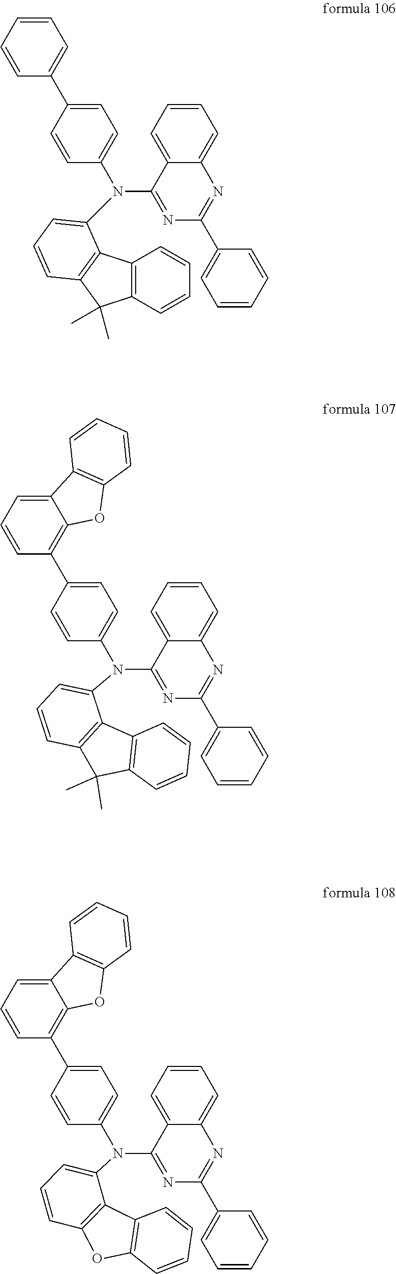 [SMILES: CC1(C)c2ccccc2-c2c(N(c3ccc(-c4ccccc4)cc3)c3nc(-c4ccccc4)nc4ccccc34)cccc21 ; CC1(C)c2ccccc2-c2c(N(c3ccc(-c4cccc5c4oc4ccccc45)cc3)c3nc(-c4ccccc4)nc4ccccc34)cccc21 ; c1ccc(-c2nc(N(c3ccc(-c4cccc5c4oc4ccccc45)cc3)c3cccc4oc5ccccc5c34)c3ccccc3n2)cc1]

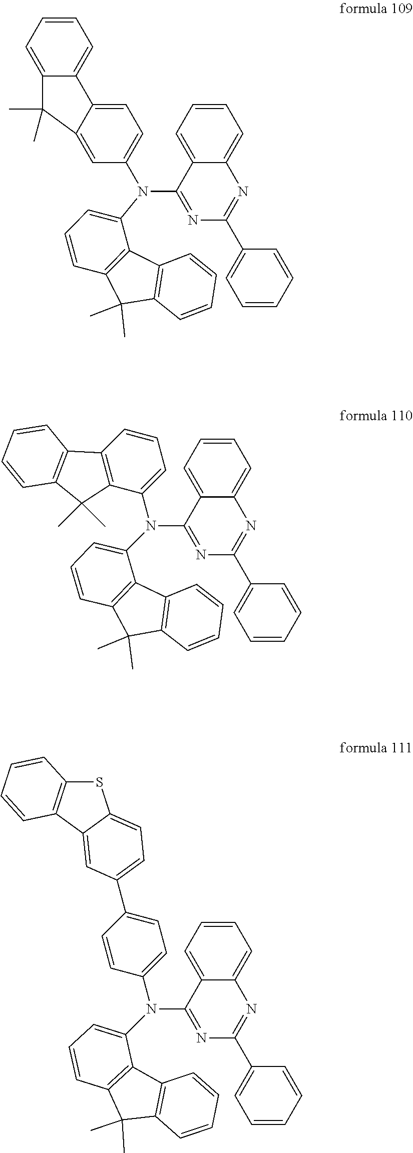 [SMILES: CC1(C)c2ccccc2-c2ccc(N(c3cccc4c3-c3ccccc3C4(C)C)c3nc(-c4ccccc4)nc4ccccc34)cc21 ; CC1(C)c2ccccc2-c2c(N(c3cccc4c3C(C)(C)c3ccccc3-4)c3nc(-c4ccccc4)nc4ccccc34)cccc21 ; CC1(C)c2ccccc2-c2c(N(c3ccc(-c4ccc5sc6ccccc6c5c4)cc3)c3nc(-c4ccccc4)nc4ccccc34)cccc21]

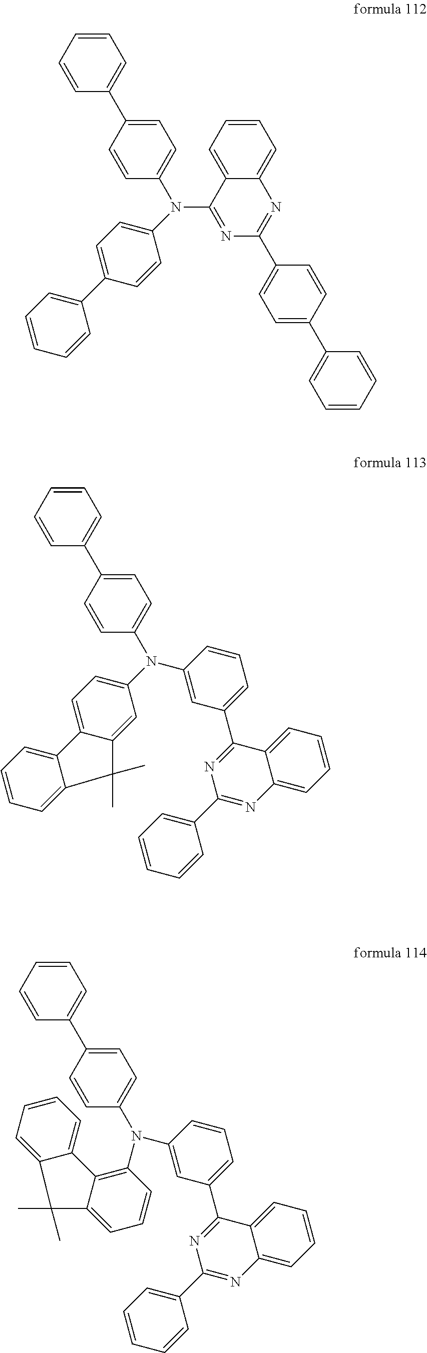 [SMILES: c1ccc(-c2ccc(-c3nc(N(c4ccc(-c5ccccc5)cc4)c4ccc(-c5ccccc5)cc4)c4ccccc4n3)cc2)cc1 ; CC1(C)c2ccccc2-c2ccc(N(c3ccc(-c4ccccc4)cc3)c3cccc(-c4nc(-c5ccccc5)nc5ccccc45)c3)cc21 ; CC1(C)c2ccccc2-c2c(N(c3ccc(-c4ccccc4)cc3)c3cccc(-c4nc(-c5ccccc5)nc5ccccc45)c3)cccc21]

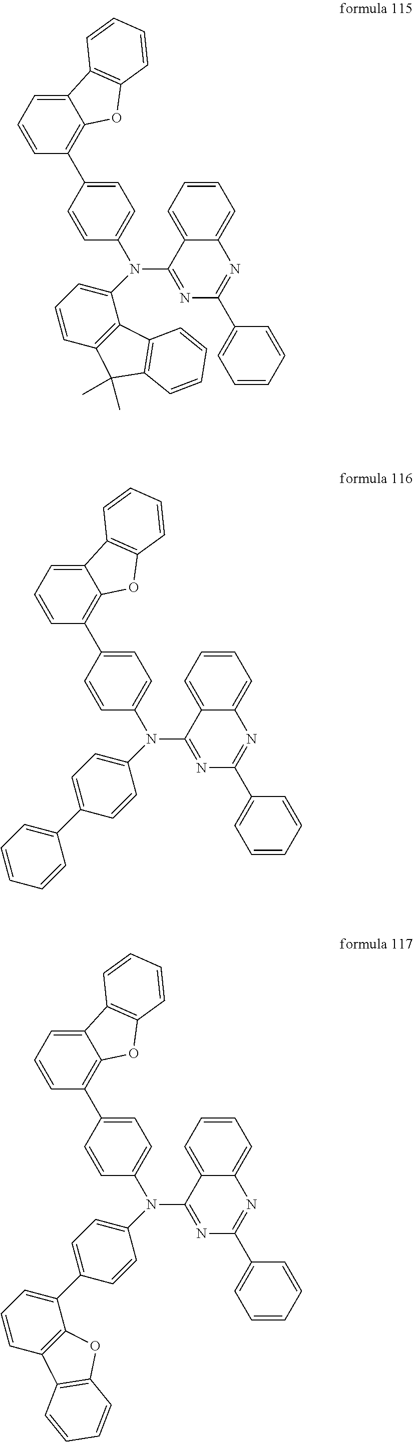 [SMILES: CC1(C)c2ccccc2-c2c(N(c3ccc(-c4cccc5c4oc4ccccc45)cc3)c3nc(-c4ccccc4)nc4ccccc34)cccc21 ; c1ccc(-c2ccc(N(c3ccc(-c4cccc5c4oc4ccccc45)cc3)c3nc(-c4ccccc4)nc4ccccc34)cc2)cc1 ; c1ccc(-c2nc(N(c3ccc(-c4cccc5c4oc4ccccc45)cc3)c3ccc(-c4cccc5c4oc4ccccc45)cc3)c3ccccc3n2)cc1]

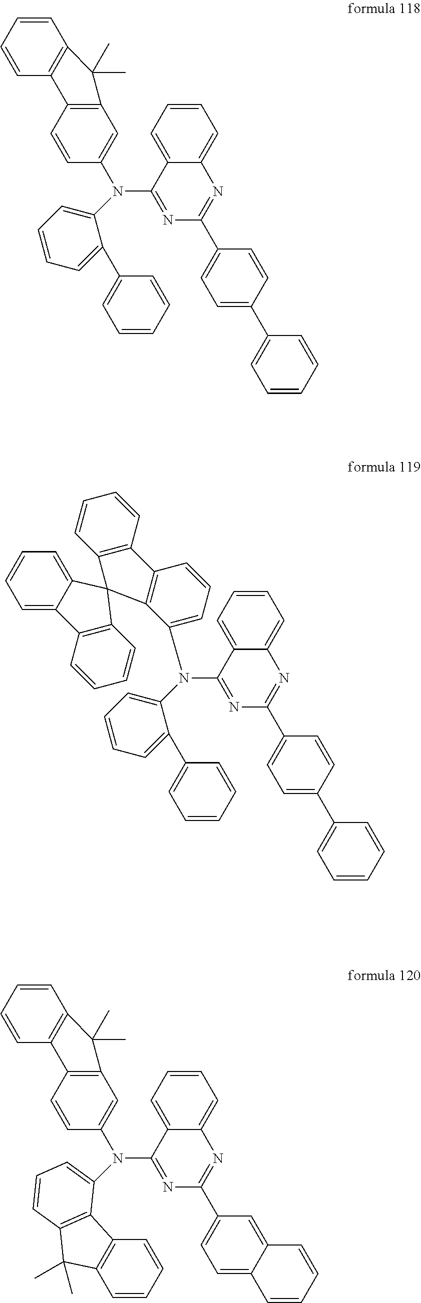 [SMILES: CC1(C)c2ccccc2-c2ccc(N(c3ccccc3-c3ccccc3)c3nc(-c4ccc(-c5ccccc5)cc4)nc4ccccc34)cc21 ; c1ccc(-c2ccc(-c3nc(N(c4ccccc4-c4ccccc4)c4cccc5c4C4(c6ccccc6-c6ccccc64)c4ccccc4-5)c4ccccc4n3)cc2)cc1 ; CC1(C)c2ccccc2-c2ccc(N(c3cccc4c3-c3ccccc3C4(C)C)c3nc(-c4ccc5ccccc5c4)nc4ccccc34)cc21]

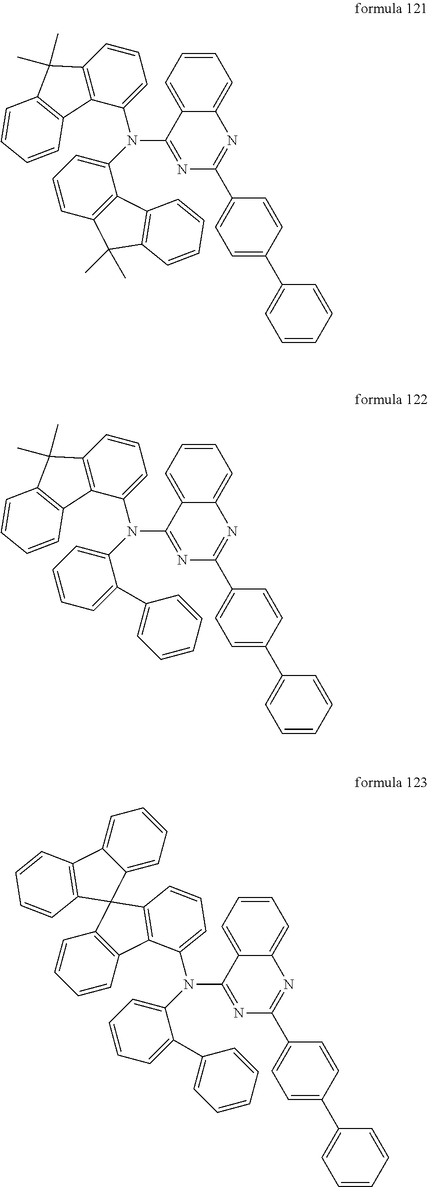 [SMILES: CC1(C)c2ccccc2-c2c(N(c3cccc4c3-c3ccccc3C4(C)C)c3nc(-c4ccc(-c5ccccc5)cc4)nc4ccccc34)cccc21 ; CC1(C)c2ccccc2-c2c(N(c3ccccc3-c3ccccc3)c3nc(-c4ccc(-c5ccccc5)cc4)nc4ccccc34)cccc21 ; c1ccc(-c2ccc(-c3nc(N(c4ccccc4-c4ccccc4)c4cccc5c4-c4ccccc4C54c5ccccc5-c5ccccc54)c4ccccc4n3)cc2)cc1]

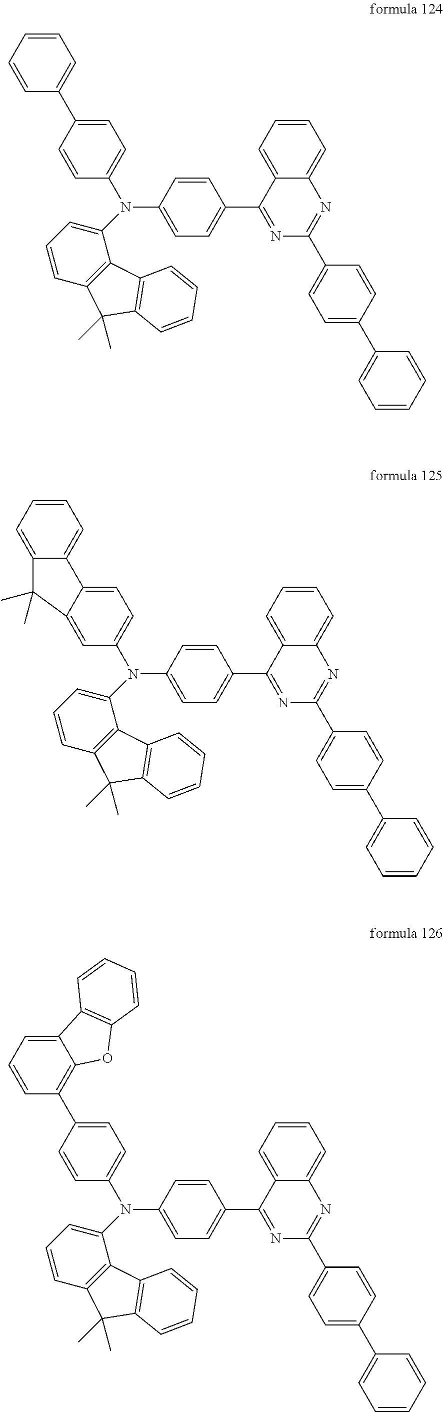 [SMILES: CC1(C)c2ccccc2-c2c(N(c3ccc(-c4ccccc4)cc3)c3ccc(-c4nc(-c5ccc(-c6ccccc6)cc5)nc5ccccc45)cc3)cccc21 ; CC1(C)c2ccccc2-c2ccc(N(c3ccc(-c4nc(-c5ccc(-c6ccccc6)cc5)nc5ccccc45)cc3)c3cccc4c3-c3ccccc3C4(C)C)cc21 ; CC1(C)c2ccccc2-c2c(N(c3ccc(-c4nc(-c5ccc(-c6ccccc6)cc5)nc5ccccc45)cc3)c3ccc(-c4cccc5c4oc4ccccc45)cc3)cccc21]

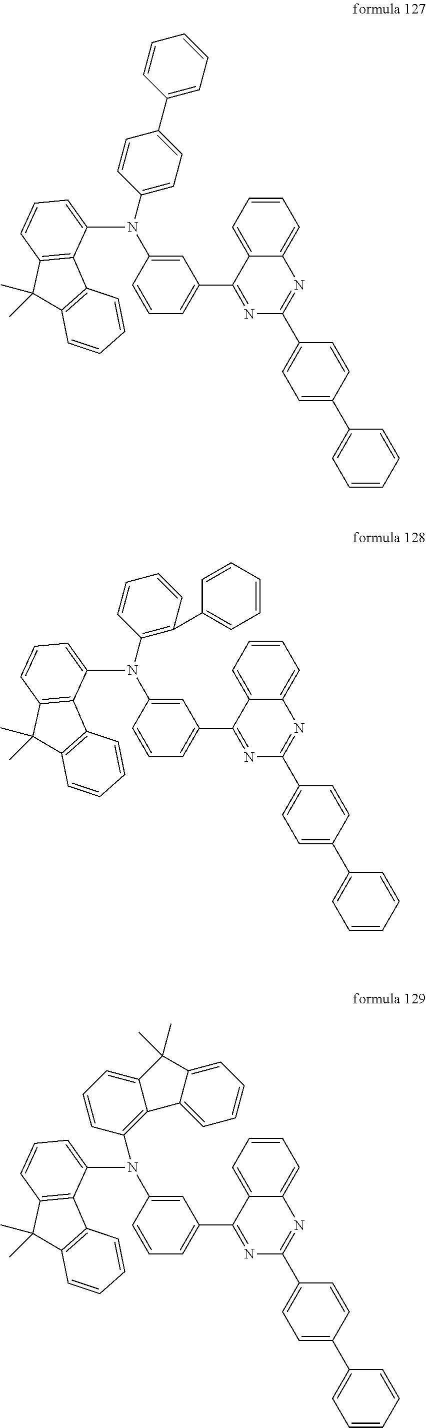 [SMILES: CC1(C)c2ccccc2-c2c(N(c3ccc(-c4ccccc4)cc3)c3cccc(-c4nc(-c5ccc(-c6ccccc6)cc5)nc5ccccc45)c3)cccc21 ; CC1(C)c2ccccc2-c2c(N(c3cccc(-c4nc(-c5ccc(-c6ccccc6)cc5)nc5ccccc45)c3)c3ccccc3-c3ccccc3)cccc21 ; CC1(C)c2ccccc2-c2c(N(c3cccc(-c4nc(-c5ccc(-c6ccccc6)cc5)nc5ccccc45)c3)c3cccc4c3-c3ccccc3C4(C)C)cccc21]

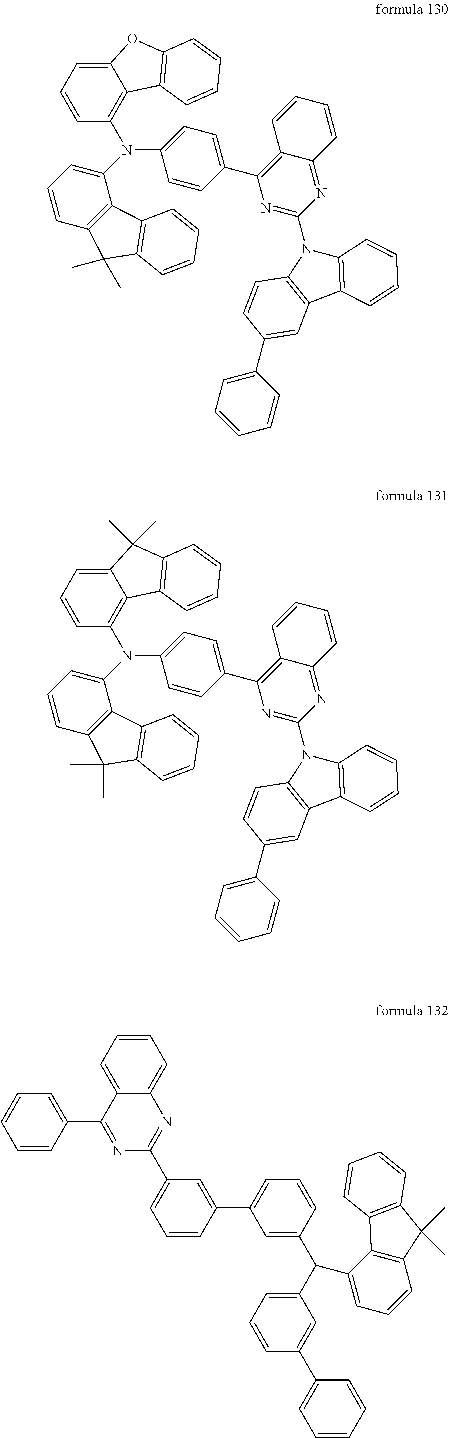 [SMILES: CC1(C)c2ccccc2-c2c(N(c3ccc(-c4nc(-n5c6ccccc6c6cc(-c7ccccc7)ccc65)nc5ccccc45)cc3)c3cccc4oc5ccccc5c34)cccc21 ; CC1(C)c2ccccc2-c2c(N(c3ccc(-c4nc(-n5c6ccccc6c6cc(-c7ccccc7)ccc65)nc5ccccc45)cc3)c3cccc4c3-c3ccccc3C4(C)C)cccc21 ; CC1(C)c2ccccc2-c2c(C(c3cccc(-c4ccccc4)c3)c3cccc(-c4cccc(-c5nc(-c6ccccc6)c6ccccc6n5)c4)c3)cccc21]

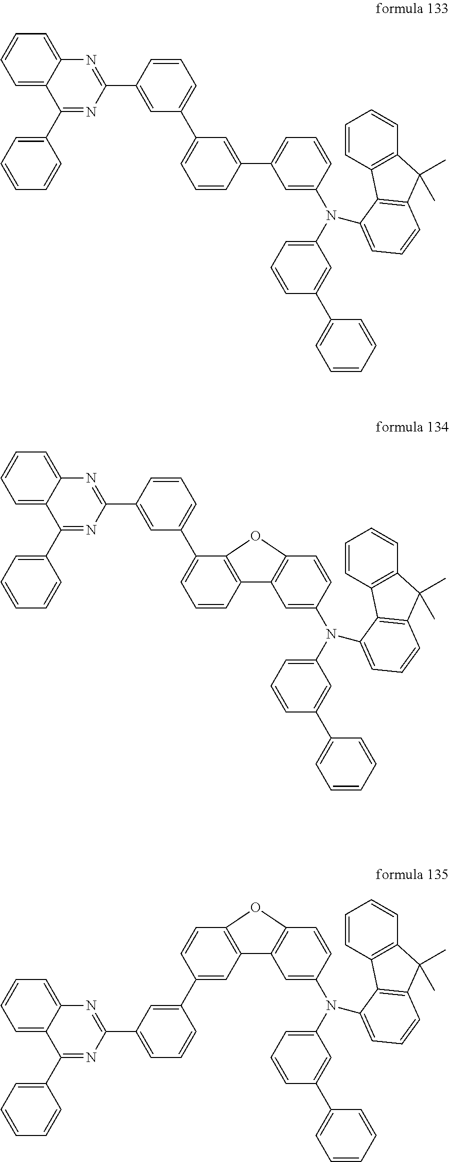 [SMILES: CC1(C)c2ccccc2-c2c(N(c3cccc(-c4ccccc4)c3)c3cccc(-c4cccc(-c5cccc(-c6nc(-c7ccccc7)c7ccccc7n6)c5)c4)c3)cccc21 ; CC1(C)c2ccccc2-c2c(N(c3cccc(-c4ccccc4)c3)c3ccc4oc5c(-c6cccc(-c7nc(-c8ccccc8)c8ccccc8n7)c6)cccc5c4c3)cccc21 ; CC1(C)c2ccccc2-c2c(N(c3cccc(-c4ccccc4)c3)c3ccc4oc5ccc(-c6cccc(-c7nc(-c8ccccc8)c8ccccc8n7)c6)cc5c4c3)cccc21]

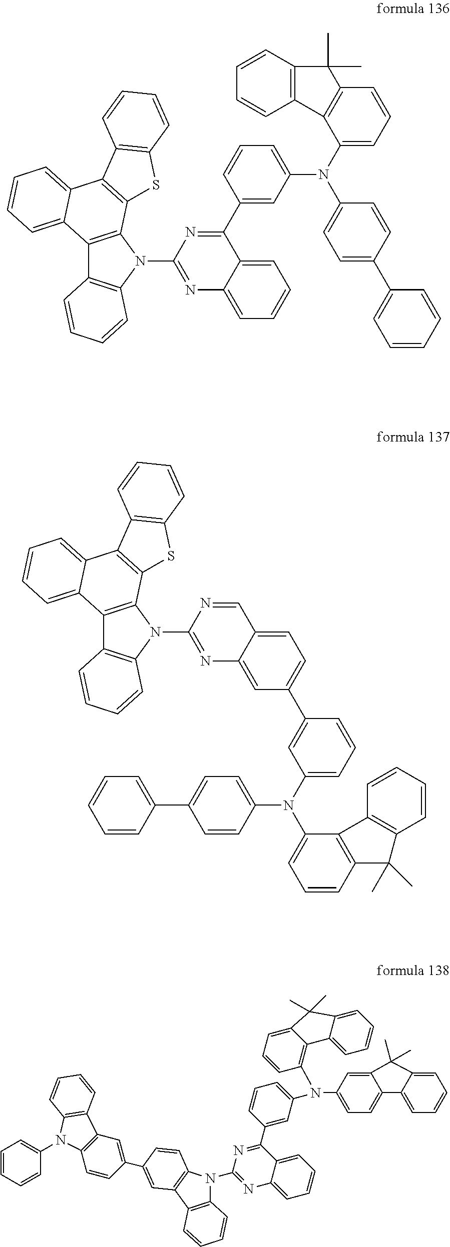 [SMILES: CC1(C)c2ccccc2-c2c(N(c3ccc(-c4ccccc4)cc3)c3cccc(-c4nc(-n5c6ccccc6c6c7ccccc7c7c8ccccc8sc7c65)nc5ccccc45)c3)cccc21 ; CC1(C)c2ccccc2-c2c(N(c3ccc(-c4ccccc4)cc3)c3cccc(-c4ccc5cnc(-n6c7ccccc7c7c8ccccc8c8c9ccccc9sc8c76)nc5c4)c3)cccc21 ; CC1(C)c2ccccc2-c2ccc(N(c3cccc(-c4nc(-n5c6ccccc6c6cc(-c7ccc8c(c7)c7ccccc7n8-c7ccccc7)ccc65)nc5ccccc45)c3)c3cccc4c3-c3ccccc3C4(C)C)cc21]

Preferred embodiments of the compounds according to the invention are explained in greater detail in the examples, it being possible for these compounds to be employed alone or in combination with further compounds for all applications according to the invention.

Provided that the conditions mentioned in claim 1 are observed, the preferred embodiments mentioned above can be combined with one another as desired. In a particularly preferred embodiment of the invention, the preferred embodiments mentioned above apply simultaneously.

The compounds according to the invention can in principle be prepared by various processes. However, the processes described below have proven particularly suitable.

The present invention therefore furthermore relates to a process for the preparation of the compounds containing structures of the formula (I) in which a diarylamine compound is coupled to a compound containing at least one diazanaphthyl group in a coupling reaction.

Suitable compounds containing a diazanaphthyl group or diarylamine compounds are in many cases commercially available, the starting compounds described in the examples being obtainable by known processes, to which reference is therefore made.

These compounds can be reacted with further aryl compounds by known coupling reactions, with the requisite conditions for this purpose being known to the person skilled in the art and detailed information in the examples supporting the person skilled in the art in carrying out these reactions. Particularly suitable and preferred coupling reactions, all of which lead to C—C links and/or C—N links, are BUCHWALD, SUZUKI, YAMAMOTO, STILLE, HECK, NEGISHI, SONOGASHIRA and HIYAMA coupling reactions. These reactions are widely known, with the examples providing further information to the person skilled in the art.

In all synthesis schemes below, the compounds are shown with a small number of substituents in order to simplify the structures. This does not exclude the presence of any desired further substituents in the process.

The following schemes show one reaction by way of example, without this being intended to be a restriction. The part-steps of the individual schemes can be combined as desired here.

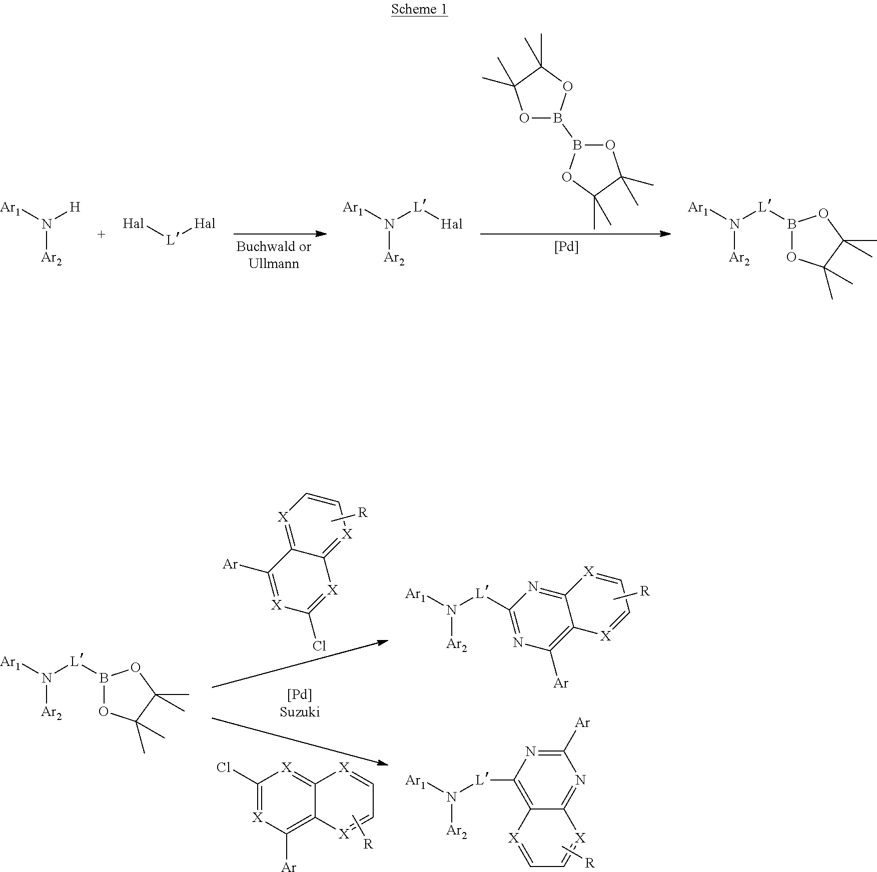

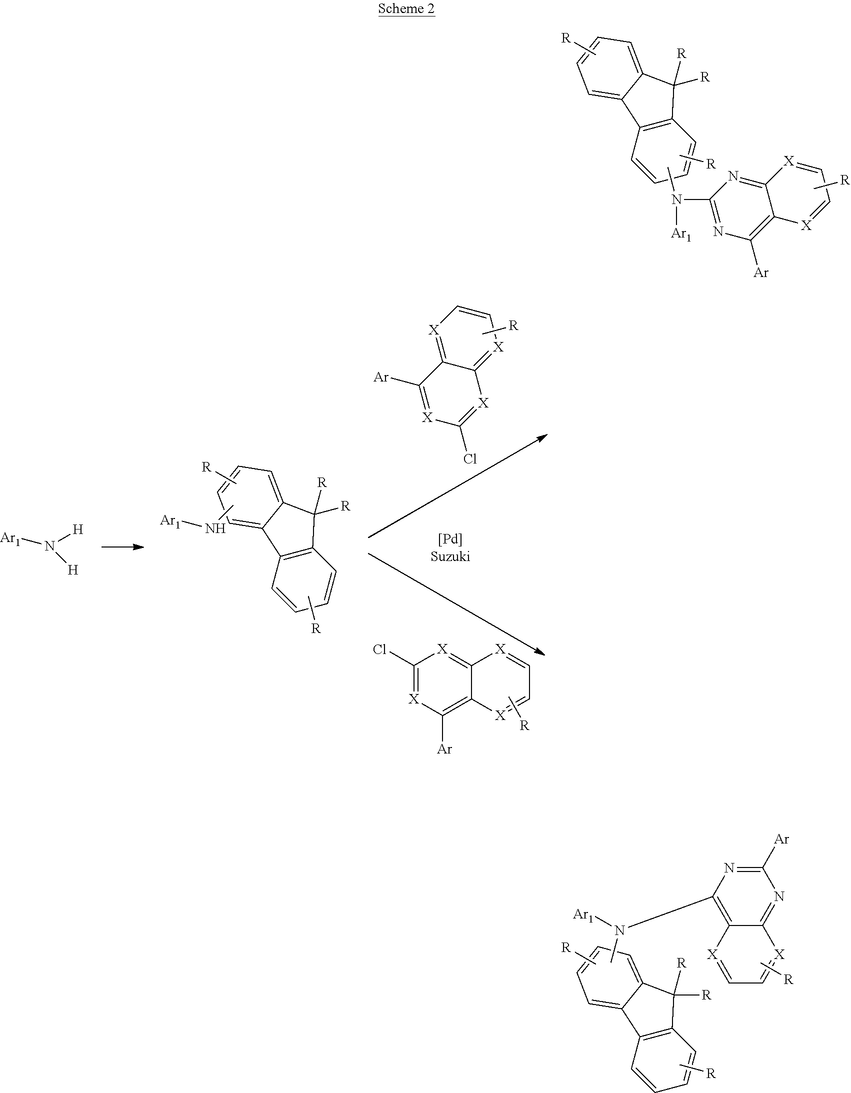

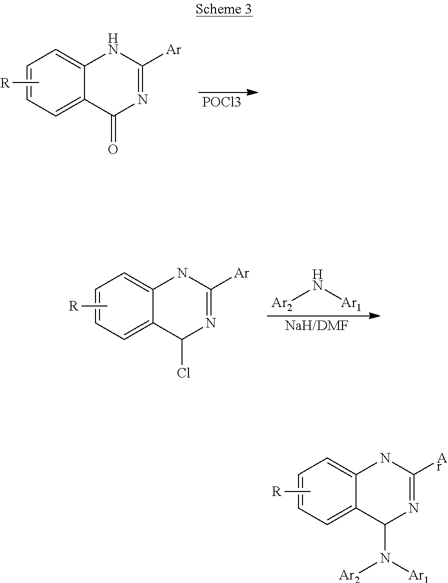

### Journal of Materials Chemistry, 22(14), 6878-6884; 2012.

The meaning of the symbols used in Schemes 1 to 3 essentially corresponds to those defined for formula (I), where, for reasons of clarity, numbering has in many cases been omitted, but the diarylamine or triarylamine structure has been shown with the symbols Ar¹, Ar² and in some cases L′ for better legibility in order to make it clear that the aryl groups may be different.

The processes shown for the synthesis of the compounds according to the invention should be understood as being illustrative. The person skilled in the art will be able to develop alternative synthetic routes within the scope of his general expert knowledge. The basic principles of the preparation processes described above are known in principle from the literature for similar compounds and can easily be adapted by the person skilled in the art for the preparation of the compounds according to the invention. Further information can be obtained from the examples.

These processes, optionally followed by purification, such as, for example, recrystallisation or sublimation, enable the compounds according to the invention containing structures of the formula (I) to be obtained in high purity, preferably greater than 99% (determined by means of 1H-NMR and/or HPLC).

The compounds according to the invention may also contain suitable substituents, for example relatively long alkyl groups (about 4 to 20 C atoms), in particular branched alkyl groups, or optionally substituted aryl groups, for example xylyl, mesityl or branched terphenyl or quaterphenyl groups, which effect solubility in common organic solvents, such as, for example, toluene or xylene, at room temperature in adequate concentration in order to be able to process the compounds from solution. These soluble compounds are particularly suitable for processing from solution, for example by printing processes. Furthermore, it should be noted that the compounds according to the invention containing at least one structure of the formula (I) already have increased solubility in these solvents.

The compounds according to the invention can also be mixed with a polymer. It is likewise possible to incorporate these compounds covalently into a polymer. This is possible, in particular, with compounds which are substituted by reactive leaving groups, such as bromine, iodine, chlorine, boronic acid or boronic acid ester, or by reactive, polymerisable groups, such as olefins or oxetanes. These can be used as monomers for the preparation of corresponding oligomers, dendrimers or polymers. The oligomerisation or polymerisation here preferably takes place via the halogen functionality or the boronic acid functionality or via the polymerisable group. It is furthermore possible to crosslink the polymers via such groups. The compounds and polymers according to the invention can be employed as crosslinked or uncrosslinked layer.

The invention therefore furthermore relates to oligomers, polymers or dendrimers containing one or more of the structures of the formula (I) shown above or compounds according to the invention, where one or more bonds are present from the compounds according to the invention or the structures of the formula (I) to the polymer, oligomer or dendrimer. Depending on the linking of the structures of the formula (I) or the compounds, these therefore form a side chain of the oligomer or polymer or are linked in the main chain. The polymers, oligomers or dendrimers may be conjugated, partially conjugated or non-conjugated. The oligomers or polymers may be linear, branched or dendritic. The same preferences as described above apply to the recurring units of the compounds according to the invention in oligomers, dendrimers and polymers.

For the preparation of the oligomers or polymers, the monomers according to the invention are homopolymerised or copolymerised with further monomers. Preference is given to copolymers, where the units of the formula (I) or the preferred embodiments indicated above and below are present to the extent of 0.01 to 99.9 mol %, preferably 5 to 90 mol %, particularly preferably 20 to 80 mol %. Suitable and preferred comonomers which form the polymer backbone are selected from fluorenes (for example in accordance with EP 842208 or WO 2000/022026), spirobifluorenes (for example in accordance with EP 707020, EP 894107 or WO 2006/061181), paraphenylenes (for example in accordance with WO 92/18552), carbazoles (for example in accordance with WO 2004/070772 or WO 2004/113468), thiophenes (for example in accordance with EP 1028136), dihydrophenanthrenes (for example in accordance with WO 2005/014689), cis- and trans-indenofluorenes (for example in accordance with WO 2004/041901 or WO 2004/113412), ketones (for example in accordance with WO 2005/040302), phenanthrenes (for example in accordance with WO 2005/104264 or WO 2007/017066) or also a plurality of these units. The polymers, oligomers and dendrimers may also contain further units, for example hole-transport units, in particular those based on triarylamines, and/or electron-transport units.

Of particular interest are furthermore compounds according to the invention which are distinguished by a high glass-transition temperature. In this connection, particular preference is given to compounds according to the invention containing structures of the general formula (I) or the preferred embodiments indicated above and below which have a glass-transition temperature of at least 70° C., particularly preferably at least 110° C., very particularly preferably at least 125° C. and especially preferably at least 150° C., determined in accordance with DIN 51005 (version 2005-08).

For the processing of the compounds according to the invention from the liquid phase, for example by spin coating or by printing processes, formulations of the compounds according to the invention are necessary. These formulations can be, for example, solutions, dispersions or emulsions. It may be preferred to use mixtures of two or more solvents for this purpose. Suitable and preferred solvents are, for example, toluene, anisole, o-, m- or p-xylene, methyl benzoate, mesitylene, tetralin, veratrol, THF, methyl-THF, THP, chlorobenzene, dioxane, phenoxytoluene, in particular 3-phenoxytoluene, (-)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetol, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, hexamethylindane or mixtures of these solvents.

The present invention therefore furthermore relates to a formulation comprising a compound according to the invention and at least one further compound. The further compound can be, for example, a solvent, in particular one of the above-mentioned solvents or a mixture of these solvents. However, the further compound can also be at least one further organic or inorganic compound which is likewise employed in the electronic device, for example an emitting compound, in particular a phosphorescent dopant, and/or a further matrix material. This further compound may also be polymeric.

The present invention still furthermore relates to a composition comprising a compound according to the invention and at least one further organofunctional material. Functional materials are generally the organic or inorganic materials which are introduced between the anode and the cathode. The organofunctional material is preferably selected from the group consisting of fluorescent emitters, phosphorescent emitters, emitters which exhibit TADF (thermally activated delayed fluorescence), host materials, electron-transport materials, electron-injection materials, hole-conductor materials, hole-injection materials, electron-blocking materials, hole-blocking materials, wide band gap materials and n-dopants.

The present invention therefore also relates to a composition comprising at least one compound containing structures of the formula (I) or the preferred embodiments indicated above and below and at least one further matrix material. According to a particular aspect of the present invention, the further matrix material has hole-transporting properties.

The present invention furthermore relates to a composition comprising at least one compound containing at least one structure of the formula (I) or the preferred embodiments indicated above and below and at least one wide band gap material, where a wide band gap material is taken to mean a material in the sense of the disclosure of U.S. Pat. No. 7,294,849. These systems exhibit particularly advantageous performance data in electroluminescent devices.

The additional compound can preferably have a band gap of 2.5 eV or more, preferably 3.0 eV or more, very preferably 3.5 eV or more. The band gap can be calculated, inter alia, by means of the energy levels of the highest occupied molecular orbital (HOMO) and the lowest unoccupied molecular orbital (LUMO).

Molecular orbitals, in particular also the highest occupied molecular orbital (HOMO) and the lowest unoccupied molecular orbital (LUMO), their energy levels and the energy of the lowest triplet state T₁ or of the lowest excited singlet state S₁ of the materials are determined via quantum-chemical calculations. In order to calculate organic substances without metals, firstly a geometry optimisation is carried out using the “Ground State/Semi-empirical/Default Spin/AM1/Charge 0/Spin Singlet” method. An energy calculation is subsequently carried out on the basis of the optimised geometry. The “TD-SCF/DFT/Default Spin/B3PW91” method with the “6-31G(d)” base set (charge 0, spin singlet) is used here. For metal-containing compounds, the geometry is optimised via the “Ground State/Hartree-Fock/Default Spin/LanL2 MB/Charge 0/Spin Singlet” method. The energy calculation is carried out analogously to the above-described method for the organic substances, with the difference that the “LanL2DZ” base set is used for the metal atom and the “6-31G(d)” base set is used for the ligands. The energy calculation gives the HOMO energy level HEh or LUMO energy level LEh in hartree units. The HOMO and LUMO energy levels in electron volts calibrated with reference to cyclic voltammetry measurements are determined therefrom as follows:

$$HOMO ( eV ) = ( ( HEh * 27.212 ) - 0.9899 ) / 1 \text{.1206}$$

$$LUMO ( eV ) = ( ( LEh * 27.212 ) - 2.0041 ) / 1.385$$

For the purposes of this application, these values are to be regarded as HOMO and LUMO energy levels respectively of the materials.

The lowest triplet state T₁ is defined as the energy of the triplet state having the lowest energy which arises from the quantum-chemical calculation described.

The lowest excited singlet state S₁ is defined as the energy of the excited singlet state having the lowest energy which arises from the quantum-chemical calculation described.

The method described herein is independent of the software package used and always gives the same results. Examples of frequently used programs for this purpose are “Gaussian09 W” (Gaussian Inc.) and Q-Chem 4.1 (Q-Chem, Inc.).

The present invention also relates to a composition comprising at least one compound containing structures of the formula (I) or the preferred embodiments indicated above and below and at least one phosphorescent emitter, where the term phosphorescent emitter is also taken to mean phosphorescent dopants.

A dopant in a system comprising a matrix material and a dopant is taken to mean the component whose proportion in the mixture is the smaller. Correspondingly, a matrix material in a system comprising a matrix material and a dopant is taken to mean the component whose proportion in the mixture is the larger.

Preferred phosphorescent dopants for use in matrix systems, preferably mixed-matrix systems, are the preferred phosphorescent dopants indicated below.

The term phosphorescent dopants typically encompasses compounds in the case of which the light emission takes place through a spin-forbidden transition, for example a transition from an excited triplet state or a state having a higher spin quantum number, for example a quintet state.

Suitable phosphorescent compounds (=triplet emitters) are, in particular, compounds which emit light, preferably in the visible region, on suitable excitation and in addition contain at least one atom having an atomic number greater than 20, preferably greater than 38 and less than 84, particularly preferably greater than 56 and less than 80, in particular a metal having this atomic number. The phosphorescent emitters used are preferably compounds which contain copper, molybdenum, tungsten, rhenium, ruthenium, osmium, rhodium, iridium, palladium, platinum, silver, gold or europium, in particular compounds which contain iridium or platinum. For the purposes of the present invention, all luminescent compounds which contain the above-mentioned metals are regarded as phosphorescent compounds.

Examples of the emitters described above are revealed by the applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439 and the as yet unpublished applications EP16179378.1 and EP16186313.9. In general, suitable phosphorescent complexes are all those as are used in accordance with the prior art for phosphorescent OLEDs and as are known to the person skilled in the art in the area of organic electroluminescence, and the person skilled in the art will be able to use further phosphorescent complexes without inventive activity.

Examples of phosphorescent dopants are shown below.

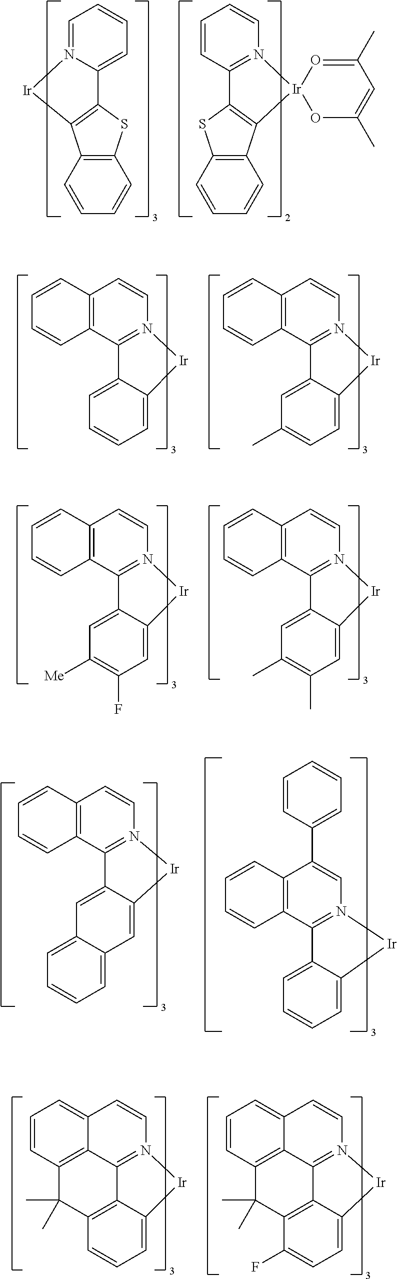

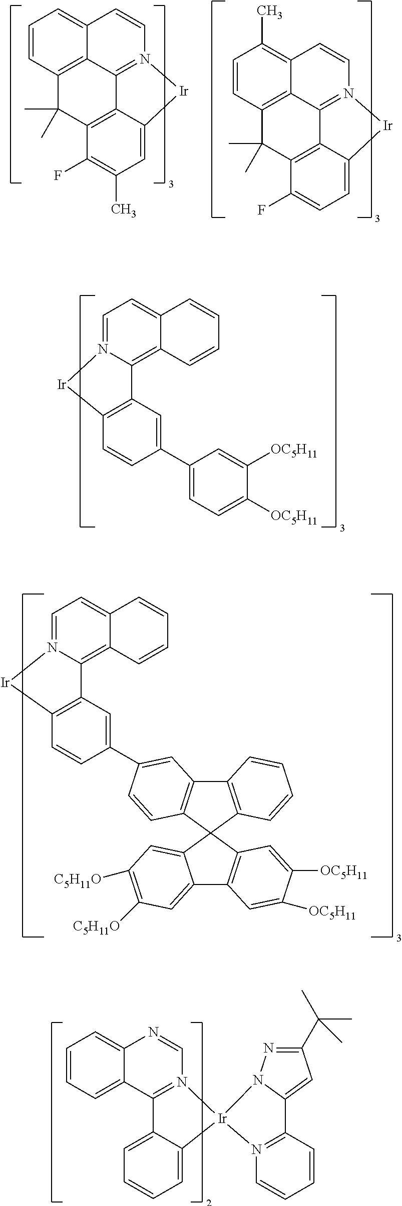

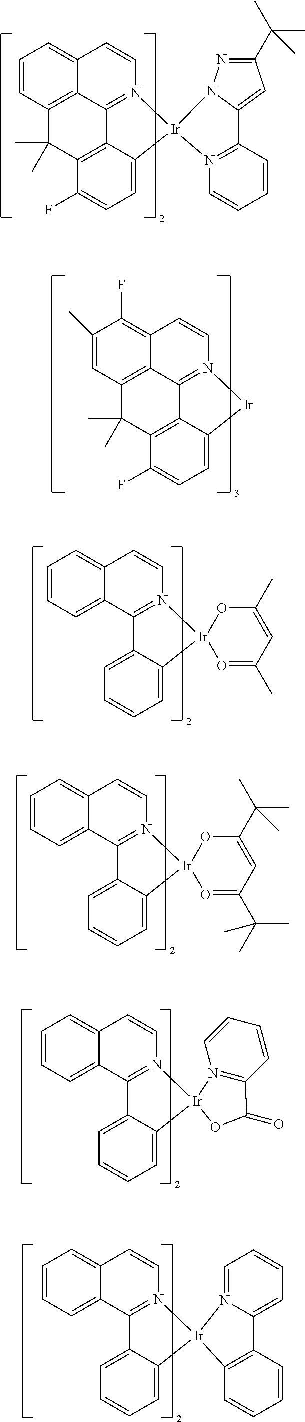

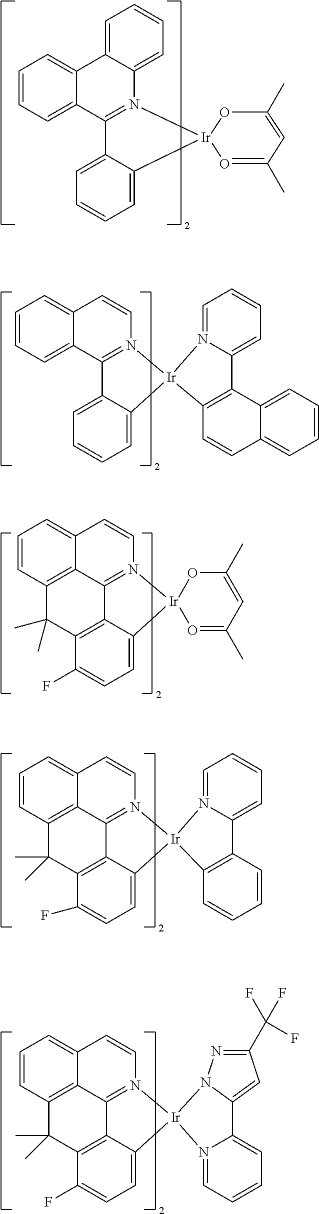

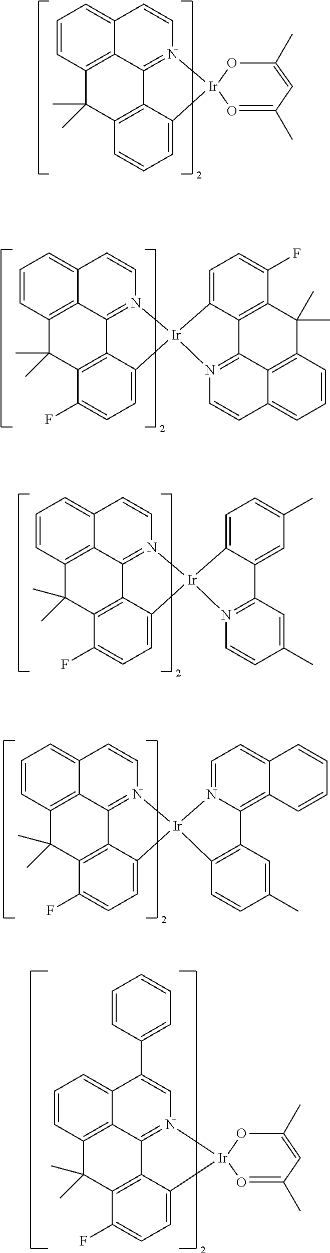

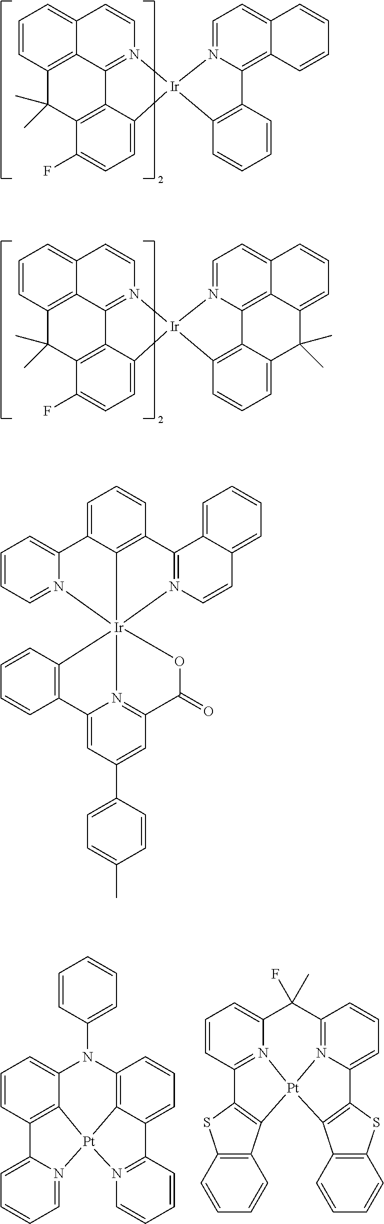

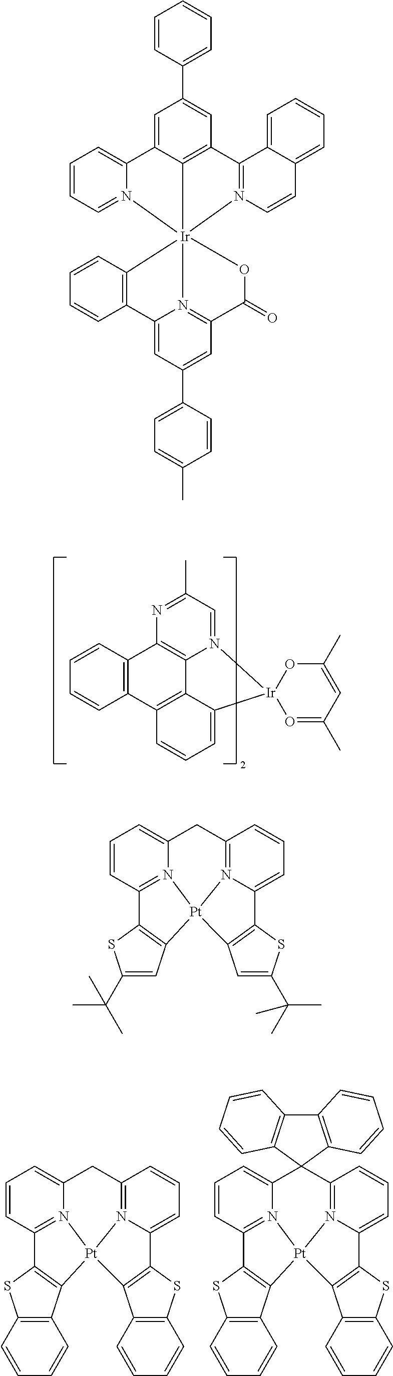

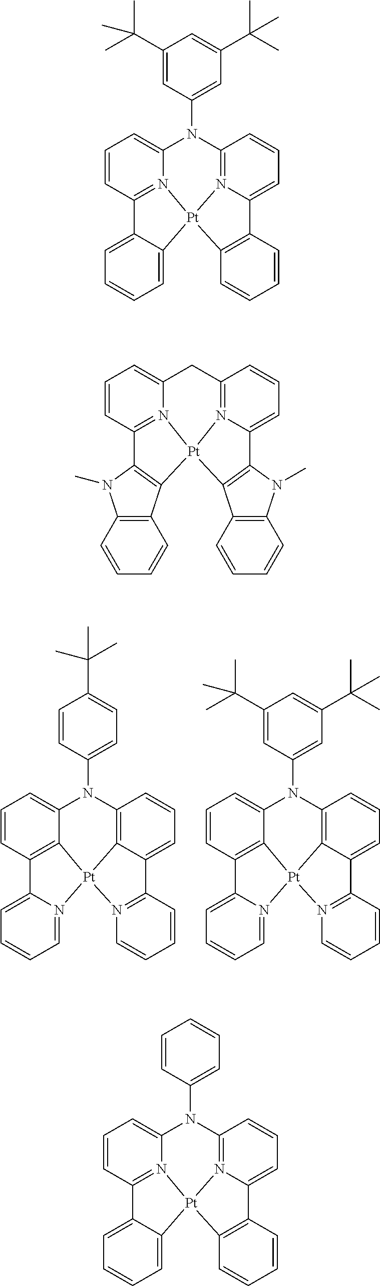

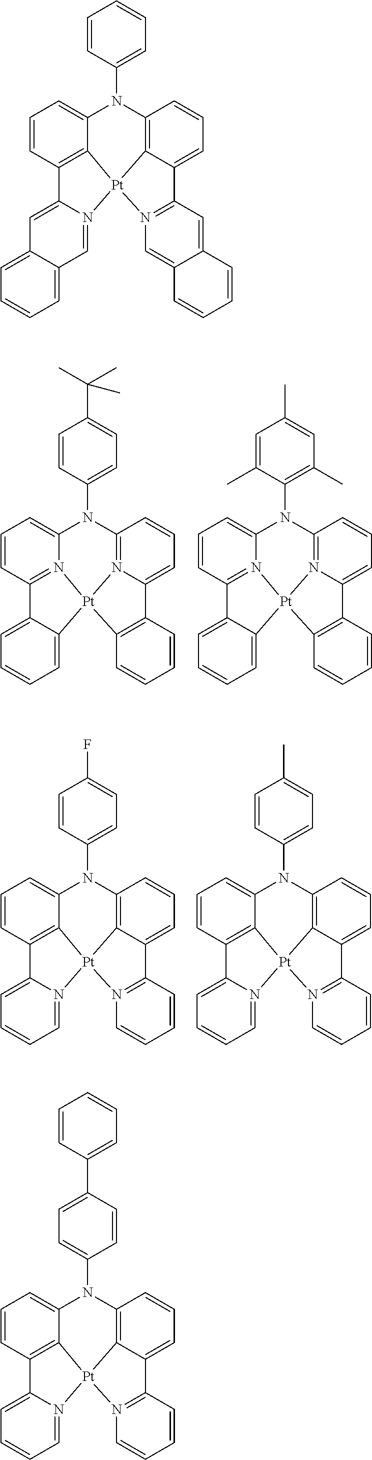

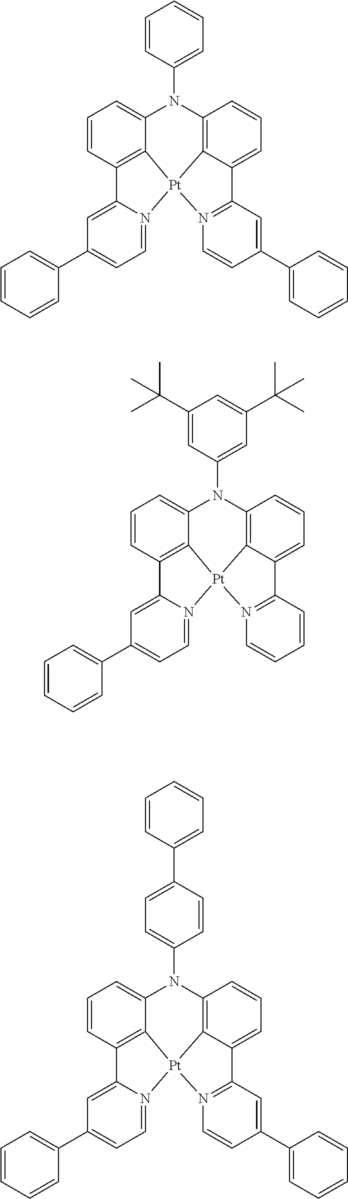

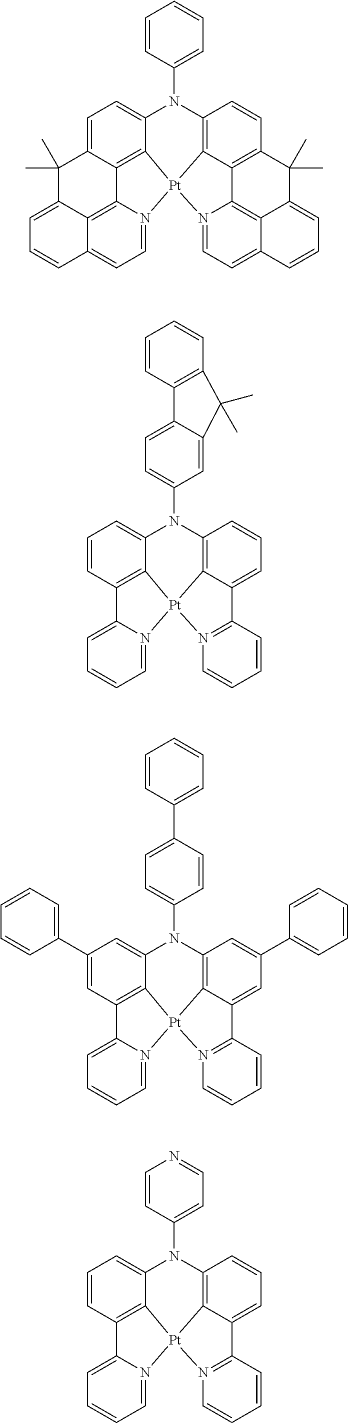

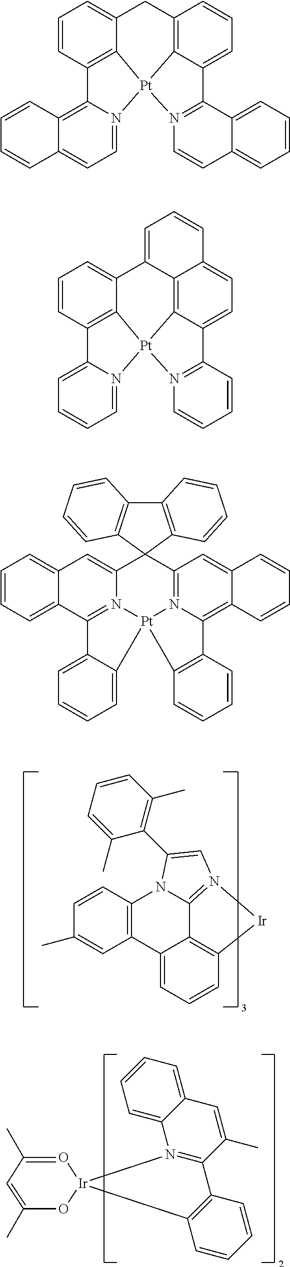

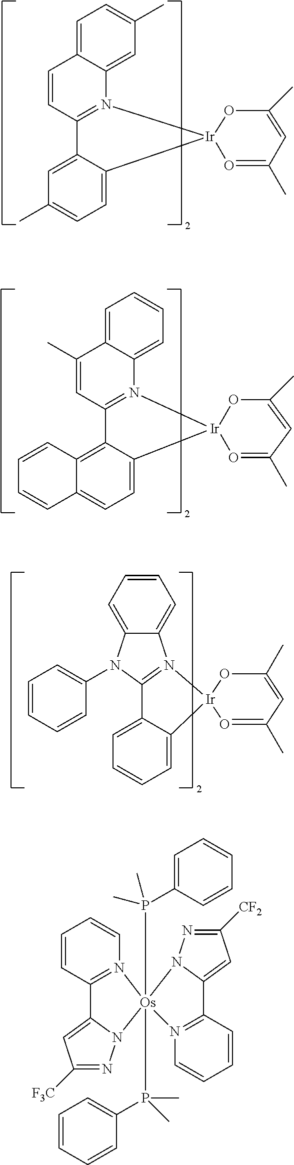

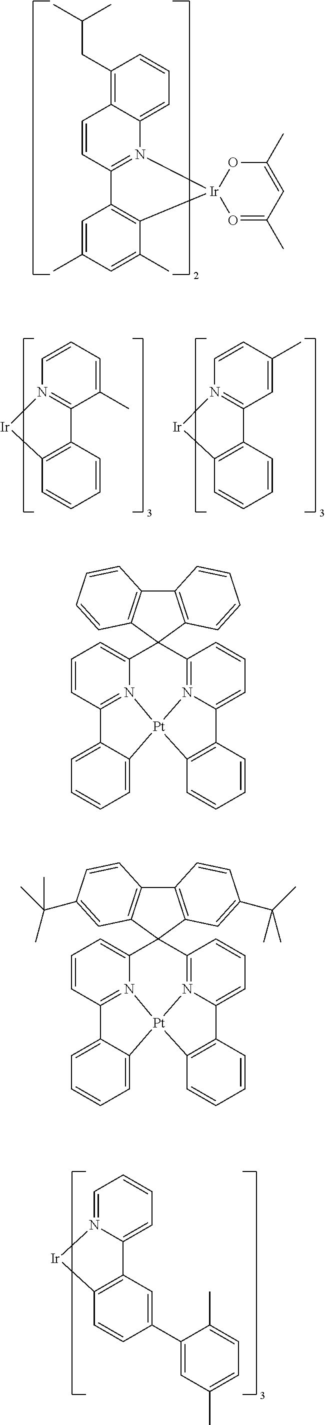

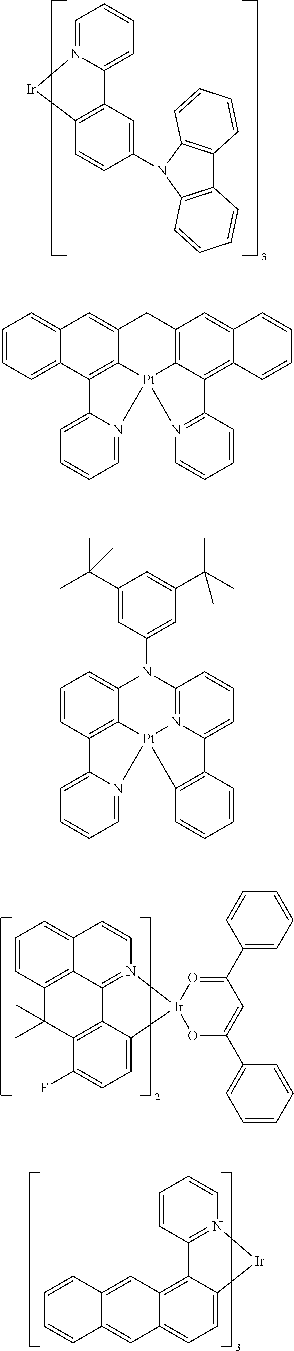

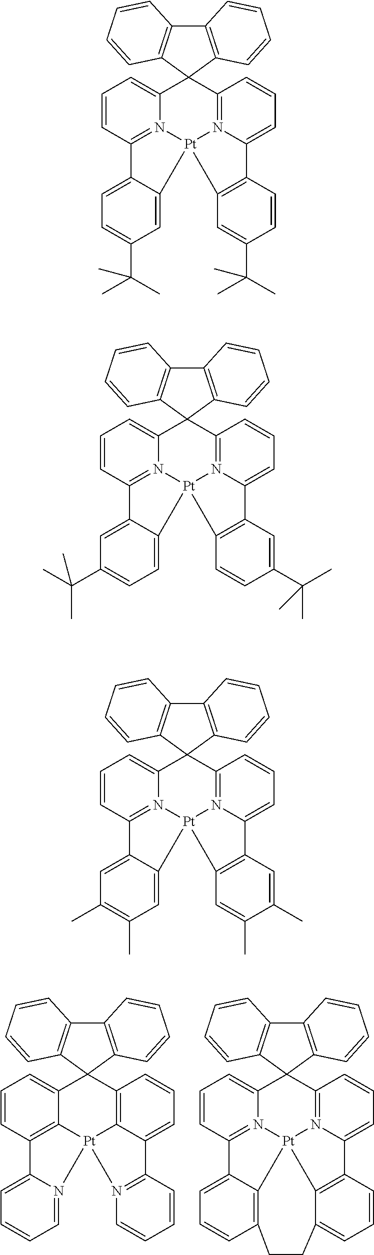

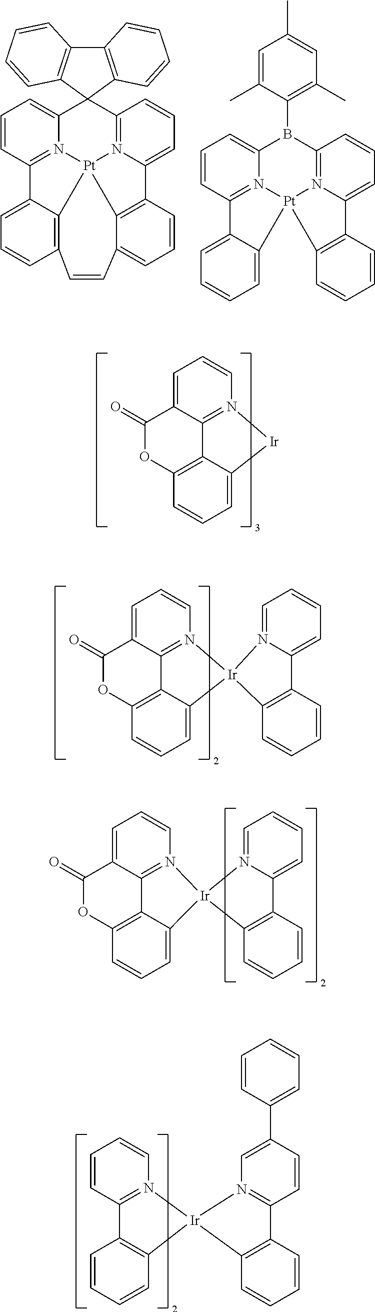

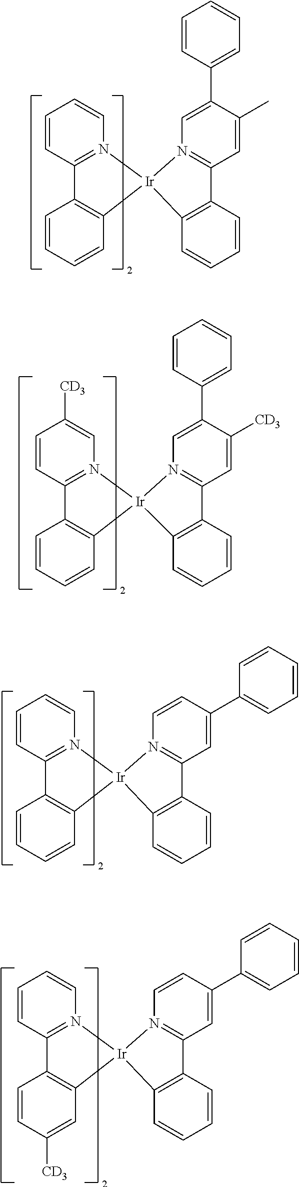

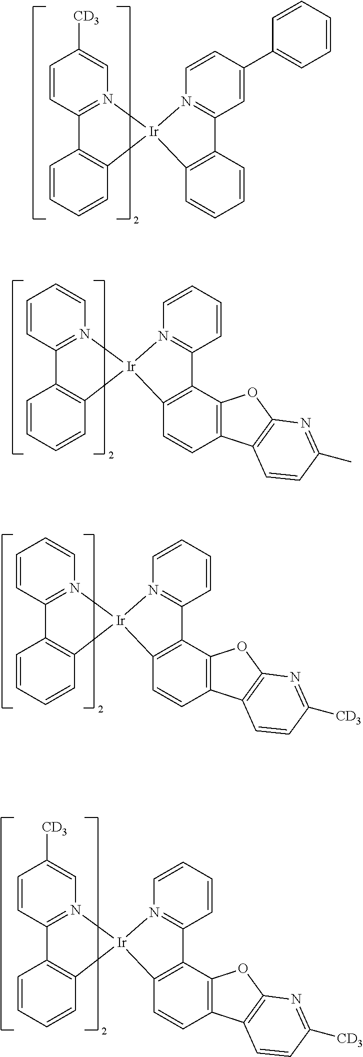

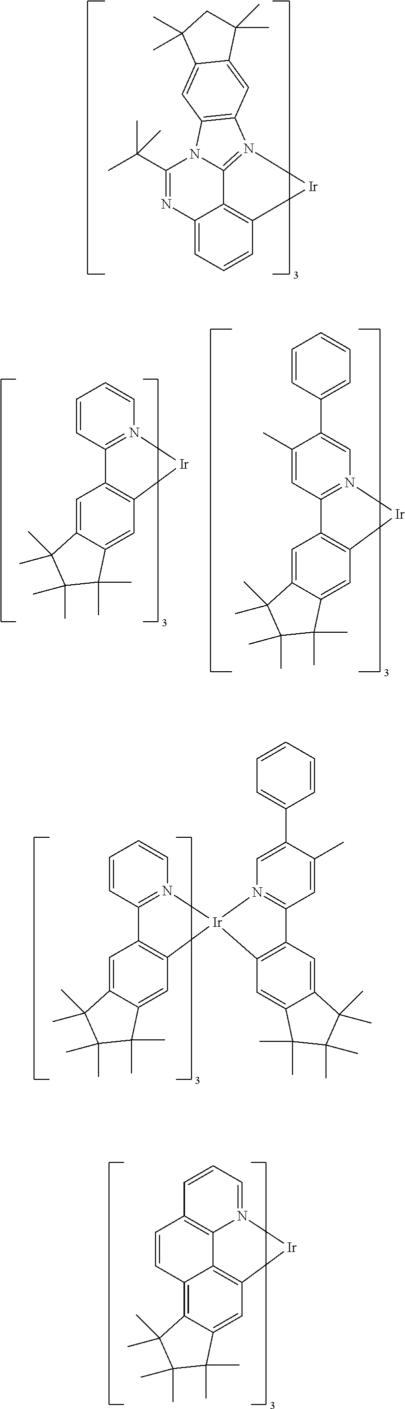

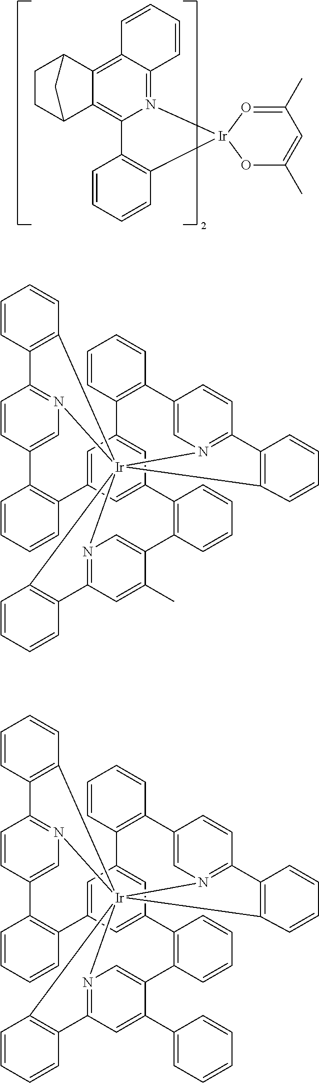

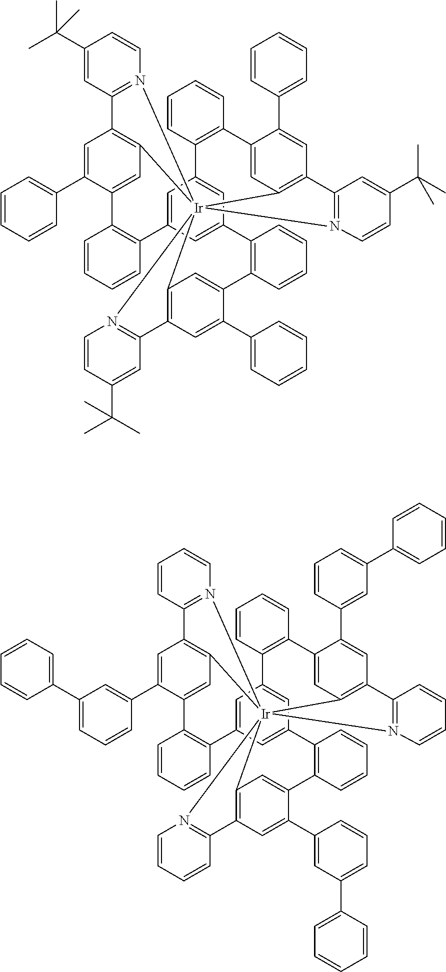

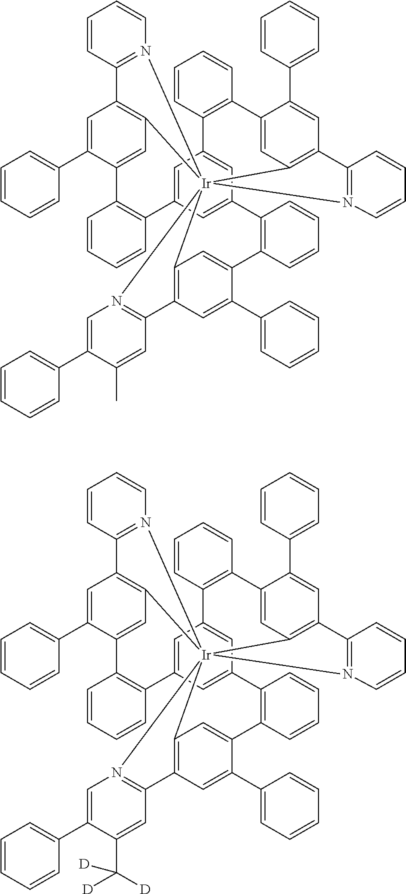

The compounds described above containing structures of the formula (I) or the preferred embodiments indicated above can preferably be used as active component in an electronic device. An electronic device is taken to mean a device which comprises an anode, a cathode and at least one layer lying between anode and cathode, where this layer comprises at least one organic or organometallic compound. The electronic device according to the invention thus comprises an anode, a cathode and at least one layer lying between them which comprises at least one compound containing structures of the formula (I). Preferred electronic devices here are selected from the group consisting of organic electroluminescent devices (OLEDs, PLEDs), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs), organic electrical sensors, light-emitting electrochemical cells (LECs), organic laser diodes (O-lasers) and organic plasmon emitting devices, preferably organic electroluminescent devices (OLEDs, PLEDs), in particular phosphorescent OLEDs, comprising at least one compound containing structures of the formula (I) in at least one layer. Particular preference is given to organic electroluminescent devices. Active components are generally the organic or inorganic materials which have been introduced between the anode and cathode, for example charge-injection, charge-transport or charge-blocking materials, but in particular emission materials and matrix materials.

A preferred embodiment of the invention are organic electroluminescent devices. The organic electroluminescent device comprises a cathode, an anode and at least one emitting layer. Apart from these layers, it may also comprise further layers, for example in each case one or more hole-injection layers, hole-transport layers, hole-blocking layers, electron-transport layers, electron-injection layers, exciton-blocking layers, electron-blocking layers, charge-generation layers and/or organic or inorganic p/n junctions. It is possible here for one or more hole-transport layers to be p-doped, for example with metal oxides, such as MoO₃ or WO₃, or with (per)fluorinated electron-deficient aromatic compounds, and/or for one or more electron-transport layers to be n-doped. Interlayers which have, for example, an exciton-blocking function and/or control the charge balance in the electroluminescent device may likewise be introduced between two emitting layers. However, it should be pointed out that each of these layers does not necessarily have to be present.

The organic electroluminescent device here may comprise one emitting layer or a plurality of emitting layers. If a plurality of emission layers are present, these preferably have in total a plurality of emission maxima between 380 nm and 750 nm, resulting overall in white emission, i.e. various emitting compounds which are able to fluoresce or phosphoresce are used in the emitting layers. Particular preference is given to three-layer systems, where the three layers exhibit blue, green and orange or red emission (for the basic structure see, for example, WO 2005/011013), or systems which have more than three emitting layers. Preference is furthermore given to tandem OLEDs. It may also be a hybrid system, in which one or more layers fluoresce and one or more other layers phosphoresce.

In a preferred embodiment of the invention, the organic electroluminescent device comprises the compound according to the invention containing structures of the formula (I) or the preferred embodiments indicated above as matrix material, preferably as electron-conducting matrix material in one or more emitting layers, preferably in combination with a further matrix material, preferably a hole-conducting matrix material. In a further preferred embodiment of the invention, the further matrix material is an electron-transporting compound. In still a further preferred embodiment, the further matrix material is a compound having a large band gap which does not participate in hole and electron transport in the layer, or only does so to an insignificant extent. An emitting layer comprises at least one emitting compound.

Suitable matrix materials which can be employed in combination with the compounds of the formula (I) or in accordance with the preferred embodiments are aromatic ketones, aromatic phosphine oxides or aromatic sulfoxides or sulfones, for example in accordance with WO 2004/013080, WO 2004/093207, WO 2006/005627 or WO 2010/006680, triarylamines, in particular monoamines, for example in accordance with WO 2014/015935, carbazole derivatives, for example CBP (N,N-biscarbazolylbiphenyl) or the carbazole derivatives disclosed in WO 2005/039246, US 2005/0069729, JP 2004/288381, EP 1205527 or WO 2008/086851, indolocarbazole derivatives, for example in accordance with WO 2007/063754 or WO 2008/056746, indenocarbazole derivatives, for example in accordance with WO 2010/136109 and WO 2011/000455, azacarbazole derivatives, for example in accordance with EP 1617710, EP 1617711, EP 1731584, JP 2005/347160, bipolar matrix materials, for example in accordance with WO 2007/137725, silanes, for example in accordance with WO 005/111172, azaboroles or boronic esters, for example in accordance with WO 2006/117052, triazine derivatives, for example in accordance with WO 2010/015306, WO 2007/063754 or WO 2008/056746, zinc complexes, for example in accordance with EP 652273 or WO 2009/062578, diazasilole or tetraazasilole derivatives, for example in accordance with WO 2010/054729, diazaphosphole derivatives, for example in accordance with WO 2010/054730, bridged carbazole derivatives, for example in accordance with US 2009/0136779, WO 2010/050778, WO 2011/042107, WO 2011/088877 or WO 2012/143080, triphenylene derivatives, for example in accordance with WO 2012/048781, lactams, for example in accordance with WO 2011/116865, WO 2011/137951 or WO 2013/064206, 4-spirocarbazole derivatives, for example in accordance with WO 2014/094963 or WO 2015/192939, or dibenzofuran derivatives, for example in accordance with WO 2015/169412, WO 2016/015810, WO 2016/023608, WO 2017/148564 or WO 2017/148565. A further phosphorescent emitter which emits at shorter wavelength than the actual emitter may likewise be present in the mixture as co-host.

Preferred co-host materials are triarylamine derivatives, in particular monoamines, indenocarbazole derivatives, 4-spirocarbazole derivatives, lactams and carbazole derivatives.

Preferred triarylamine derivatives which are employed as co-host materials together with the compounds according to the invention are selected from the compounds of the following formula (TA-1),

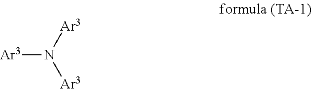

where Ar³, identically or differently on each occurrence, represents an aromatic or heteroaromatic ring system having 6 to 40 C atoms, which may in each case be substituted by one or more radicals R², where two or more adjacent substituents R² may optionally form a mono- or polycyclic, aliphatic ring system, which may be substituted by one or more radicals R³, where the symbol R² has the meaning given above, in particular for formula (I). Ar³, identically or differently on each occurrence, preferably represents an aryl or heteroaryl group having 5 to 24, preferably 5 to 12 aromatic ring atoms, which may in each case be substituted by one or more radicals R², but is preferably unsubstituted.

Examples of suitable groups Ar³ are selected from the group consisting of phenyl, ortho-, meta- or para-biphenyl, terphenyl, in particular branched terphenyl, quaterphenyl, in particular branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl and 1-, 2-, 3- or 4-carbazolyl, which may in each case be substituted by one or more radicals R², but are preferably unsubstituted.

The groups Ar³ are preferably selected, identically or differently on each occurrence, from the above-mentioned groups R¹-1 to R¹-80, particularly preferably R¹-1 to R¹-51.

In a preferred embodiment of the compounds of the formula (TA-1), at least one group Ar¹ is selected from a biphenyl group, which can be an ortho-, meta- or para-biphenyl group. In a further preferred embodiment of the compounds of the formula (TA-1), at least one group Ar¹ is selected from a fluorene group or spirobifluorene group, where these groups may in each case be bonded to the nitrogen atom via the 1-, 2-, 3- or 4-position. In still a further preferred embodiment of the compounds of the formula (TA-1), at least one group Ar³ is selected from a phenylene or biphenyl group, which can be an ortho-, meta- or para-linked group which is substituted by a dibenzothiophene group or a carbazole group, in particular a dibenzofuran group, where the dibenzofuran or dibenzothiophene group is linked to the phenylene or biphenyl group via the 1-, 2-, 3- or 4-position and where the carbazole group is linked to the phenylene or biphenyl group via the 1-, 2-, 3- or 4-position or via the nitrogen atom.

In a particularly preferred embodiment of the compounds of the formula (TA-1), one group Ar³ is selected from a fluorene or spirobifluorene group, in particular a 4-fluorene or 4-spirobifluorene group, and one group Ar³ is selected from a biphenyl group, in particular a para-biphenyl group, or a fluorene group, in particular a 2-fluorene group, and the third group Ar³ is selected from a para-phenylene group or a para-biphenyl group which is substituted by a dibenzofuran group, in particular a 4-dibenzofuran group, or a carbazole group, in particular an N-carbazole group or a 3-carbazole group.

Preferred indenocarbazole derivatives which are employed as co-host materials together with the compounds according to the invention are selected from the compounds of the following formula (TA-2),

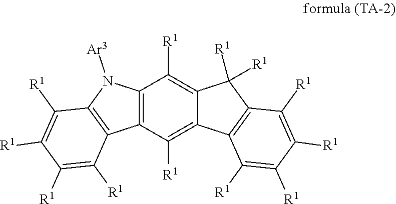

where Ar³ and R¹ have the meanings given above, in particular for formulae (I) and/or (TA-3). Preferred embodiments of the group Ar³ here are the above-mentioned structures R¹-1 to R¹-80, particularly preferably R¹-1 to R¹-51.

A preferred embodiment of the compounds of the formula (TA-2) are the compounds of the following formula (TA-2a),

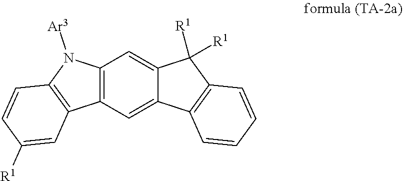

where Ar³ and R¹ have the meanings given above, in particular for formulae (I) and/or (TA-1). The two groups R¹ which are bonded to the indeno carbon atom preferably stand, identically or differently, for an alkyl group having 1 to 4 C atoms, in particular for methyl groups, or for an aromatic ring system having 6 to 12 C atoms, in particular for phenyl groups. The two groups R¹ which are bonded to the indeno carbon atom particularly preferably stand for methyl groups. Furthermore, the substituent R¹ which is bonded to the indenocarbazole skeleton in formula (TA-2a) preferably stands for H or for a carbazole group, which can be bonded to the indenocarbazole skeleton via the 1-, 2-, 3- or 4-position or via the N atom, in particular via the 3-position.

Preferred 4-spirocarbazole derivatives which are employed as co-host materials together with the compounds according to the invention are selected from the compounds of the following formula (TA-3),

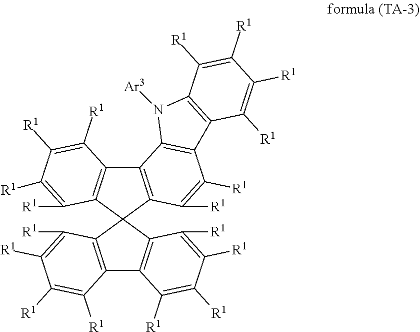

where Ar³ and R¹ have the meanings given above, in particular for formulae (TA-1), (I), (II) and/or (Q-1). Preferred embodiments of the group Ar³ here are the above-mentioned structures R¹-1 to R¹-80, particularly preferably R¹-1 to R¹-51.

A preferred embodiment of the compounds of the formula (TA-3) are the compounds of the following formula (TA-3a),

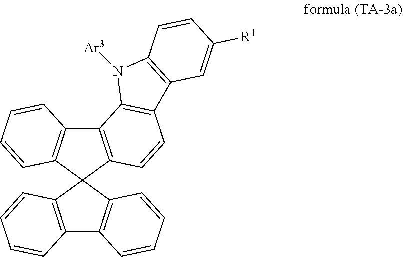

where Ar³ and R¹ have the meanings given above, in particular for formulae (TA-1), (I), (II) and/or (Q-1). Preferred embodiments of the group Ar³ here are the above-mentioned structures R¹-1 to R¹-80, particularly preferably R¹-1 to R¹-51.

Preferred lactams which are employed as co-host materials together with the compounds according to the invention are selected from the compounds of the following formula (LAC-1),

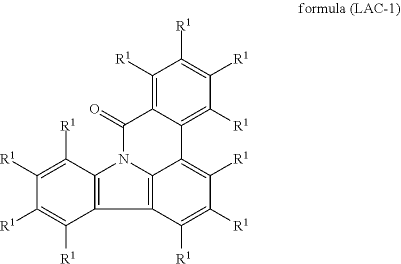

where R¹ has the meaning given above, in particular for formula (I).

A preferred embodiment of the compounds of the formula (LAC-1) are the compounds of the following formula (LAC-1a),

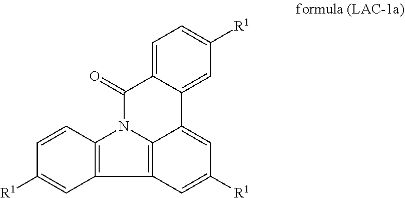

where R¹ has the meaning given above, in particular for formula (I). R¹ here preferably stands, identically or differently on each occurrence, for H or an aromatic or heteroaromatic ring system having 5 to 40 aromatic ring atoms, which may be substituted by one or more radicals R², where R² can have the meaning given above, in particular for formula (I). The substituents R¹ are very particularly preferably selected from the group consisting of H or an aromatic or heteroaromatic ring system having 6 to 18 aromatic ring atoms, preferably having 6 to 13 aromatic ring atoms, which may in each case be substituted by one or more non-aromatic radicals R², but is preferably unsubstituted. Examples of suitable substituents R¹ are selected from the group consisting of phenyl, ortho-, meta- or para-biphenyl, terphenyl, in particular branched terphenyl, quaterphenyl, in particular branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl and 1-, 2-, 3- or 4-carbazolyl, which may in each case be substituted by one or more radicals R², but are preferably unsubstituted. Suitable structures R¹ here are the same structures as depicted above for R-1 to R-79, particularly preferably R¹-1 to R¹-51.

It may also be preferred to employ a plurality of different matrix materials as a mixture, in particular at least one electron-conducting matrix material and at least one hole-conducting matrix material. Preference is likewise given to the use of a mixture of a charge-transporting matrix material and an electrically inert matrix material which is not involved or not essentially involved in charge transport, as described, for example, in WO 2010/108579.

It is furthermore preferred to employ a mixture of two or more triplet emitters together with a matrix. The triplet emitter having the shorter-wave emission spectrum serves as co-matrix for the triplet-emitter having the longer-wavelength emission spectrum.

In a preferred embodiment, a compound according to the invention containing structures of the formula (I) can particularly preferably be employed as matrix material in an emission layer of an organic electronic device, in particular in an organic electroluminescent device, for example in an OLED or OLEC. The matrix material comprising a compound containing structures of the formula (I) or the preferred embodiments indicated above and below is present in the electronic device in combination with one or more dopants, preferably phosphorescent dopants.

The proportion of the matrix material in the emitting layer is in this case between 50.0 and 99.9% by vol., preferably between 80.0 and 99.5% by vol. and particularly preferably between 92.0 and 99.5% by vol. for fluorescent emitting layers and between 85.0 and 97.0% by vol. for phosphorescent emitting layers.

Correspondingly, the proportion of the dopant is between 0.1 and 50.0% by vol., preferably between 0.5 and 20.0% by vol. and particularly preferably between 0.5 and 8.0% by vol. for fluorescent emitting layers and between 3.0 and 15.0% by vol. for phosphorescent emitting layers.

An emitting layer of an organic electroluminescent device may also comprise systems comprising a plurality of matrix materials (mixed-matrix systems) and/or a plurality of dopants. In this case too, the dopants are generally the materials whose proportion in the system is the smaller and the matrix materials are the materials whose proportion in the system is the greater. In individual cases, however, the proportion of an individual matrix material in the system may be smaller than the proportion of an individual dopant.

In a further preferred embodiment of the invention, the compound containing structures of the formula (I) or the preferred embodiments indicated above and below is used as a component of mixed-matrix systems. The mixed-matrix systems preferably comprise two or three different matrix materials, particularly preferably two different matrix materials. Preferably, one of the two matrix materials is a material having hole-transporting properties and the other material is a material having electron-transporting properties. The desired electron-transporting and hole-transporting properties of the mixed-matrix components may, however, also be combined mainly or completely in a single mixed-matrix component, where the further mixed-matrix component(s) fulfil(s) other functions. The two different matrix materials here may be present in a ratio of 1:50 to 1:1, preferably 1:20 to 1:1, particularly preferably 1:10 to 1:1 and very particularly preferably 1:4 to 1:1. Mixed-matrix systems are preferably employed in phosphorescent organic electroluminescent devices. More precise information on mixed-matrix systems is given, inter alia, in the application WO 2010/108579.

Furthermore, the present invention relates to an electronic device, preferably an organic electroluminescent device, which comprises one or more compounds according to the invention and/or at least one oligomer, polymer or dendrimer according to the invention as electron-conducting compound in one or more electron-conducting layers.

The cathode preferably comprises metals having a low work function, metal alloys or multilayered structures comprising various metals, such as, for example, alkaline-earth metals, alkali metals, main-group metals or lanthanoids (for example Ca, Ba, Mg, Al, In, Mg, Yb, Sm, etc.). Also suitable are alloys comprising an alkali metal or alkaline-earth metal and silver, for example an alloy comprising magnesium and silver. In the case of multilayered structures, further metals which have a relatively high work function, such as, for example, Ag, may also be used in addition to the said metals, in which case combinations of the metals, such as, for example, Mg/Ag, Ca/Ag or Ba/Ag, are generally used. It may also be preferred to introduce a thin interlayer of a material having a high dielectric constant between a metallic cathode and the organic semiconductor. Suitable for this purpose are, for example, alkali metal or alkaline-earth metal fluorides, but also the corresponding oxides or carbonates (for example LiF, LizO, BaF₂, MgO, NaF, CsF, Cs₂CO₃, etc.). Organic alkali-metal complexes, for example Liq (lithium quinolinate), are likewise suitable for this purpose. The layer thickness of this layer is preferably between 0.5 and 5 nm.

The anode preferably comprises materials having a high work function. The anode preferably has a work function of greater than 4.5 eV vs. vacuum. Suitable for this purpose are on the one hand metals having a high redox potential, such as, for example, Ag, Pt or Au. On the other hand, metal/metal oxide electrodes (for example Al/Ni/NiOₓ, Al/PtOₓ) may also be preferred. For some applications, at least one of the electrodes must be transparent or partially transparent in order either to facilitate irradiation of the organic material (O-SCs) or the coupling-out of light (OLEDs/PLEDs, O-LASERs). Preferred anode materials here are conductive mixed metal oxides. Particular preference is given to indium tin oxide (ITO) or indium zinc oxide (IZO). Preference is furthermore given to conductive, doped organic materials, in particular conductive doped polymers, for example PEDOT, PANI or derivatives of these polymers. It is furthermore preferred for a p-doped hole-transport material to be applied to the anode as hole-injection layer, where suitable p-dopants are metal oxides, for example MoO₃ or WO₃, or (per)fluorinated electron-deficient aromatic compounds. Further suitable p-dopants are HAT-CN (hexacyanohexaazatriphenylene) or the compound NPD9 from Novaled. A layer of this type simplifies hole injection in materials having a low HOMO, i.e. a large value of the HOMO.

All materials as are used in accordance with the prior art for the layers can generally be used in the further layers, and the person skilled in the art will be able to combine each of these materials with the materials according to the invention in an electronic device without inventive step.

The device is correspondingly structured (depending on the application), provided with contacts and finally hermetically sealed, since the lifetime of such devices is drastically shortened in the presence of water and/or air.

Preference is furthermore given to an electronic device, in particular an organic electroluminescent device, which is characterised in that one or more layers are applied by means of a sublimation process, in which the materials are vapour-deposited in vacuum sublimation units at an initial pressure of usually less than 10⁻⁵ mbar, preferably less than 10⁻⁶ mbar. It is also possible for the initial pressure to be even lower or even higher, for example less than 10⁻⁷ mbar.

Preference is furthermore given to an electronic device, in particular an organic electroluminescent device, which is characterised in that one or more layers are applied by means of the OVPD (organic vapour phase deposition) process or with the aid of carrier-gas sublimation, in which the materials are applied at a pressure of between 10⁻⁵ mbar and 1 bar. A special case of this process is the OVJP (organic vapour jet printing) process, in which the materials are applied directly through a nozzle and thus structured.

Preference is furthermore given to an electronic device, in particular an organic electroluminescent device, which is characterised in that one or more layers are produced from solution, such as, for example, by spin coating, or by means of any desired printing process, such as, for example, screen printing, flexographic printing, offset printing or nozzle printing, but particularly preferably LITI (light induced thermal imaging, thermal transfer printing) or ink-jet printing. Soluble compounds are necessary for this purpose, which are obtained, for example, through suitable substitution.

The electronic device, in particular the organic electroluminescent device, may also be produced as a hybrid system by applying one or more layers from solution and applying one or more other layers by vapour deposition. Thus, for example, it is possible to apply an emitting layer comprising a compound according to the invention containing structures of the formula (I) and a matrix material from solution and to apply a hole-blocking layer and/or an electron-transport layer on top by vacuum vapour deposition.

These processes are generally known to the person skilled in the art and can be applied by him without problems to electronic devices, in particular organic electroluminescent devices, comprising compounds containing structures of the formula (I) or the preferred embodiments indicated above.

The electronic devices according to the invention, in particular organic electroluminescent devices, are distinguished over the prior art by one or more of the following surprising advantages:
1. Electronic devices, in particular organic electroluminescent devices, containing compounds, oligomers, polymers or dendrimers containing structures of the formula (I) or the preferred embodiments shown above and below, in particular as electron-conducting materials and/or hole-conductor materials and/or as matrix materials, have a very good lifetime.
2. Electronic devices, in particular organic electroluminescent devices, containing compounds, oligomers, polymers or dendrimers containing structures of the formula (I) or the preferred embodiments shown above and below, in particular as electron-transport materials, hole-conductor materials and/or as host materials, have excellent efficiency. In particular, the efficiency is significantly higher than analogous compounds which do not contain a structural unit of the formula (I). Compounds, oligomers, polymers or dendrimers according to the invention containing structures of the formula (I) or the preferred embodiments shown above and below effect a low operating voltage on use in electronic devices. These compounds effect, in particular, low roll-off, i.e. a small drop in the power efficiency of the device at high luminous densities.
3. Electronic devices, in particular organic electroluminescent devices, containing compounds, oligomers, polymers or dendrimers containing structures of the formula (I) or the preferred embodiments shown above and below as electron-transport materials, hole-conductor materials and/or as host materials have excellent colour purity.
4. The compounds, oligomers, polymers or dendrimers according to the invention containing structures of the formula (I) or the preferred embodiments shown above and below exhibit very high thermal and photochemical stability and lead to compounds having a very long lifetime.
5. The use of compounds, oligomers, polymers or dendrimers according to the invention containing structures of the formula (I) or the preferred embodiments shown above and below in layers of electronic devices, in particular organic electroluminescent devices, leads to high mobility of the electron-conductor structures.
6. Compounds, oligomers, polymers or dendrimers according to the invention containing structures of the formula (I) or the preferred embodiments shown above and below are distinguished by excellent thermal stability, where compounds having a molecular weight of less than about 1200 g/mol are readily sublimable.
7. Compounds, oligomers, polymers or dendrimers according to the invention containing structures of the formula (I) or the preferred embodiments shown above and below have excellent glass film formation.
8. Compounds, oligomers, polymers or dendrimers according to the invention containing structures of the formula (I) or the preferred embodiments shown above and below form very good films from solutions.

These above-mentioned advantages are not accompanied by an impairment of the other electronic properties.

The compounds and mixtures according to the invention are suitable for use in an electronic device. An electronic device here is taken to mean a device which contains at least one layer which comprises at least one organic compound. The component may, however, also comprise inorganic materials or also layers which are built up entirely from inorganic materials.

The present invention therefore furthermore relates to the use of the compounds or mixtures according to the invention in an electronic device, in particular in an organic electroluminescent device.

The present invention still furthermore relates to the use of a compound according to the invention and/or an oligomer, polymer or dendrimer according to the invention in an electronic device as host material, hole-conduction material, electron-injection material and/or electron-transport material, preferably as host material and/or electron-transport material.

The present invention still furthermore relates to an electronic device containing at least one of the above-mentioned compounds or mixtures according to the invention. The preferences given above for the compound also apply here to the electronic devices. The electronic device is particularly preferably selected from the group consisting of organic electroluminescent devices (OLEDs, PLEDs), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs), organic electrical sensors, light-emitting electrochemical cells (LECs), organic laser diodes (O-lasers) and organic plasmon emitting devices, preferably organic electroluminescent devices (OLEDs, PLEDs), in particular phosphorescent OLEDs.

In a further embodiment of the invention, the organic electroluminescent device according to the invention does not contain a separate hole-injection layer and/or hole-transport layer and/or hole-blocking layer and/or electron-transport layer, i.e. the emitting layer is directly adjacent to the hole-injection layer or the anode, and/or the emitting layer is directly adjacent to the electron-transport layer or the electron-injection layer or the cathode, as described, for example, in WO 2005/053051. Furthermore, it is possible to use a metal complex which is identical or similar to the metal complex in the emitting layer as hole-transport or hole-injection material directly adjacent to the emitting layer, as described, for example, in WO 2009/030981.

All materials as are usually employed in accordance with the prior art can be used in the further layers of the organic electroluminescent device according to the invention. The person skilled in the art will therefore be able, without inventive activity, to employ all materials known for organic electroluminescent devices in combination with the compounds of the formula (I) according to the invention or the preferred embodiments.

The compounds according to the invention generally have very good properties on use in organic electroluminescent devices. In particular, the lifetime on use of the compounds according to the invention in organic electroluminescent devices is significantly better compared with similar compounds in accordance with the prior art. The further properties of the organic electroluminescent device, in particular the efficiency and the voltage, are likewise better or at least comparable here.

It should be pointed out that variations of the embodiments described in the present invention fall within the scope of this invention. Each feature disclosed in the present invention can, unless this is explicitly excluded, be replaced by alternative features which serve the same, an equivalent or a similar purpose. Thus, each feature disclosed in the present invention is, unless stated otherwise, to be regarded as an example of a generic series or as an equivalent or similar feature.

All features of the present invention can be combined with one another in any way, unless certain features and/or steps are mutually exclusive. This applies, in particular, to preferred features of the present invention. Equally, features of non-essential combinations can be used separately (and not in combination).

It should furthermore be pointed out that many of the features, and in particular those of the preferred embodiments of the present invention, are themselves inventive and are not to be regarded merely as part of the embodiments of the present invention. For these features, independent protection can be sought in addition or as an alternative to each invention presently claimed.

The teaching on technical action disclosed with the present invention can be abstracted and combined with other examples.

The invention is explained in greater detail by the following examples, without wishing to restrict them thereby. The person skilled in the art will be able to use the descriptions to produce further electronic devices according to the invention without inventive step and thus carry out the invention throughout the range claimed.

### EXAMPLES

### Synthesis Examples

The following syntheses are carried out, unless indicated otherwise, in dried solvents under a protective-gas atmosphere. The compounds according to the invention can be prepared by synthetic methods known to the person skilled in the art.

### a) Biphenyl-4-yl-(9,9-dimethyl-9H-fluoren-1-yl)amine

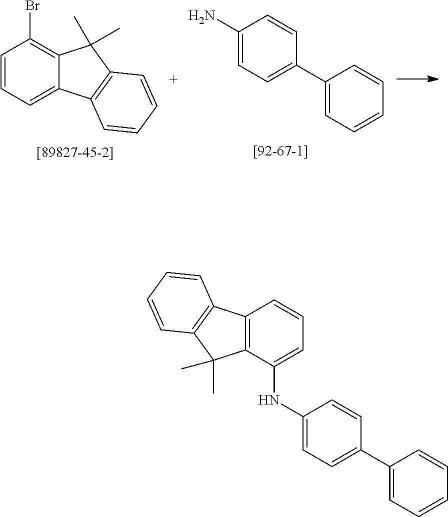 [SMILES: CC1(C)c2ccccc2-c2cccc(Br)c21 ; Nc1ccc(-c2ccccc2)cc1 ; CC1(C)c2ccccc2-c2cccc(Nc3ccc(-c4ccccc4)cc3)c21]

36 g (212 mmol, 1.0 eq) of 4-aminobiphenyl are initially introduced together with 57.8 g (177 mmol, 1.0 eq) of 1-bromodimethylfluorene and 2.4 g (212 mmol, 1.20 eq) of sodium t-pentoxide [14593-46-5] in 600 ml of absolute toluene and degassed for 30 minutes. 398 mg (1.77 mmol, 0.01 eq) of palladium(II) acetate [3375-31-3] and 1.46 g (3.56 mmol, 0.02 eq) of 2-dicyclohexylphosphino-2′,6′-dimethoxybiphenyl (SPhos) [657408-07-6] are subsequently added, and the batch is heated under reflux overnight. When the reaction is complete, the batch is cooled to room temperature and extracted with 500 ml of water. The aqueous phase is subsequently washed three times with toluene, the combined organic phases are dried over sodium sulfate, and the solvent is removed in a rotary evaporator. The brown residue is taken up in about 200 ml of toluene and filtered through silica gel. For further purification, a recrystallisation is carried out from toluene/heptane. Yield: 59 g (164 mmol), 79% of theory.

The following compounds can be prepared analogously:

| | | Starting | | Yield |
|---|---|---|---|---|
| | Starting material 1 | material 2 | Product | [%] |
| 1a | 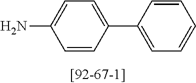 [SMILES: Nc1ccc(-c2ccccc2)cc1] | 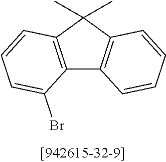 [SMILES: CC1(C)c2ccccc2-c2c(Br)cccc21] | 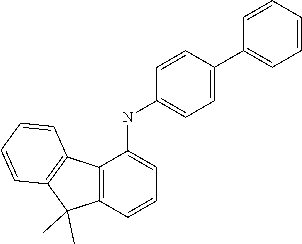 [SMILES: CC1(C)c2ccccc2-c2c(Nc3ccc(-c4ccccc4)cc3)cccc21] | 65 |
| 2a | 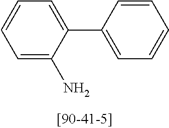 [SMILES: Nc1ccccc1-c1ccccc1] | 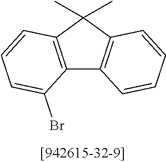 [SMILES: CC1(C)c2ccccc2-c2c(Br)cccc21] | 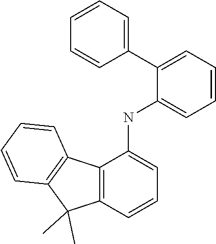 [SMILES: CC1(C)c2ccccc2-c2c(Nc3ccccc3-c3ccccc3)cccc21] | 63 |
| 3a | 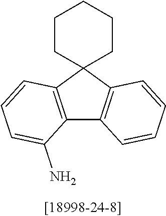 [SMILES: Nc1cccc2c1-c1ccccc1C21CCCCC1] | 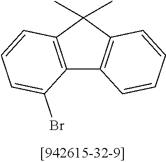 [SMILES: CC1(C)c2ccccc2-c2c(Br)cccc21] | 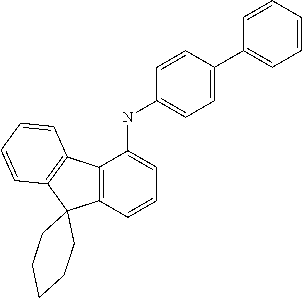 [SMILES: c1ccc(-c2ccc(Nc3cccc4c3-c3ccccc3C43CCCCC3)cc2)cc1] | 60 |
| 4a | 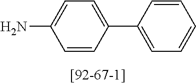 [SMILES: Nc1ccc(-c2ccccc2)cc1] | 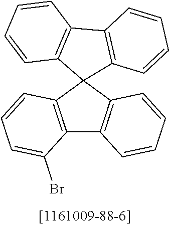 [SMILES: Brc1cccc2c1-c1ccccc1C21c2ccccc2-c2ccccc21] | 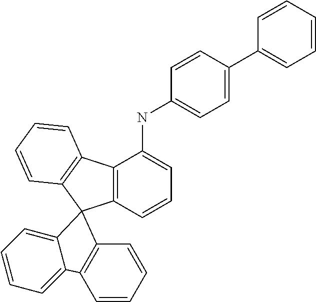 [SMILES: c1ccc(-c2ccc(Nc3cccc4c3-c3ccccc3C43c4ccccc4-c4ccccc43)cc2)cc1] | 62 |
| 5a | 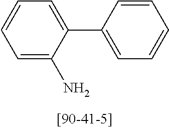 [SMILES: Nc1ccccc1-c1ccccc1] | 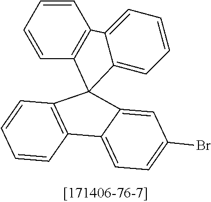 [SMILES: Brc1ccc2c(c1)C1(c3ccccc3-c3ccccc31)c1ccccc1-2] | 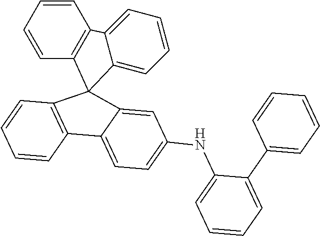 [SMILES: c1ccc(-c2ccccc2Nc2ccc3c(c2)C2(c4ccccc4-c4ccccc42)c2ccccc2-3)cc1] | 64 |
| 6a | 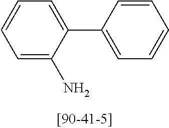 [SMILES: Nc1ccccc1-c1ccccc1] | 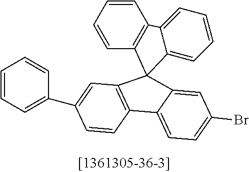 [SMILES: Brc1ccc2c(c1)C1(c3ccccc3-c3ccccc31)c1cc(-c3ccccc3)ccc1-2] | 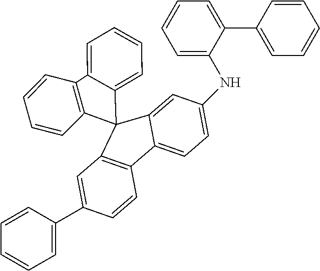 [SMILES: c1ccc(-c2ccc3c(c2)C2(c4ccccc4-c4ccccc42)c2cc(Nc4ccccc4-c4ccccc4)ccc2-3)cc1] | 68 |
| 7a | 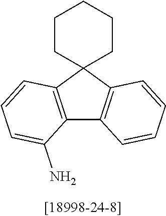 [SMILES: Nc1cccc2c1-c1ccccc1C21CCCCC1] | 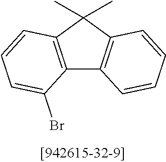 [SMILES: CC1(C)c2ccccc2-c2c(Br)cccc21] | 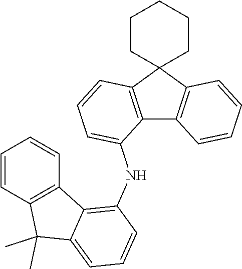 [SMILES: CC1(C)c2ccccc2-c2c(Nc3cccc4c3-c3ccccc3C43CCCCC3)cccc21] | 71 |
| 8a | 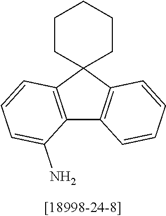 [SMILES: Nc1cccc2c1-c1ccccc1C21CCCCC1] | 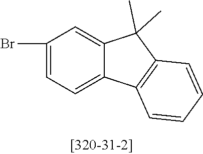 [SMILES: CC1(C)c2ccccc2-c2ccc(Br)cc21] | 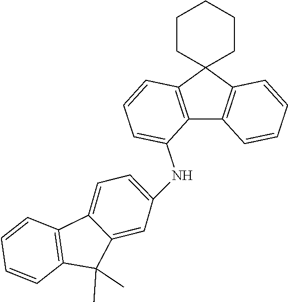 [SMILES: CC1(C)c2ccccc2-c2ccc(Nc3cccc4c3-c3ccccc3C43CCCCC3)cc21] | 72 |
| 9a | 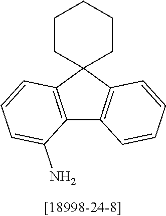 [SMILES: Nc1cccc2c1-c1ccccc1C21CCCCC1] | 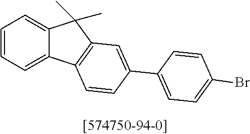 [SMILES: CC1(C)c2ccccc2-c2ccc(-c3ccc(Br)cc3)cc21] | 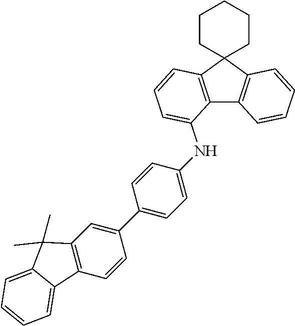 [SMILES: CC1(C)c2ccccc2-c2ccc(-c3ccc(Nc4cccc5c4-c4ccccc4C54CCCCC4)cc3)cc21] | 83 |
| 10a | 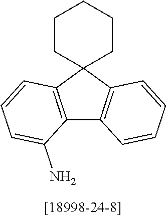 [SMILES: Nc1cccc2c1-c1ccccc1C21CCCCC1] | 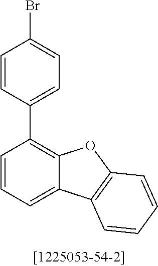 [SMILES: Brc1ccc(-c2cccc3c2oc2ccccc23)cc1] | 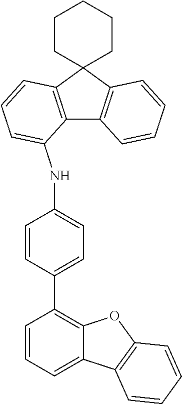 [SMILES: c1ccc2c(c1)-c1c(Nc3ccc(-c4cccc5c4oc4ccccc45)cc3)cccc1C21CCCCC1] | 64 |
| 11a | 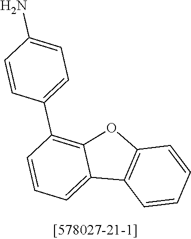 [SMILES: Nc1ccc(-c2cccc3c2oc2ccccc23)cc1] | 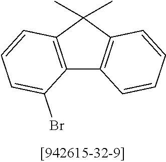 [SMILES: CC1(C)c2ccccc2-c2c(Br)cccc21] | 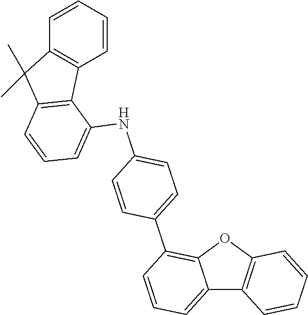 [SMILES: CC1(C)c2ccccc2-c2c(Nc3ccc(-c4cccc5c4oc4ccccc45)cc3)cccc21] | 67 |
| 12a | 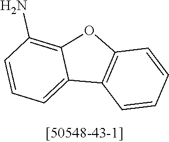 [SMILES: Nc1cccc2c1oc1ccccc12] | 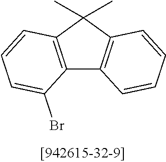 [SMILES: CC1(C)c2ccccc2-c2c(Br)cccc21] | 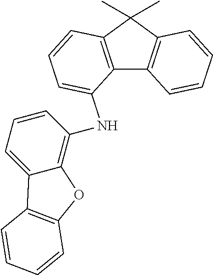 [SMILES: CC1(C)c2ccccc2-c2c(Nc3cccc4c3oc3ccccc34)cccc21] | 56 |
| 13a | 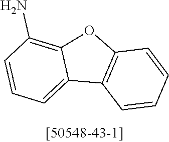 [SMILES: Nc1cccc2c1oc1ccccc12] | 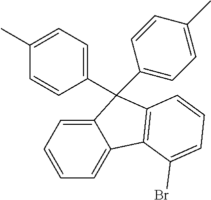 | 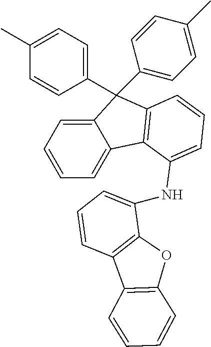 | 75 |
| 14a | 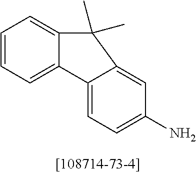 [SMILES: CC1(C)c2ccccc2-c2ccc(N)cc21] | 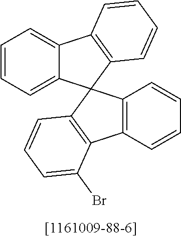 [SMILES: Brc1cccc2c1-c1ccccc1C21c2ccccc2-c2ccccc21] | 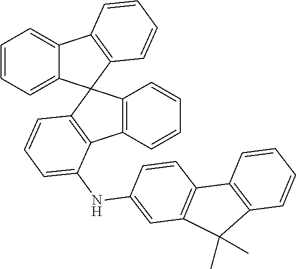 [SMILES: CC1(C)c2ccccc2-c2ccc(Nc3cccc4c3-c3ccccc3C43c4ccccc4-c4ccccc43)cc21] | 85 |
| 15a | 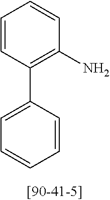 [SMILES: Nc1ccccc1-c1ccccc1] | 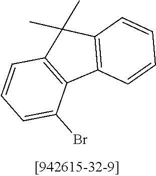 [SMILES: CC1(C)c2ccccc2-c2c(Br)cccc21] | 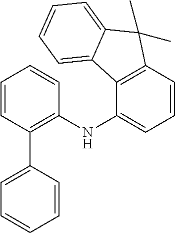 [SMILES: CC1(C)c2ccccc2-c2c(Nc3ccccc3-c3ccccc3)cccc21] | 69 |
| 16a | 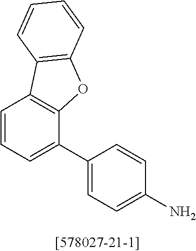 [SMILES: Nc1ccc(-c2cccc3c2oc2ccccc23)cc1] | 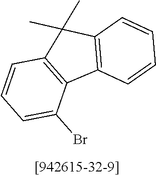 [SMILES: CC1(C)c2ccccc2-c2c(Br)cccc21] | 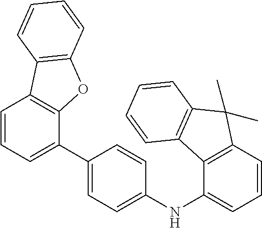 [SMILES: CC1(C)c2ccccc2-c2c(Nc3ccc(-c4cccc5c4oc4ccccc45)cc3)cccc21] | 67 |
| 17a | 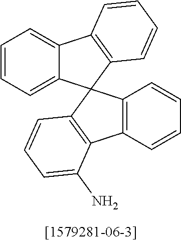 [SMILES: Nc1cccc2c1-c1ccccc1C21c2ccccc2-c2ccccc21] | 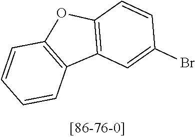 [SMILES: Brc1ccc2oc3ccccc3c2c1] | 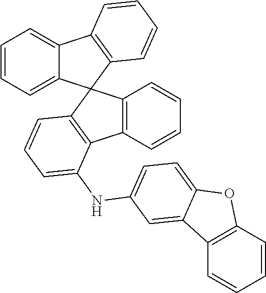 [SMILES: c1ccc2c(c1)-c1ccccc1C21c2ccccc2-c2c(Nc3ccc4oc5ccccc5c4c3)cccc21] | 88 |
| 18a | 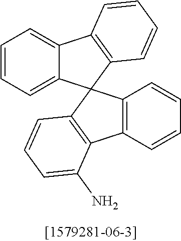 [SMILES: Nc1cccc2c1-c1ccccc1C21c2ccccc2-c2ccccc21] | 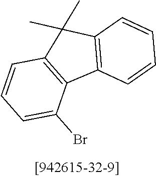 [SMILES: CC1(C)c2ccccc2-c2c(Br)cccc21] | 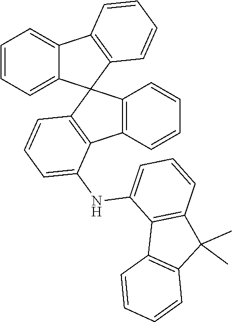 [SMILES: CC1(C)c2ccccc2-c2c(Nc3cccc4c3-c3ccccc3C43c4ccccc4-c4ccccc43)cccc21] | 81 |
| 19a | 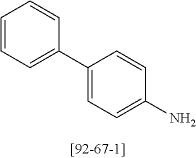 [SMILES: Nc1ccc(-c2ccccc2)cc1] | 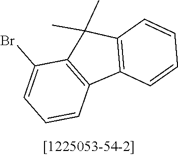 [SMILES: CC1(C)c2ccccc2-c2cccc(Br)c21] | 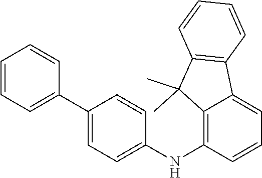 [SMILES: CC1(C)c2ccccc2-c2cccc(Nc3ccc(-c4ccccc4)cc3)c21] | 77 |
| 20a | 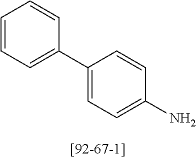 [SMILES: Nc1ccc(-c2ccccc2)cc1] | 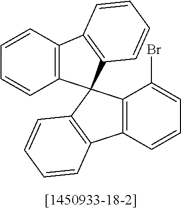 | 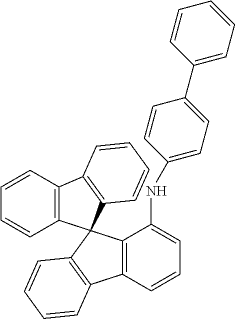 | 70 |
| 21a | 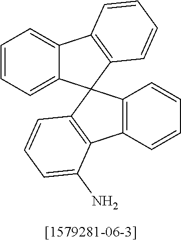 [SMILES: Nc1cccc2c1-c1ccccc1C21c2ccccc2-c2ccccc21] | 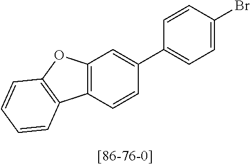 [SMILES: Brc1ccc(-c2ccc3c(c2)oc2ccccc23)cc1] | 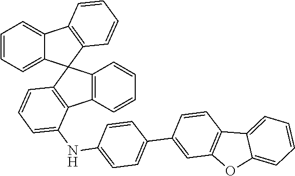 [SMILES: c1ccc2c(c1)-c1ccccc1C21c2ccccc2-c2c(Nc3ccc(-c4ccc5c(c4)oc4ccccc45)cc3)cccc21] | 84 |
| 22a | 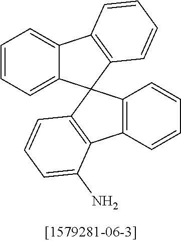 [SMILES: Nc1cccc2c1-c1ccccc1C21c2ccccc2-c2ccccc21] | 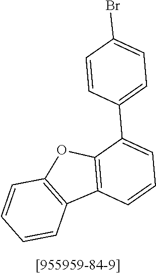 [SMILES: Brc1ccc(-c2cccc3c2oc2ccccc23)cc1] | 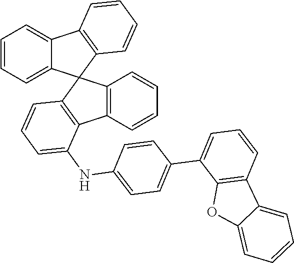 [SMILES: c1ccc2c(c1)-c1ccccc1C21c2ccccc2-c2c(Nc3ccc(-c4cccc5c4oc4ccccc45)cc3)cccc21] | 91 |
| 23a | 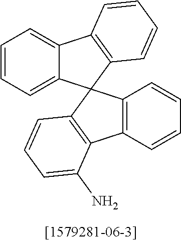 [SMILES: Nc1cccc2c1-c1ccccc1C21c2ccccc2-c2ccccc21] | 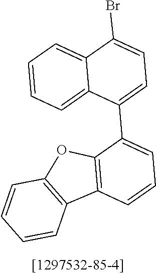 [SMILES: Brc1ccc(-c2cccc3c2oc2ccccc23)c2ccccc12] | 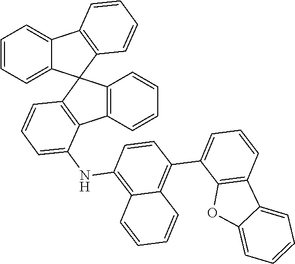 [SMILES: c1ccc2c(c1)-c1ccccc1C21c2ccccc2-c2c(Nc3ccc(-c4cccc5c4oc4ccccc45)c4ccccc34)cccc21] | 74 |
| 24a | 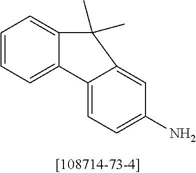 [SMILES: CC1(C)c2ccccc2-c2ccc(N)cc21] | 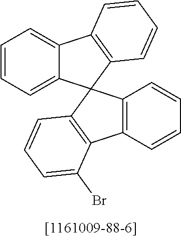 [SMILES: Brc1cccc2c1-c1ccccc1C21c2ccccc2-c2ccccc21] | 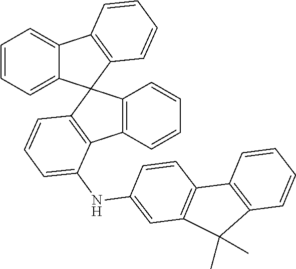 [SMILES: CC1(C)c2ccccc2-c2ccc(Nc3cccc4c3-c3ccccc3C43c4ccccc4-c4ccccc43)cc21] | 85 |
| 25a | 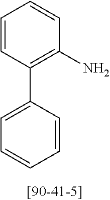 [SMILES: Nc1ccccc1-c1ccccc1] | 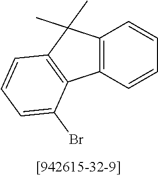 [SMILES: CC1(C)c2ccccc2-c2c(Br)cccc21] | 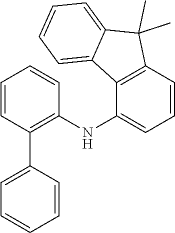 [SMILES: CC1(C)c2ccccc2-c2c(Nc3ccccc3-c3ccccc3)cccc21] | 69 |
| 26a | 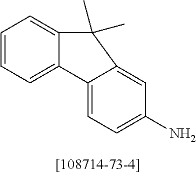 [SMILES: CC1(C)c2ccccc2-c2ccc(N)cc21] | 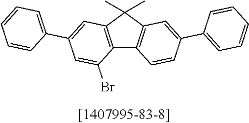 [SMILES: CC1(C)c2cc(-c3ccccc3)ccc2-c2c(Br)cc(-c3ccccc3)cc21] | 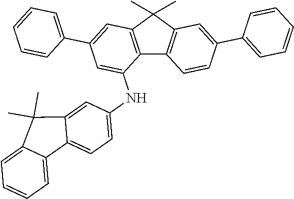 [SMILES: CC1(C)c2ccccc2-c2ccc(Nc3cc(-c4ccccc4)cc4c3-c3ccc(-c5ccccc5)cc3C4(C)C)cc21] | 67 |
| 27a | 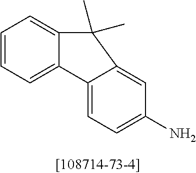 [SMILES: CC1(C)c2ccccc2-c2ccc(N)cc21] | 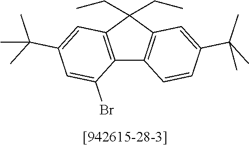 [SMILES: CCC1(CC)c2cc(C(C)(C)C)ccc2-c2c(Br)cc(C(C)(C)C)cc21] | 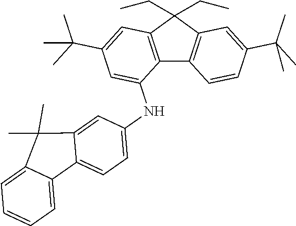 [SMILES: CCC1(CC)c2cc(C(C)(C)C)ccc2-c2c(Nc3ccc4c(c3)C(C)(C)c3ccccc3-4)cc(C(C)(C)C)cc21] | 71 |
| 28a | 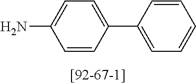 [SMILES: Nc1ccc(-c2ccccc2)cc1] | 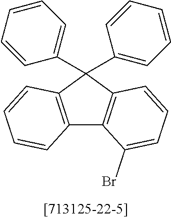 [SMILES: Brc1cccc2c1-c1ccccc1C2(c1ccccc1)c1ccccc1] | 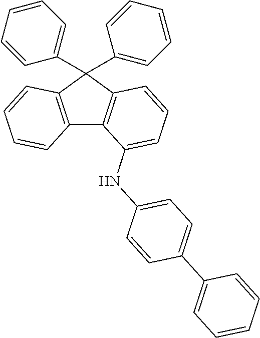 [SMILES: c1ccc(-c2ccc(Nc3cccc4c3-c3ccccc3C4(c3ccccc3)c3ccccc3)cc2)cc1] | 70 |

### b) Biphenyl-4-yl-(4-bromophenyl)-(9,9-dimethyl-9H-fluoren-4-yl)amine

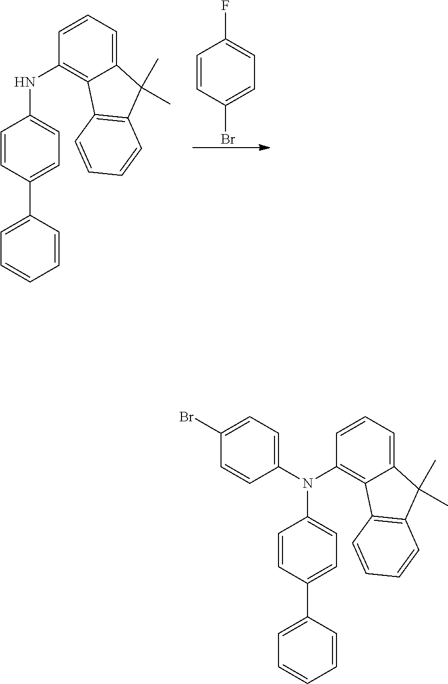

51.3 g (142 mmol, 1.00 eq) of biphenyl-4-yl-(9,9-dimethyl-9H-fluoren-4-yl)amine, 75.6 g (426 mmol, 3.00 eq) of 1-bromo-4-fluorobenzene [460-00-4] and 92.5 g (284 mmol, 2.00 eq) of caesium carbonate [534-17-8] are initially introduced in a 1 I four-necked flask, and 500 ml of dimethylacetamide are added. The reaction mixture is stirred at 150° C. for three days. When the reaction is complete, the batch is cooled to room temperature, and the solid is filtered off through Celite. The mother liquor is evaporated, and the precipitated solid is filtered and washed by stirring with hot methanol. Yield: 43 g (135 mmol), 95% of theory.

The following compounds can be prepared analogously:

| | Starting material | Product | Yield [%] |
|---|---|---|---|
| 1b | 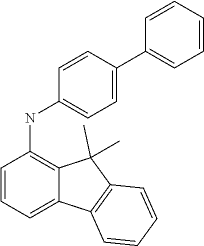 [SMILES: CC1(C)c2ccccc2-c2cccc(Nc3ccc(-c4ccccc4)cc3)c21] | 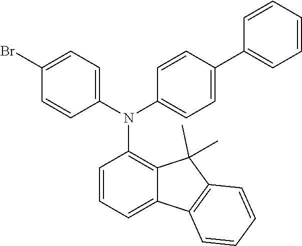 [SMILES: CC1(C)c2ccccc2-c2cccc(N(c3ccc(Br)cc3)c3ccc(-c4ccccc4)cc3)c21] | 78 |
| 2b | 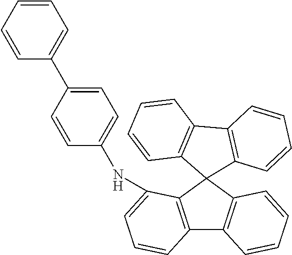 [SMILES: c1ccc(-c2ccc(Nc3cccc4c3C3(c5ccccc5-c5ccccc53)c3ccccc3-4)cc2)cc1] | 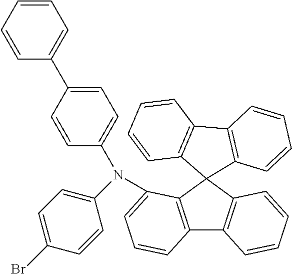 [SMILES: Brc1ccc(N(c2ccc(-c3ccccc3)cc2)c2cccc3c2C2(c4ccccc4-c4ccccc42)c2ccccc2-3)cc1] | 26 |
| 3b | 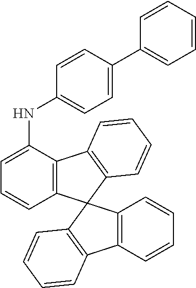 [SMILES: c1ccc(-c2ccc(Nc3cccc4c3-c3ccccc3C43c4ccccc4-c4ccccc43)cc2)cc1] | 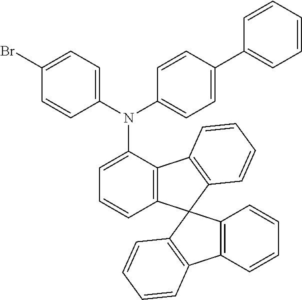 [SMILES: Brc1ccc(N(c2ccc(-c3ccccc3)cc2)c2cccc3c2-c2ccccc2C32c3ccccc3-c3ccccc32)cc1] | 84 |
| 4b | 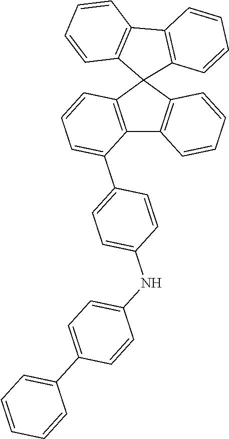 [SMILES: c1ccc(-c2ccc(Nc3ccc(-c4cccc5c4-c4ccccc4C54c5ccccc5-c5ccccc54)cc3)cc2)cc1] | 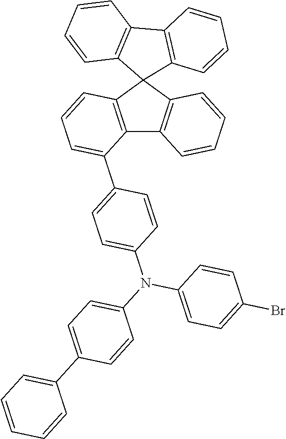 [SMILES: Brc1ccc(N(c2ccc(-c3ccccc3)cc2)c2ccc(-c3cccc4c3-c3ccccc3C43c4ccccc4-c4ccccc43)cc2)cc1] | 68 |
| 5b | 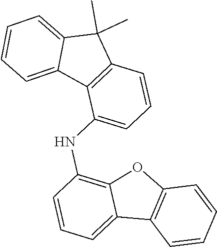 [SMILES: CC1(C)c2ccccc2-c2c(Nc3cccc4c3oc3ccccc34)cccc21] | 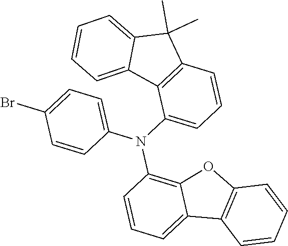 [SMILES: CC1(C)c2ccccc2-c2c(N(c3ccc(Br)cc3)c3cccc4c3oc3ccccc34)cccc21] | 67 |
| 6b | 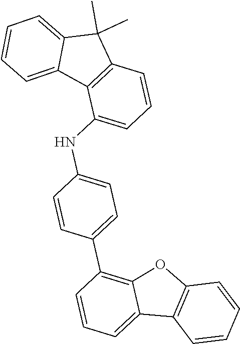 [SMILES: CC1(C)c2ccccc2-c2c(Nc3ccc(-c4cccc5c4oc4ccccc45)cc3)cccc21] | 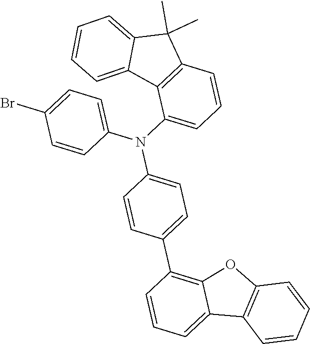 [SMILES: CC1(C)c2ccccc2-c2c(N(c3ccc(Br)cc3)c3ccc(-c4cccc5c4oc4ccccc45)cc3)cccc21] | 44 |
| 7b | 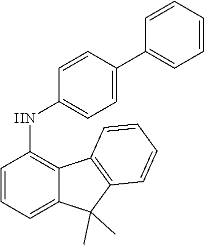 [SMILES: CC1(C)c2ccccc2-c2c(Nc3ccc(-c4ccccc4)cc3)cccc21] | 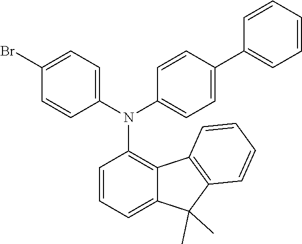 [SMILES: CC1(C)c2ccccc2-c2c(N(c3ccc(Br)cc3)c3ccc(-c4ccccc4)cc3)cccc21] | 59 |
| 8b | 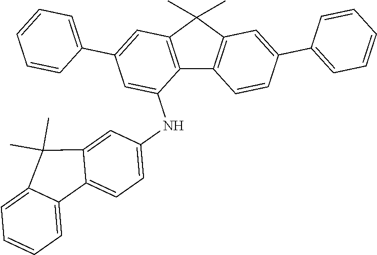 [SMILES: CC1(C)c2ccccc2-c2ccc(Nc3cc(-c4ccccc4)cc4c3-c3ccc(-c5ccccc5)cc3C4(C)C)cc21] | 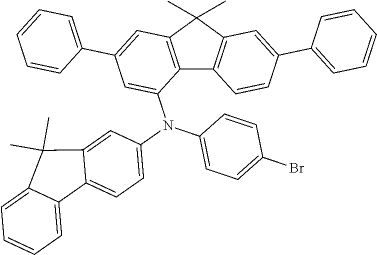 [SMILES: CC1(C)c2ccccc2-c2ccc(N(c3ccc(Br)cc3)c3cc(-c4ccccc4)cc4c3-c3ccc(-c5ccccc5)cc3C4(C)C)cc21] | 65 |
| 9b | 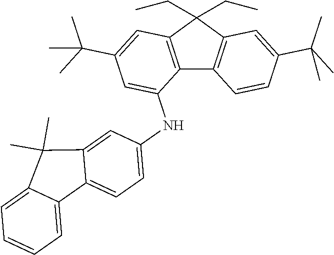 [SMILES: CCC1(CC)c2cc(C(C)(C)C)ccc2-c2c(Nc3ccc4c(c3)C(C)(C)c3ccccc3-4)cc(C(C)(C)C)cc21] | 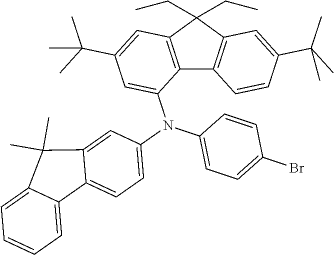 [SMILES: CCC1(CC)c2cc(C(C)(C)C)ccc2-c2c(N(c3ccc(Br)cc3)c3ccc4c(c3)C(C)(C)c3ccccc3-4)cc(C(C)(C)C)cc21] | 67 |
| 10b | 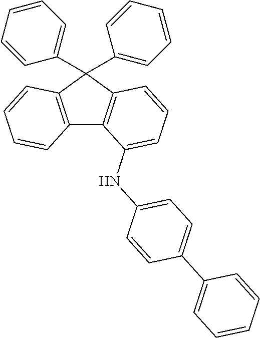 [SMILES: c1ccc(-c2ccc(Nc3cccc4c3-c3ccccc3C4(c3ccccc3)c3ccccc3)cc2)cc1] | 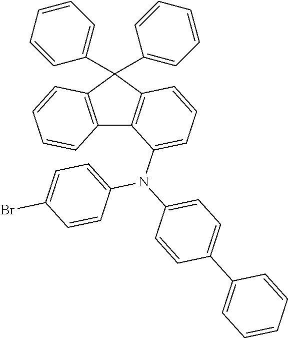 [SMILES: Brc1ccc(N(c2ccc(-c3ccccc3)cc2)c2cccc3c2-c2ccccc2C3(c2ccccc2)c2ccccc2)cc1] | 71 |
| 11b | 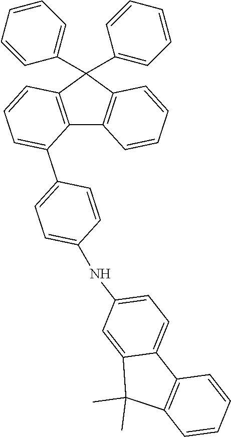 [SMILES: CC1(C)c2ccccc2-c2ccc(Nc3ccc(-c4cccc5c4-c4ccccc4C5(c4ccccc4)c4ccccc4)cc3)cc21] ]1852534-95-2] | 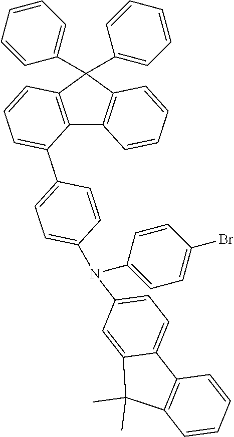 [SMILES: CC1(C)c2ccccc2-c2ccc(N(c3ccc(Br)cc3)c3ccc(-c4cccc5c4-c4ccccc4C5(c4ccccc4)c4ccccc4)cc3)cc21] | 70 |

### c) Biphenyl-4-yl-(3′-bromobiphenyl-3-yl)-(9,9-dimethyl-9H-fluoren-2-yl)amine

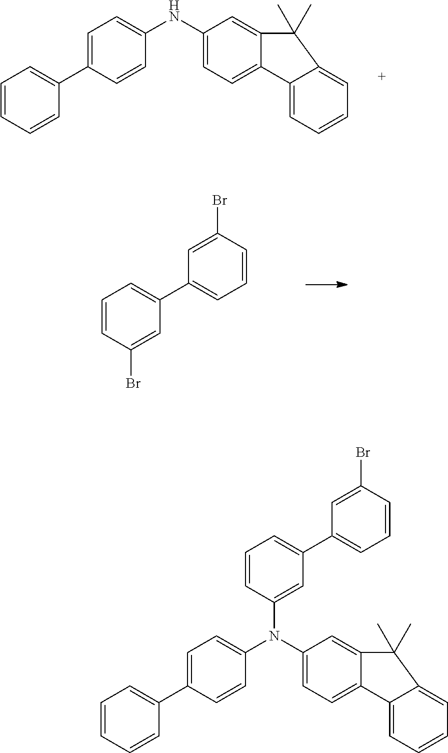 [SMILES: CC1(C)c2ccccc2-c2ccc(Nc3ccc(-c4ccccc4)cc3)cc21 ; Brc1cccc(-c2cccc(Br)c2)c1 ; CC1(C)c2ccccc2-c2ccc(N(c3ccc(-c4ccccc4)cc3)c3cccc(-c4cccc(Br)c4)c3)cc21]

29 g (80 mmol, 1.0 eq) of the intermediate from reaction a) are dissolved in 600 ml of toluene together with 25 g (80 mmol, 1.0 eq) of 3,3′-dibromo-1,1′-biphenyl (CAS 16400-51-4) and degassed for 30 minutes. 45 g (240 mmol, 3.0 eq) of sodium tert-butoxide, 890 mg (0.40 mmol, 0.050 eq) of palladium(II) acetate and 8 ml (8.0 mmol, 0.10 eq.) of a 1 M tri-tert-butylphosphine solution are subsequently added. The batch is heated under reflux overnight and, when the reaction is complete, filtered twice through aluminium oxide with toluene. After removal of the solvent in a rotary evaporator, the oil is dissolved in a little THF and introduced into heptane. The solid formed is filtered off with suction and purified by means of hot extraction in heptane/toluene 1:1, giving 16.6 g (28 mmol, 35%) of the desired product.

The following compounds can be prepared analogously:

| | Starting | Starting |
|---|---|---|
| | material 3 | material 4 |
| 1c | 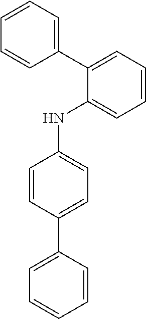 [SMILES: c1ccc(-c2ccc(Nc3ccccc3-c3ccccc3)cc2)cc1] | 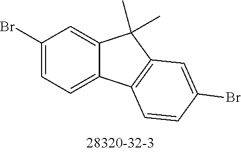 [SMILES: CC1(C)c2cc(Br)ccc2-c2ccc(Br)cc21] |
| 2c | 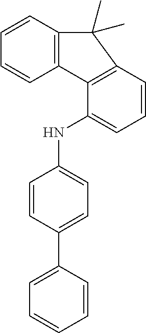 [SMILES: CC1(C)c2ccccc2-c2c(Nc3ccc(-c4ccccc4)cc3)cccc21] | 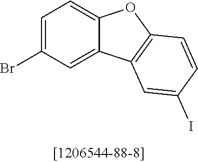 [SMILES: Brc1ccc2oc3ccc(I)cc3c2c1] |
| 3c | 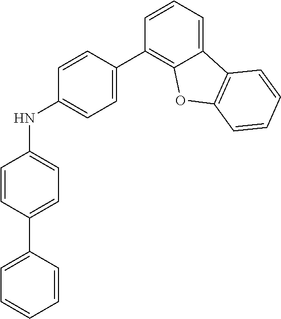 [SMILES: c1ccc(-c2ccc(Nc3ccc(-c4cccc5c4oc4ccccc45)cc3)cc2)cc1] | 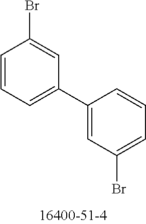 [SMILES: Brc1cccc(-c2cccc(Br)c2)c1] |
| 4c | 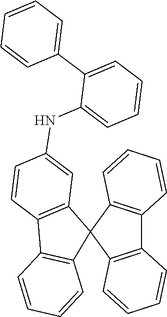 [SMILES: c1ccc(-c2ccccc2Nc2ccc3c(c2)C2(c4ccccc4-c4ccccc42)c2ccccc2-3)cc1] | 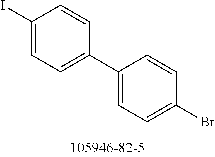 [SMILES: Brc1ccc(-c2ccc(I)cc2)cc1] |
| 5c | 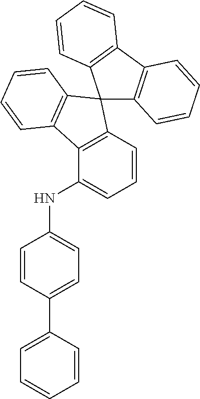 [SMILES: c1ccc(-c2ccc(Nc3cccc4c3-c3ccccc3C43c4ccccc4-c4ccccc43)cc2)cc1] | 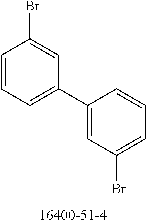 [SMILES: Brc1cccc(-c2cccc(Br)c2)c1] |
| 6c | 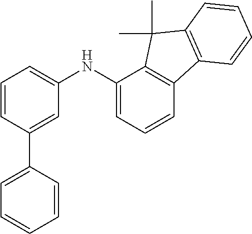 [SMILES: CC1(C)c2ccccc2-c2cccc(Nc3cccc(-c4ccccc4)c3)c21] | 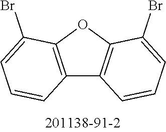 [SMILES: Brc1cccc2c1oc1c(Br)cccc12] |
| 7c | 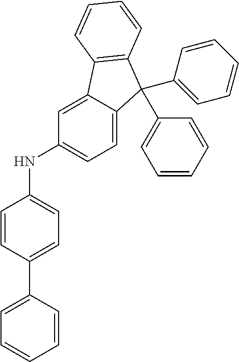 [SMILES: c1ccc(-c2ccc(Nc3ccc4c(c3)-c3ccccc3C4(c3ccccc3)c3ccccc3)cc2)cc1] | 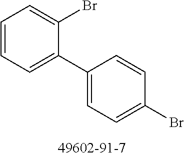 [SMILES: Brc1ccc(-c2ccccc2Br)cc1] |
| 8c | 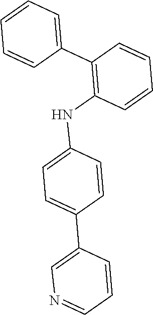 [SMILES: c1ccc(-c2ccccc2Nc2ccc(-c3cccnc3)cc2)cc1] | 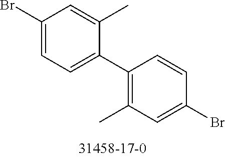 [SMILES: Cc1cc(Br)ccc1-c1ccc(Br)cc1C] |
| 9c | 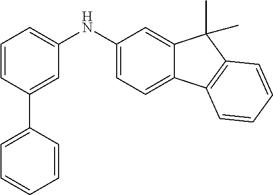 [SMILES: CC1(C)c2ccccc2-c2ccc(Nc3cccc(-c4ccccc4)c3)cc21] | 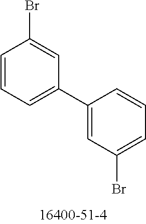 [SMILES: Brc1cccc(-c2cccc(Br)c2)c1] |
| 10c | 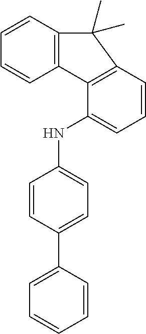 [SMILES: CC1(C)c2ccccc2-c2c(Nc3ccc(-c4ccccc4)cc3)cccc21] | 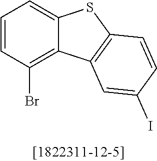 [SMILES: Brc1cccc2sc3ccc(I)cc3c12] |
| 11c | 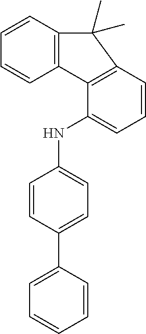 [SMILES: CC1(C)c2ccccc2-c2c(Nc3ccc(-c4ccccc4)cc3)cccc21] | 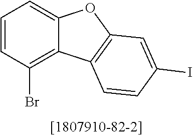 [SMILES: Brc1cccc2oc3cc(I)ccc3c12] |
| 12c | 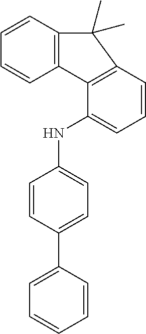 [SMILES: CC1(C)c2ccccc2-c2c(Nc3ccc(-c4ccccc4)cc3)cccc21] | 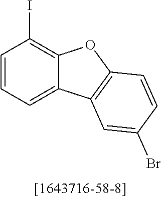 [SMILES: Brc1ccc2oc3c(I)cccc3c2c1] |
| 13c | 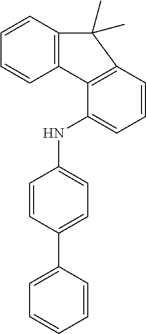 [SMILES: CC1(C)c2ccccc2-c2c(Nc3ccc(-c4ccccc4)cc3)cccc21] | 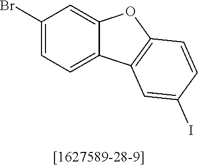 [SMILES: Brc1ccc2c(c1)oc1ccc(I)cc12] |

| | Product | Yield |
|---|---|---|
| 1c | 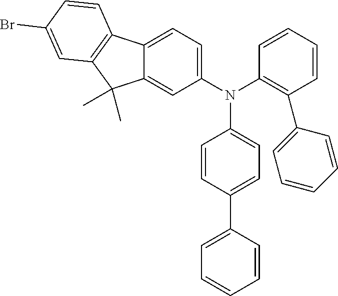 [SMILES: CC1(C)c2cc(Br)ccc2-c2ccc(N(c3ccc(-c4ccccc4)cc3)c3ccccc3-c3ccccc3)cc21] | 49% |
| 2c | 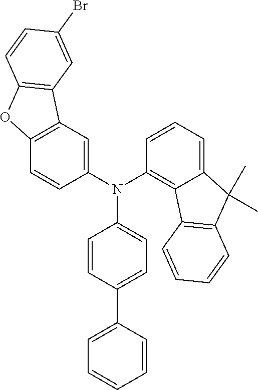 [SMILES: CC1(C)c2ccccc2-c2c(N(c3ccc(-c4ccccc4)cc3)c3ccc4oc5ccc(Br)cc5c4c3)cccc21] | 65% |
| 3c | 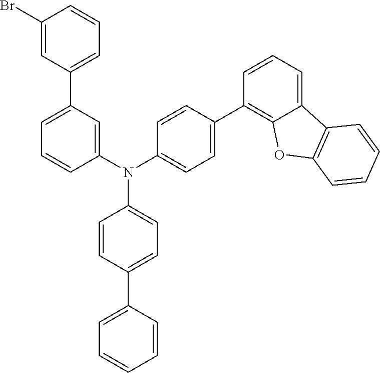 [SMILES: Brc1cccc(-c2cccc(N(c3ccc(-c4ccccc4)cc3)c3ccc(-c4cccc5c4oc4ccccc45)cc3)c2)c1] | 72% |
| 4c | 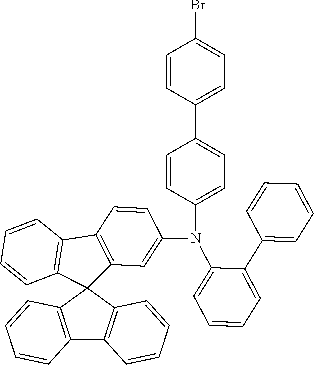 [SMILES: Brc1ccc(-c2ccc(N(c3ccc4c(c3)C3(c5ccccc5-c5ccccc53)c3ccccc3-4)c3ccccc3-c3ccccc3)cc2)cc1] | 82% |
| 5c | 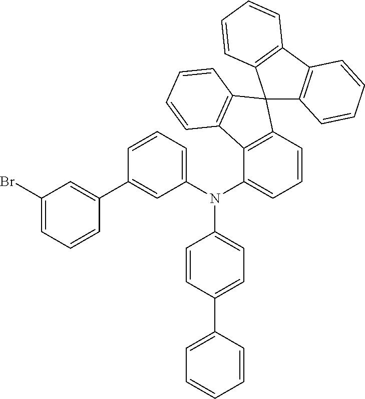 [SMILES: Brc1cccc(-c2cccc(N(c3ccc(-c4ccccc4)cc3)c3cccc4c3-c3ccccc3C43c4ccccc4-c4ccccc43)c2)c1] | 41% |
| 6c | 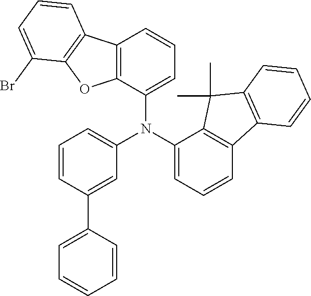 [SMILES: CC1(C)c2ccccc2-c2cccc(N(c3cccc(-c4ccccc4)c3)c3cccc4c3oc3c(Br)cccc34)c21] | 27% |
| 7c | 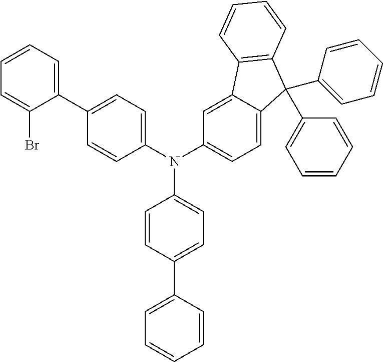 [SMILES: Brc1ccccc1-c1ccc(N(c2ccc(-c3ccccc3)cc2)c2ccc3c(c2)-c2ccccc2C3(c2ccccc2)c2ccccc2)cc1] | 56% |
| 8c | 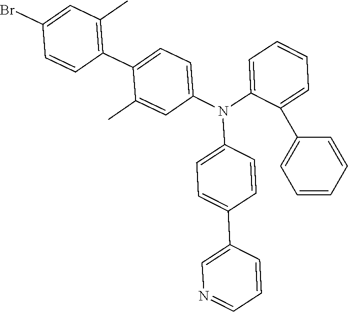 [SMILES: Cc1cc(Br)ccc1-c1ccc(N(c2ccc(-c3cccnc3)cc2)c2ccccc2-c2ccccc2)cc1C] | 47% |
| 9c | 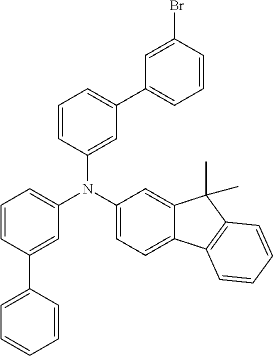 [SMILES: CC1(C)c2ccccc2-c2ccc(N(c3cccc(-c4ccccc4)c3)c3cccc(-c4cccc(Br)c4)c3)cc21] | 57% |
| 10c | 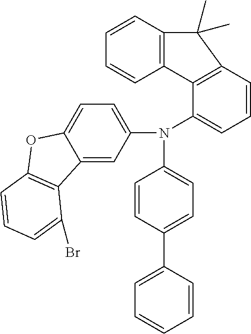 [SMILES: CC1(C)c2ccccc2-c2c(N(c3ccc(-c4ccccc4)cc3)c3ccc4oc5cccc(Br)c5c4c3)cccc21] | 64 |
| 11c | 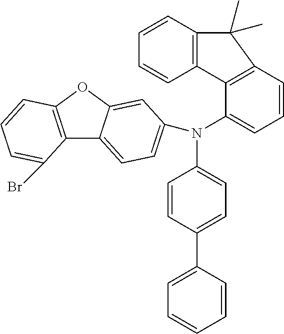 [SMILES: CC1(C)c2ccccc2-c2c(N(c3ccc(-c4ccccc4)cc3)c3ccc4c(c3)oc3cccc(Br)c34)cccc21] | 67 |
| 12c | 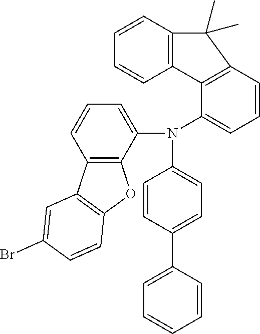 [SMILES: CC1(C)c2ccccc2-c2c(N(c3ccc(-c4ccccc4)cc3)c3cccc4c3oc3ccc(Br)cc34)cccc21] | 72 |
| 13c | 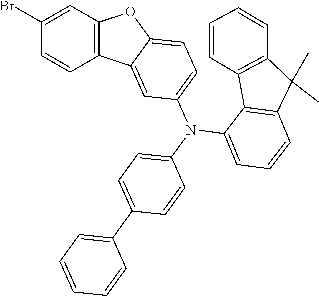 [SMILES: CC1(C)c2ccccc2-c2c(N(c3ccc(-c4ccccc4)cc3)c3ccc4oc5cc(Br)ccc5c4c3)cccc21] | 59 |

### d) Biphenyl-4-yl-(9,9-dimethyl-9H-fluoren-1-yl)-[4-(4-phenyl-quinazolin-2-yl)phenyl]amine

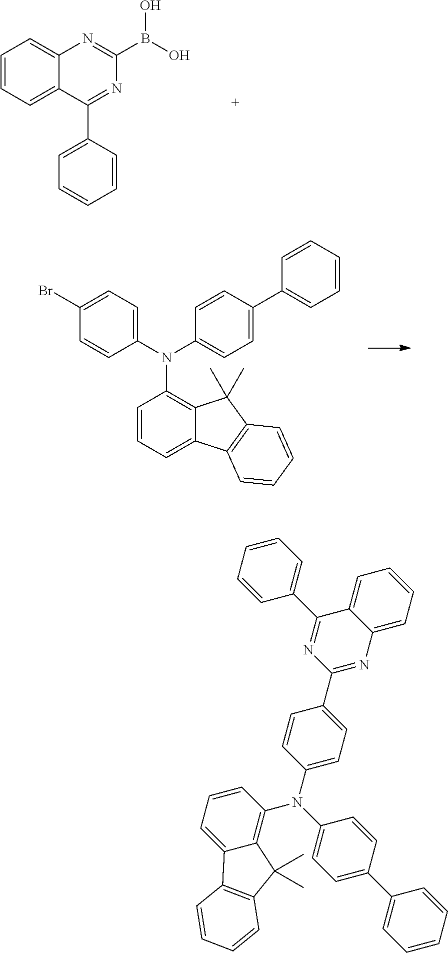 [SMILES: OB(O)c1nc(-c2ccccc2)c2ccccc2n1 ; CC1(C)c2ccccc2-c2cccc(N(c3ccc(Br)cc3)c3ccc(-c4ccccc4)cc3)c21 ; CC1(C)c2ccccc2-c2cccc(N(c3ccc(-c4ccccc4)cc3)c3ccc(-c4nc(-c5ccccc5)c5ccccc5n4)cc3)c21]

27.5 g (110.0 mmol) of 4-phenylquinazoline-2-boronic acid, 56 g (110.0 mmol) of biphenyl-4-yl-(4-bromophenyl)-(9,9-dimethyl-9H-fluoren-1-yl)amine and 26 g (210.0 mmol) of sodium carbonate are suspended in 500 ml of ethylene glycol dimethyl ether and 500 ml of water. 913 mg (3.0 mmol) of tri-o-tolylphosphine and then 112 mg (0.5 mmol) of palladium(II) acetate are added, and the reaction mixture is heated under reflux for 16 h. After cooling, the organic phase is separated off, filtered through silica gel, washed three times with 200 ml of water and subsequently evaporated to dryness. The product is purified by column chromatography on silica gel with toluene/heptane (1:2) and finally sublimed in a high vacuum (p=5×10⁻⁷ mbar) (purity 99.9%). The yield is 53 g (83 mmol), corresponding to 73% of theory.

The following compounds can be prepared analogously:

| | Starting | Starting material | | Yield |
|---|---|---|---|---|
| | material 1 | 2 | Product | [%] |
| 1d | 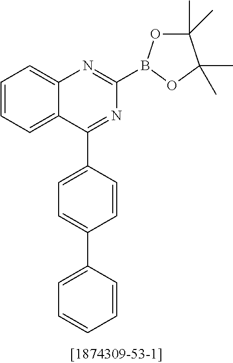 [SMILES: CC1(C)OB(c2nc(-c3ccc(-c4ccccc4)cc3)c3ccccc3n2)OC1(C)C] | 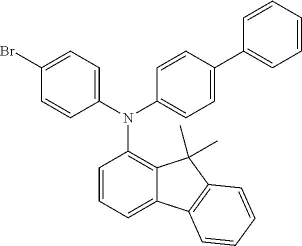 [SMILES: CC1(C)c2ccccc2-c2cccc(N(c3ccc(Br)cc3)c3ccc(-c4ccccc4)cc3)c21] | 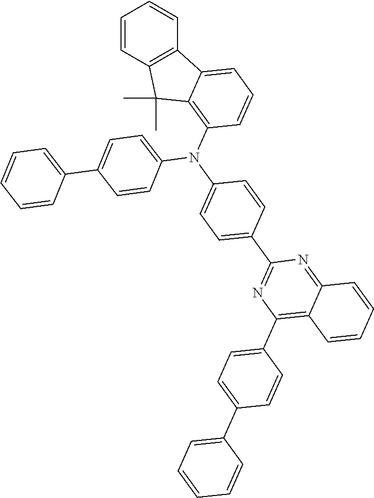 [SMILES: CC1(C)c2ccccc2-c2cccc(N(c3ccc(-c4ccccc4)cc3)c3ccc(-c4nc(-c5ccc(-c6ccccc6)cc5)c5ccccc5n4)cc3)c21] | 71 |
| 2d | 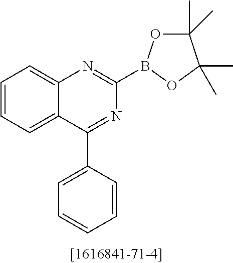 [SMILES: CC1(C)OB(c2nc(-c3ccccc3)c3ccccc3n2)OC1(C)C] | 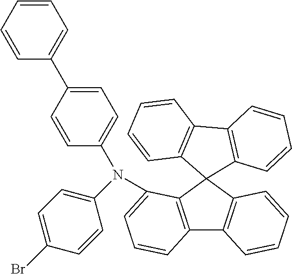 [SMILES: Brc1ccc(N(c2ccc(-c3ccccc3)cc2)c2cccc3c2C2(c4ccccc4-c4ccccc42)c2ccccc2-3)cc1] | 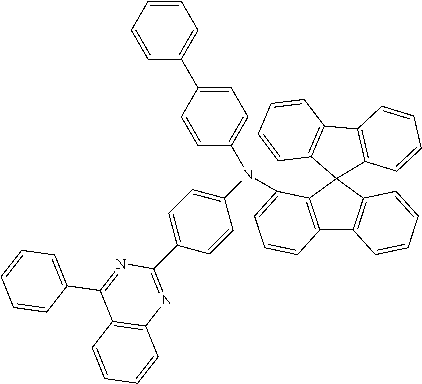 [SMILES: c1ccc(-c2ccc(N(c3ccc(-c4nc(-c5ccccc5)c5ccccc5n4)cc3)c3cccc4c3C3(c5ccccc5-c5ccccc53)c3ccccc3-4)cc2)cc1] | 70 |
| 3d | 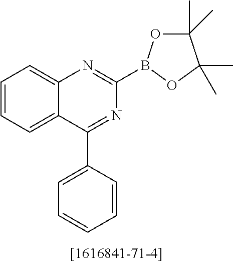 [SMILES: CC1(C)OB(c2nc(-c3ccccc3)c3ccccc3n2)OC1(C)C] | 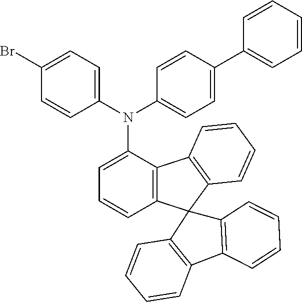 [SMILES: Brc1ccc(N(c2ccc(-c3ccccc3)cc2)c2cccc3c2-c2ccccc2C32c3ccccc3-c3ccccc32)cc1] | 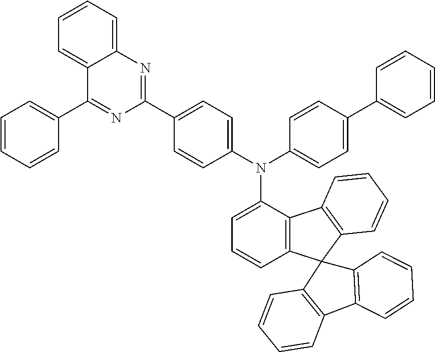 [SMILES: c1ccc(-c2ccc(N(c3ccc(-c4nc(-c5ccccc5)c5ccccc5n4)cc3)c3cccc4c3-c3ccccc3C43c4ccccc4-c4ccccc43)cc2)cc1] | 67 |
| 4d | 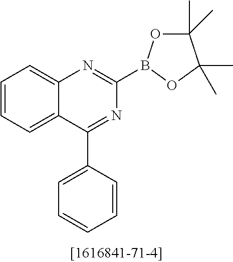 [SMILES: CC1(C)OB(c2nc(-c3ccccc3)c3ccccc3n2)OC1(C)C] | 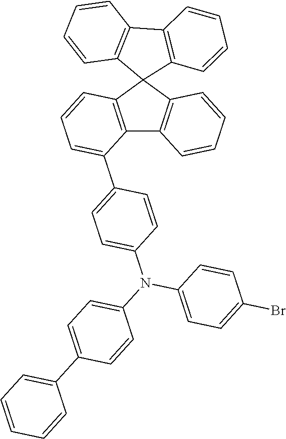 [SMILES: Brc1ccc(N(c2ccc(-c3ccccc3)cc2)c2ccc(-c3cccc4c3-c3ccccc3C43c4ccccc4-c4ccccc43)cc2)cc1] | 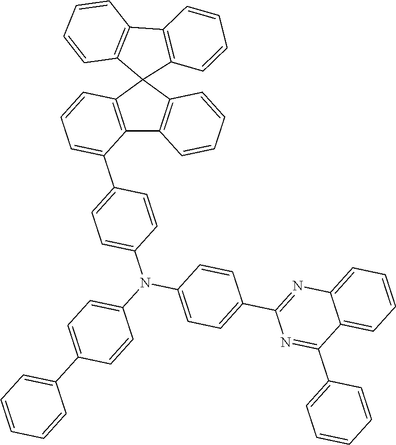 [SMILES: c1ccc(-c2ccc(N(c3ccc(-c4nc(-c5ccccc5)c5ccccc5n4)cc3)c3ccc(-c4cccc5c4-c4ccccc4C54c5ccccc5-c5ccccc54)cc3)cc2)cc1] | 65 |
| 5d | 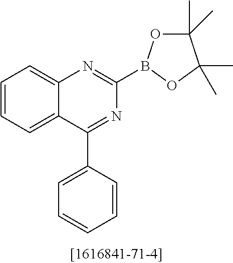 [SMILES: CC1(C)OB(c2nc(-c3ccccc3)c3ccccc3n2)OC1(C)C] | 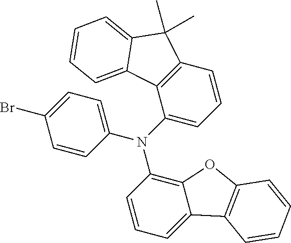 [SMILES: CC1(C)c2ccccc2-c2c(N(c3ccc(Br)cc3)c3cccc4c3oc3ccccc34)cccc21] | 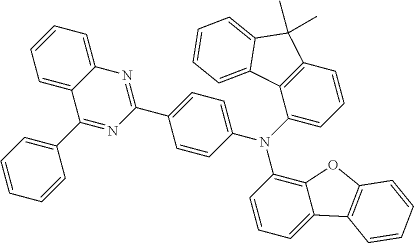 [SMILES: CC1(C)c2ccccc2-c2c(N(c3ccc(-c4nc(-c5ccccc5)c5ccccc5n4)cc3)c3cccc4c3oc3ccccc34)cccc21] | 68 |
| 6d | 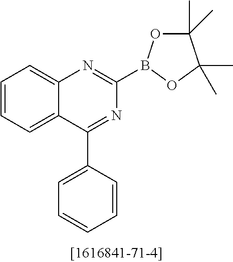 [SMILES: CC1(C)OB(c2nc(-c3ccccc3)c3ccccc3n2)OC1(C)C] | 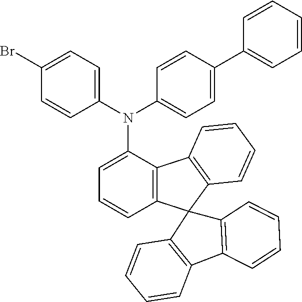 [SMILES: Brc1ccc(N(c2ccc(-c3ccccc3)cc2)c2cccc3c2-c2ccccc2C32c3ccccc3-c3ccccc32)cc1] | 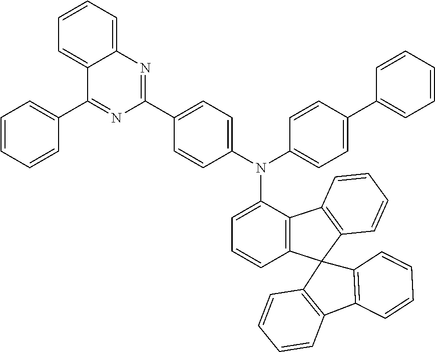 [SMILES: c1ccc(-c2ccc(N(c3ccc(-c4nc(-c5ccccc5)c5ccccc5n4)cc3)c3cccc4c3-c3ccccc3C43c4ccccc4-c4ccccc43)cc2)cc1] | 69 |
| 7d | 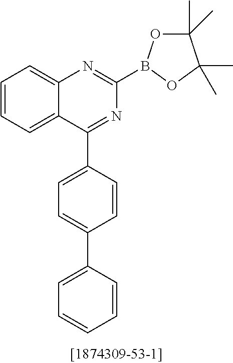 [SMILES: CC1(C)OB(c2nc(-c3ccc(-c4ccccc4)cc3)c3ccccc3n2)OC1(C)C] | 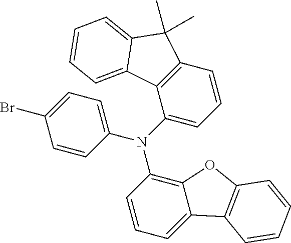 [SMILES: CC1(C)c2ccccc2-c2c(N(c3ccc(Br)cc3)c3cccc4c3oc3ccccc34)cccc21] | 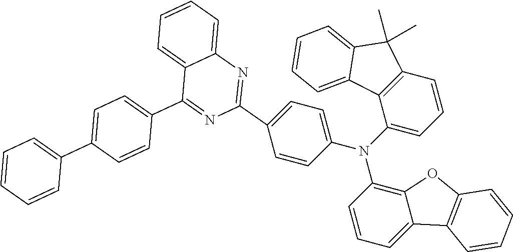 [SMILES: CC1(C)c2ccccc2-c2c(N(c3ccc(-c4nc(-c5ccc(-c6ccccc6)cc5)c5ccccc5n4)cc3)c3cccc4c3oc3ccccc34)cccc21] | 66 |
| 8d | 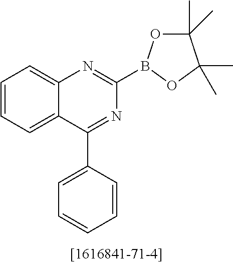 [SMILES: CC1(C)OB(c2nc(-c3ccccc3)c3ccccc3n2)OC1(C)C] | 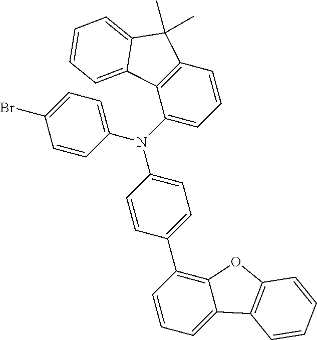 [SMILES: CC1(C)c2ccccc2-c2c(N(c3ccc(Br)cc3)c3ccc(-c4cccc5c4oc4ccccc45)cc3)cccc21] | 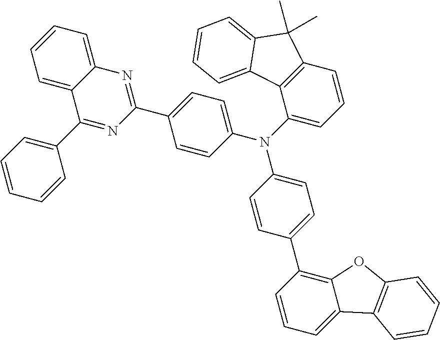 [SMILES: CC1(C)c2ccccc2-c2c(N(c3ccc(-c4nc(-c5ccccc5)c5ccccc5n4)cc3)c3ccc(-c4cccc5c4oc4ccccc45)cc3)cccc21] | 62 |
| 9d | 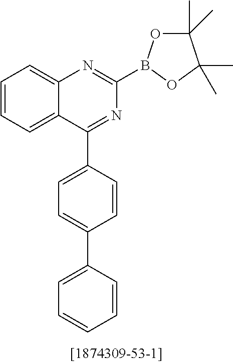 [SMILES: CC1(C)OB(c2nc(-c3ccc(-c4ccccc4)cc3)c3ccccc3n2)OC1(C)C] | 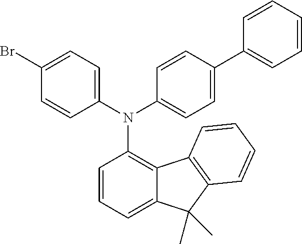 [SMILES: CC1(C)c2ccccc2-c2c(N(c3ccc(Br)cc3)c3ccc(-c4ccccc4)cc3)cccc21] | 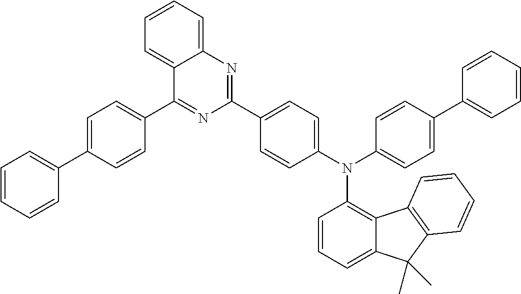 [SMILES: CC1(C)c2ccccc2-c2c(N(c3ccc(-c4ccccc4)cc3)c3ccc(-c4nc(-c5ccc(-c6ccccc6)cc5)c5ccccc5n4)cc3)cccc21] | 68 |
| 10d | 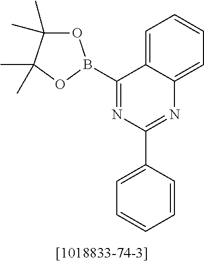 [SMILES: CC1(C)OB(c2nc(-c3ccccc3)nc3ccccc23)OC1(C)C] | 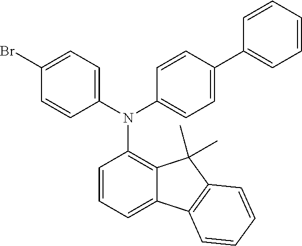 [SMILES: CC1(C)c2ccccc2-c2cccc(N(c3ccc(Br)cc3)c3ccc(-c4ccccc4)cc3)c21] | 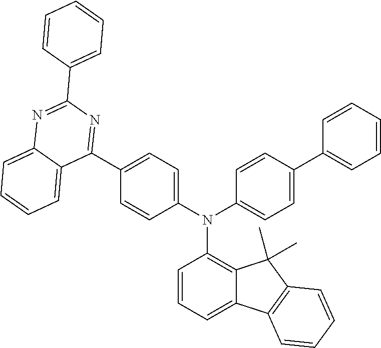 [SMILES: CC1(C)c2ccccc2-c2cccc(N(c3ccc(-c4ccccc4)cc3)c3ccc(-c4nc(-c5ccccc5)nc5ccccc45)cc3)c21] | 68 |
| 11d | 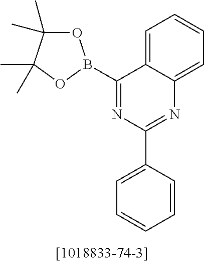 [SMILES: CC1(C)OB(c2nc(-c3ccccc3)nc3ccccc23)OC1(C)C] | 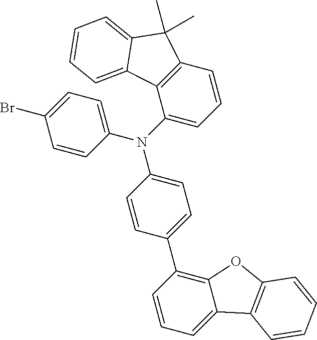 [SMILES: CC1(C)c2ccccc2-c2c(N(c3ccc(Br)cc3)c3ccc(-c4cccc5c4oc4ccccc45)cc3)cccc21] | 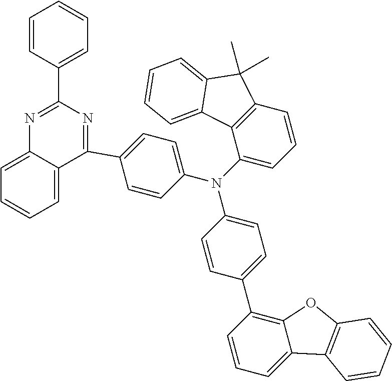 [SMILES: CC1(C)c2ccccc2-c2c(N(c3ccc(-c4nc(-c5ccccc5)nc5ccccc45)cc3)c3ccc(-c4cccc5c4oc4ccccc45)cc3)cccc21] | 71 |
| 12d | 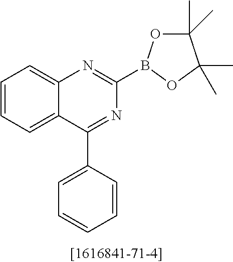 [SMILES: CC1(C)OB(c2nc(-c3ccccc3)c3ccccc3n2)OC1(C)C] | 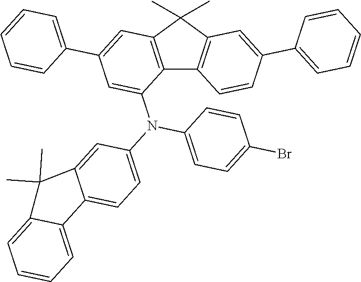 [SMILES: CC1(C)c2ccccc2-c2ccc(N(c3ccc(Br)cc3)c3cc(-c4ccccc4)cc4c3-c3ccc(-c5ccccc5)cc3C4(C)C)cc21] | 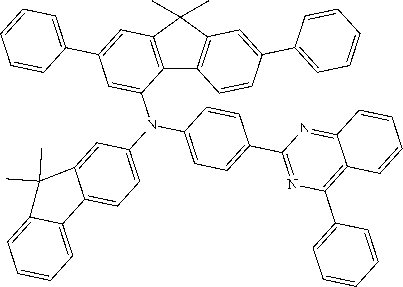 [SMILES: CC1(C)c2ccccc2-c2ccc(N(c3ccc(-c4nc(-c5ccccc5)c5ccccc5n4)cc3)c3cc(-c4ccccc4)cc4c3-c3ccc(-c5ccccc5)cc3C4(C)C)cc21] | 70 |
| 13d | 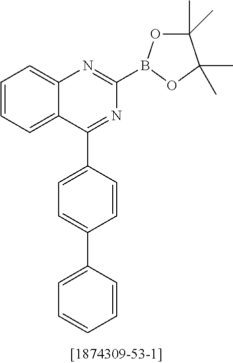 [SMILES: CC1(C)OB(c2nc(-c3ccc(-c4ccccc4)cc3)c3ccccc3n2)OC1(C)C] | 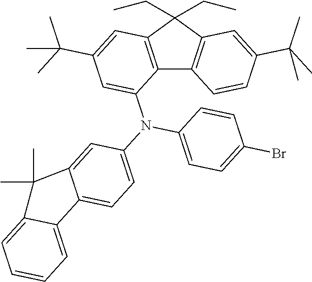 [SMILES: CCC1(CC)c2cc(C(C)(C)C)ccc2-c2c(N(c3ccc(Br)cc3)c3ccc4c(c3)C(C)(C)c3ccccc3-4)cc(C(C)(C)C)cc21] | 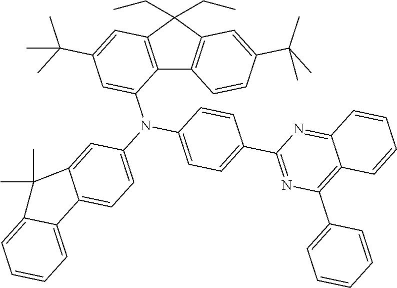 [SMILES: CCC1(CC)c2cc(C(C)(C)C)ccc2-c2c(N(c3ccc(-c4nc(-c5ccccc5)c5ccccc5n4)cc3)c3ccc4c(c3)C(C)(C)c3ccccc3-4)cc(C(C)(C)C)cc21] | 69 |
| 14d | 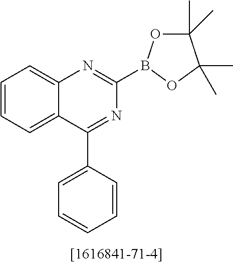 [SMILES: CC1(C)OB(c2nc(-c3ccccc3)c3ccccc3n2)OC1(C)C] | 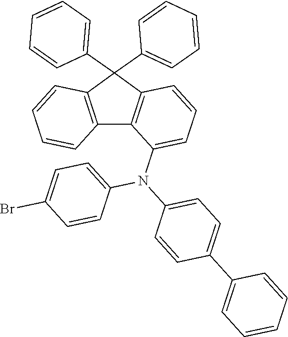 [SMILES: Brc1ccc(N(c2ccc(-c3ccccc3)cc2)c2cccc3c2-c2ccccc2C3(c2ccccc2)c2ccccc2)cc1] | 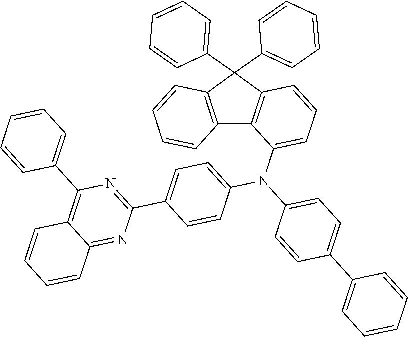 [SMILES: c1ccc(-c2ccc(N(c3ccc(-c4nc(-c5ccccc5)c5ccccc5n4)cc3)c3cccc4c3-c3ccccc3C4(c3ccccc3)c3ccccc3)cc2)cc1] | 67 |
| 15d | 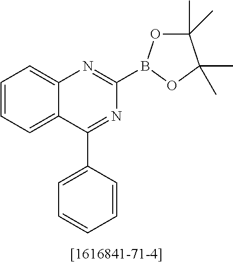 [SMILES: CC1(C)OB(c2nc(-c3ccccc3)c3ccccc3n2)OC1(C)C] | 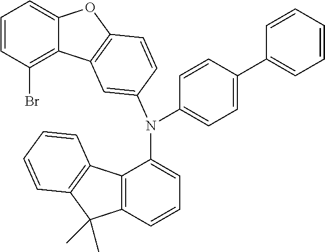 [SMILES: CC1(C)c2ccccc2-c2c(N(c3ccc(-c4ccccc4)cc3)c3ccc4oc5cccc(Br)c5c4c3)cccc21] | 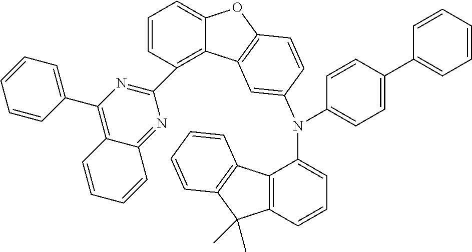 [SMILES: CC1(C)c2ccccc2-c2c(N(c3ccc(-c4ccccc4)cc3)c3ccc4oc5cccc(-c6nc(-c7ccccc7)c7ccccc7n6)c5c4c3)cccc21] | 63 |
| 16d | 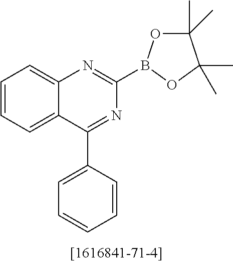 [SMILES: CC1(C)OB(c2nc(-c3ccccc3)c3ccccc3n2)OC1(C)C] | 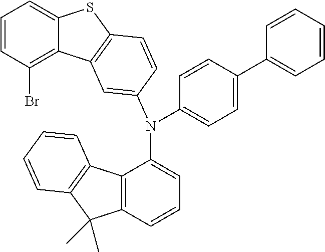 [SMILES: CC1(C)c2ccccc2-c2c(N(c3ccc(-c4ccccc4)cc3)c3ccc4sc5cccc(Br)c5c4c3)cccc21] | 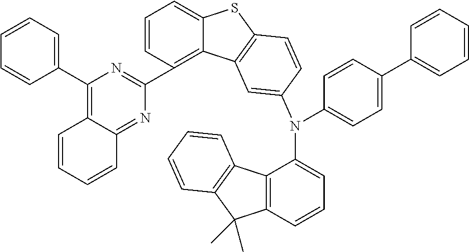 [SMILES: CC1(C)c2ccccc2-c2c(N(c3ccc(-c4ccccc4)cc3)c3ccc4sc5cccc(-c6nc(-c7ccccc7)c7ccccc7n6)c5c4c3)cccc21] | 64 |
| 17d | 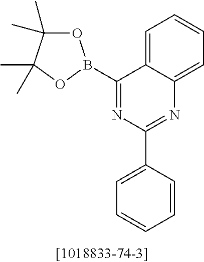 [SMILES: CC1(C)OB(c2nc(-c3ccccc3)nc3ccccc23)OC1(C)C] | 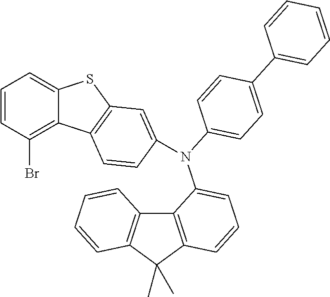 [SMILES: CC1(C)c2ccccc2-c2c(N(c3ccc(-c4ccccc4)cc3)c3ccc4c(c3)sc3cccc(Br)c34)cccc21] | 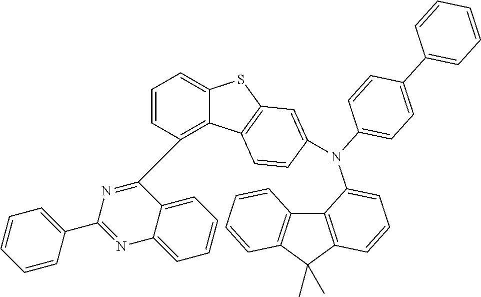 [SMILES: CC1(C)c2ccccc2-c2c(N(c3ccc(-c4ccccc4)cc3)c3ccc4c(c3)sc3cccc(-c5nc(-c6ccccc6)nc6ccccc56)c34)cccc21] | 67 |
| 18d | 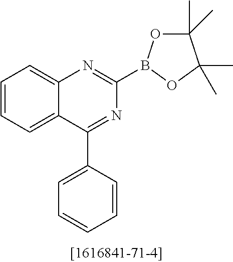 [SMILES: CC1(C)OB(c2nc(-c3ccccc3)c3ccccc3n2)OC1(C)C] | 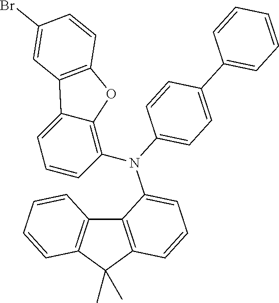 [SMILES: CC1(C)c2ccccc2-c2c(N(c3ccc(-c4ccccc4)cc3)c3cccc4c3oc3ccc(Br)cc34)cccc21] | 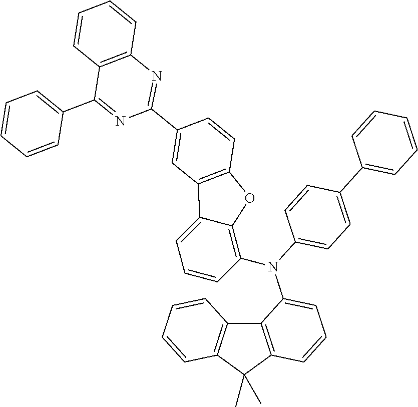 [SMILES: CC1(C)c2ccccc2-c2c(N(c3ccc(-c4ccccc4)cc3)c3cccc4c3oc3ccc(-c5nc(-c6ccccc6)c6ccccc6n5)cc34)cccc21] | 60 |
| 19d | 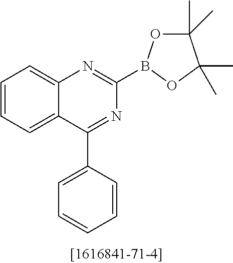 [SMILES: CC1(C)OB(c2nc(-c3ccccc3)c3ccccc3n2)OC1(C)C] | 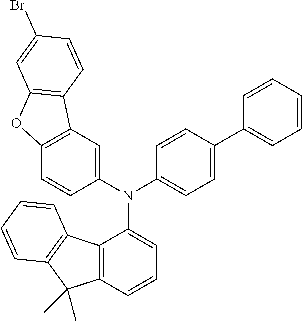 [SMILES: CC1(C)c2ccccc2-c2c(N(c3ccc(-c4ccccc4)cc3)c3ccc4oc5cc(Br)ccc5c4c3)cccc21] | 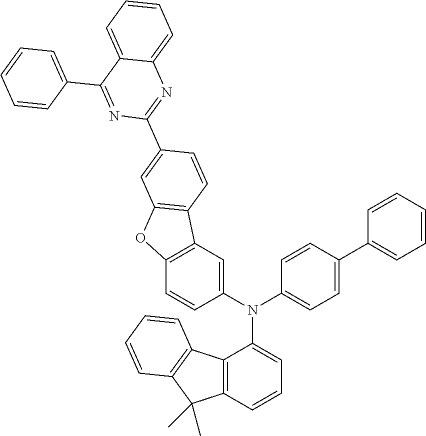 [SMILES: CC1(C)c2ccccc2-c2c(N(c3ccc(-c4ccccc4)cc3)c3ccc4oc5cc(-c6nc(-c7ccccc7)c7ccccc7n6)ccc5c4c3)cccc21] | 68 |
| 20d | 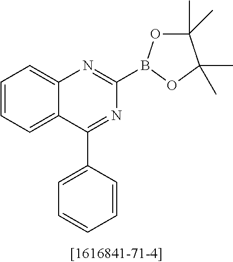 [SMILES: CC1(C)OB(c2nc(-c3ccccc3)c3ccccc3n2)OC1(C)C] | 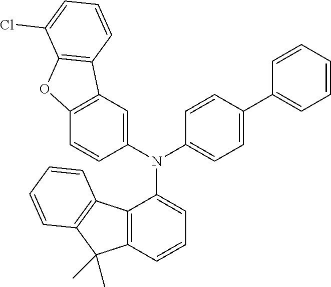 [SMILES: CC1(C)c2ccccc2-c2c(N(c3ccc(-c4ccccc4)cc3)c3ccc4oc5c(Cl)cccc5c4c3)cccc21] | 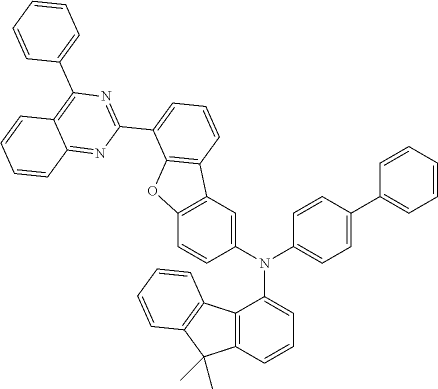 [SMILES: CC1(C)c2ccccc2-c2c(N(c3ccc(-c4ccccc4)cc3)c3ccc4oc5c(-c6nc(-c7ccccc7)c7ccccc7n6)cccc5c4c3)cccc21] | 67 |
| 21d | 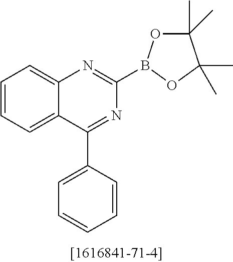 [SMILES: CC1(C)OB(c2nc(-c3ccccc3)c3ccccc3n2)OC1(C)C] | 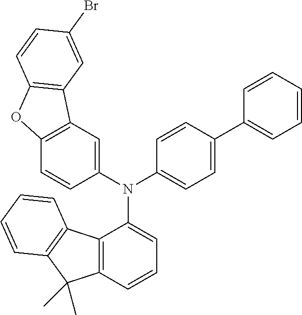 [SMILES: CC1(C)c2ccccc2-c2c(N(c3ccc(-c4ccccc4)cc3)c3ccc4oc5ccc(Br)cc5c4c3)cccc21] | 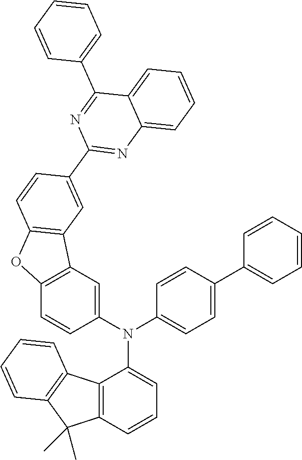 [SMILES: CC1(C)c2ccccc2-c2c(N(c3ccc(-c4ccccc4)cc3)c3ccc4oc5ccc(-c6nc(-c7ccccc7)c7ccccc7n6)cc5c4c3)cccc21] | 62 |
| 22d | 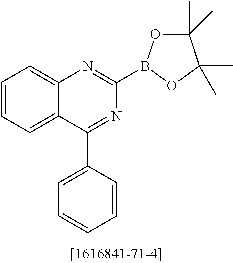 [SMILES: CC1(C)OB(c2nc(-c3ccccc3)c3ccccc3n2)OC1(C)C] | 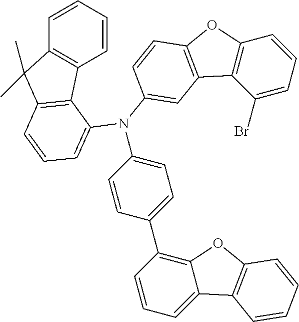 [SMILES: CC1(C)c2ccccc2-c2c(N(c3ccc(-c4cccc5c4oc4ccccc45)cc3)c3ccc4oc5cccc(Br)c5c4c3)cccc21] | 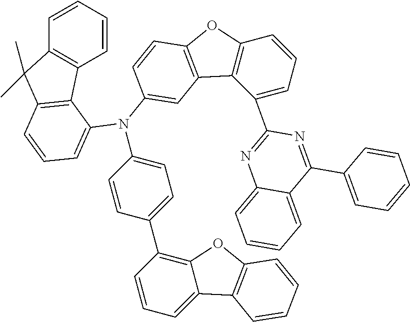 [SMILES: CC1(C)c2ccccc2-c2c(N(c3ccc(-c4cccc5c4oc4ccccc45)cc3)c3ccc4oc5cccc(-c6nc(-c7ccccc7)c7ccccc7n6)c5c4c3)cccc21] | 64 |
| 23d | 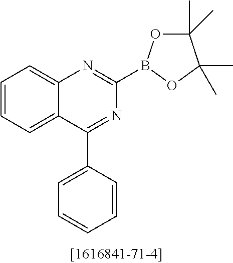 [SMILES: CC1(C)OB(c2nc(-c3ccccc3)c3ccccc3n2)OC1(C)C] | 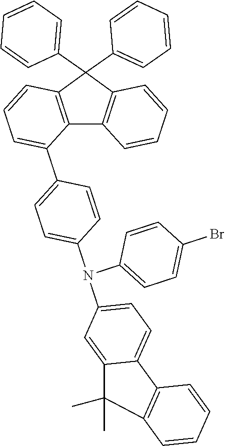 [SMILES: CC1(C)c2ccccc2-c2ccc(N(c3ccc(Br)cc3)c3ccc(-c4cccc5c4-c4ccccc4C5(c4ccccc4)c4ccccc4)cc3)cc21] | 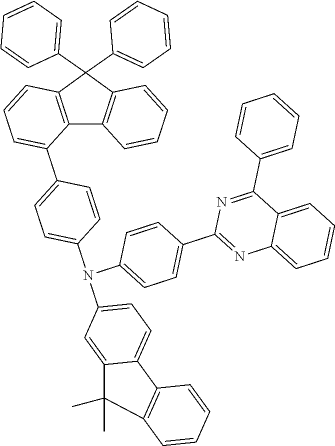 [SMILES: CC1(C)c2ccccc2-c2ccc(N(c3ccc(-c4nc(-c5ccccc5)c5ccccc5n4)cc3)c3ccc(-c4cccc5c4-c4ccccc4C5(c4ccccc4)c4ccccc4)cc3)cc21] | 61 |
| 24d | 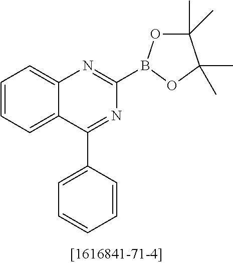 [SMILES: CC1(C)OB(c2nc(-c3ccccc3)c3ccccc3n2)OC1(C)C] | 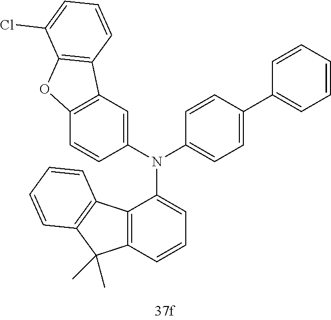 [SMILES: CC1(C)c2ccccc2-c2c(N(c3ccc(-c4ccccc4)cc3)c3ccc4oc5c(Cl)cccc5c4c3)cccc21] | 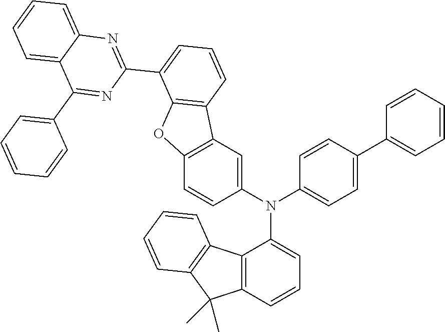 [SMILES: CC1(C)c2ccccc2-c2c(N(c3ccc(-c4ccccc4)cc3)c3ccc4oc5c(-c6nc(-c7ccccc7)c7ccccc7n6)cccc5c4c3)cccc21] | 63 |
| 25d | 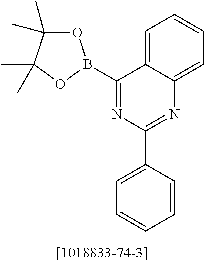 [SMILES: CC1(C)OB(c2nc(-c3ccccc3)nc3ccccc23)OC1(C)C] | 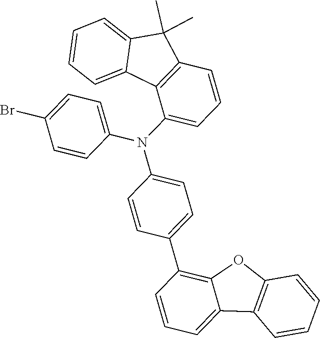 [SMILES: CC1(C)c2ccccc2-c2c(N(c3ccc(Br)cc3)c3ccc(-c4cccc5c4oc4ccccc45)cc3)cccc21] | 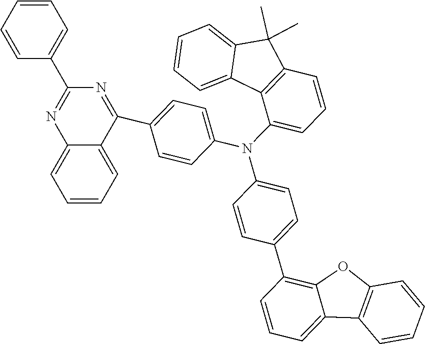 [SMILES: CC1(C)c2ccccc2-c2c(N(c3ccc(-c4nc(-c5ccccc5)nc5ccccc45)cc3)c3ccc(-c4cccc5c4oc4ccccc45)cc3)cccc21] | 67 |
| 26d | 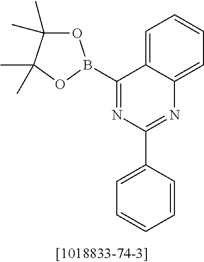 [SMILES: CC1(C)OB(c2nc(-c3ccccc3)nc3ccccc23)OC1(C)C] | 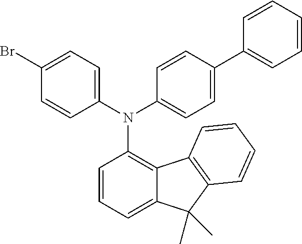 [SMILES: CC1(C)c2ccccc2-c2c(N(c3ccc(Br)cc3)c3ccc(-c4ccccc4)cc3)cccc21] | 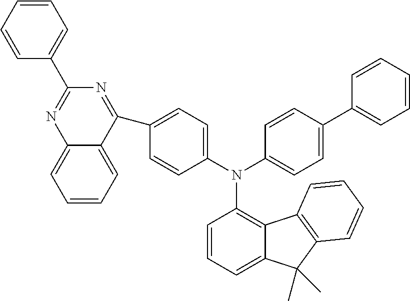 [SMILES: CC1(C)c2ccccc2-c2c(N(c3ccc(-c4ccccc4)cc3)c3ccc(-c4nc(-c5ccccc5)nc5ccccc45)cc3)cccc21] | 65 |
| 27d | 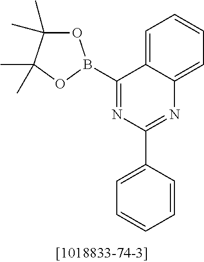 [SMILES: CC1(C)OB(c2nc(-c3ccccc3)nc3ccccc23)OC1(C)C] | 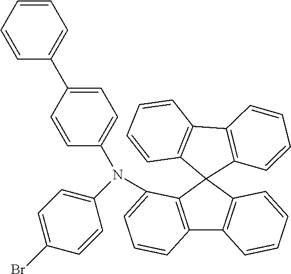 [SMILES: Brc1ccc(N(c2ccc(-c3ccccc3)cc2)c2cccc3c2C2(c4ccccc4-c4ccccc42)c2ccccc2-3)cc1] | 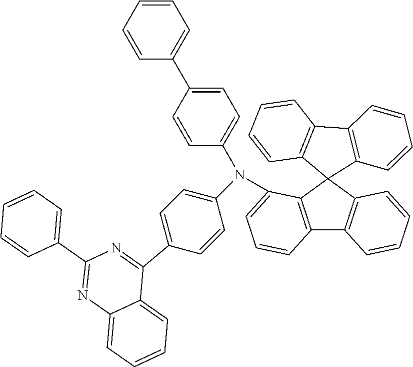 [SMILES: c1ccc(-c2ccc(N(c3ccc(-c4nc(-c5ccccc5)nc5ccccc45)cc3)c3cccc4c3C3(c5ccccc5-c5ccccc53)c3ccccc3-4)cc2)cc1] | 63 |
| 28d | 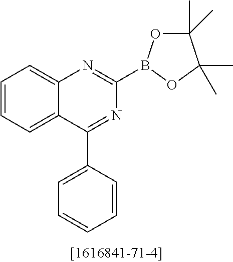 [SMILES: CC1(C)OB(c2nc(-c3ccccc3)c3ccccc3n2)OC1(C)C] | 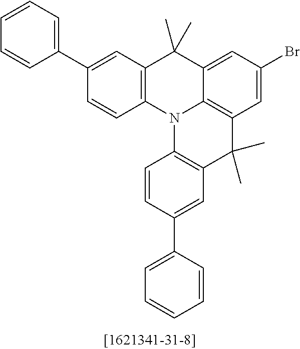 [SMILES: CC1(C)c2cc(-c3ccccc3)ccc2N2c3ccc(-c4ccccc4)cc3C(C)(C)c3cc(Br)cc1c32] | 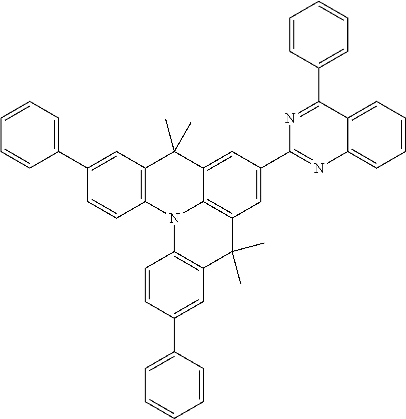 [SMILES: CC1(C)c2cc(-c3ccccc3)ccc2N2c3ccc(-c4ccccc4)cc3C(C)(C)c3cc(-c4nc(-c5ccccc5)c5ccccc5n4)cc1c32] | 62 |
| 29d | 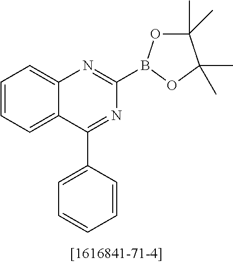 [SMILES: CC1(C)OB(c2nc(-c3ccccc3)c3ccccc3n2)OC1(C)C] | 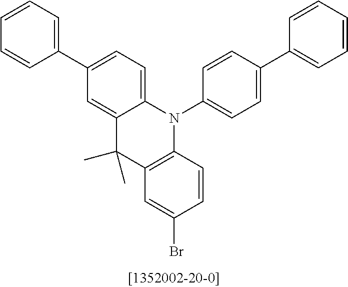 [SMILES: CC1(C)c2cc(Br)ccc2N(c2ccc(-c3ccccc3)cc2)c2ccc(-c3ccccc3)cc21] | 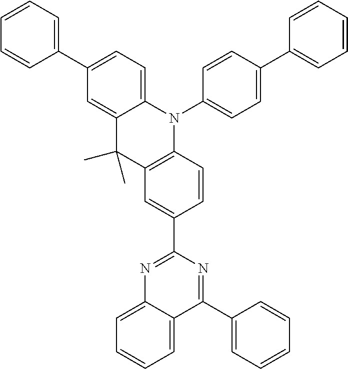 [SMILES: CC1(C)c2cc(-c3ccccc3)ccc2N(c2ccc(-c3ccccc3)cc2)c2ccc(-c3nc(-c4ccccc4)c4ccccc4n3)cc21] | 60 |
| 30d | 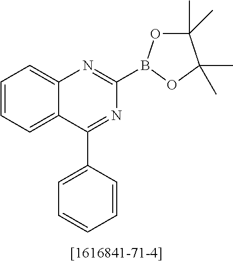 [SMILES: CC1(C)OB(c2nc(-c3ccccc3)c3ccccc3n2)OC1(C)C] | 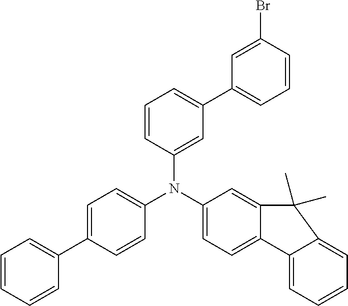 [SMILES: CC1(C)c2ccccc2-c2ccc(N(c3ccc(-c4ccccc4)cc3)c3cccc(-c4cccc(Br)c4)c3)cc21] | 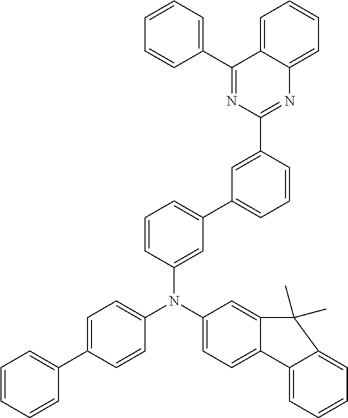 [SMILES: CC1(C)c2ccccc2-c2ccc(N(c3ccc(-c4ccccc4)cc3)c3cccc(-c4cccc(-c5nc(-c6ccccc6)c6ccccc6n5)c4)c3)cc21] | 61 |
| 31d | 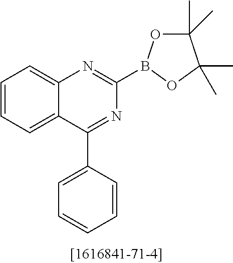 [SMILES: CC1(C)OB(c2nc(-c3ccccc3)c3ccccc3n2)OC1(C)C] | 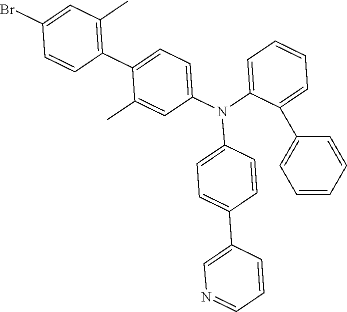 [SMILES: Cc1cc(Br)ccc1-c1ccc(N(c2ccc(-c3cccnc3)cc2)c2ccccc2-c2ccccc2)cc1C] | 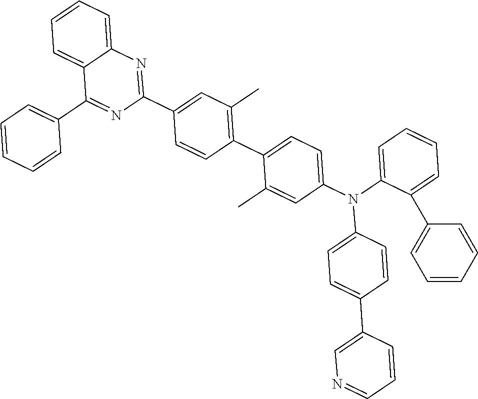 [SMILES: Cc1cc(-c2nc(-c3ccccc3)c3ccccc3n2)ccc1-c1ccc(N(c2ccc(-c3cccnc3)cc2)c2ccccc2-c2ccccc2)cc1C] | 55 |

### e) Biphenyl-4-yl-(9,9-dimethyl-9H-fluoren-4-yl)-(4-phenylquinazolin-2-yl)amine

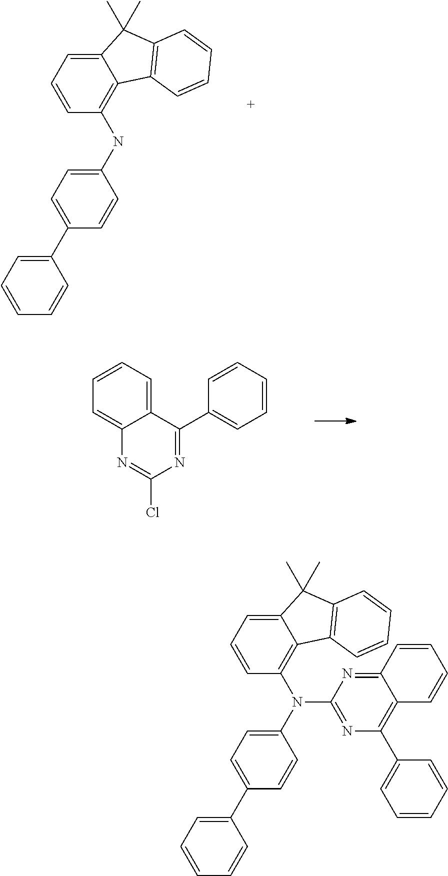 [SMILES: CC1(C)c2ccccc2-c2c(Nc3ccc(-c4ccccc4)cc3)cccc21 ; Clc1nc(-c2ccccc2)c2ccccc2n1 ; CC1(C)c2ccccc2-c2c(N(c3ccc(-c4ccccc4)cc3)c3nc(-c4ccccc4)c4ccccc4n3)cccc21]

A mixture of 14.4 g (60 mmol) of 2-chloro-4-phenylquinazoline, 18.7 g (60 mmol) of biphenyl-4-yl-(9,9-dimethyl-9H-fluoren-4-yl)amine, 7.7 g (80 mmol) of sodium tert-butoxide, 1.4 g (5 mmol) of tricyclohexylamine, 561 mg (2.5 mmol) of palladium(II) acetate and 300 ml of mesitylene is heated under reflux for 24 h. After cooling, 200 ml of water are added, the mixture is stirred for a further 30 min., the organic phase is separated off and filtered through a short Celite bed, and the solvent is then removed in vacuo. The residue is recrystallised five times from DMF and finally subjected to fractional sublimation twice (p about 10⁻⁶ mbar, T=350-380° C.). Yield: 23 g (40 mmol), 69% of theory: 99.9% according to HPLC.

The following compounds can be prepared analogously:

| | Starting | Starting | | Yield |
|---|---|---|---|---|
| Ex. | material 1 | material 2 | Product | [%] |
| 1e | 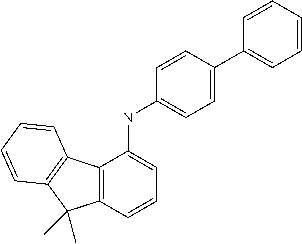 [SMILES: CC1(C)c2ccccc2-c2c(Nc3ccc(-c4ccccc4)cc3)cccc21] | 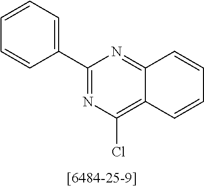 [SMILES: Clc1nc(-c2ccccc2)nc2ccccc12] | 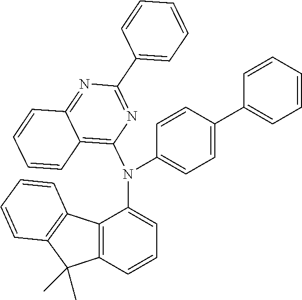 [SMILES: CC1(C)c2ccccc2-c2c(N(c3ccc(-c4ccccc4)cc3)c3nc(-c4ccccc4)nc4ccccc34)cccc21] | 65 |
| 2e | 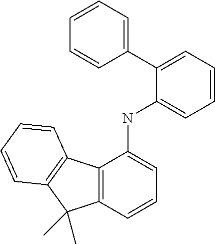 [SMILES: CC1(C)c2ccccc2-c2c(Nc3ccccc3-c3ccccc3)cccc21] | 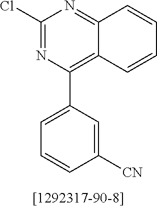 [SMILES: N#Cc1cccc(-c2nc(Cl)nc3ccccc23)c1] | 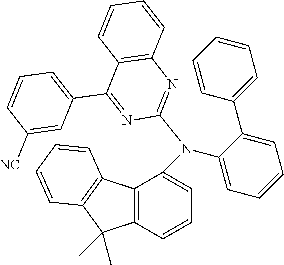 [SMILES: CC1(C)c2ccccc2-c2c(N(c3nc(-c4cccc(C#N)c4)c4ccccc4n3)c3ccccc3-c3ccccc3)cccc21] | 71 |
| 3e | 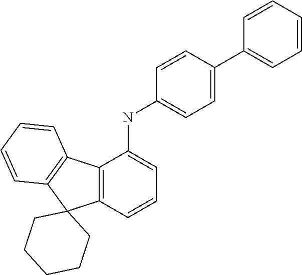 [SMILES: c1ccc(-c2ccc(Nc3cccc4c3-c3ccccc3C43CCCCC3)cc2)cc1] | 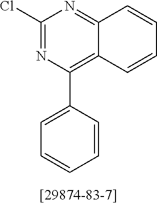 [SMILES: Clc1nc(-c2ccccc2)c2ccccc2n1] | 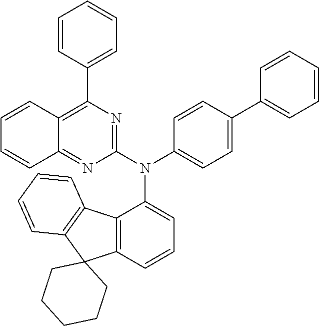 [SMILES: c1ccc(-c2ccc(N(c3nc(-c4ccccc4)c4ccccc4n3)c3cccc4c3-c3ccccc3C43CCCCC3)cc2)cc1] | 70 |
| 4e | 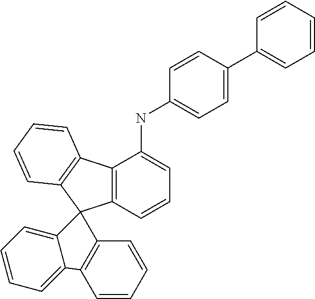 [SMILES: c1ccc(-c2ccc(Nc3cccc4c3-c3ccccc3C43c4ccccc4-c4ccccc43)cc2)cc1] | 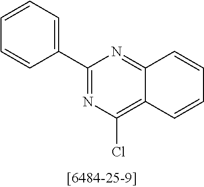 [SMILES: Clc1nc(-c2ccccc2)nc2ccccc12] | 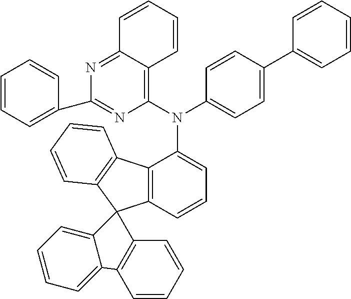 [SMILES: c1ccc(-c2ccc(N(c3cccc4c3-c3ccccc3C43c4ccccc4-c4ccccc43)c3nc(-c4ccccc4)nc4ccccc34)cc2)cc1] | 64 |
| 5e | 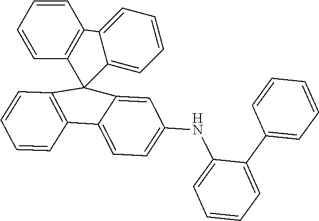 [SMILES: c1ccc(-c2ccccc2Nc2ccc3c(c2)C2(c4ccccc4-c4ccccc42)c2ccccc2-3)cc1] | 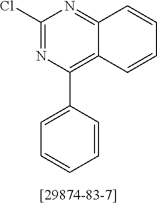 [SMILES: Clc1nc(-c2ccccc2)c2ccccc2n1] | 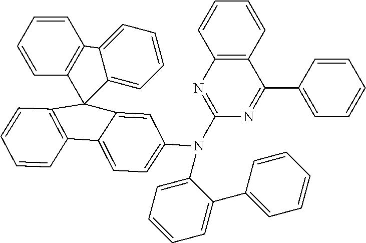 [SMILES: c1ccc(-c2ccccc2N(c2ccc3c(c2)C2(c4ccccc4-c4ccccc42)c2ccccc2-3)c2nc(-c3ccccc3)c3ccccc3n2)cc1] | 64 |
| 6e | 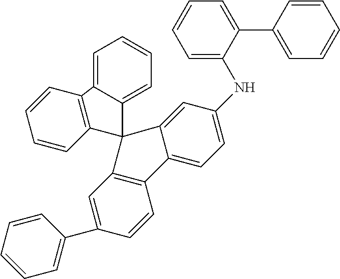 [SMILES: c1ccc(-c2ccc3c(c2)C2(c4ccccc4-c4ccccc42)c2cc(Nc4ccccc4-c4ccccc4)ccc2-3)cc1] | 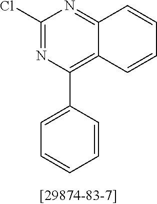 [SMILES: Clc1nc(-c2ccccc2)c2ccccc2n1] | 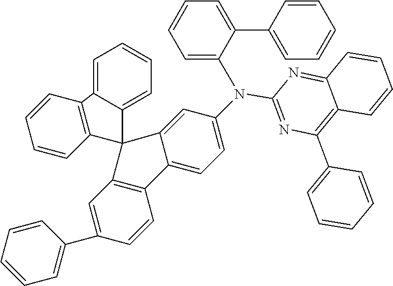 [SMILES: c1ccc(-c2ccc3c(c2)C2(c4ccccc4-c4ccccc42)c2cc(N(c4nc(-c5ccccc5)c5ccccc5n4)c4ccccc4-c4ccccc4)ccc2-3)cc1] | 71 |
| 7e | 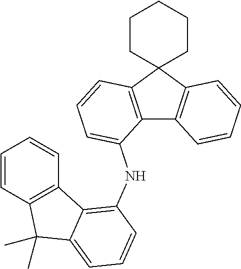 [SMILES: CC1(C)c2ccccc2-c2c(Nc3cccc4c3-c3ccccc3C43CCCCC3)cccc21] | 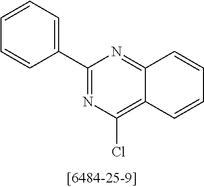 [SMILES: Clc1nc(-c2ccccc2)nc2ccccc12] | 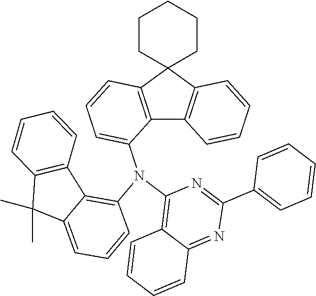 [SMILES: CC1(C)c2ccccc2-c2c(N(c3cccc4c3-c3ccccc3C43CCCCC3)c3nc(-c4ccccc4)nc4ccccc34)cccc21] | 62 |
| 8e | 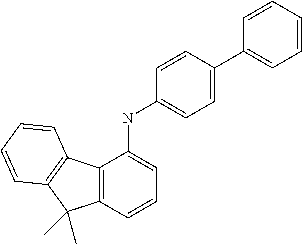 [SMILES: CC1(C)c2ccccc2-c2c(Nc3ccc(-c4ccccc4)cc3)cccc21] | 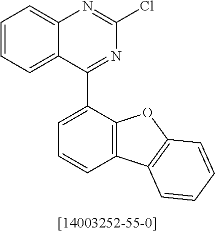 [SMILES: Clc1nc(-c2cccc3c2oc2ccccc23)c2ccccc2n1] | 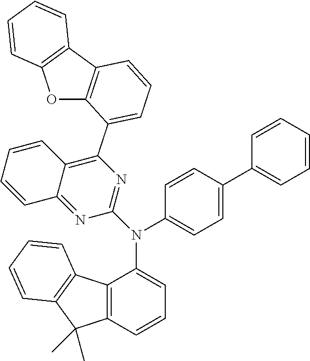 [SMILES: CC1(C)c2ccccc2-c2c(N(c3ccc(-c4ccccc4)cc3)c3nc(-c4cccc5c4oc4ccccc45)c4ccccc4n3)cccc21] | 71 |
| 9e | 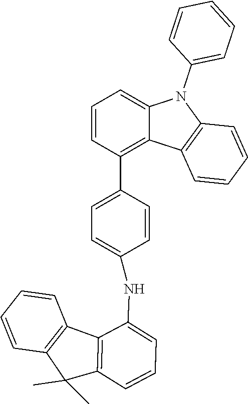 [SMILES: CC1(C)c2ccccc2-c2c(Nc3ccc(-c4cccc5c4c4ccccc4n5-c4ccccc4)cc3)cccc21] | 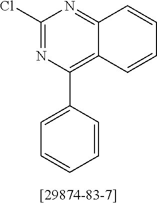 [SMILES: Clc1nc(-c2ccccc2)c2ccccc2n1] | 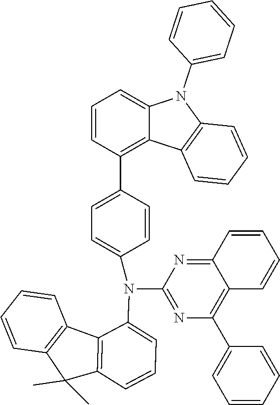 [SMILES: CC1(C)c2ccccc2-c2c(N(c3ccc(-c4cccc5c4c4ccccc4n5-c4ccccc4)cc3)c3nc(-c4ccccc4)c4ccccc4n3)cccc21] | 64 |
| 10e | 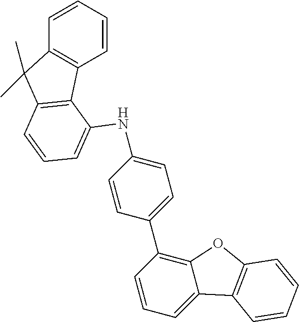 [SMILES: CC1(C)c2ccccc2-c2c(Nc3ccc(-c4cccc5c4oc4ccccc45)cc3)cccc21] | 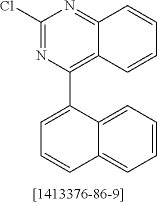 [SMILES: Clc1nc(-c2cccc3ccccc23)c2ccccc2n1] | 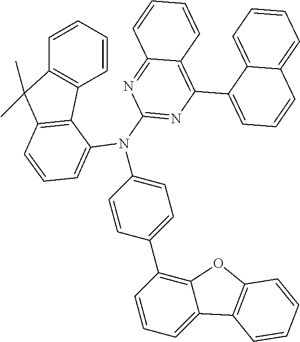 [SMILES: CC1(C)c2ccccc2-c2c(N(c3ccc(-c4cccc5c4oc4ccccc45)cc3)c3nc(-c4cccc5ccccc45)c4ccccc4n3)cccc21] | 65 |
| 11e | 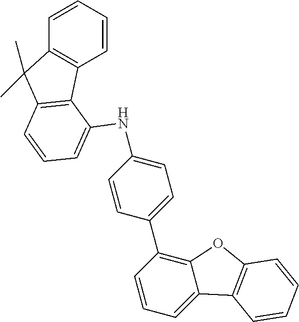 [SMILES: CC1(C)c2ccccc2-c2c(Nc3ccc(-c4cccc5c4oc4ccccc45)cc3)cccc21] | 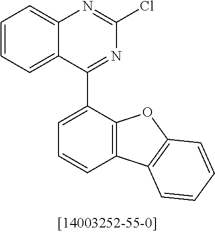 [SMILES: Clc1nc(-c2cccc3c2oc2ccccc23)c2ccccc2n1] | 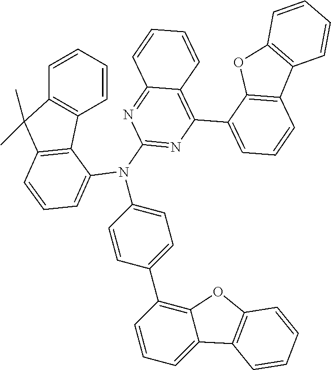 [SMILES: CC1(C)c2ccccc2-c2c(N(c3ccc(-c4cccc5c4oc4ccccc45)cc3)c3nc(-c4cccc5c4oc4ccccc45)c4ccccc4n3)cccc21] | 54 |
| 12e | 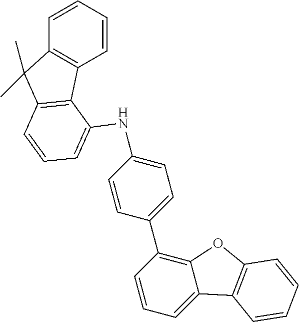 [SMILES: CC1(C)c2ccccc2-c2c(Nc3ccc(-c4cccc5c4oc4ccccc45)cc3)cccc21] | 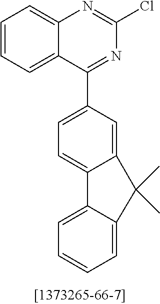 [SMILES: CC1(C)c2ccccc2-c2ccc(-c3nc(Cl)nc4ccccc34)cc21] | 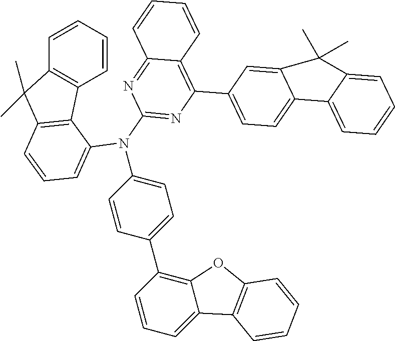 [SMILES: CC1(C)c2ccccc2-c2ccc(-c3nc(N(c4ccc(-c5cccc6c5oc5ccccc56)cc4)c4cccc5c4-c4ccccc4C5(C)C)nc4ccccc34)cc21] | 63 |
| 13e | 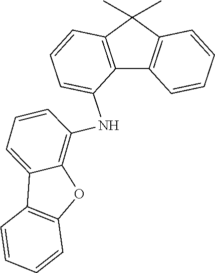 [SMILES: CC1(C)c2ccccc2-c2c(Nc3cccc4c3oc3ccccc34)cccc21] | 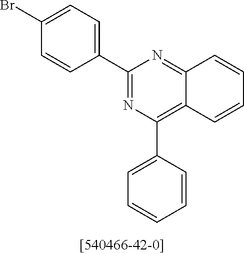 [SMILES: Brc1ccc(-c2nc(-c3ccccc3)c3ccccc3n2)cc1] | 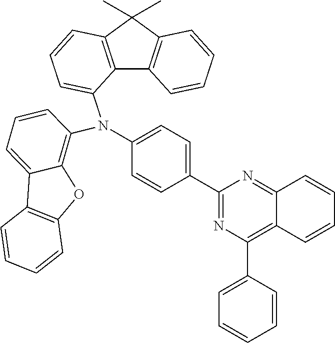 [SMILES: CC1(C)c2ccccc2-c2c(N(c3ccc(-c4nc(-c5ccccc5)c5ccccc5n4)cc3)c3cccc4c3oc3ccccc34)cccc21] | 62 |
| 14e | 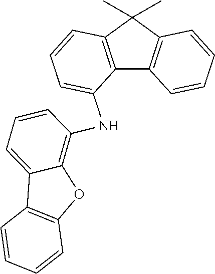 [SMILES: CC1(C)c2ccccc2-c2c(Nc3cccc4c3oc3ccccc34)cccc21] | 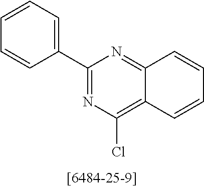 [SMILES: Clc1nc(-c2ccccc2)nc2ccccc12] | 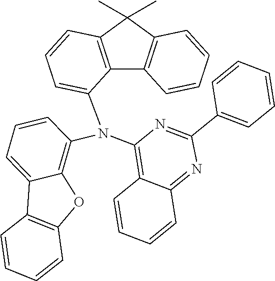 [SMILES: CC1(C)c2ccccc2-c2c(N(c3nc(-c4ccccc4)nc4ccccc34)c3cccc4c3oc3ccccc34)cccc21] | 57 |
| 15e | 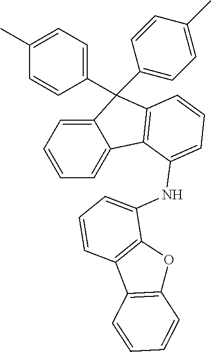 | 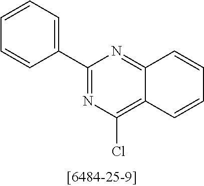 [SMILES: Clc1nc(-c2ccccc2)nc2ccccc12] | 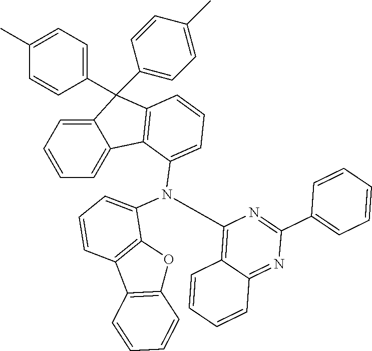 [SMILES: Cc1ccc(C2(c3ccc(C)cc3)c3ccccc3-c3c(N(c4nc(-c5ccccc5)nc5ccccc45)c4cccc5c4oc4ccccc45)cccc32)cc1] | 61 |
| 16e | 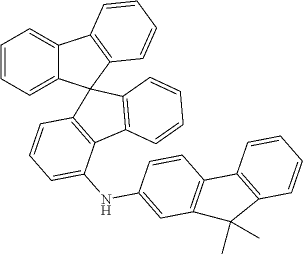 [SMILES: CC1(C)c2ccccc2-c2ccc(Nc3cccc4c3-c3ccccc3C43c4ccccc4-c4ccccc43)cc21] | 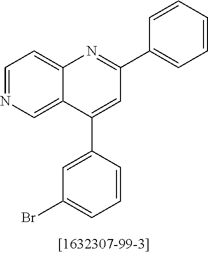 [SMILES: Brc1cccc(-c2cc(-c3ccccc3)nc3ccncc23)c1] | 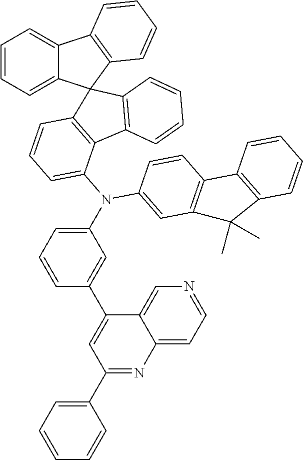 [SMILES: CC1(C)c2ccccc2-c2ccc(N(c3cccc(-c4cc(-c5ccccc5)nc5ccncc45)c3)c3cccc4c3-c3ccccc3C43c4ccccc4-c4ccccc43)cc21] | 62 |
| 17e | 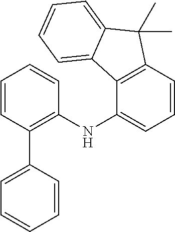 [SMILES: CC1(C)c2ccccc2-c2c(Nc3ccccc3-c3ccccc3)cccc21] | 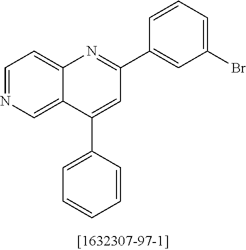 [SMILES: Brc1cccc(-c2cc(-c3ccccc3)c3cnccc3n2)c1] | 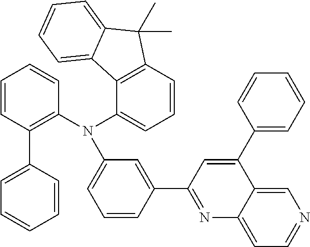 [SMILES: CC1(C)c2ccccc2-c2c(N(c3cccc(-c4cc(-c5ccccc5)c5cnccc5n4)c3)c3ccccc3-c3ccccc3)cccc21] | 57 |
| 18e | 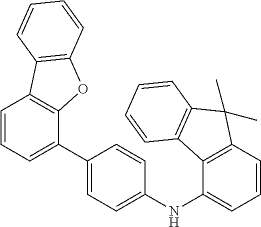 [SMILES: CC1(C)c2ccccc2-c2c(Nc3ccc(-c4cccc5c4oc4ccccc45)cc3)cccc21] | 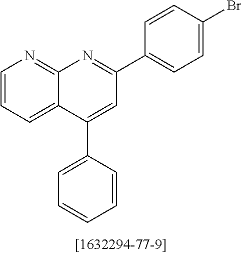 [SMILES: Brc1ccc(-c2cc(-c3ccccc3)c3cccnc3n2)cc1] | 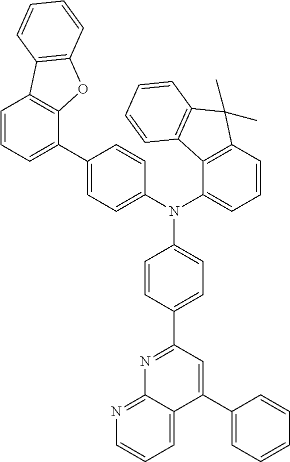 [SMILES: CC1(C)c2ccccc2-c2c(N(c3ccc(-c4cc(-c5ccccc5)c5cccnc5n4)cc3)c3ccc(-c4cccc5c4oc4ccccc45)cc3)cccc21] | 66 |
| 19e | 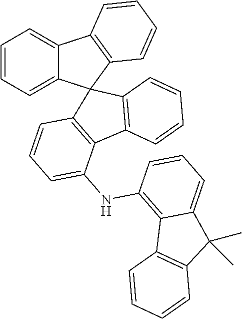 [SMILES: CC1(C)c2ccccc2-c2c(Nc3cccc4c3-c3ccccc3C43c4ccccc4-c4ccccc43)cccc21] | 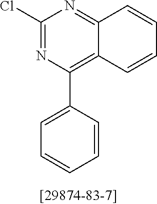 [SMILES: Clc1nc(-c2ccccc2)c2ccccc2n1] | 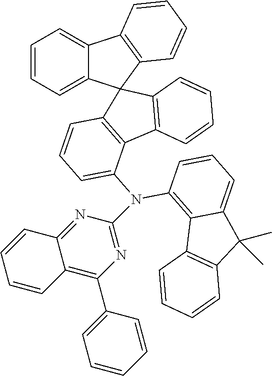 [SMILES: CC1(C)c2ccccc2-c2c(N(c3nc(-c4ccccc4)c4ccccc4n3)c3cccc4c3-c3ccccc3C43c4ccccc4-c4ccccc43)cccc21] | 58 |
| 20e | 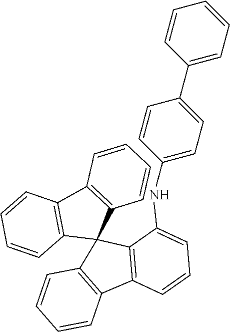 | 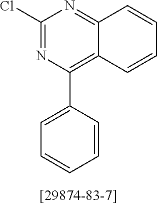 [SMILES: Clc1nc(-c2ccccc2)c2ccccc2n1] | 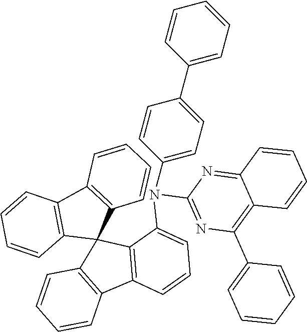 | 67 |
| 21e | 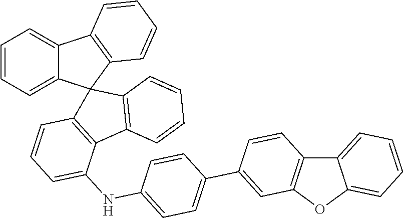 [SMILES: c1ccc2c(c1)-c1ccccc1C21c2ccccc2-c2c(Nc3ccc(-c4ccc5c(c4)oc4ccccc45)cc3)cccc21] | 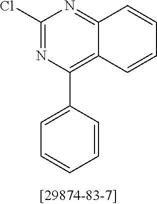 [SMILES: Clc1nc(-c2ccccc2)c2ccccc2n1] | 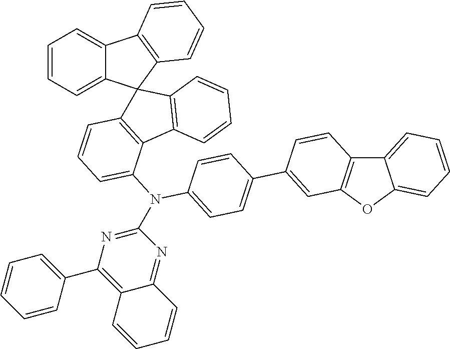 [SMILES: c1ccc(-c2nc(N(c3ccc(-c4ccc5c(c4)oc4ccccc45)cc3)c3cccc4c3-c3ccccc3C43c4ccccc4-c4ccccc43)nc3ccccc23)cc1] | 76 |
| 22e | 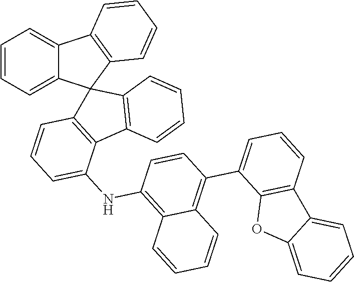 [SMILES: c1ccc2c(c1)-c1ccccc1C21c2ccccc2-c2c(Nc3ccc(-c4cccc5c4oc4ccccc45)c4ccccc34)cccc21] | 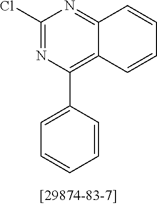 [SMILES: Clc1nc(-c2ccccc2)c2ccccc2n1] | 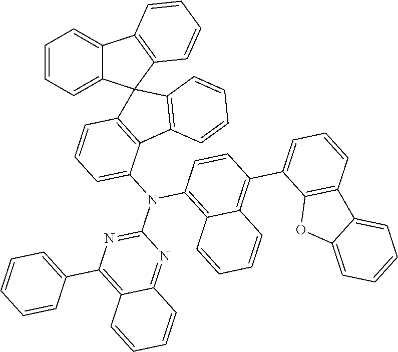 [SMILES: c1ccc(-c2nc(N(c3cccc4c3-c3ccccc3C43c4ccccc4-c4ccccc43)c3ccc(-c4cccc5c4oc4ccccc45)c4ccccc34)nc3ccccc23)cc1] | 70 |
| 23e | 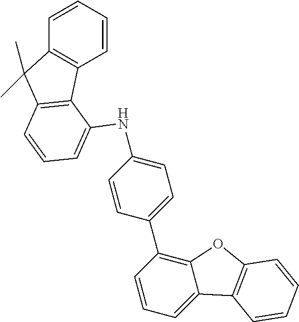 [SMILES: CC1(C)c2ccccc2-c2c(Nc3ccc(-c4cccc5c4oc4ccccc45)cc3)cccc21] | 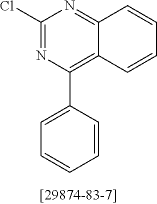 [SMILES: Clc1nc(-c2ccccc2)c2ccccc2n1] | 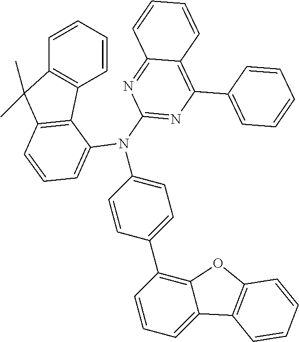 [SMILES: CC1(C)c2ccccc2-c2c(N(c3ccc(-c4cccc5c4oc4ccccc45)cc3)c3nc(-c4ccccc4)c4ccccc4n3)cccc21] | 68 |
| 24e | 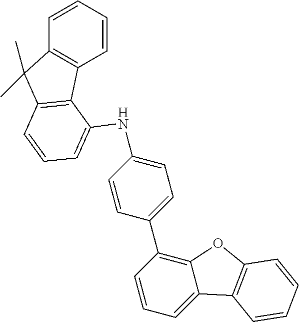 [SMILES: CC1(C)c2ccccc2-c2c(Nc3ccc(-c4cccc5c4oc4ccccc45)cc3)cccc21] | 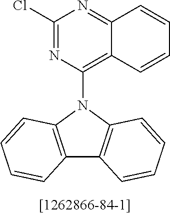 [SMILES: Clc1nc(-n2c3ccccc3c3ccccc32)c2ccccc2n1] | 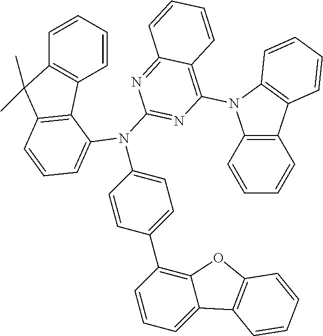 [SMILES: CC1(C)c2ccccc2-c2c(N(c3ccc(-c4cccc5c4oc4ccccc45)cc3)c3nc(-n4c5ccccc5c5ccccc54)c4ccccc4n3)cccc21] | 71 |
| 25e | 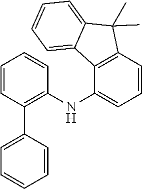 [SMILES: CC1(C)c2ccccc2-c2c(Nc3ccccc3-c3ccccc3)cccc21] | 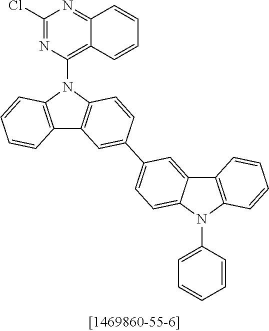 [SMILES: Clc1nc(-n2c3ccccc3c3cc(-c4ccc5c(c4)c4ccccc4n5-c4ccccc4)ccc32)c2ccccc2n1] | 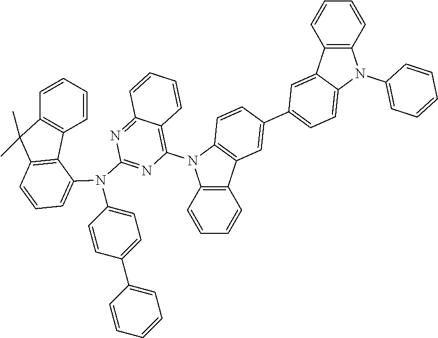 [SMILES: CC1(C)c2ccccc2-c2c(N(c3ccc(-c4ccccc4)cc3)c3nc(-n4c5ccccc5c5cc(-c6ccc7c(c6)c6ccccc6n7-c6ccccc6)ccc54)c4ccccc4n3)cccc21] | 64 |
| 26e | 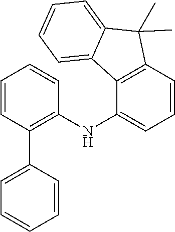 [SMILES: CC1(C)c2ccccc2-c2c(Nc3ccccc3-c3ccccc3)cccc21] | 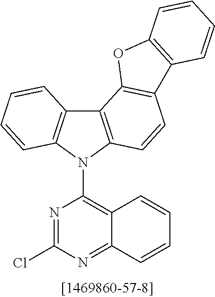 [SMILES: Clc1nc(-n2c3ccccc3c3c4oc5ccccc5c4ccc32)c2ccccc2n1] | 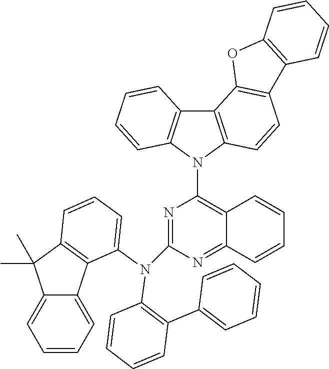 [SMILES: CC1(C)c2ccccc2-c2c(N(c3nc(-n4c5ccccc5c5c6oc7ccccc7c6ccc54)c4ccccc4n3)c3ccccc3-c3ccccc3)cccc21] | 58 |
| 27e | 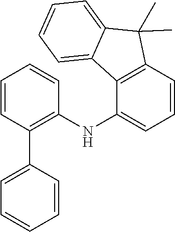 [SMILES: CC1(C)c2ccccc2-c2c(Nc3ccccc3-c3ccccc3)cccc21] | 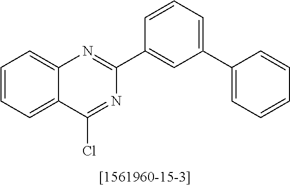 [SMILES: Clc1nc(-c2cccc(-c3ccccc3)c2)nc2ccccc12] | 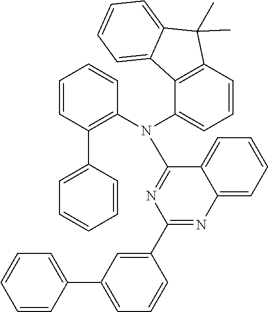 [SMILES: CC1(C)c2ccccc2-c2c(N(c3ccccc3-c3ccccc3)c3nc(-c4cccc(-c5ccccc5)c4)nc4ccccc34)cccc21] | 63 |
| 28e | 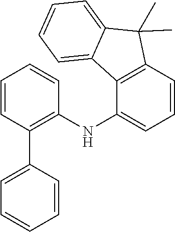 [SMILES: CC1(C)c2ccccc2-c2c(Nc3ccccc3-c3ccccc3)cccc21] | 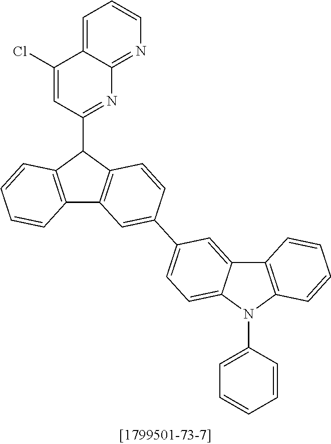 [SMILES: Clc1cc(C2c3ccccc3-c3cc(-c4ccc5c(c4)c4ccccc4n5-c4ccccc4)ccc32)nc2ncccc12] | 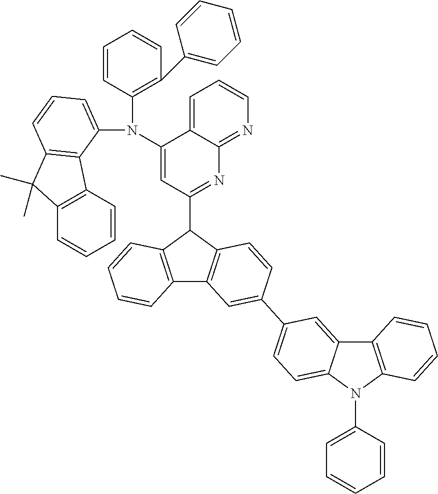 [SMILES: CC1(C)c2ccccc2-c2c(N(c3ccccc3-c3ccccc3)c3cc(C4c5ccccc5-c5cc(-c6ccc7c(c6)c6ccccc6n7-c6ccccc6)ccc54)nc4ncccc34)cccc21] | 67 |

### f) 9-(2-Chloroquinazolin-4-yl)-3-phenyl-9H-carbazole

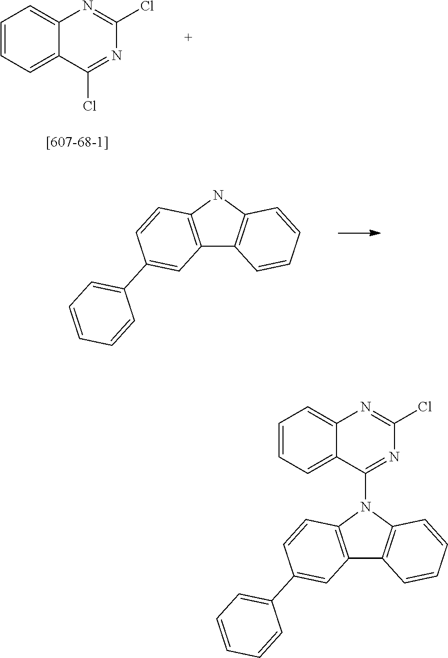 [SMILES: Clc1nc(Cl)c2ccccc2n1 ; c1ccc(-c2ccc3[nH]c4ccccc4c3c2)cc1 ; Clc1nc(-n2c3ccccc3c3cc(-c4ccccc4)ccc32)c2ccccc2n1]

14.4 g (60 mmol) of 3-phenyl-9H-carbazole are dissolved in 300 ml of dimethylformamide under a protective-gas atmosphere, and 3 g of NaH, 60% in mineral oil (75 mmol), are added. After 1 h at room temperature, a solution of 12.3 g (62 mmol) of 2,4-dichloroquinazoline in 150 ml of dimethylformamide is added dropwise. The reaction mixture is then stirred at room temperature for 12 h, poured onto ice and extracted three times with dichloromethane. The combined organic phases are dried over Na₂SO₄ and evaporated. The residue is recrystallised from toluene. The yield is 21 g (51 mmol), corresponding to 75% of theory.

The following compounds can be prepared analogously:

| | Starting | Starting | | Yield |
|---|---|---|---|---|
| Ex. | material 1 | material 2 | Product | [%] |
| 1f | 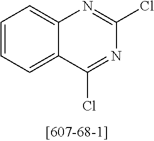 [SMILES: Clc1nc(Cl)c2ccccc2n1] | 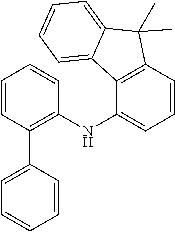 [SMILES: CC1(C)c2ccccc2-c2c(Nc3ccccc3-c3ccccc3)cccc21] | 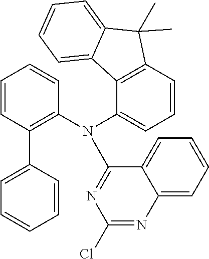 [SMILES: CC1(C)c2ccccc2-c2c(N(c3ccccc3-c3ccccc3)c3nc(Cl)nc4ccccc34)cccc21] | 55 |
| 2f | 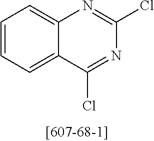 [SMILES: Clc1nc(Cl)c2ccccc2n1] | 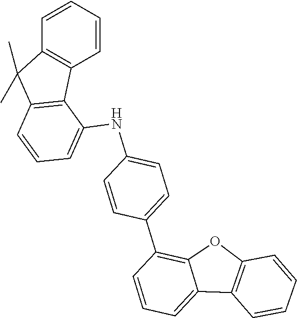 [SMILES: CC1(C)c2ccccc2-c2c(Nc3ccc(-c4cccc5c4oc4ccccc45)cc3)cccc21] | 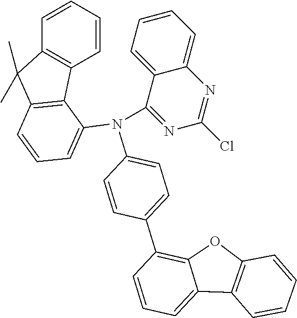 [SMILES: CC1(C)c2ccccc2-c2c(N(c3ccc(-c4cccc5c4oc4ccccc45)cc3)c3nc(Cl)nc4ccccc34)cccc21] | 57 |
| 3f | 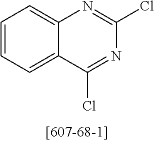 [SMILES: Clc1nc(Cl)c2ccccc2n1] | 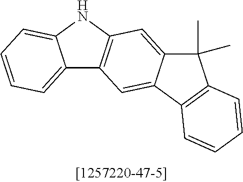 [SMILES: CC1(C)c2ccccc2-c2cc3c(cc21)[nH]c1ccccc13] | 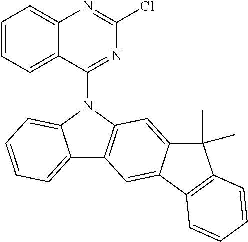 [SMILES: CC1(C)c2ccccc2-c2cc3c4ccccc4n(-c4nc(Cl)nc5ccccc45)c3cc21] | 57 |
| 4f | 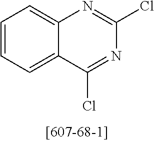 [SMILES: Clc1nc(Cl)c2ccccc2n1] | 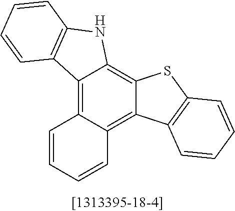 [SMILES: c1ccc2c(c1)[nH]c1c3sc4ccccc4c3c3ccccc3c21] | 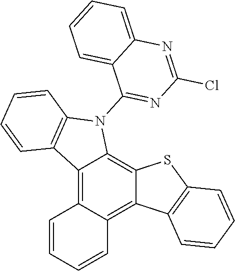 [SMILES: Clc1nc(-n2c3ccccc3c3c4ccccc4c4c5ccccc5sc4c32)c2ccccc2n1] | 55 |

The following compounds can be prepared analogously:

| | Starting | Starting |
|---|---|---|
| Ex. | material 1 | material 2 |
| 5f | 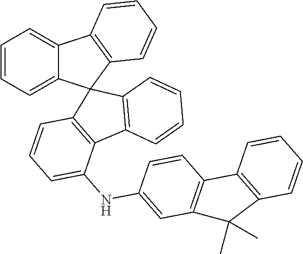 [SMILES: CC1(C)c2ccccc2-c2ccc(Nc3cccc4c3-c3ccccc3C43c4ccccc4-c4ccccc43)cc21] | 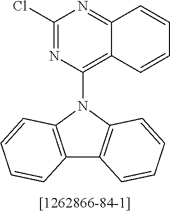 [SMILES: Clc1nc(-n2c3ccccc3c3ccccc32)c2ccccc2n1] |
| 6f | 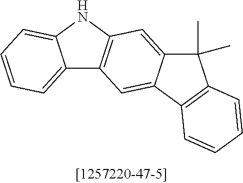 [SMILES: CC1(C)c2ccccc2-c2cc3c(cc21)[nH]c1ccccc13] | 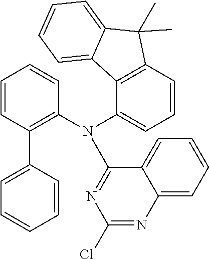 [SMILES: CC1(C)c2ccccc2-c2c(N(c3ccccc3-c3ccccc3)c3nc(Cl)nc4ccccc34)cccc21] |
| 7f | 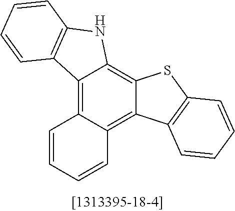 [SMILES: c1ccc2c(c1)[nH]c1c3sc4ccccc4c3c3ccccc3c21] | 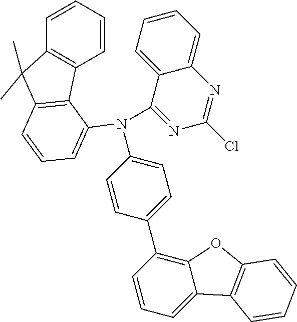 [SMILES: CC1(C)c2ccccc2-c2c(N(c3ccc(-c4cccc5c4oc4ccccc45)cc3)c3nc(Cl)nc4ccccc34)cccc21] |
| 8f | 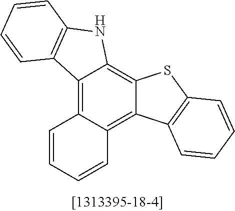 [SMILES: c1ccc2c(c1)[nH]c1c3sc4ccccc4c3c3ccccc3c21] | 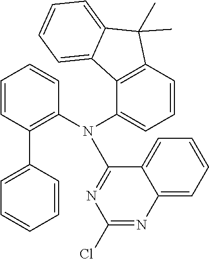 [SMILES: CC1(C)c2ccccc2-c2c(N(c3ccccc3-c3ccccc3)c3nc(Cl)nc4ccccc34)cccc21] |
| 9f | 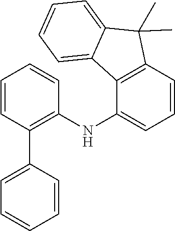 [SMILES: CC1(C)c2ccccc2-c2c(Nc3ccccc3-c3ccccc3)cccc21] | 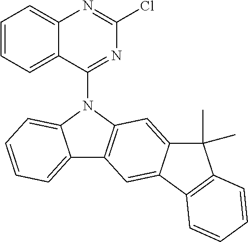 [SMILES: CC1(C)c2ccccc2-c2cc3c4ccccc4n(-c4nc(Cl)nc5ccccc45)c3cc21] |
| 10f | 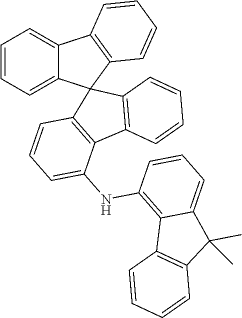 [SMILES: CC1(C)c2ccccc2-c2c(Nc3cccc4c3-c3ccccc3C43c4ccccc4-c4ccccc43)cccc21] | 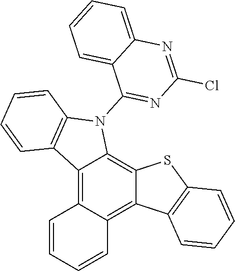 [SMILES: Clc1nc(-n2c3ccccc3c3c4ccccc4c4c5ccccc5sc4c32)c2ccccc2n1] |
| 11f | 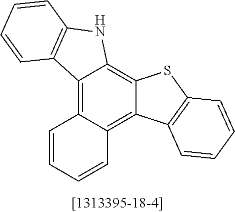 [SMILES: c1ccc2c(c1)[nH]c1c3sc4ccccc4c3c3ccccc3c21] | 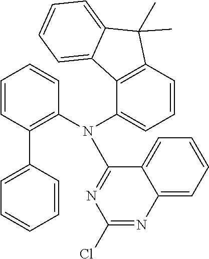 [SMILES: CC1(C)c2ccccc2-c2c(N(c3ccccc3-c3ccccc3)c3nc(Cl)nc4ccccc34)cccc21] |
| 12f | 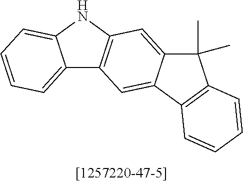 [SMILES: CC1(C)c2ccccc2-c2cc3c(cc21)[nH]c1ccccc13] | 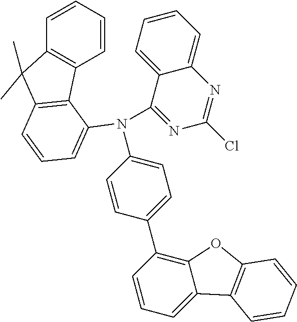 [SMILES: CC1(C)c2ccccc2-c2c(N(c3ccc(-c4cccc5c4oc4ccccc45)cc3)c3nc(Cl)nc4ccccc34)cccc21] |
| 13f | 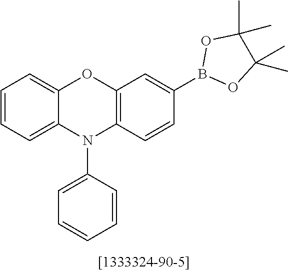 [SMILES: CC1(C)OB(c2ccc3c(c2)Oc2ccccc2N3c2ccccc2)OC1(C)C] | 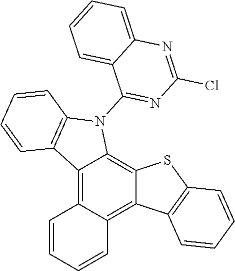 [SMILES: Clc1nc(-n2c3ccccc3c3c4ccccc4c4c5ccccc5sc4c32)c2ccccc2n1] |
| 14f | 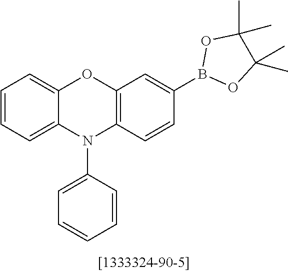 [SMILES: CC1(C)OB(c2ccc3c(c2)Oc2ccccc2N3c2ccccc2)OC1(C)C] | 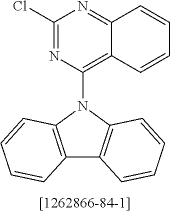 [SMILES: Clc1nc(-n2c3ccccc3c3ccccc32)c2ccccc2n1] |

| | | Yield |
|---|---|---|
| Ex. | Product | [%] |
| 5f | 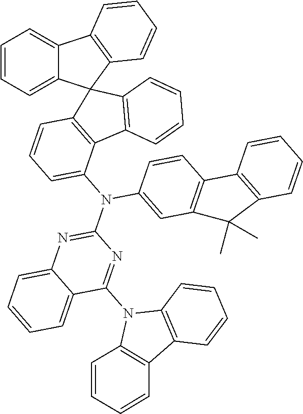 [SMILES: CC1(C)c2ccccc2-c2ccc(N(c3nc(-n4c5ccccc5c5ccccc54)c4ccccc4n3)c3cccc4c3-c3ccccc3C43c4ccccc4-c4ccccc43)cc21] | 66 |
| 6f | 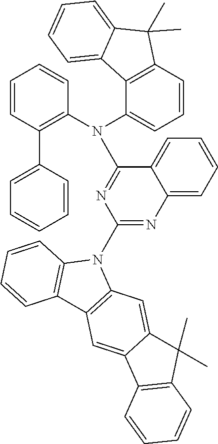 [SMILES: CC1(C)c2ccccc2-c2cc3c4ccccc4n(-c4nc(N(c5ccccc5-c5ccccc5)c5cccc6c5-c5ccccc5C6(C)C)c5ccccc5n4)c3cc21] | 65 |
| 7f | 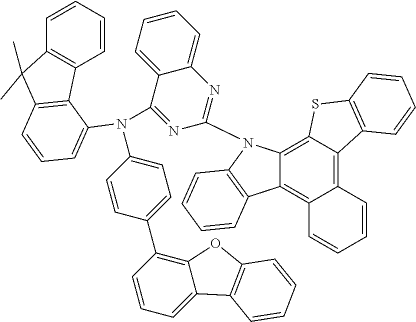 [SMILES: CC1(C)c2ccccc2-c2c(N(c3ccc(-c4cccc5c4oc4ccccc45)cc3)c3nc(-n4c5ccccc5c5c6ccccc6c6c7ccccc7sc6c54)nc4ccccc34)cccc21] | 57 |
| 8f | 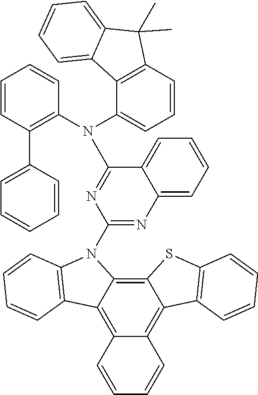 [SMILES: CC1(C)c2ccccc2-c2c(N(c3ccccc3-c3ccccc3)c3nc(-n4c5ccccc5c5c6ccccc6c6c7ccccc7sc6c54)nc4ccccc34)cccc21] | 53 |
| 9f | 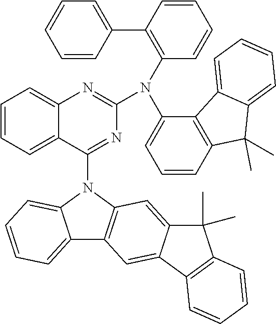 [SMILES: CC1(C)c2ccccc2-c2cc3c4ccccc4n(-c4nc(N(c5ccccc5-c5ccccc5)c5cccc6c5-c5ccccc5C6(C)C)nc5ccccc45)c3cc21] | 54 |
| 10f | 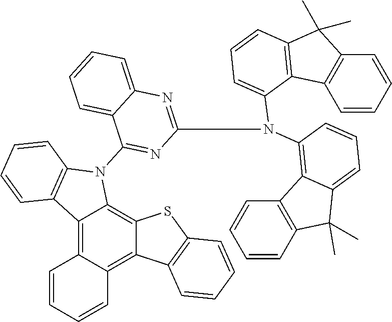 [SMILES: CC1(C)c2ccccc2-c2c(N(c3nc(-n4c5ccccc5c5c6ccccc6c6c7ccccc7sc6c54)c4ccccc4n3)c3cccc4c3-c3ccccc3C4(C)C)cccc21] | 50 |
| 11f | 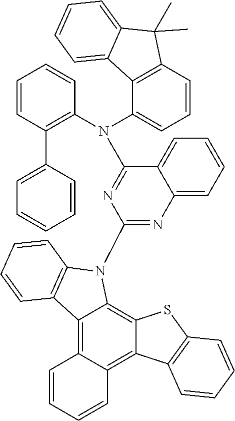 [SMILES: CC1(C)c2ccccc2-c2c(N(c3ccccc3-c3ccccc3)c3nc(-n4c5ccccc5c5c6ccccc6c6c7ccccc7sc6c54)nc4ccccc34)cccc21] | 51 |
| 12f | 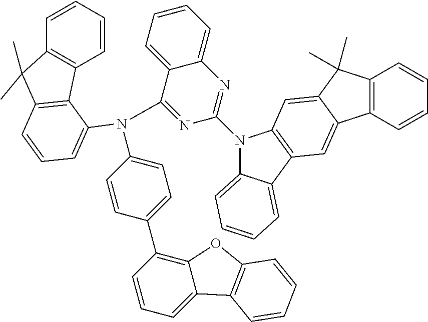 [SMILES: CC1(C)c2ccccc2-c2cc3c4ccccc4n(-c4nc(N(c5ccc(-c6cccc7c6oc6ccccc67)cc5)c5cccc6c5-c5ccccc5C6(C)C)c5ccccc5n4)c3cc21] | 55 |
| 13f | 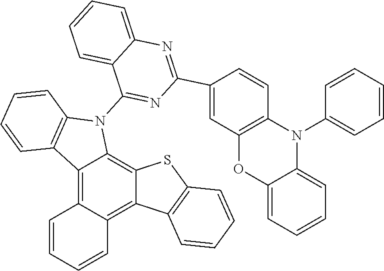 [SMILES: c1ccc(N2c3ccccc3Oc3cc(-c4nc(-n5c6ccccc6c6c7ccccc7c7c8ccccc8sc7c65)c5ccccc5n4)ccc32)cc1] | 51 |
| 14f | 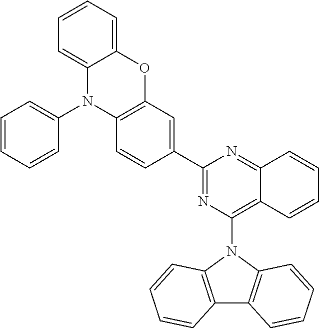 [SMILES: c1ccc(N2c3ccccc3Oc3cc(-c4nc(-n5c6ccccc6c6ccccc65)c5ccccc5n4)ccc32)cc1] | 56 |

Compounds 5f to 14f are recrystallised and sublimed (p about 10⁻⁶ mbar, T=350-390° C.).

### Production of the OLEDs

The use of the materials according to the invention OLEDs is presented in the Examples E1 to E20 below (see Table 1).

Pre-treatment for Examples E1-E20: Glass plates coated with structured ITO (indium tin oxide) in a thickness of 50 nm are treated before the coating with an oxygen plasma, followed by an argon plasma. These plasma-treated glass plates form the substrates to which the OLEDs are applied.

The OLEDs have in principle the following layer structure: substrate/hole-injection layer (HIL)/hole-transport layer (HTL)/electron-blocking layer (EBL)/emission layer (EML)/optional hole-blocking layer (HBL)/electron-transport layer (ETL)/optional electron-injection layer (EIL) and finally a cathode. The cathode is formed by an aluminium layer with a thickness of 100 nm. The precise structure of the OLEDs is shown in Table 1. The materials required for the production of the OLEDs are shown in Table 3.

All materials are applied by thermal vapour deposition in a vacuum chamber. The emission layer here always consists of at least one matrix material (host material) and an emitting dopant (emitter), which is admixed with the matrix material or matrix materials in a certain proportion by volume by co-evaporation. An expression such as EG1:TER (95%:5%) here means that material EG1 is present in the layer in a proportion by volume of 95% and TER is present in the layer in a proportion of 5%. Analogously, the electron-transport layer may also consist of a mixture of two materials.

The OLEDs are characterised by standard methods. For this purpose, the electroluminescence spectra, the current efficiency (measured in cd/A) and the external quantum efficiency (EQE, measured in percent) as a function of the luminous density, calculated from current/voltage/luminous density characteristic lines (IUL characteristic lines) assuming Lambert emission characteristics, are determined. The electroluminescence spectra are determined at a luminous density of 1000 cd/m², and the CIE 1931 x and y colour coordinates are calculated therefrom.

### Use of Mixtures According to the Invention in OLEDs

Compound EG1 according to the invention exhibits a voltage of 3.9 V and an efficiency of 24 cd/A at a colour coordinate of CIEx=0.67 and CIEy=0.33 at a luminous density of 1000 cd/m² in the construction of an OLED according to Example E1.

Compounds EG2 to EG17 according to the invention are employed in Examples E2 to E17 as matrix material in the emission layer. The colour coordinates of the electroluminescence spectra of the OLEDs from these experiments are at CIEx=0.67 and CIEy=0.33. The materials are thus suitable for use in the emission layer of red OLEDs.

Furthermore, the materials according to the invention can successfully be employed in the electron-transport layer (ETL) or the hole-blocking layer (HBL). This is shown in experiments E18-E20. Here too, the colour coordinates of the spectrum of the OLED are at CIEx=0.67 and CIEy=0.33.

TABLE 1
Structure of the OLEDs

| | HIL | HTL | EBL | EML | HBL | ETL | EIL |
|---|---|---|---|---|---|---|---|
| Ex | Thickness | Thickness | Thickness | Thickness | Thickness | Thickness | Thickness |
| E1 | HATCN | SpMA1 | SpMA2 | EG1:TER | — | ST:LiQ | — |
| | 5 nm | 125 nm | 10 nm | (95%:5%) | | (50%:50%) | |
| | | | | 40 nm | | 35 nm | |
| E2 | HATCN | SpMA1 | SpMA2 | EG2:TER | — | ST:LiQ | — |
| | 5 nm | 125 nm | 10 nm | (95%:5%) | | (50%:50%) | |
| | | | | 40 nm | | 35 nm | |
| E3 | HATCN | SpMA1 | SpMA2 | EG3:TER | — | ST:LiQ | — |
| | 5 nm | 125 nm | 10 nm | (95%:5%) | | (50%:50%) | |
| | | | | 40 nm | | 35 nm | |
| E4 | HATCN | SpMA1 | SpMA2 | EG4:TER | — | ST:LiQ | — |
| | 5 nm | 125 nm | 10 nm | (95%:5%) | | (50%:50%) | |
| | | | | 40 nm | | 35 nm | |
| E5 | HATCN | SpMA1 | SpMA2 | EG5:TER | — | ST:LiQ | — |
| | 5 nm | 125 nm | 10 nm | (95%:5%) | | (50%:50%) | |
| | | | | 40 nm | | 35 nm | |
| E6 | HATCN | SpMA1 | SpMA2 | EG6:TER | — | ST:LiQ | — |
| | 5 nm | 125 nm | 10 nm | (95%:5%) | | (50%:50%) | |
| | | | | 40 nm | | 35 nm | |
| E7 | HATCN | SpMA1 | SpMA2 | EG7:TER | — | ST:LiQ | — |
| | 5 nm | 125 nm | 10 nm | (95%:5%) | | (50%:50%) | |
| | | | | 40 nm | | 35 nm | |
| E8 | HATCN | SpMA1 | SpMA2 | EG8:TER | — | ST:LiQ | — |
| | 5 nm | 125 nm | 10 nm | (95%:5%) | | (50%:50%) | |
| | | | | 40 nm | | 35 nm | |
| E9 | HATCN | SpMA1 | SpMA2 | EG9:TER | — | ST:LiQ | — |
| | 5 nm | 125 nm | 10 nm | (95%:5%) | | (50%:50%) | |
| | | | | 40 nm | | 35 nm | |
| E10 | HATCN | SpMA1 | SpMA2 | EG10:TER | — | ST:LiQ | — |
| | 5 nm | 125 nm | 10 nm | (95%:5%) | | (50%:50%) | |
| | | | | 40 nm | | 35 nm | |
| E11 | HATCN | SpMA1 | SpMA2 | EG11:TER | — | ST:LiQ | — |
| | 5 nm | 125 nm | 10 nm | (95%:5%) | | (50%:50%) | |
| | | | | 40 nm | | 35 nm | |
| E12 | HATCN | SpMA1 | SpMA2 | EG12:TER | — | ST:LiQ | — |
| | 5 nm | 125 nm | 10 nm | (95%:5%) | | (50%:50%) | |
| | | | | 40 nm | | 35 nm | |
| E13 | HATCN | SpMA1 | SpMA2 | EG13:TER | — | ST:LiQ | — |
| | 5 nm | 125 nm | 10 nm | (95%:5%) | | (50%:50%) | |
| | | | | 40 nm | | 35 nm | |
| E14 | HATCN | SpMA1 | SpMA2 | EG14:TER | — | ST:LiQ | — |
| | 5 nm | 125 nm | 10 nm | (95%:5%) | | (50%:50%) | |
| | | | | 40 nm | | 35 nm | |
| E15 | HATCN | SpMA1 | SpMA2 | EG15:TER | — | ST:LiQ | — |
| | 5 nm | 125 nm | 10 nm | (95%:5%) | | (50%:50%) | |
| | | | | 40 nm | | 35 nm | |
| E16 | HATCN | SpMA1 | SpMA2 | EG16:TER | — | ST:LiQ | — |
| | 5 nm | 125 nm | 10 nm | (95%:5%) | | (50%:50%) | |
| | | | | 40 nm | | 35 nm | |
| E17 | HATCN | SpMA1 | SpMA2 | EG17:TER | — | ST:LiQ | — |
| | 5 nm | 125 nm | 10 nm | (95%:5%) | | (50%:50%) | |
| | | | | 40 nm | | 35 nm | |
| E18 | HATCN | SpMA1 | SpMA2 | EG1:TER | — | EG18:LiQ | — |
| | 5 nm | 125 nm | 10 nm | (95%:5%) | | (50%:50%) | |
| | | | | 40 nm | | 35 nm | |
| E19 | HATCN | SpMA1 | SpMA2 | EG1:TER | EG19 | ST:LiQ | — |
| | 5 nm | 125 nm | 10 nm | (95%:5%) | 5 nm | (50%:50%) | |
| | | | | 40 nm | | 35 nm | |
| E20 | HATCN | SpMA1 | SpMA2 | EG1:TER | EG20 | ST:LiQ | — |
| | 5 nm | 125 nm | 10 nm | (95%:5%) | 5 nm | (50%:50%) | |
| | | | | 40 nm | | 35 nm | |

TABLE 2
Structural formulae of the materials for the OLEDs

| | |
|---|---|
| 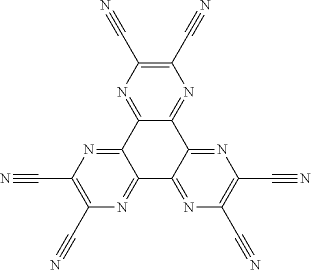 [SMILES: N#Cc1nc2c3nc(C#N)c(C#N)nc3c3nc(C#N)c(C#N)nc3c2nc1C#N] | HATCN |
| 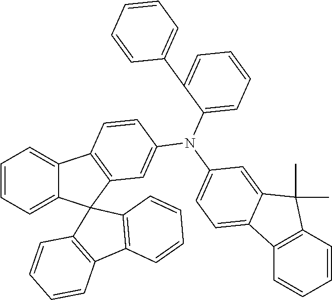 [SMILES: CC1(C)c2ccccc2-c2ccc(N(c3ccc4c(c3)C3(c5ccccc5-c5ccccc53)c3ccccc3-4)c3ccccc3-c3ccccc3)cc21] | SpMA1 |
| 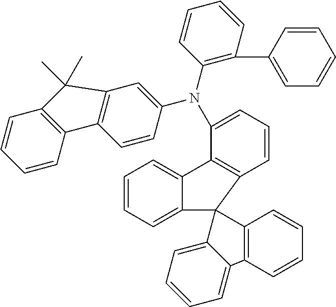 [SMILES: CC1(C)c2ccccc2-c2ccc(N(c3ccccc3-c3ccccc3)c3cccc4c3-c3ccccc3C43c4ccccc4-c4ccccc43)cc21] | SpMA2 |
| 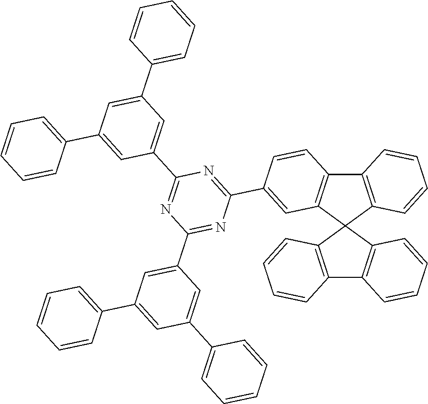 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1] | ST |
| 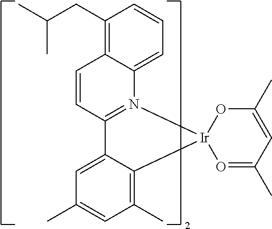 | TER |
| 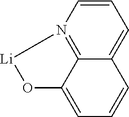 | LiQ |
| 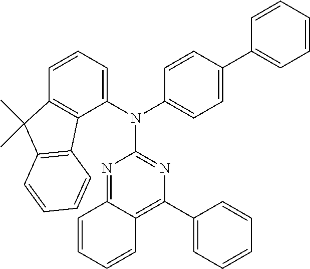 [SMILES: CC1(C)c2ccccc2-c2c(N(c3ccc(-c4ccccc4)cc3)c3nc(-c4ccccc4)c4ccccc4n3)cccc21] | EG1 |
| 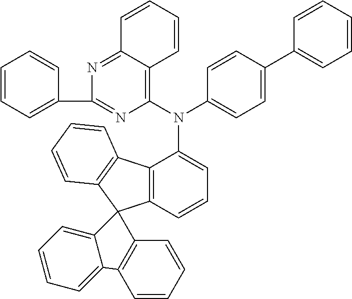 [SMILES: c1ccc(-c2ccc(N(c3cccc4c3-c3ccccc3C43c4ccccc4-c4ccccc43)c3nc(-c4ccccc4)nc4ccccc34)cc2)cc1] | EG2 |
| 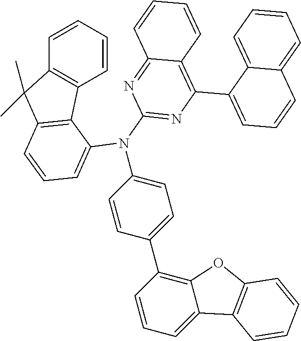 [SMILES: CC1(C)c2ccccc2-c2c(N(c3ccc(-c4cccc5c4oc4ccccc45)cc3)c3nc(-c4cccc5ccccc45)c4ccccc4n3)cccc21] | EG3 |
| 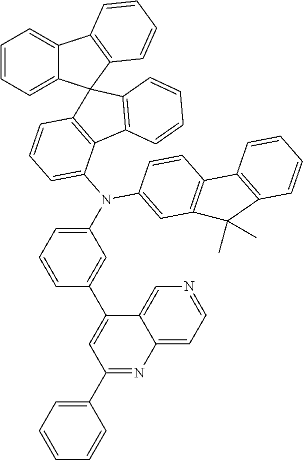 [SMILES: CC1(C)c2ccccc2-c2ccc(N(c3cccc(-c4cc(-c5ccccc5)nc5ccncc45)c3)c3cccc4c3-c3ccccc3C43c4ccccc4-c4ccccc43)cc21] | EG4 |
| 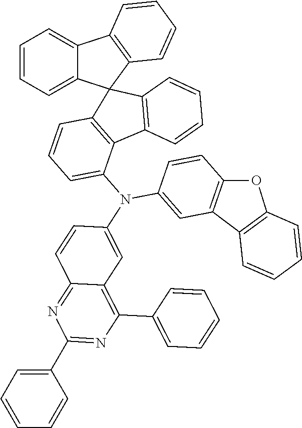 [SMILES: c1ccc(-c2nc(-c3ccccc3)c3cc(N(c4ccc5oc6ccccc6c5c4)c4cccc5c4-c4ccccc4C54c5ccccc5-c5ccccc54)ccc3n2)cc1] | EG5 |
| 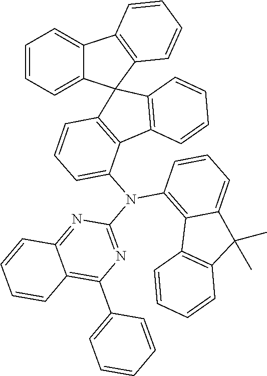 [SMILES: CC1(C)c2ccccc2-c2c(N(c3nc(-c4ccccc4)c4ccccc4n3)c3cccc4c3-c3ccccc3C43c4ccccc4-c4ccccc43)cccc21] | EG6 |
| 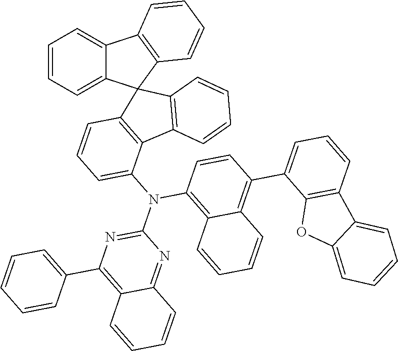 [SMILES: c1ccc(-c2nc(N(c3cccc4c3-c3ccccc3C43c4ccccc4-c4ccccc43)c3ccc(-c4cccc5c4oc4ccccc45)c4ccccc34)nc3ccccc23)cc1] | EG7 |
| 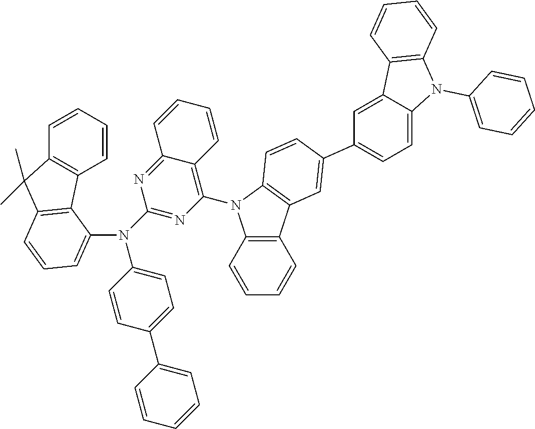 [SMILES: CC1(C)c2ccccc2-c2c(N(c3ccc(-c4ccccc4)cc3)c3nc(-n4c5ccccc5c5cc(-c6ccc7c(c6)c6ccccc6n7-c6ccccc6)ccc54)c4ccccc4n3)cccc21] | EG8 |
| 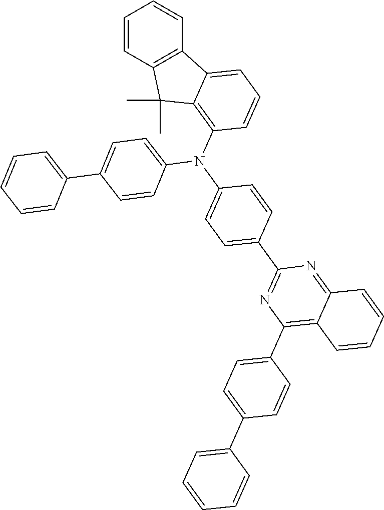 [SMILES: CC1(C)c2ccccc2-c2cccc(N(c3ccc(-c4ccccc4)cc3)c3ccc(-c4nc(-c5ccc(-c6ccccc6)cc5)c5ccccc5n4)cc3)c21] | EG9 |
| 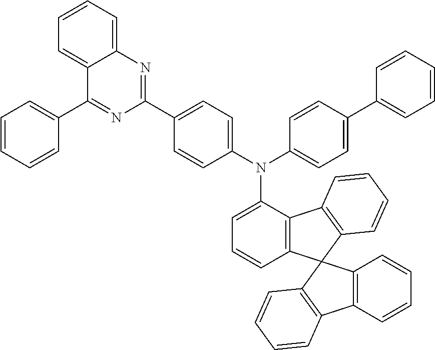 [SMILES: c1ccc(-c2ccc(N(c3ccc(-c4nc(-c5ccccc5)c5ccccc5n4)cc3)c3cccc4c3-c3ccccc3C43c4ccccc4-c4ccccc43)cc2)cc1] | EG10 |
| 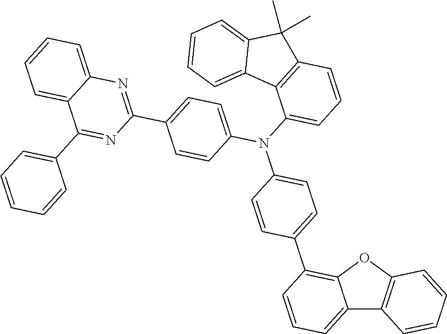 [SMILES: CC1(C)c2ccccc2-c2c(N(c3ccc(-c4nc(-c5ccccc5)c5ccccc5n4)cc3)c3ccc(-c4cccc5c4oc4ccccc45)cc3)cccc21] | EG11 |
| 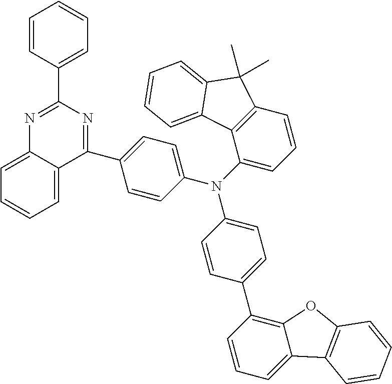 [SMILES: CC1(C)c2ccccc2-c2c(N(c3ccc(-c4nc(-c5ccccc5)nc5ccccc45)cc3)c3ccc(-c4cccc5c4oc4ccccc45)cc3)cccc21] | EG12 |
| 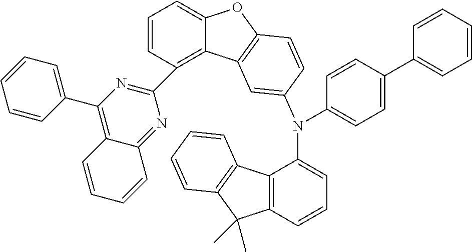 [SMILES: CC1(C)c2ccccc2-c2c(N(c3ccc(-c4ccccc4)cc3)c3ccc4oc5cccc(-c6nc(-c7ccccc7)c7ccccc7n6)c5c4c3)cccc21] | EG13 |
| 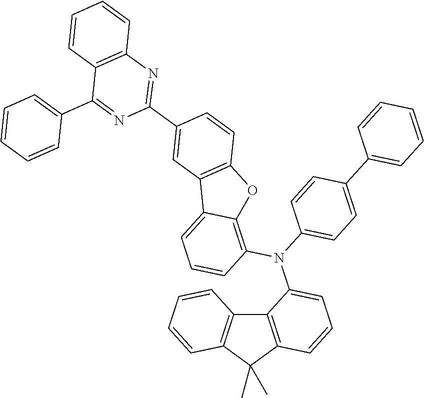 [SMILES: CC1(C)c2ccccc2-c2c(N(c3ccc(-c4ccccc4)cc3)c3cccc4c3oc3ccc(-c5nc(-c6ccccc6)c6ccccc6n5)cc34)cccc21] | EG14 |
| 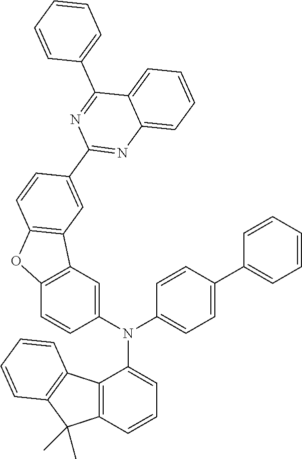 [SMILES: CC1(C)c2ccccc2-c2c(N(c3ccc(-c4ccccc4)cc3)c3ccc4oc5ccc(-c6nc(-c7ccccc7)c7ccccc7n6)cc5c4c3)cccc21] | EG15 |
| 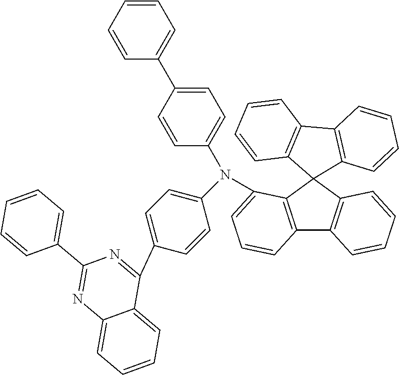 [SMILES: c1ccc(-c2ccc(N(c3ccc(-c4nc(-c5ccccc5)nc5ccccc45)cc3)c3cccc4c3C3(c5ccccc5-c5ccccc53)c3ccccc3-4)cc2)cc1] | EG16 |
| 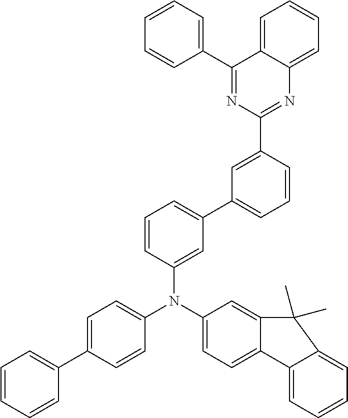 [SMILES: CC1(C)c2ccccc2-c2ccc(N(c3ccc(-c4ccccc4)cc3)c3cccc(-c4cccc(-c5nc(-c6ccccc6)c6ccccc6n5)c4)c3)cc21] | EG17 |
| 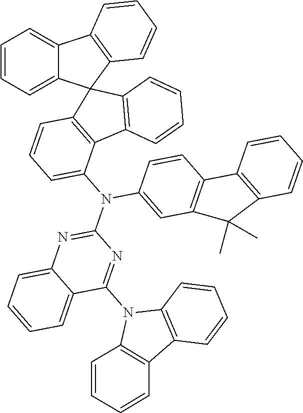 [SMILES: CC1(C)c2ccccc2-c2ccc(N(c3nc(-n4c5ccccc5c5ccccc54)c4ccccc4n3)c3cccc4c3-c3ccccc3C43c4ccccc4-c4ccccc43)cc21] | EG18 |
| 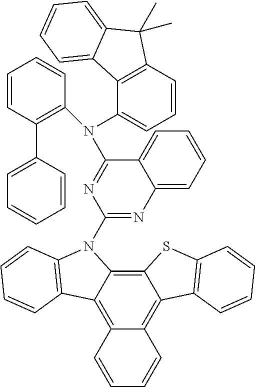 [SMILES: CC1(C)c2ccccc2-c2c(N(c3ccccc3-c3ccccc3)c3nc(-n4c5ccccc5c5c6ccccc6c6c7ccccc7sc6c54)nc4ccccc34)cccc21] | EG19 |
| 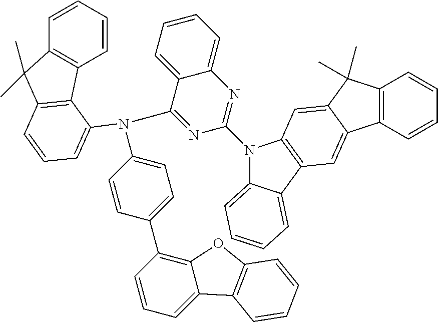 [SMILES: CC1(C)c2ccccc2-c2cc3c4ccccc4n(-c4nc(N(c5ccc(-c6cccc7c6oc6ccccc67)cc5)c5cccc6c5-c5ccccc5C6(C)C)c5ccccc5n4)c3cc21] | EG20 |

1.-19. (canceled)

20. A compound containing at least one structure of the formula (I′), 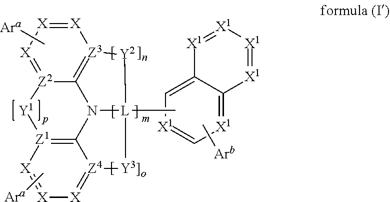
where the following applies to the symbols used:
L is an aromatic or heteroaromatic ring system having 5 to 30 aromatic ring atoms, which is optionally substituted by one or more non-aromatic radicals R¹;
Z¹, Z², Z³, Z⁴ are X;
Y¹, Y², Y³ are no longer present;
X is on each occurrence, identically or differently, N or CR¹ or C if a radical Ar^{a} is bonded to X;
X¹ is on each occurrence, identically or differently, N or CR¹;
m is 0 or 1;
n, o, p are 0;
i stands for 0, 1 or 2;
Ar^{a} is on each occurrence, identically or differently, an aromatic ring system having 5 to 30 aromatic ring atoms which is optionally substituted by one or more radicals R¹, or heteroaromatic ring system having 5 to 30 aromatic ring atoms, which is optionally substituted by one or more radicals R¹,
where the radical Ar^{a} does not contain a carbazole group or form a carbazole group with the aryl or heteroaryl group to which Ar^{a} is bonded, including substituents R¹, R² and R³ which is optionally bonded to the radical Ar^{a};
Ar^{b} is an aromatic ring system having 5 to 45 aromatic ring atoms which is optionally substituted by one or more radicals R¹, or heteroaromatic ring system having 5 to 45 aromatic ring atoms, which is optionally substituted by one or more radicals R¹;
R¹ is on each occurrence, identically or differently,
H;
D;
F;
Cl;
Br;
I;
CN;
NO₂;
N(AR¹)₂;
N(R²)₂;
C(═O)Ar¹;
P(═O)(Ar¹)₂;
P(Ar¹)₂;
B(Ar¹)₂;
Si(Ar¹)₃;
Si(R²)₃;
a straight-chain alkyl group having 1 to 40 C atoms, which is optionally substituted by one or more radicals R², wherein one or more non-adjacent CH₂ groups in said straight-chain alkyl group is optionally replaced by —R²C═CR²—, —C≡C—, Si(R²)₂, C—O, C═S, C═NR², —C(═O)O—, —C(═O)NR²—, NR³, P(═O)(R²), —O—, —S—, SO or SO₂ and where one or more H atoms in said straight-chain alkyl group, is optionally replaced by D, F, Cl, Br, I, CN or NO₂;
a straight-chain alkoxy group having 1 to 40 C atoms, which is optionally substituted by one or more radicals R², wherein one or more non-adjacent CH₂ groups in said straight-chain alkoxy group is optionally replaced by —R²C═CR²—, C≡C—, Si(R²)₂, C—O, C═S, C═NR², —C(═O)O—, —C(═O)NR²—, NR³, P(═O)(R²), —O—, —S—, SO or SO₂ and where one or more H atoms in said straight-chain alkoxy group, is optionally replaced by D, F, Cl, Br, I, CN or NO₂;
a straight-chain thioalkoxy group having 1 to 40 C atoms, which is optionally substituted by one or more radicals R², wherein one or more non-adjacent CH₂ groups in said straight-chain alkyl group is optionally replaced by —R²C—CR²—, C≡C—, Si(R²)₂, C═O, C═S, C═NR², —C(═O)O—, —C(═O)NR²—, NR³, P(═O)(R²), —O—, —S—, SO or SO₂ and where one or more H atoms in said straight-chain-thioalkoxy group, is optionally replaced by D, F, Cl, Br, I, CN or NO₂;
a branched alkyl group having 3 to 40 C atoms, which is optionally substituted by one or more radicals R², wherein one or more non-adjacent CH₂ groups in said branched alkyl group is optionally replaced by —R²C—CR²—, C≡C—, Si(R²)₂, C═O, C═S, C═NR², —C(═O)O—, —C(═O)NR²—, NR³, P(═O)(R²), —O—, —S—, SO or SO₂ and where one or more H atoms in said alkyl group, is optionally replaced by D, F, Cl, Br, I, CN or NO₂;
a branched alkoxy group having 3 to 40 C atoms, which is optionally substituted by one or more radicals R², wherein one or more non-adjacent CH₂ groups in said branched alkoxy group is optionally replaced by —R²C═CR², C≡C—, Si(R²)₂, C═O, C═S, C═NR², —C(═O)O—, —C(═O)NR²—, NR², P(═O)(R²), —O—, —S—, SO or SO₂ and where one or more H atoms in said branched alkoxy group, is optionally replaced by D, F, Cl, Br, I, CN or NO₂;
a branched thioalkoxy group having 3 to 40 C atoms, which is optionally substituted by one or more radicals R², wherein one or more non-adjacent CH₂ groups in said branched thioalkoxy group is optionally replaced by —R²C═CR²—, —C═C—, Si(R²)₂, C═O, C═S, C═NR², —C(═O)O—,—C(═O)NR²—, NR², P(═O)(R²), —O—, —S—, SO or SO₂ and where one or more H atoms in said branched thioalkoxy group, is optionally replaced by D, F, Cl, Br, I, CN or NO₂;
a cyclic alkyl group having 3 to 40 C atoms, which is optionally substituted by one or more radicals R², wherein one or more non-adjacent CH₂ groups in said —R²C═CR²—, C≡C—, Si(R²)₂, C═O, C═S, C═NR², —C(═O)O—,—C(═O)NR²—, NR², P(═O)(R²), —O—, —S—, SO or SO₂ and where one or more H atoms in said cyclic alkyl, is optionally replaced by D, F, Cl, Br, I, CN or NO₂;
a cyclic alkoxy group having 3 to 40 C atoms, which is optionally substituted by one or more radicals R², wherein one or more non-adjacent CH₂ groups in said cyclic alkoxy group is optionally replaced by —R²C═CR²—, C≡C—, Si(R²)₂, C—O, C═S, C═NR², —C(═O)O—, —C(═O)NR²—, NR², P(═O)(R²), —O—, —S—, SO or SO₂ and where one or more H atoms in said cyclic alkoxy group, is optionally replaced by D, F, Cl, Br, I, CN or NO₂;
a cyclic thioalkoxy group having 3 to 40 C atoms, which is optionally substituted by one or more radicals R², wherein one or more non-adjacent CH₂ groups in said cyclic thioalkoxy group is optionally replaced by —R²C═CR²—, C≡C—, Si(R²)₂, C═O, C═S, C═NR², —C(═O)O—, —C(═O)NR²—, NR², P(═O)(R²), —O—, —S—, SO or SO₂ and where one or more H atoms in said cyclic thioalkoxy group, is optionally replaced by D, F, Cl, Br, I, CN or NO₂;
an alkenyl group having 2 to 40 C atoms, which is optionally substituted by one or more radicals R²;
an aromatic ring system having 5 to 40 aromatic ring atoms, which is optionally substituted by one or more radicals R²;
a heteroaromatic ring system having 5 to 40 aromatic ring atoms, which is optionally substituted by one or more radicals R²;
an aryloxy group having 5 to 40 aromatic ring atoms, which is optionally substituted by one or more radicals R²;
heteroaryloxy group having 5 to 40 aromatic ring atoms, which is optionally substituted by one or more radicals R²;
an aralkyl group having 5 to 40 aromatic ring atoms, which is optionally substituted by one or more radicals R²;
a heteroaralkyl group having 5 to 40 aromatic ring atoms, which is optionally substituted by one or more radicals R²;
a combination of said aromatic ring system, said heteroaromatic ring system, said aryloxy group, said heteroaryloxy group said aralkyl group or said heteroaralkyl group;
two or more substituents R¹ may optionally form a mono aliphatic ring system, polycyclic aliphatic ring system, mono aromatic ring system or poly aromatic ring system with one another;
Ar¹ is on each occurrence, identically or differently, an aromatic or heteroaromatic ring system having 5 to 30 aromatic ring atoms, which is optionally substituted by one or more non-aromatic radicals R²; two radicals Ar¹ which are bonded to the same Si atom, N atom, P atom or B atom may also be bridged to one another by a single bond or a bridge selected from B(R²), C(R²)₂, Si(R²)₂, C═O, C—NR², C—C(R²)₂, O, S, S—O, SO₂, N(R²), P(R²) and P(═O)R²;
R² is on each occurrence, identically or differently,
H;
D;
F;
Cl;
Br;
I;
CN;
B(OR³)₂;
NO₂;
CR³═C(R³)₂;
C(═O)OR³;
C(═O)N(R³)₂;
Si(R³)₃;
P(R³)₂;
B(R³)₂;
N(R³)₂;
NO₂;
P(═O)(R³)₂;
OSO₂R³;
OR³;
S(═O)R³;
S(═O)₂R³;
a straight-chain alkyl group having 1 to 40 C atoms, which is optionally substituted by one or more radicals R³, wherein one or more non-adjacent CH₂ groups in said straight-chain alkyl group is optionally replaced by —R³C═CR³—, C≡C—, Si(R³)₂, C═O, C═S, C═NR³, —C(═O)O—, —C(═O)NR³—, NR³, P(═O)(R³), —O—, —S—, SO or SO₂ and where one or more H atoms in said straight-chain alkyl group, is optionally replaced by D, F, Cl, Br, I, CN or NO₂;
a straight-chain alkoxy group having 1 to 40 C atoms, which is optionally substituted by one or more radicals R³, wherein one or more non-adjacent CH₂ groups in said straight-chain alkoxy group is optionally replaced by —R³C═CR³—, C≡C—, Si(R³)₂, C—O, C═S, C═NR³, —C(═O)O—, —C(═O)NR³—, NR³, P(═O)(R³), —O—, —S—, SO or SO₂ and where one or more H atoms in said straight-chain alkoxy group, is optionally replaced by D, F, Cl, Br, I, CN or NO₂;
a straight-chain thioalkoxy group having 1 to 40 C atoms, which is optionally substituted by one or more radicals R³, wherein one or more non-adjacent CH₂ groups in said straight-chain alkyl group is optionally replaced by —R³C═CR³—, —C═C—, Si(R³)₂, C═O, C═S, C═NR³, —C(═O)O—, —C(═O)NR³—, NR³, P(═O)(R³), —O—, —S—, SO or SO₂ and where one or more H atoms in said straight-chain thioalkoxy group, is optionally replaced by D, F, Cl, Br, I, CN or NO₂;
a branched alkyl group having 3 to 40 C atoms, which is optionally substituted by one or more radicals R³, wherein one or more non-adjacent CH₂ groups in said branched alkyl group is optionally replaced by —R³C—CR³—, C≡C—, Si(R³)₂, C—O, C═S, C═NR³, —C(═O)O—, —C(═O)NR³—, NR³, P(═O)(R³), —O—, —S—, SO or SO₂ and where one or more H atoms in said branched alkyl group, is optionally replaced by D, F, Cl, Br, I, CN or NO₂;
a branched alkoxy group having 3 to 40 C atoms, which is optionally substituted by one or more radicals R³, wherein one or more non-adjacent CH₂ groups in said branched alkoxy group is optionally replaced by —R³C—CR³, C≡C—, Si(R³)₂, C═O, C—S, C═NR³, —C(═O)O—, —C(═O)NR³—, NR³, P(═O)(R³), —O—, —S—, SO or SO₂ and where one or more H atoms in said branched alkoxy group, is optionally replaced by D, F, Cl, Br, I, CN or NO₂;
a branched thioalkoxy group having 3 to 40 C atoms, which is optionally substituted by one or more radicals R³, wherein one or more non-adjacent CH₂ groups in said branched thioalkoxy group is optionally replaced by —R³C═CR³—, —C═C—, Si(R³)₂, C═O, C═S, C═NR³, —C(═O)O—, —C(═O)NR³—, NR³, P(═O)(R³), —O—, —S—, SO or SO₂ and where one or more H atoms in said branched thioalkoxy group, is optionally replaced by D, F, Cl, Br, I, CN or NO₂;
a cyclic alkyl group having 3 to 40 C atoms, which is optionally substituted by one or more radicals R³, wherein one or more non-adjacent CH₂ groups in said cyclic alkyl group is optionally replaced by —R³C═CR³—, —C═C—, Si(R³)₂, C—O, C═S, C═NR³, —C(═O)O—, —C(═O)NR³—, NR³, P(═O)(R³), —O—, —S—, SO or SO₂ and where one or more H atoms in said cyclic alkyl, is optionally replaced by D, F, Cl, Br, I, CN or NO₂;
a cyclic alkoxy group having 3 to 40 C atoms, which is optionally substituted by one or more radicals R³, wherein one or more non-adjacent CH₂ groups in said cyclic alkyl group is optionally replaced by —R³C═CR³, C≡C—, Si(R³)₂, C═O, C═S, C═NR³, —C(═O)O—, —C(═O)NR³—, NR³, P(═O)(R³), —O—, —S—, SO or SO₂ and where one or more H atoms in said cyclic alkoxy group, is optionally replaced by D, F, Cl, Br, I, CN or NO₂;
a cyclic thioalkoxy group having 3 to 40 C atoms, which is optionally substituted by one or more radicals R³, wherein one or more non-adjacent CH₂ groups in said cyclic alkyl group is optionally replaced by —R³C═CR³—, —C═C—, Si(R³)₂, C═O, C—S, C═NR³, —C(═O)O—, —C(═O)NR³—, NR³, P(═O)(R³), —O—, —S—, SO or SO₂ and where one or more H atoms in said cyclic thioalkoxy group, is optionally replaced by D, F, Cl, Br, I, CN or NO₂;
an aromatic ring system having 5 to 40 aromatic ring atoms, which is optionally substituted by one or more radicals R³;
heteroaromatic ring system having 5 to 40 aromatic ring atoms, which is optionally substituted by one or more radicals R³;
an aryloxy group having 5 to 40 aromatic ring atoms, which is optionally substituted by one or more radicals R³;
a heteroaryloxy group having 5 to 40 aromatic ring atoms, which is optionally substituted by one or more radicals R³;
a combination of said aromatic ring system, said heteroaromatic ring system, said aryloxy or heteroaryloxy groups; or
two or more substituents R² may optionally form a mono aliphatic ring system, polycyclic aliphatic ring system, mono aromatic ring system or poly aromatic ring system with one another;
R³ is selected on each occurrence, identically or differently, from the group consisting of
H;
D;
F;
CN;
an aliphatic hydrocarbon radical having 1 to 20 C atoms;
an aromatic ring system having 5 to 30 aromatic ring atoms;
in which one or more H atoms in said aromatic ring system is optionally replaced by D, F, Cl, Br, I or CN and
which is optionally substituted by one or more alkyl groups, each having 1 to 4 carbon atoms;
a heteroaromatic ring system having 5 to 30 aromatic ring atoms,
in which one or more H atoms in said heteroaromatic ring system is optionally replaced by D, F, Cl, Br, I or CN and
which is optionally substituted by one or more alkyl groups, each having 1 to 4 carbon atoms;
two or more substituents R³ may form a mono aliphatic ring system or polycyclic, aliphatic ring system with one another;
with the proviso that at least two groups X¹ in formula (I′) stand for N,
since the index n, o, p=0, the corresponding bridge Y¹, Y², Y³ is not present and
where m=0 means that a single bond is present between the nitrogen atom and the heteroaromatic ring system.

21. The compound according to claim 20,

22. The compound according to claim 20, wherein the compound is containing at least one structure of the formula (Vb), 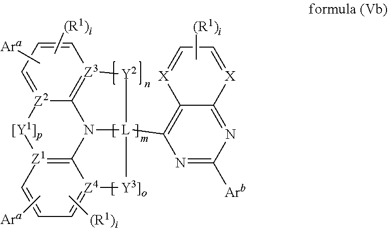
wherein at least one group X¹ stands for N and i stands for 0, 1 or 2.

23. The compound according to claim 20, wherein the compound is containing at least one structure of the formula (XVIIIb), 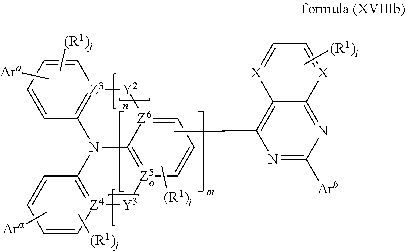
wherein i stands for 0, 1 or 2 and j stands for 0, 1, 2 or 3.

24. The compound according to claim 20, wherein the compound is containing at least one structure of the formula (XIXb), 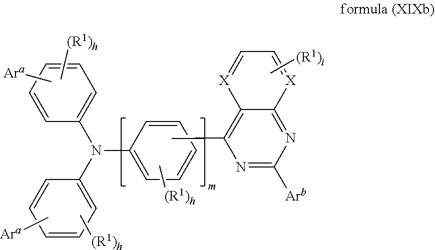
wherein i stands for 0, 1 or 2 and h stands for 0, 1, 2, 3 or 4.

25. The compound according to claim 20, wherein the compound is containing at least one structure of the formula (XXIb), 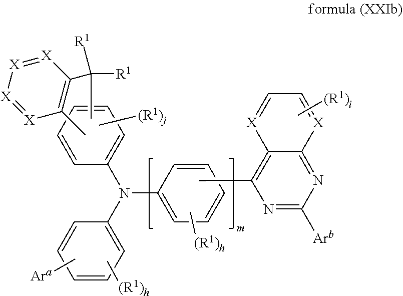
wherein j stands for 0, 1, 2 or 3 and h stands for 0, 1, 2, 3 or 4.

26. The compound according to claim 20, wherein the compound is containing at least one structure of the formula (XXIIb), 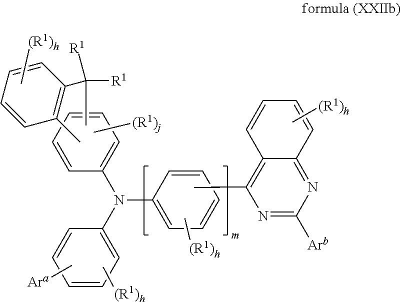
wherein j stands for 0, 1, 2 or 3 and h stands for 0, 1, 2, 3 or 4.

27. The compound according to claim 25, wherein the compound is containing at least one structure of the formula (XXIIIb), (XXIIId), or (XXIIIf), 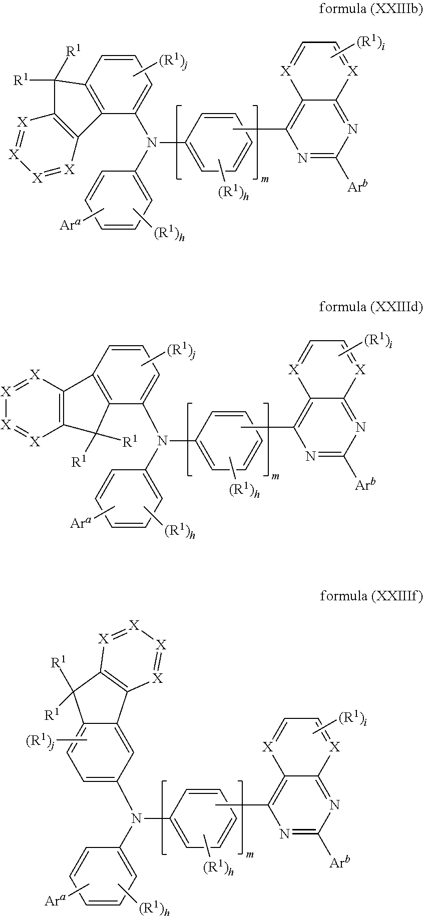 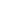
wherein i stands for 0, 1 or 2, j stands for 0, 1, 2 or 3 and h stands for 0, 1, 2, 3 or 4.

28. The compound according to claim 26, wherein the compound is containing at least one structure of the formula (XXIVb), 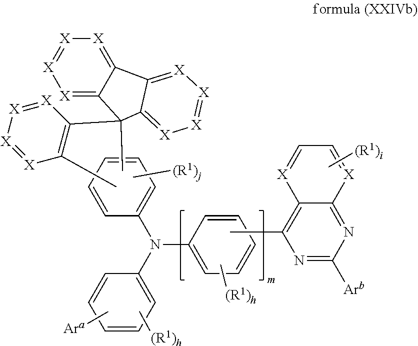
wherein i stands for 0, 1 or 2, j stands for 0, 1, 2 or 3 and h stands for 0, 1, 2, 3 or 4.

29. The compound according to claim 26, wherein the compound is containing at least one structure of the formula (XXVb), (XXVd), (XXVf), or (XXVh), 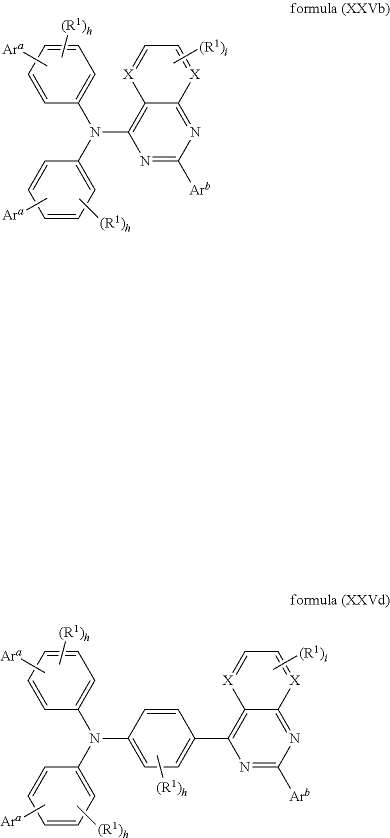 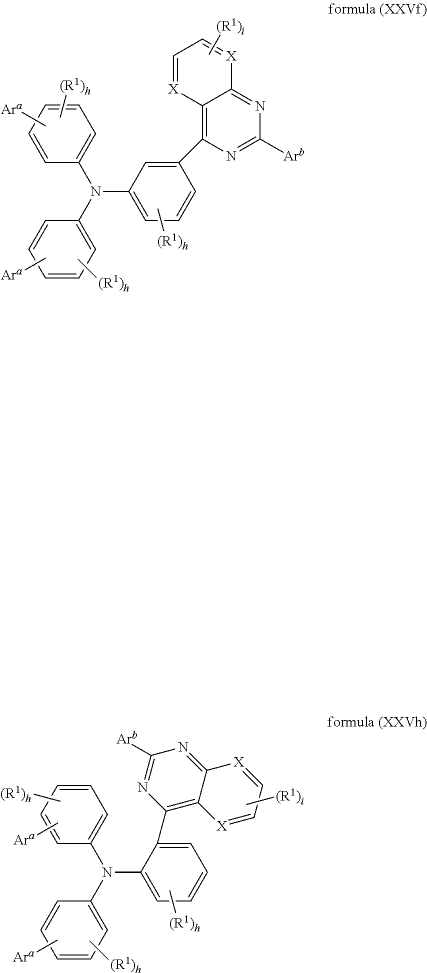
wherein i stands for 0, 1 or 2 and h stands for 0, 1, 2, 3 or 4.

30. The compound according to claim 20, wherein the group Ar^{b} represents a group of the formula (Ar^{b}-1), 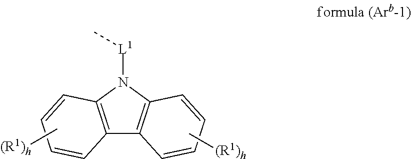
in which L1 is a bond or an aromatic or heteroaromatic ring system having 5 to 30 aromatic ring atoms, which is optionally substituted by one or more radicals R¹, h is 0, 1, 2, 3 or 4 and the dashed line represents the bond.

31. An oligomer, polymer or dendrimer containing one or more compounds according to claim 20, where, instead of a hydrogen atom or a substituent, one or more bonds are present from the compounds to the polymer, oligomer or dendrimer.

32. A composition comprising at least one compound according to claim 20 and at least one further compound selected from the group consisting of fluorescent emitters, phosphorescent emitters, emitters which exhibit TADF, host materials, electron-transport materials, electron-injection materials, hole-conductor materials, hole-injection materials, electron-blocking materials and hole-blocking materials.

33. A formulation comprising at least one compound according to claim 20 and at least one solvent.

34. An electronic device comprising at least one compound according to claim 20.

35. A host material or electron-transport material comprising at least one compound according to claim 20.

36. A process for the preparation of the compound according to claim 20 which comprises coupling a diarylamine compound to a compound containing at least one diazanaphthyl group in a coupling reaction.

37. The electronic device according to claim 36, wherein the electronic device is selected from the group consisting of organic electroluminescent devices, organic integrated circuits, organic field-effect transistors, organic thin-film transistors, organic light-emitting transistors, organic solar cells, organic optical detectors, organic photoreceptors, organic field-quench devices, light-emitting electrochemical cells or organic laser diodes.

38. The compound according to claim 20, wherein m is 0.

39. The compound according to claim 20, wherein m is 1.
